(19) United States
(12) Patent Application Publication
(10) Pub. No.: US 2025/0162981 A1
(43) Pub. Date: May 22, 2025

(54) IONIZABLE LIPIDOID COMPOSITIONS AND THERAPEUTIC USES THEREOF

(71) Applicant: Flagship Pioneering Innovations VII, LLC, Cambridge, MA (US)

(72) Inventors: Ulugbek Barotov, Everett, MA (US); Puneet Batra, Cambridge, MA (US); Timothy Ray Blake, New York City, NY (US); Nolan Gallagher, Cambridge, MA (US); Louisa Marie Helms, Seattle, WA (US); Vandan Paresh Jakharia, Allston, MA (US); Edward Matthew Kennedy, Bedford, MA (US); Stephanie Marie Knapp, Wayland, MA (US); Chelsea RamEl Martinez, Cambridge, MA (US); Brian Patrick Mead, Brookline, MA (US); Jae Hyeon Park, Belmont, MA (US); Jeffrey Pimentel, Swansea, MA (US); Dean Peter Stamos, Lexington, MA (US)

(21) Appl. No.: 18/947,822

(22) Filed: Nov. 14, 2024

Related U.S. Application Data
(60) Provisional application No. 63/548,473, filed on Nov. 14, 2023.

(51) Int. Cl. C07C 219/06; A61K 9/51; A61K 39/00; A61K 39/215; C07C 229/16; C07C 271/12; C07C 271/20; C07C 323/52; C07C 327/34; C07C 329/06

(52) U.S. Cl. CPC C07C 219/06; A61K 9/5123; A61K 39/215; C07C 229/16; C07C 271/12; C07C 271/20; C07C 323/52; C07C 327/34; C07C 329/06; A61K 2039/53; C07C 2601/04

(57) ABSTRACT

Disclosed are lipidoid compounds having the structure of formula (I), or a pharmaceutically acceptable salt thereof:

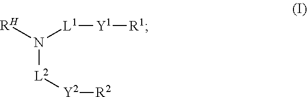

wherein the groups are as defined in the application. Also disclosed are nanoparticle compositions comprising a lipidoid of the invention that are capable of delivering a therapeutic agent. The application also discloses pharmaceutical compositions comprising a lipidoid composition of the invention.

### RELATED APPLICATIONS

This application claims the benefit of priority to U.S. Provisional Patent Application Ser. No. 63/548,473, filed Nov. 14, 2024, the contents of which are incorporated herein by reference.

### BACKGROUND

The effective targeted delivery of biologically active substances such as small molecule drugs, proteins, and nucleic acids represents a continuing medical challenge. In particular, the delivery of nucleic acids to cells is made difficult by their relative instability and low cell permeability. Thus, there exists a need to develop methods and compositions to facilitate the delivery of therapeutic and/or prophylactics, such as nucleic acids, to cells.

Lipid-containing nanoparticle compositions, liposomes, and lipoplexes can be effective transport vehicles into cells and/or intracellular compartments for biologically active substances such as small molecule drugs, proteins, and nucleic acids. Such compositions generally include one or more cationic and/or ionizable lipids, phospholipids including polyunsaturated lipids, structural lipids (e.g., sterols), and/or lipids containing polyethylene glycol (PEG lipids). There is a need to develop lipidoids useful in lipid-nanoparticle compositions that can deliver therapeutic agents, such as nucleic acid molecules, proteins, and small molecule drugs with safety, efficacy, and specificity.

### SUMMARY

In certain embodiments, the present disclosure provides a lipidoid of formula (VIII), or a pharmaceutically acceptable salt thereof;

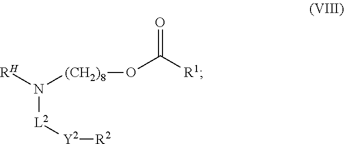

wherein:
R^{H} is hydroxyalkyl or (hydroxyalkyl)-O-alkylene-;
L² represents —(CH₂)ₘ—;
m is an integer selected from 8-10;
Y² is *—O(C═O)O—, *—O(C═O)NH—, or *—(C═O)O—;
   wherein the asterisk (*) indicates the point of attachment to L²;
R¹ is linear (C₆-C₁₀)alkyl, branched (C₁₀-C₂₀)alkyl, or linear (C₁₀-C₂₀)alkenyl; and
R² is branched (C₁₀-C₂₀)alkyl; wherein:
   a branched (C₁₀-C₂₀)alkyl may comprise more than one branch point; and
   a linear (C₁₀-C₂₀)alkenyl may comprise more than one carbon-carbon double bond.

In certain aspects, R^{H} is hydroxyalkyl.

In certain aspects, Y² is *—O(C═O)O—.

In certain aspects, L² is —(CH₂)₈—.

In certain aspects, R¹ is linear (C₆-C₁₀)alkyl.

In certain aspects, the present disclosure provides a lipidoid of formula (I), or a pharmaceutically acceptable salt thereof;

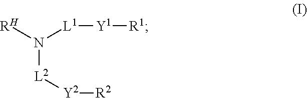

wherein:
R^{H} is hydroxyalkyl, (hydroxyalkyl)-O-alkylene-, or alkyl substituted by

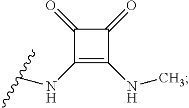 [SMILES: *Nc1c(NC)c(=O)c1=O]

L¹ and L² each represent —(CH₂)ₘ—;
m is independently for each occurrence an integer selected from 1-20;
Y¹ and Y² are each independently *—O(C═O)—, *—S(C═O)—, *—NH(C═O)—, *—NH(C═S)—, *—O(C═O)O—, *—O(C═S)O—, *—S(C═O)O—, *—O(C═O)S—, *—S(C═S)O—, *—NH(C═O)O—, *—O(C═O)NH—, *—(C═O)O—, *—(C═S)O—, *—O(P(O)OR²⁰)O—,

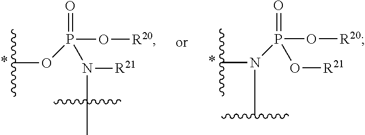

wherein the asterisk (*) indicates the point of attachment to L¹ or L²
R²⁰ is independently for each occurrence an optionally substituted linear or branched (C₁-C₁₅)alkyl;
R²¹ is independently for each occurrence H, optionally substituted (C₁-C₅)alkyl, or optionally substituted (C₃-C₅)cycloalkyl; and
R¹ and R² are each independently optionally substituted linear (C₄-C₃₀)alkyl, branched (C₄-C₃₀)alkyl, linear (C₄-C₃₀)alkenyl, branched (C₄-C₃₀)alkenyl, linear (C₄-C₃₀)alkynyl, or branched (C₄-C₃₀)alkynyl; wherein:
   a branched (C₄-C₃₀)alkyl, a branched (C₄-C₃₀)alkenyl, or a branched (C₄-C₃₀)alkynyl may comprise more than one branch point;
   a linear or branched (C₄-C₃₀)alkenyl may comprise more than one carbon-carbon double bond;
   a linear or branched (C₄-C₃₀)alkynyl may comprise more than one carbon-carbon triple bond; and
   in any linear (C₄-C₃₀)alkyl, branched (C₄-C₃₀)alkyl, linear (C₄-C₃₀)alkenyl, branched (C₄-C₃₀)alkenyl, linear (C₄-C₃₀)alkynyl, or branched (C₄-C₃₀)alkynyl group, a —CH₂CH₂— moiety may be replaced by a —S—S-moiety.

In certain aspects, R^{H} is hydroxyalkyl.

In certain aspects, R^{H} is —CH₂CH₂OH.

In certain aspects, m is independently for each occurrence an integer selected from 4-20, preferably 8-10.

In certain aspects, L¹ and L² each represent —(CH₂)₈—.

In certain aspects, Y¹ and Y² are each independently *—O(C═O)—, *—S(C═O)—, *—NH(C═O)—, *—O(C═O)O—, *—S(C═O)O—, *—O(C═O)S—, *—NH(C═O)O—, *—O(C═O)NH—, or *—(C═O)O—.

In certain aspects, Y¹ is *—O(C═O)—, *—S(C═O)—, *—NH(C═O)—, or *—(C═O)O—.

In certain aspects, Y¹ is *—O(C═O)—.

In certain aspects, Y² is *—O(C═O)O—, *—S(C═O)O—, *—O(C═O)S—, *—NH(C═O)O—, or *—O(C═O)NH—.

In certain aspects, Y² is *—O(C═O)O—.

In certain aspects, Y¹ and Y² are different.

In certain aspects, Y¹ and Y² are the same.

In certain aspects, R¹ is linear (C₄-C₃₀)alkyl or branched (C₄-C₃₀)alkyl.

In certain aspects, R¹ is linear (C₆-C₂₀)alkyl or branched (C₆-C₂₀)alkyl.

In certain aspects, R¹ is linear (C₆-C₁₄)alkyl or branched (C₆-C₁₄)alkyl.

In certain aspects, R¹ is linear (C₈)alkyl.

In certain aspects, R¹ is linear (C₈-C₂₀)alkenyl.

In certain aspects, R² is linear (C₄-C₃₀)alkyl or branched (C₄-C₃₀)alkyl.

In certain aspects, R² is branched (C₆-C₃₀)alkyl.

In certain aspects, R² is branched (C₁₆-C₂₆)alkyl.

In certain aspects, R² is branched (C₁₈-C₂₄)alkyl.

In certain aspects, R² is branched (C₁₈)alkyl.

In certain aspects, the lipidoid is selected from the following table:

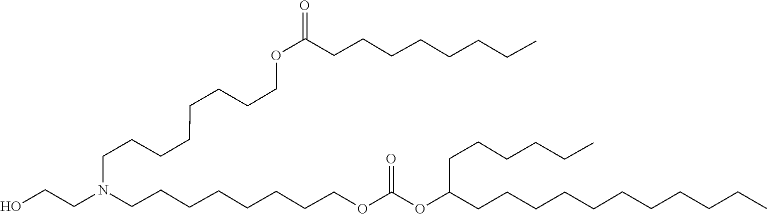 [SMILES: CCCCCCCCCCCC(CCCCCC)OC(=O)OCCCCCCCCN(CCO)CCCCCCCCOC(=O)CCCCCCCC]

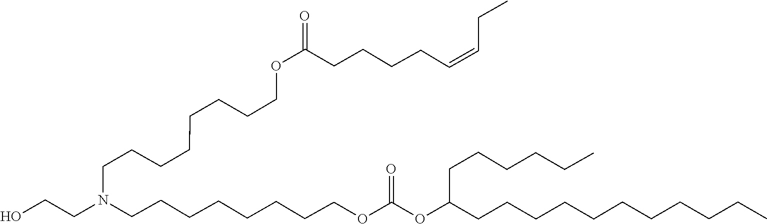 [SMILES: CC/C=C\CCCCC(=O)OCCCCCCCCN(CCO)CCCCCCCCOC(=O)OC(CCCCCC)CCCCCCCCCCC]

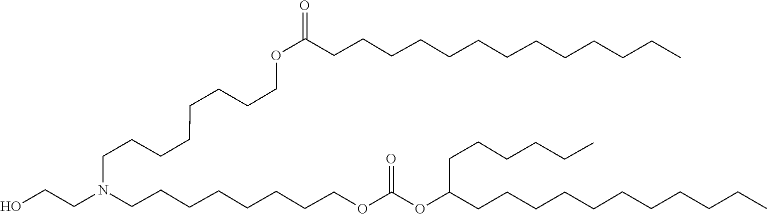 [SMILES: CCCCCCCCCCCCCC(=O)OCCCCCCCCN(CCO)CCCCCCCCOC(=O)OC(CCCCCC)CCCCCCCCCCC]

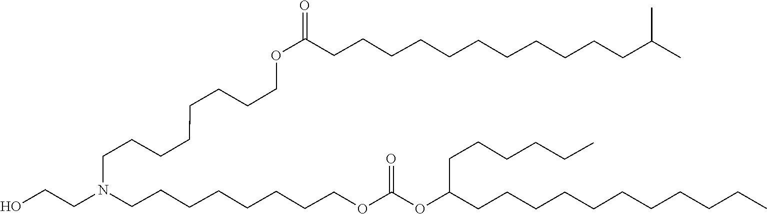 [SMILES: CCCCCCCCCCCC(CCCCCC)OC(=O)OCCCCCCCCN(CCO)CCCCCCCCOC(=O)CCCCCCCCCCCC(C)C]

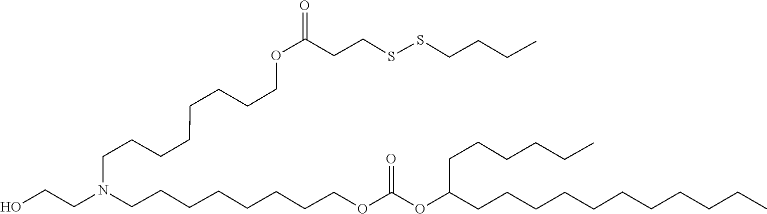 [SMILES: CCCCCCCCCCCC(CCCCCC)OC(=O)OCCCCCCCCN(CCO)CCCCCCCCOC(=O)CCSSCCCC]

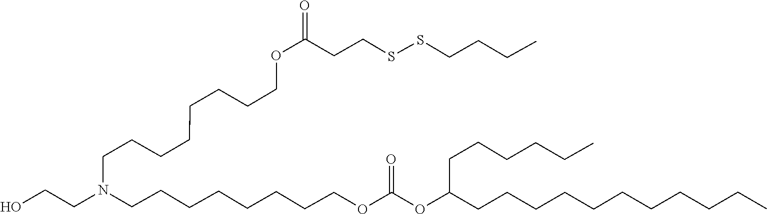 [SMILES: CCCCCCCCCCCC(CCCCCC)OC(=O)OCCCCCCCCN(CCO)CCCCCCCCOC(=O)CCSSCCCC]

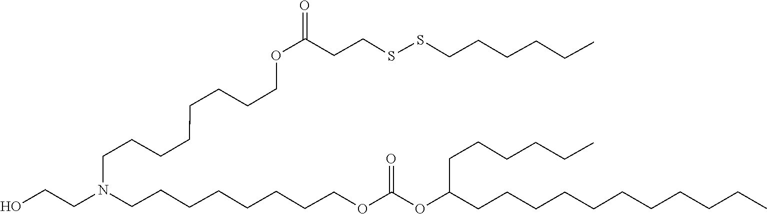 [SMILES: CCCCCCCCCCCC(CCCCCC)OC(=O)OCCCCCCCCN(CCO)CCCCCCCCOC(=O)CCSSCCCCCC]

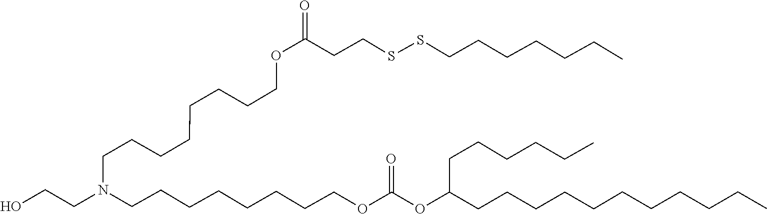 [SMILES: CCCCCCCCCCCC(CCCCCC)OC(=O)OCCCCCCCCN(CCO)CCCCCCCCOC(=O)CCSSCCCCCCC]

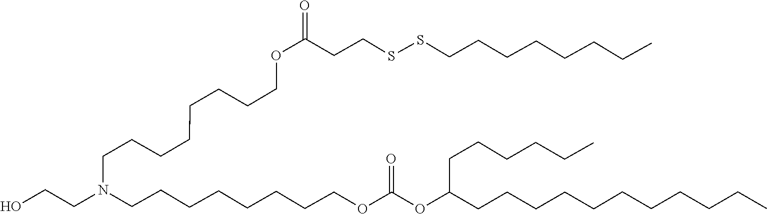 [SMILES: CCCCCCCCCCCC(CCCCCC)OC(=O)OCCCCCCCCN(CCO)CCCCCCCCOC(=O)CCSSCCCCCCCC]

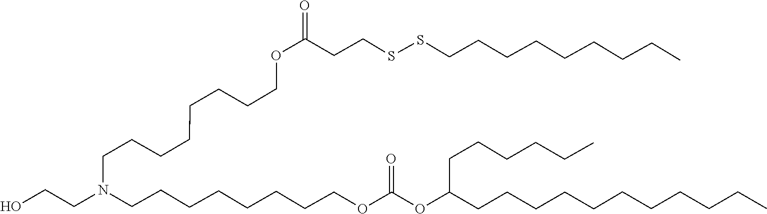 [SMILES: CCCCCCCCCCCC(CCCCCC)OC(=O)OCCCCCCCCN(CCO)CCCCCCCCOC(=O)CCSSCCCCCCCCC]

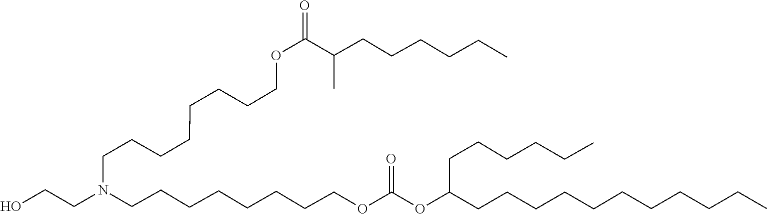 [SMILES: CCCCCCCCCCCC(CCCCCC)OC(=O)OCCCCCCCCN(CCO)CCCCCCCCOC(=O)C(C)CCCCCC]

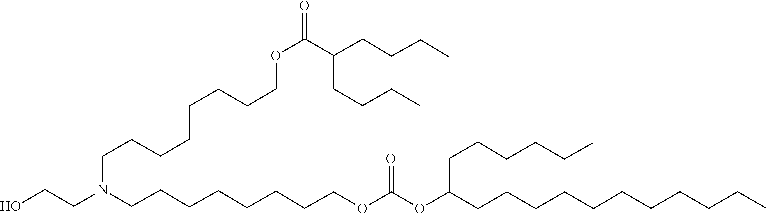 [SMILES: CCCCCCCCCCCC(CCCCCC)OC(=O)OCCCCCCCCN(CCO)CCCCCCCCOC(=O)C(CCCC)CCCC]

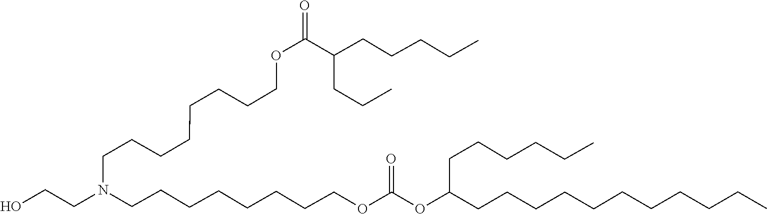 [SMILES: CCCCCCCCCCCC(CCCCCC)OC(=O)OCCCCCCCCN(CCO)CCCCCCCCOC(=O)C(CCC)CCCCC]

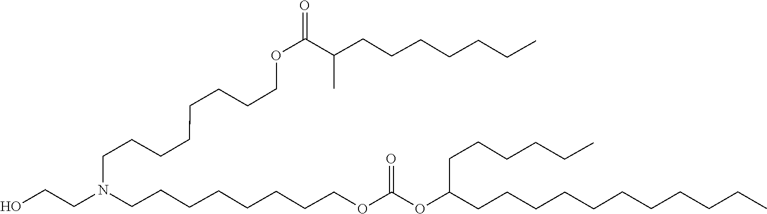 [SMILES: CCCCCCCCCCCC(CCCCCC)OC(=O)OCCCCCCCCN(CCO)CCCCCCCCOC(=O)C(C)CCCCCCC]

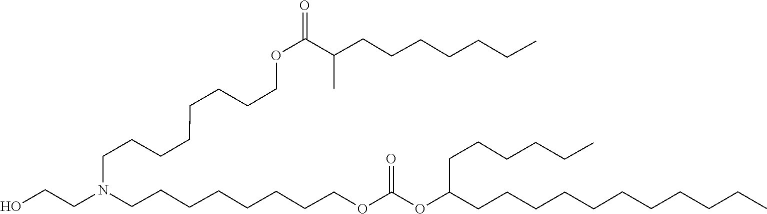 [SMILES: CCCCCCCCCCCC(CCCCCC)OC(=O)OCCCCCCCCN(CCO)CCCCCCCCOC(=O)C(C)CCCCCCC]

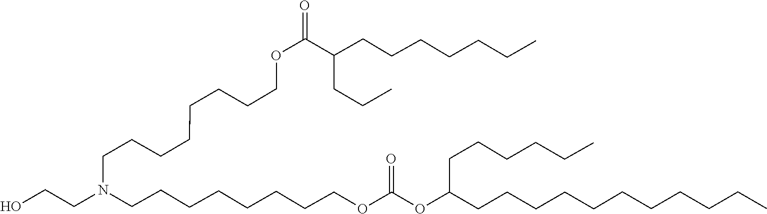 [SMILES: CCCCCCCCCCCC(CCCCCC)OC(=O)OCCCCCCCCN(CCO)CCCCCCCCOC(=O)C(CCC)CCCCCCC]

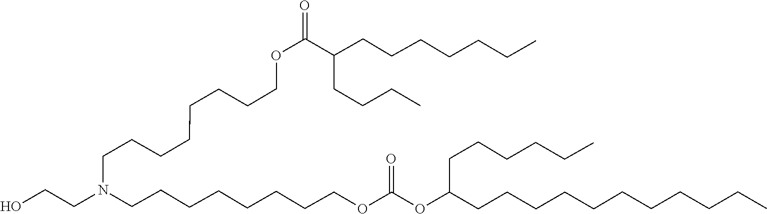 [SMILES: CCCCCCCCCCCC(CCCCCC)OC(=O)OCCCCCCCCN(CCO)CCCCCCCCOC(=O)C(CCCC)CCCCCCC]

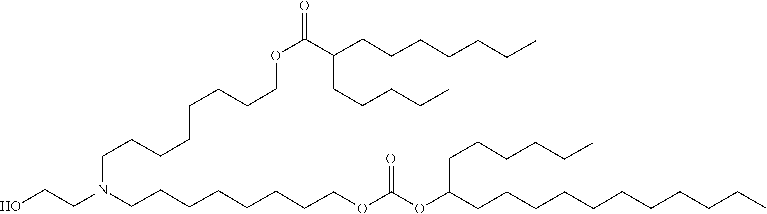 [SMILES: CCCCCCCCCCCC(CCCCCC)OC(=O)OCCCCCCCCN(CCO)CCCCCCCCOC(=O)C(CCCCC)CCCCCCC]

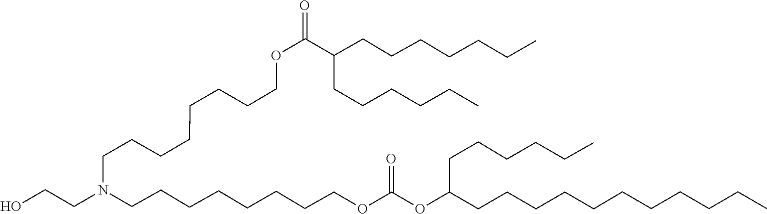 [SMILES: CCCCCCCCCCCC(CCCCCC)OC(=O)OCCCCCCCCN(CCO)CCCCCCCCOC(=O)C(CCCCCC)CCCCCCC]

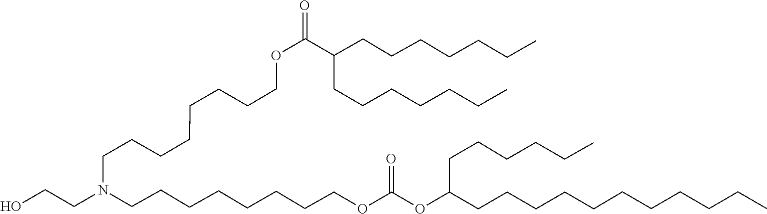 [SMILES: CCCCCCCCCCCC(CCCCCC)OC(=O)OCCCCCCCCN(CCO)CCCCCCCCOC(=O)C(CCCCCCC)CCCCCCC]

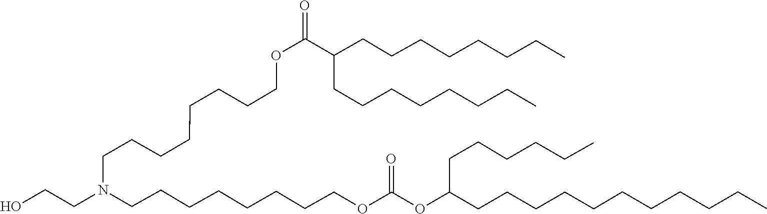 [SMILES: CCCCCCCCCCCC(CCCCCC)OC(=O)OCCCCCCCCN(CCO)CCCCCCCCOC(=O)C(CCCCCCCC)CCCCCCCC]

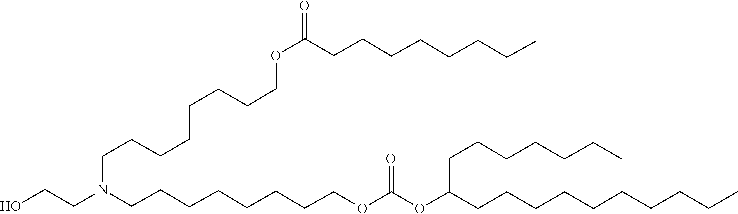 [SMILES: CCCCCCCCCCC(CCCCCCC)OC(=O)OCCCCCCCCN(CCO)CCCCCCCCOC(=O)CCCCCCCC]

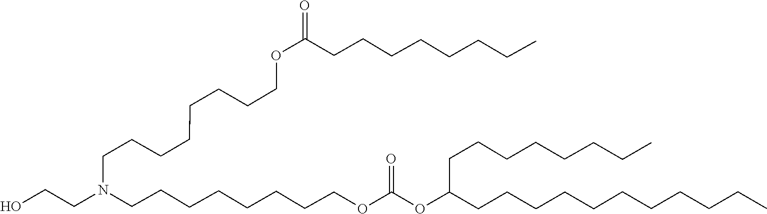 [SMILES: CCCCCCCCCCCC(CCCCCCCC)OC(=O)OCCCCCCCCN(CCO)CCCCCCCCOC(=O)CCCCCCCC]

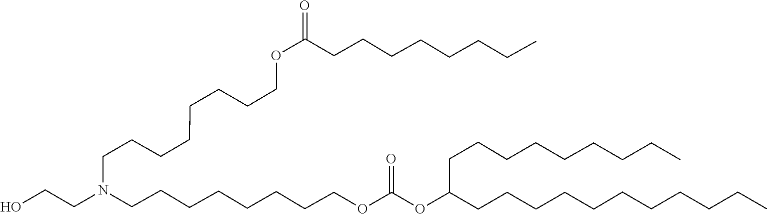 [SMILES: CCCCCCCCCCCC(CCCCCCCCC)OC(=O)OCCCCCCCCN(CCO)CCCCCCCCOC(=O)CCCCCCCC]

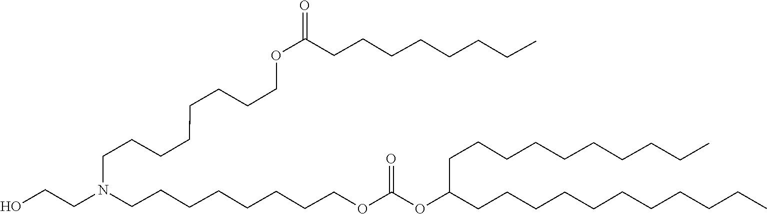 [SMILES: CCCCCCCCCCCC(CCCCCCCCCC)OC(=O)OCCCCCCCCN(CCO)CCCCCCCCOC(=O)CCCCCCCC]

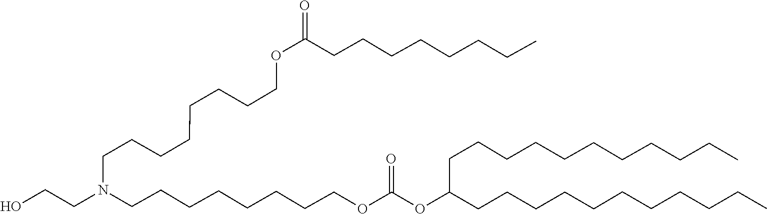 [SMILES: CCCCCCCCCCCC(CCCCCCCCCCC)OC(=O)OCCCCCCCCN(CCO)CCCCCCCCOC(=O)CCCCCCCC]

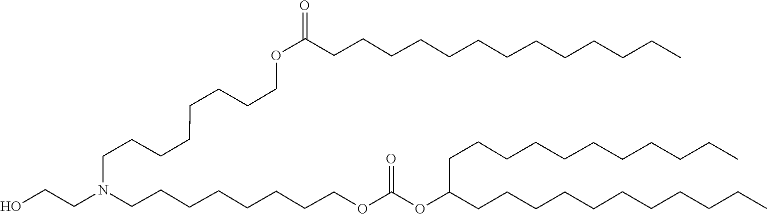 [SMILES: CCCCCCCCCCCCCC(=O)OCCCCCCCCN(CCO)CCCCCCCCOC(=O)OC(CCCCCCCCCCC)CCCCCCCCCCC]

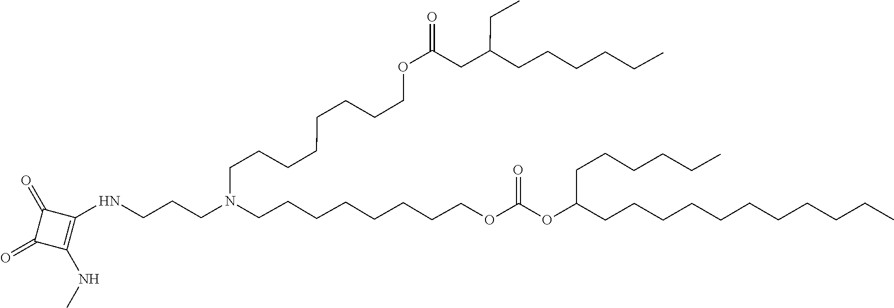 [SMILES: CCCCCCCCCCCC(CCCCCC)OC(=O)OCCCCCCCCN(CCCCCCCCOC(=O)CC(CC)CCCCCC)CCCNc1c(NC)c(=O)c1=O]

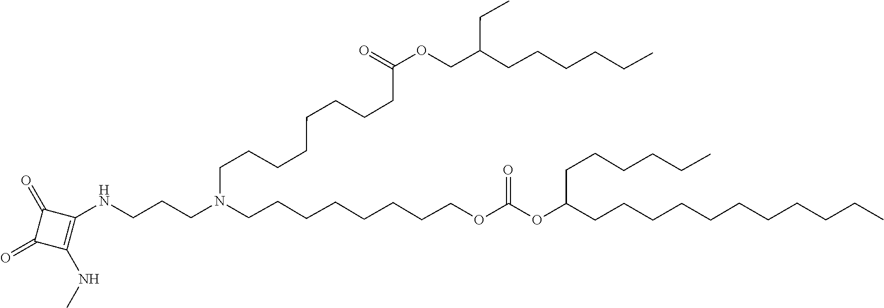 [SMILES: CCCCCCCCCCCC(CCCCCC)OC(=O)OCCCCCCCCN(CCCCCCCCC(=O)OCC(CC)CCCCCC)CCCNc1c(NC)c(=O)c1=O]

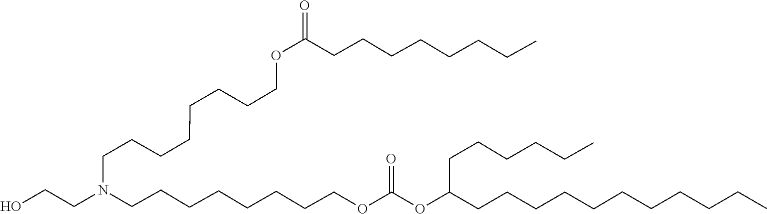 [SMILES: CCCCCCCCCCCC(CCCCCC)OC(=O)OCCCCCCCCN(CCO)CCCCCCCCOC(=O)CCCCCCCC]

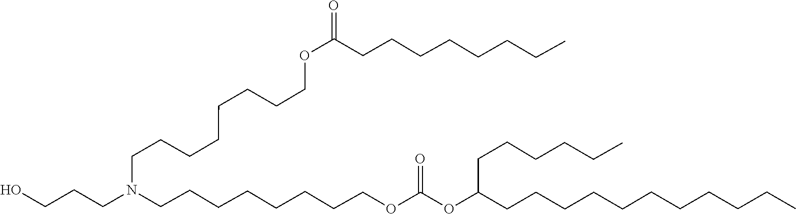 [SMILES: CCCCCCCCCCCC(CCCCCC)OC(=O)OCCCCCCCCN(CCCO)CCCCCCCCOC(=O)CCCCCCCC]

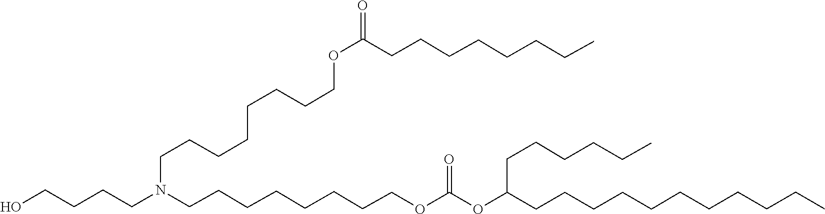 [SMILES: CCCCCCCCCCCC(CCCCCC)OC(=O)OCCCCCCCCN(CCCCO)CCCCCCCCOC(=O)CCCCCCCC]

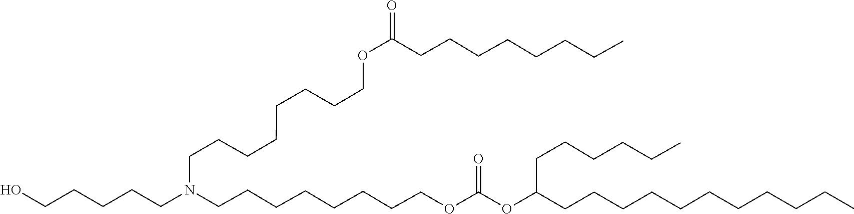 [SMILES: CCCCCCCCCCCC(CCCCCC)OC(=O)OCCCCCCCCN(CCCCCO)CCCCCCCCOC(=O)CCCCCCCC]

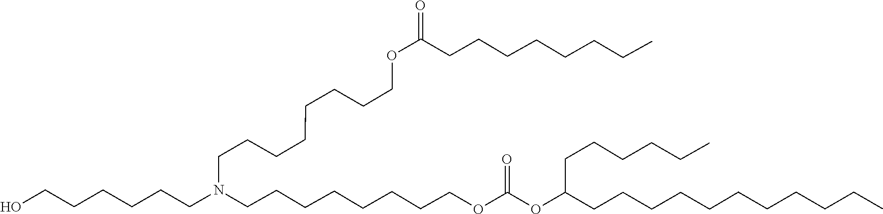 [SMILES: CCCCCCCCCCCC(CCCCCC)OC(=O)OCCCCCCCCN(CCCCCCO)CCCCCCCCOC(=O)CCCCCCCC]

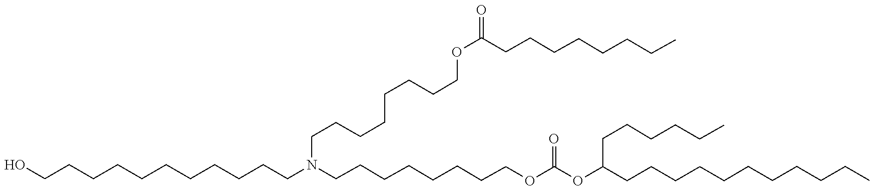 [SMILES: CCCCCCCCCCCC(CCCCCC)OC(=O)OCCCCCCCCN(CCCCCCCCCCCO)CCCCCCCCOC(=O)CCCCCCCC]

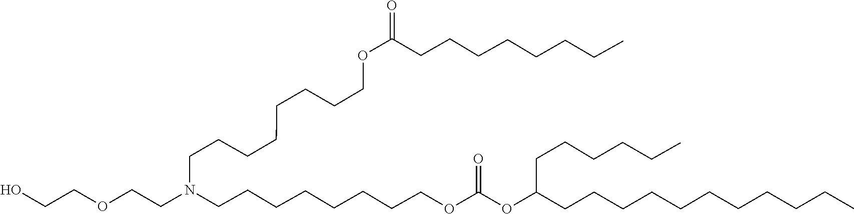 [SMILES: CCCCCCCCCCCC(CCCCCC)OC(=O)OCCCCCCCCN(CCCCCCCCOC(=O)CCCCCCCC)CCOCCO]

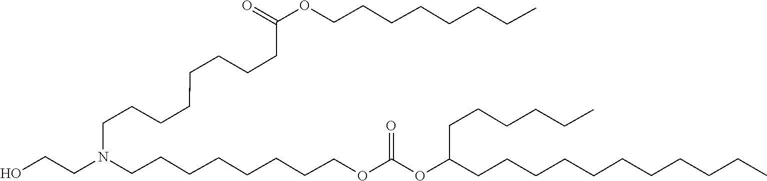 [SMILES: CCCCCCCCCCCC(CCCCCC)OC(=O)OCCCCCCCCN(CCO)CCCCCCCCC(=O)OCCCCCCCC]

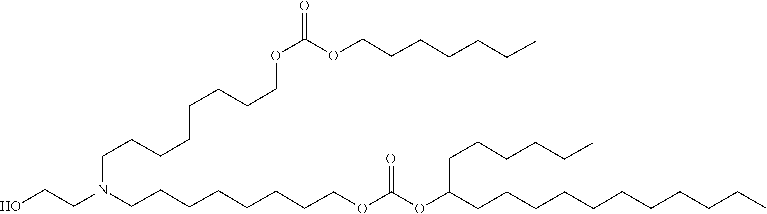 [SMILES: CCCCCCCCCCCC(CCCCCC)OC(=O)OCCCCCCCCN(CCO)CCCCCCCCOC(=O)OCCCCCCC]

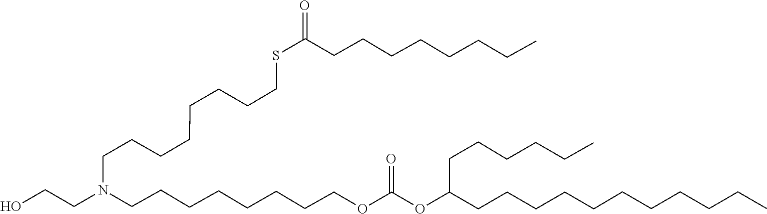 [SMILES: CCCCCCCCCCCC(CCCCCC)OC(=O)OCCCCCCCCN(CCO)CCCCCCCCSC(=O)CCCCCCCC]

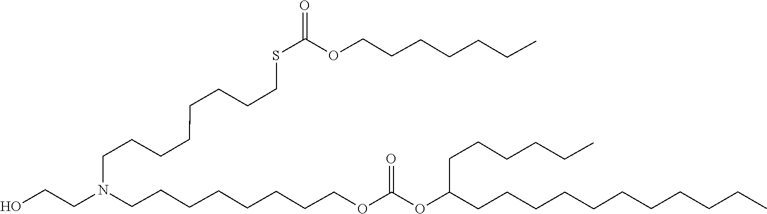 [SMILES: CCCCCCCCCCCC(CCCCCC)OC(=O)OCCCCCCCCN(CCO)CCCCCCCCSC(=O)OCCCCCCC]

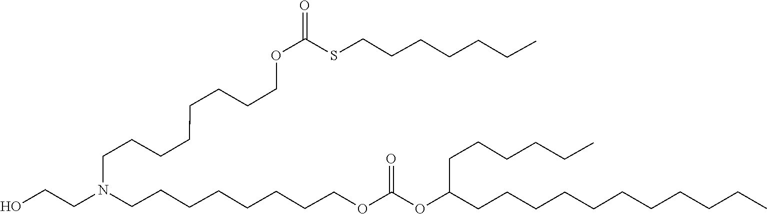 [SMILES: CCCCCCCCCCCC(CCCCCC)OC(=O)OCCCCCCCCN(CCO)CCCCCCCCOC(=O)SCCCCCCC]

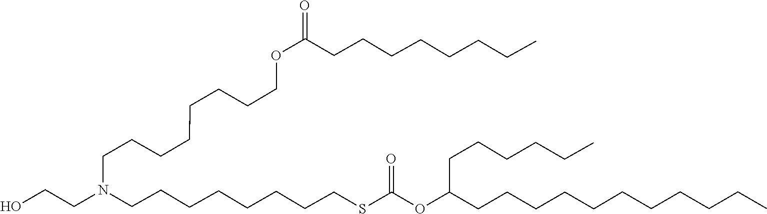 [SMILES: CCCCCCCCCCCC(CCCCCC)OC(=O)SCCCCCCCCN(CCO)CCCCCCCCOC(=O)CCCCCCCC]

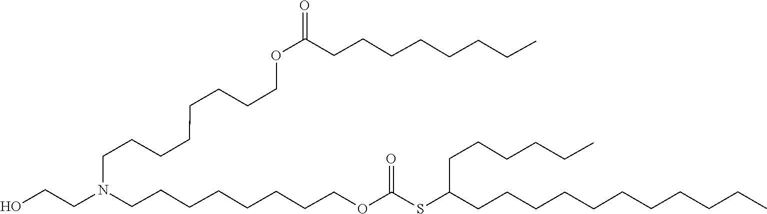 [SMILES: CCCCCCCCCCCC(CCCCCC)SC(=O)OCCCCCCCCN(CCO)CCCCCCCCOC(=O)CCCCCCCC]

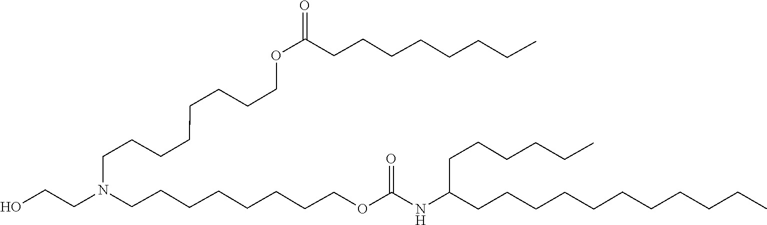 [SMILES: CCCCCCCCCCCC(CCCCCC)NC(=O)OCCCCCCCCN(CCO)CCCCCCCCOC(=O)CCCCCCCC]

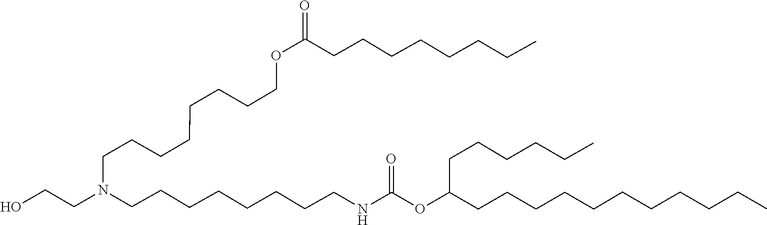 [SMILES: CCCCCCCCCCCC(CCCCCC)OC(=O)NCCCCCCCCN(CCO)CCCCCCCCOC(=O)CCCCCCCC]

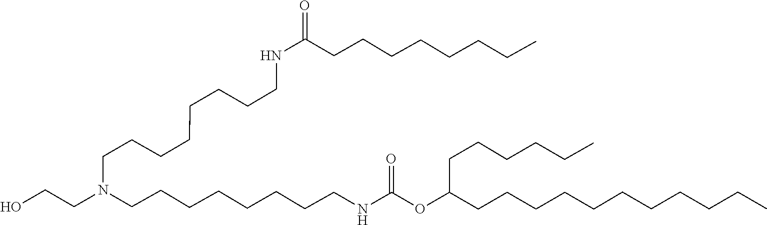 [SMILES: CCCCCCCCCCCC(CCCCCC)OC(=O)NCCCCCCCCN(CCO)CCCCCCCCNC(=O)CCCCCCCC]

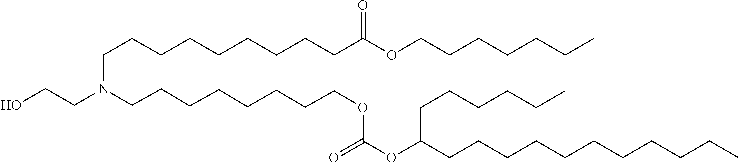 [SMILES: CCCCCCCCCCCC(CCCCCC)OC(=O)OCCCCCCCCN(CCO)CCCCCCCCCC(=O)OCCCCCCC]

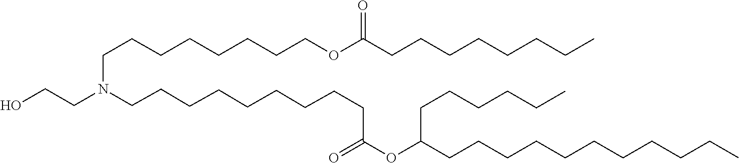 [SMILES: CCCCCCCCCCCC(CCCCCC)OC(=O)CCCCCCCCCN(CCO)CCCCCCCCOC(=O)CCCCCCCC]

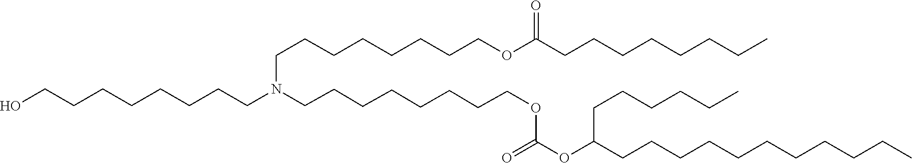 [SMILES: CCCCCCCCCCCC(CCCCCC)OC(=O)OCCCCCCCCN(CCCCCCCCO)CCCCCCCCOC(=O)CCCCCCCC]

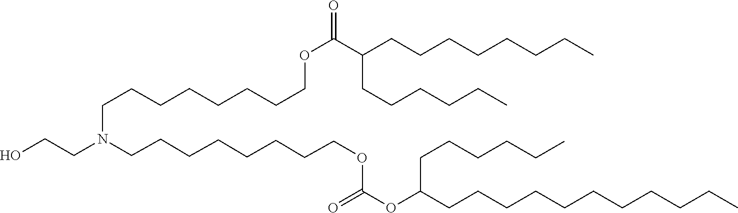 [SMILES: CCCCCCCCCCCC(CCCCCC)OC(=O)OCCCCCCCCN(CCO)CCCCCCCCOC(=O)C(CCCCCC)CCCCCCCC]

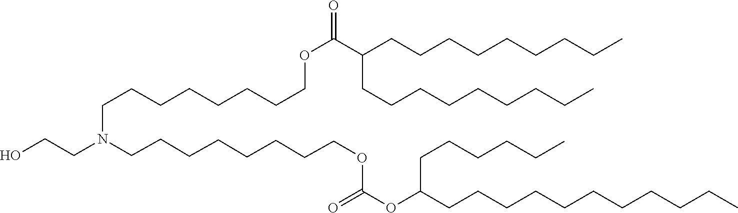 [SMILES: CCCCCCCCCCCC(CCCCCC)OC(=O)OCCCCCCCCN(CCO)CCCCCCCCOC(=O)C(CCCCCCCCC)CCCCCCCCC]

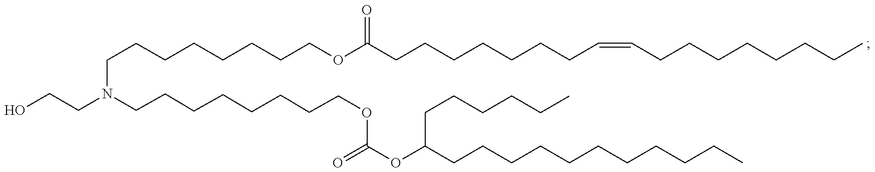 [SMILES: CCCCCCCC/C=C\CCCCCCCC(=O)OCCCCCCCCN(CCO)CCCCCCCCOC(=O)OC(CCCCCC)CCCCCCCCCCC]

or a salt thereof.

In further aspects, provided herein is a nanoparticle composition, comprising a plurality of a lipidoid of the disclosure, or a pharmaceutically acceptable salt thereof.

In certain aspects, the nanoparticle composition further comprises a PEGylated lipid, a sterol, a phospholipid, and/or a neutral lipid.

In certain aspects, the nanoparticle composition further comprises a therapeutic agent.

In certain aspects, the nanoparticle composition further comprises an antigen; wherein the antigen is a protein or a nucleic acid; the antigen is a protein; or the antigen is a nucleic acid.

In certain aspects, the nanoparticle composition further comprises an mRNA molecule that encodes an antigen.

In certain aspects, the nanoparticle composition comprises:
i. a nucleic acid;
ii. a lipidoid of any one of formulae (I), (II), (III), (IV), (V), (VI), or (VII), comprising from 32 mol % to 39 mol % of the total lipid component present in the nanoparticle;
iii. a phospholipid or derivative thereof comprising from 25 mol % to 30 mol % of the total lipid component present in the nanoparticle;
iv. a cholesterol or derivative thereof comprising from 31 mol % to 34 mol % of the total lipid component present in the nanoparticle; and
v. a conjugated lipid that inhibits aggregation of particles comprising from 0.7 mol % to 1.1 mol % of the total lipid component present in the nanoparticle.

In certain aspects, the nanoparticle composition comprises:
i. a nucleic acid;
ii. a cationic lipid comprising 35.7 mol % of the total lipid component present in the nanoparticle;
iii. a phospholipid comprising 29.9 mol % of the total lipid component present in the nanoparticle;
iv. a cholesterol comprising 33.1 mol % of the total lipid component present in the nanoparticle;
v. a conjugated lipid comprising 0.8 mol % of the total lipid component present in the nanoparticle; and
vi. a conjugated lipid comprising GalNAc that comprises 0.5 mol % of the total lipid component present in the nanoparticle.

In further aspects, provided herein is a pharmaceutical composition, comprising a nanoparticle composition of the disclosure, and a pharmaceutically acceptable carrier.

Also provided are methods of delivering a therapeutic agent, comprising administering to a subject in need thereof an effective amount of the nanoparticle composition of the disclosure, wherein the nanoparticle composition further comprises a therapeutic agent.

Also provided herein are methods of vaccination, comprising administering to a subject in need thereof an effective amount of the nanoparticle composition of the disclosure, wherein the nanoparticle composition further comprises an antigen.

### BRIEF DESCRIPTION OF THE DRAWINGS

FIG. 1 shows the hEPO expression level at 6 hours for exemplary Lipid 1 lipid nanoparticle (LNP) as compared to a control LNP comprising SM-102.

FIG. 2 shows the hEPO expression level at 6 hours for exemplary Lipid 1 LNP as compared to a control LNP comprising SM-102 at three different doses.

FIG. 3 shows the ALT and AST levels at 6 hours and 24 hours after administration of Lipid 1 LNP as compared to a control LNP comprising SM-102.

FIG. 4 shows a cytokine analysis at 6 hours and 24 hours following administration of Lipid 1 LNP as compared to a control LNP comprising SM-102.

FIG. 5 is a bar graph comparing cytokine levels after administration of Lipid 1 LNP as compared to a control LNP comprising SM-102.

FIG. 6 is a bar graph showing liver flux after administration of various LNP formulations comprising exemplary Lipid 1 and circular double stranded DNA (cdsDNA) molecules encoding fLuc and GFP proteins.

FIG. 7 is a bar graph showing spleen flux after administration of various LNP formulations comprising exemplary Lipid 1 and circular double stranded DNA (cdsDNA) molecules encoding fLuc and GFP proteins.

FIG. 8 is a bar graph showing the antibody titer on Day 29 after administration of a Lipid 1 LNP comprising mRNA molecules encoding SARS-CoV-2 spike protein.

FIG. 9 shows the hEPO expression level at 6 hours for exemplary LNPs comprising an mRNA encoding hEPO as compared to a control LNP comprising SM-102.

### DETAILED DESCRIPTION

The present invention is based on the surprising discovery of a class of lipidoid compounds useful for forming lipid nanoparticles that can deliver therapeutic agents.

In certain embodiments, the present disclosure provides a lipidoid of formula (VIII), or a pharmaceutically acceptable salt thereof;

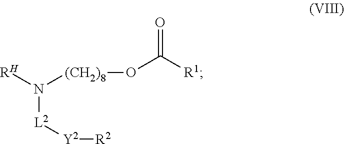

wherein:
R^{H} is hydroxyalkyl or (hydroxyalkyl)-O-alkylene-;
L² represents —(CH₂)ₘ—;
m is an integer selected from 8-10;
Y² is *—O(C═O)O—, *—O(C═O)NH—, or *—(C═O)O—;
wherein the asterisk (*) indicates the point of attachment to L²;
R¹ is linear (C₆-C₁₀)alkyl, branched (C₁₀-C₂₀)alkyl, or linear (C₁₀-C₂₀)alkenyl; and
R² is branched (C₁₀-C₂₀)alkyl; wherein:
   a branched (C₁₀-C₂₀)alkyl may comprise more than one branch point; and
   a linear (C₁₀-C₂₀)alkenyl may comprise more than one carbon-carbon double bond.

In certain such embodiments, R^{H} is hydroxyalkyl.

In certain embodiments, Y² is *—O(C═O)O—.

In certain embodiments, L² is —(CH₂)₈—.

In certain embodiments, R¹ is linear (C₆-C₁₀)alkyl.

In certain embodiments, the present disclosure provides a lipidoid of formula (I), or a pharmaceutically acceptable salt thereof;

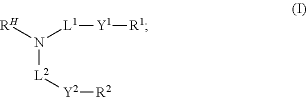

wherein:
R^{H} is hydroxyalkyl or (hydroxyalkyl)-O-alkylene-;
L¹ and L² each represent —(CH₂)ₘ—;
m is independently for each occurrence an integer selected from 8-10;
Y¹ and Y² are each independently *—O(C═O)—, *—O(C═O)O—, *—O(C═O)NH—, or *—(C═O)O—;
   wherein the asterisk (*) indicates the point of attachment to L¹ or L²
   at least one of Y¹ and Y² is *—O(C═O)O—, *—O(C═O)NH—, or *—(C═O)O—; and
   Y¹ and Y² are not both *—(C═O)O—; and
R¹ and R² are each independently optionally substituted linear (C₆-C₂₀)alkyl, branched (C₁₀-C₂₀)alkyl, or linear (C₁₀-C₂₀)alkenyl; wherein:
   at least one of R¹ and R² is branched (C₁₀-C₂₀)alkyl; and
   R¹ and R² are different;
   a branched (C₁₀-C₂₀)alkyl may comprise more than one branch point; and
   a linear (C₁₀-C₂₀)alkenyl may comprise more than one carbon-carbon double bond.

In certain embodiments, Y¹ and Y² are not identical.

In certain embodiments, Y¹ is *—O(C═O)—.

In certain embodiments, Y² is *—O(C═O)O—.

In certain embodiments, L¹ is —(CH₂)₈—.

In certain embodiments, L² is —(CH₂)₈— or —(CH₂)₉—.

In certain embodiments, R¹ is linear (C₆-C₁₀)alkyl.

In certain embodiments, R² is branched (C₁₀-C₂₀)alkyl.

In certain embodiments, R^{H} is hydroxyalkyl.

In certain embodiments, the present disclosure provides a lipidoid of formula (I), or a pharmaceutically acceptable salt thereof;

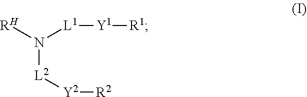

wherein:
R^{H} is hydroxyalkyl, (hydroxyalkyl)-O-alkylene-, or alkyl substituted by

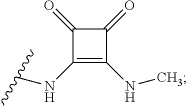 [SMILES: *Nc1c(NC)c(=O)c1=O]

L¹ and L² each represent —(CH₂)ₘ—;
m is independently for each occurrence an integer selected from 1-20;
Y¹ and Y² are each independently *—O(C═O)—, *—S(C═O)—, *—NH(C═O)—, *—NH(C═S)—, *—O(C═O)O—, *—O(C═S)O—, *—S(C═O)O—, *—O(C═O)S—, *—S(C═S)O—, *—NH(C═O)O—, *—O(C═O)NH—, *—(C═O)O—, *—(C═S)O—, *—O(P(O)OR²⁰)O—,

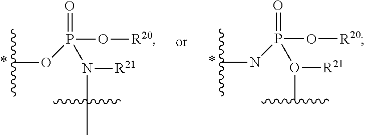

wherein the asterisk (*) indicates the point of attachment to L¹ or L²;
R²⁰ is independently for each occurrence an optionally substituted linear or branched (C₁-C₁₅)alkyl;
R²¹ is independently for each occurrence H, optionally substituted (C₁-C₅)alkyl, or optionally substituted (C₃-C₅)cycloalkyl; and
R¹ and R² are each independently optionally substituted linear (C₄-C₃₀)alkyl, branched (C₄-C₃₀)alkyl, linear (C₄-C₃₀)alkenyl, branched (C₄-C₃₀)alkenyl, linear (C₄-C₃₀)alkynyl, or branched (C₄-C₃₀)alkynyl; wherein:
   a branched (C₄-C₃₀)alkyl, a branched (C₄-C₃₀)alkenyl, or a branched (C₄-C₃₀)alkynyl may comprise more than one branch point;
   a linear or branched (C₄-C₃₀)alkenyl may comprise more than one carbon-carbon double bond;
   a linear or branched (C₄-C₃₀)alkynyl may comprise more than one carbon-carbon triple bond; and
   in any linear (C₄-C₃₀)alkyl, branched (C₄-C₃₀)alkyl, linear (C₄-C₃₀)alkenyl, branched (C₄-C₃₀)alkenyl, linear (C₄-C₃₀)alkynyl, or branched (C₄-C₃₀)alkynyl group, a —CH₂CH₂— moiety may be replaced by a —S—S-moiety.

In certain embodiments, R^{H} is hydroxyalkyl.

In certain embodiments, R^{H} is —CH₂CH₂OH.

In certain embodiments, m is independently for each occurrence an integer selected from 4-20, preferably 8-10.

In certain embodiments, L¹ and L² each represent —(CH₂)₈—.

In certain embodiments, Y¹ and Y² are each independently *—O(C═O)—, *—S(C═O)—, *—NH(C═O)—, *—O(C═O)O—, *—S(C═O)O—, *—O(C═O)S—, *1-NH(C═O)O—, *—O(C═O)NH—, or *—(C═O)O—.

In certain embodiments, Y¹ is *—O(C═O)—, *—S(C═O)—, *—NH(C═O)—, or *—(C═O)O—.

In certain embodiments, Y¹ is *—O(C═O)—.

In certain embodiments, Y² is *—O(C═O)O—, *—S(C═O)O—, *—O(C═O)S—, *—NH(C═O)O—, or *—O(C═O)NH—.

In certain embodiments, Y² is *—O(C═O)O—.

In certain embodiments, at least one of Y¹ and Y² is *—S(C═O)—, *—NH(C═O)—, *—NH(C═S)—, *—O(C═O)O—, *—O(C═S)O—, *—S(C═O)O—, *—O(C═O)S—, *—S(C═S)O—, *—NH(C═O)O—, *—O(C═O)NH—, *—(C═O)O—, *—(C═S)O—, *—O(P(O)OR²⁰)—,

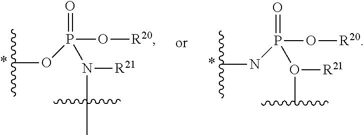

In certain embodiments, Y¹ and Y² are not both *—(C═O)O—.

In certain embodiments, Y¹ and Y² are different.

In certain embodiments, Y¹ and Y² are the same.

In certain embodiments, R¹ is linear (C₄-C₃₀)alkyl, branched (C₄-C₃₀)alkyl, linear (C₄-C₃₀)alkenyl, or linear (C₄-C₃₀)alkyl wherein a —CH₂CH₂ moiety is replaced by a —S—S— moiety.

In certain embodiments, R¹ is linear (C₄-C₃₀)alkyl or branched (C₄-C₃₀)alkyl.

In certain embodiments, R¹ is linear (C₆-C₂₀)alkyl or branched (C₆-C₂₀)alkyl.

In certain embodiments, R¹ is linear (C₆-C₁₄)alkyl or branched (C₆-C₁₄)alkyl.

In certain embodiments, R¹ is linear (C₈)alkyl.

In certain embodiments, R¹ is linear (C₄-C₃₀)alkenyl.

In certain embodiments, R¹ is linear (C₅-C₂₀)alkenyl.

In certain embodiments, R¹ is linear (C₄-C₃₀)alkyl wherein a —CH₂CH₂— moiety is replaced by a —S—S— moiety.

In certain embodiments, R¹ is linear (C₆-C₂₀)alkyl wherein a —CH₂CH₂— moiety is replaced by a —S—S— moiety.

In certain embodiments, R² is linear (C₄-C₃₀)alkyl or branched (C₄-C₃₀)alkyl.

In certain embodiments, R² is branched (C₆-C₃₀)alkyl.

In certain embodiments, R² is branched (C₁₆-C₂₆)alkyl.

In certain embodiments, R² is branched (C₁₈-C₂₄)alkyl.

In certain embodiments, R² is branched (C₁₈)alkyl.

In certain embodiments, R¹ and R² are different.

In certain embodiments, R¹ is

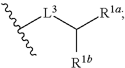

L³ is absent or (C₁-C₂₇)alkylene, (C₂-C₂₇)alkenylene, or (C₂-C₂₇)alkynylene; wherein:
a (C₂-C₂₇)alkenylene may comprise more than one carbon-carbon double bond;
a (C₂-C₂₇)alkynylene may comprise more than one carbon-carbon triple bond; and
R^{1a} and R^{1b} are each independently (C₁-C₂₈)alkyl, (C₂-C₂₈)alkenyl, or (C₃-C₂₈)alkynyl; wherein:
the sum of carbon atoms in L³, R^{1a}, and R^{1b} is from 3 to 29 total carbon atoms.

In certain embodiments, L³ is absent.

In certain embodiments, L³ is (C₁-C₁₂)alkylene.

In certain embodiments, R^{1a} and R^{1b} are each independently (C₁-C₁₂)alkyl.

In certain embodiments, one of R^{1a} and R^{1b} is (C₁-C₆)alkyl.

In certain embodiments, R² is

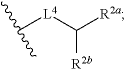

L⁴ is absent or (C₁-C₂₇)alkylene, (C₂-C₂₇)alkenylene, or (C₂-C₂₇)alkynylene; wherein:
a (C₂-C₂₇)alkenylene may comprise more than one carbon-carbon double bond;
a (C₂-C₂₇)alkynylene may comprise more than one carbon-carbon triple bond; and
R^{2a} and R^{2b} are each independently (C₁-C₂₈)alkyl, (C₂-C₂₈)alkenyl, or (C₃-C₂₈)alkynyl; wherein:
the sum of carbon atoms in L⁴, R^{2a}, and R^{2b} is from 3 to 29 total carbon atoms.

In certain embodiments, L⁴ is absent.

In certain embodiments, L⁴ is (C₁-C₁₂)alkylene.

In certain embodiments, R^{2a} and R^{2b} are each independently (C₁-C₁₂)alkyl.

In certain embodiments, one of R^{2a} and R^{2b} is (C₁-C₆)alkyl.

In certain embodiments, the present disclosure provides a lipidoid of formula (II), or a pharmaceutically acceptable salt thereof;

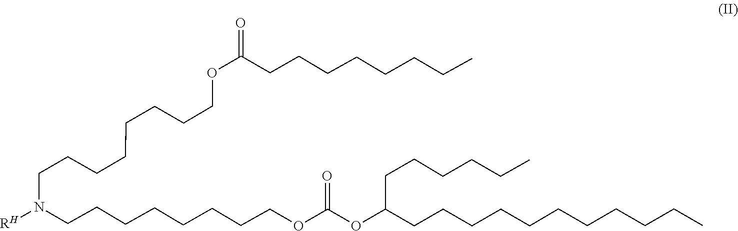

wherein:
R^{H} is hydroxyalkyl, (hydroxyalkyl)-O-alkylene-, or alkyl substituted by

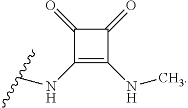 [SMILES: *Nc1c(NC)c(=O)c1=O]

In certain embodiments, R^{H} is a C₂₋₆ hydroxyalkyl.

In certain embodiments, R^{H} is alkyl substituted by

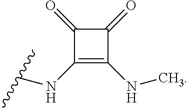 [SMILES: *Nc1c(NC)c(=O)c1=O]

In certain embodiments, the present disclosure provides a lipidoid of formula (III), or a pharmaceutically acceptable salt thereof;

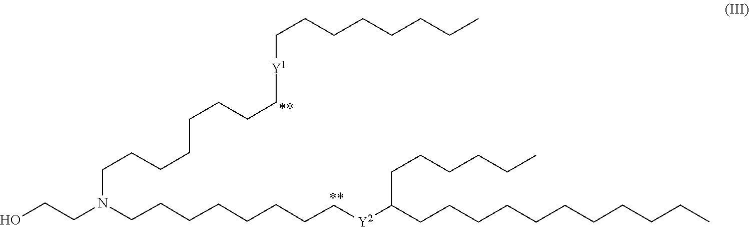

wherein:
Y¹ and Y² are each independently *—O(C═O)—, *—S(C═O)—, *—NH(C═O)—, *—NH(C═S)—, *—O(C═O)O—, *—O(C═S)O—, *—S(C═O)O—, *—O(C═O)S—, *—S(C═S)O—, *—NH(C═O)O—, *—O(C═O)NH—, *—(C═O)O—, *—(C═S)O—;
wherein the asterisk (*) indicates the point of attachment to the carbon designated by the double asterisk (**);
at least one of Y¹ and Y² is *—S(C═O)—, *—NH(C═O)—, *—NH(C═S)—, *—O(C═O)O—, *—O(C═S)O—, *—S(C═O)O—, *—O(C═O)S—, *—S(C═S)O—, *—NH(C═O)O—, *—O(C═O)NH—, *—(C═O)O—, or *—(C═S)O— and
Y¹ and Y² are not both *—(C═O)O—.

In certain embodiments, Y¹ and Y² are each independently *—O(C═O)— or *—O(C═O)O—.

In certain embodiments, at least one of Y¹ and Y² is *—O(C═O)O—.

In certain embodiments, Y¹ is *—O(C═O)— and Y² is *—O(C═O)O—.

In certain embodiments, the present disclosure provides a lipidoid of formula (IV), or a pharmaceutically acceptable salt thereof;

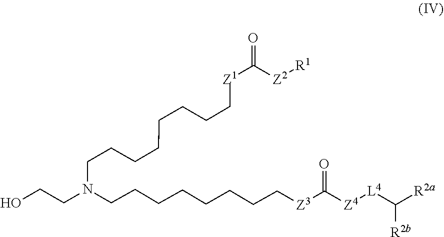

wherein:
   Z¹, Z², Z³, and Z⁴ are each independently absent, CH₂, O, NH, or S; wherein:
   at least one of Z¹ and Z² is O, NH, or S;
   at least one of Z³ and Z⁴ is O, NH, or S;
   L⁴ is absent or (C₁-C₂₇)alkylene, (C₂-C₂₇)alkenylene, or (C₂-C₂₇)alkynylene;
wherein:
   a (C₂-C₂₇)alkenylene may comprise more than one carbon-carbon double bond;
   a (C₂-C₂₇)alkynylene may comprise more than one carbon-carbon triple bond; and;
   R¹ is optionally substituted linear (C₄-C₃₀)alkyl, branched (C₄-C₃₀)alkyl, linear (C₄-C₃₀)alkenyl, branched (C₄-C₃₀)alkenyl, linear (C₄-C₃₀)alkynyl, or branched (C₄-C₃₀)alkynyl;
   R^{2a} and R^{2b} are each independently (C₁-C₂₈)alkyl, (C₂-C₂₈)alkenyl, or (C₃-C₂₈)alkynyl; wherein:
   the sum of carbon atoms in L⁴, R^{2a}, and R^{2b} is from 3 to 29 total carbon atoms.

In certain embodiments, each of Z¹, Z³, and Z⁴ are O.

In certain embodiments, Z² is absent.

In certain embodiments, at least one of Z¹ and Z³ is O.

In certain embodiments, R¹ is linear (C₄-C₁₆)alkyl.

In certain embodiments, R¹ is linear (C₆-C₁₂)alkyl.

In certain embodiments, at least one of R^{2a} and R^{2b} is (C₁-C₁₂)alkyl.

In certain embodiments, one of R^{2a} and R^{2b} is (C₄-C₁₆)alkyl.

In certain embodiments, one of R^{2a} and R^{2b} is (C₁-C₅)alkyl.

In certain embodiments, the present disclosure provides a lipidoid of formula (V), or a pharmaceutically acceptable salt thereof;

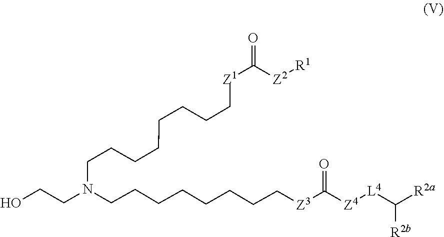

wherein:
R^{H} is hydroxyalkyl, (hydroxyalkyl)-O-alkylene-, or alkyl substituted by

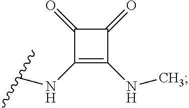 [SMILES: *Nc1c(NC)c(=O)c1=O]

Z¹, Z², Z³, and Z⁴ are each independently absent, CH₂, O, NH, or S; wherein:
at least one of Z¹ and Z² is O, NH, or S;
at least one of Z³ and Z⁴ is O, NH, or S;
L⁴ is absent or (C₁-C₂₇)alkylene, (C₂-C₂₇)alkenylene, or (C₂-C₂₇)alkynylene; wherein:
a (C₂-C₂₇)alkenylene may comprise more than one carbon-carbon double bond;
a (C₂-C₂₇)alkynylene may comprise more than one carbon-carbon triple bond; and;
R¹ is optionally substituted linear (C₄-C₃₀)alkyl, branched (C₄-C₃₀)alkyl, linear (C₄-C₃₀)alkenyl, branched (C₄-C₃₀)alkenyl, linear (C₄-C₃₀)alkynyl, or branched (C₄-C₃₀)alkynyl;
R^{2a} and R^{2b} are each independently (C₁-C₂₈)alkyl, (C₂-C₂₈)alkenyl, or (C₃-C₂₈)alkynyl; wherein:
the sum of carbon atoms in L⁴, R^{2a}, and R^{2b} is from 3 to 29 total carbon atoms.

In certain embodiments, each of Z¹, Z³, and Z⁴ are O.

In certain embodiments, Z² is absent.

In certain embodiments, at least one of Z¹ and Z³ is O.

In certain embodiments, R¹ is linear (C₄-C₁₆)alkyl.

In certain embodiments, R¹ is linear (C₆-C₁₂)alkyl.

In certain embodiments, at least one of R^{2a} and R^{2b} is (C₁-C₁₂)alkyl.

In certain embodiments, one of R^{2a} and R^{2b} is (C₄-C₁₆)alkyl.

In certain embodiments, one of R^{2a} and R^{2b} is (C₁-C₅)alkyl.

In certain embodiments, R^{H} is a C₂₋₆ hydroxyalkyl.

In certain embodiments, R^{H} is alkyl substituted by

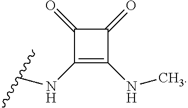 [SMILES: *Nc1c(NC)c(=O)c1=O]

In certain embodiments, the present disclosure provides a lipidoid of formula (VI), or a pharmaceutically acceptable salt thereof;

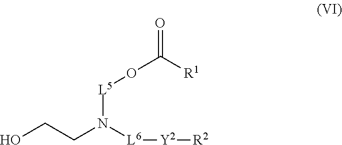

wherein:
L⁵ and L⁶ each represent —(CH₂)ₘ—;
m is independently for each occurrence an integer selected from 8-10;
Y² is *—S(C═O)—, *—NH(C═O)—, *—NH(C═S)—, *—O(C═O)O—, *—O(C═S)O—, *—S(C═O)O—, *—O(C═O)S—, *—S(C═S)O—, *—NH(C═O)O—, *—O(C═O)NH—, *—(C═O)O—, *—(C═S)O—, *—O(P(O)OR²⁰)O,

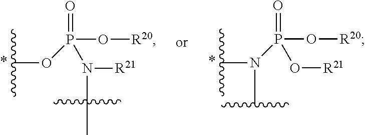

wherein the asterisk (*) indicates the point of attachment to L⁶; and
R¹ and R² are each independently optionally substituted linear (C₆-C₂₀)alkyl, branched (C₁₀-C₂₀)alkyl, or linear (C₁₀-C₂₀)alkenyl.

In certain embodiments, the present disclosure provides a lipidoid of formula (VII), or a pharmaceutically acceptable salt thereof;

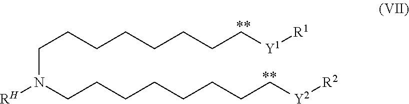

wherein:
R^{H} is hydroxyalkyl, (hydroxyalkyl)-O-alkylene-, or alkyl substituted by

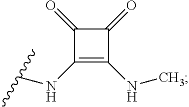 [SMILES: *Nc1c(NC)c(=O)c1=O]

Y¹ is *—O(C═O)—, *—O(C═O)O—, *—O(C═O)NH—, or *—(C═O)O—;
Y² is *—O(C═O)O—, *—O(C═O)NH—, or *—(C═O)O—;
wherein the asterisk (*) indicates the point of attachment to the carbon designated by the double asterisk (**);
R¹ and R² are each independently optionally substituted linear (C₄-C₃₀)alkyl, branched (C₄-C₃₀)alkyl, linear (C₄-C₃₀)alkenyl, branched (C₄-C₃₀)alkenyl, linear (C₄-C₃₀)alkynyl, or branched (C₄-C₃₀)alkynyl; wherein:
   a branched (C₄-C₃₀)alkyl, a branched (C₄-C₃₀)alkenyl, or a branched (C₄-C₃₀)alkynyl may comprise more than one branch point;
   a linear or branched (C₄-C₃₀)alkenyl may comprise more than one carbon-carbon double bond;
   a linear or branched (C₄-C₃₀)alkynyl may comprise more than one carbon-carbon triple bond; and
   in any linear (C₄-C₃₀)alkyl, branched (C₄-C₃₀)alkyl, linear (C₄-C₃₀)alkenyl, branched (C₄-C₃₀)alkenyl, linear (C₄-C₃₀)alkynyl, or branched (C₄-C₃₀)alkynyl group, a —CH₂CH₂— moiety may be replaced by a —S—S-moiety.

In certain embodiments, the lipidoid is selected from the following table, Table L:

TABLE L
Exemplary Lipidoids of the Disclosure

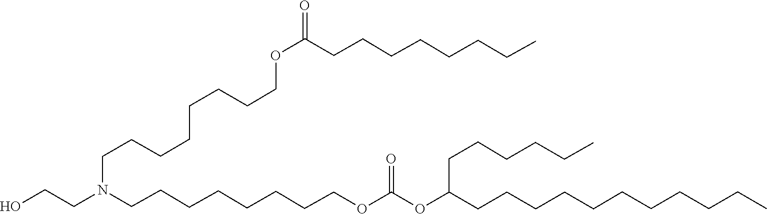 [SMILES: CCCCCCCCCCCC(CCCCCC)OC(=O)OCCCCCCCCN(CCO)CCCCCCCCOC(=O)CCCCCCCC]

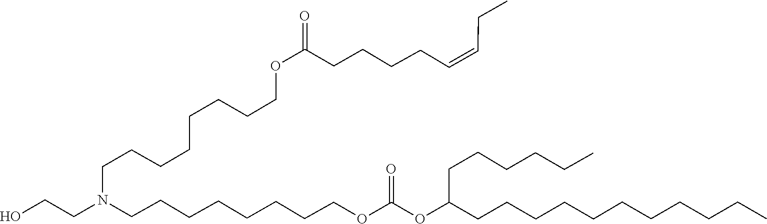 [SMILES: CC/C=C\CCCCC(=O)OCCCCCCCCN(CCO)CCCCCCCCOC(=O)OC(CCCCCC)CCCCCCCCCCC]

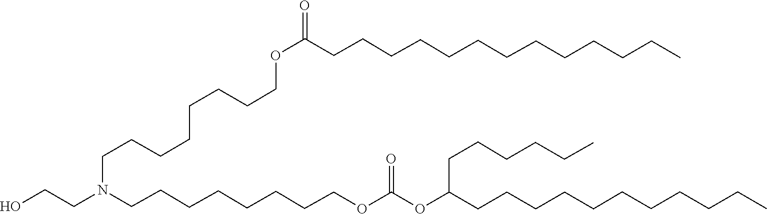 [SMILES: CCCCCCCCCCCCCC(=O)OCCCCCCCCN(CCO)CCCCCCCCOC(=O)OC(CCCCCC)CCCCCCCCCCC]

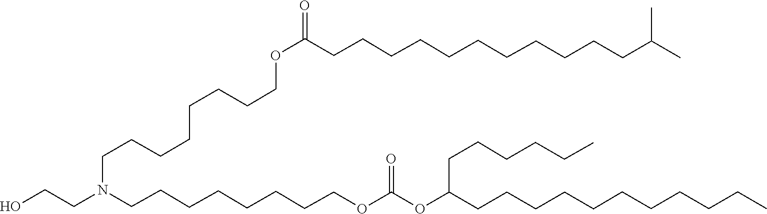 [SMILES: CCCCCCCCCCCC(CCCCCC)OC(=O)OCCCCCCCCN(CCO)CCCCCCCCOC(=O)CCCCCCCCCCCC(C)C]

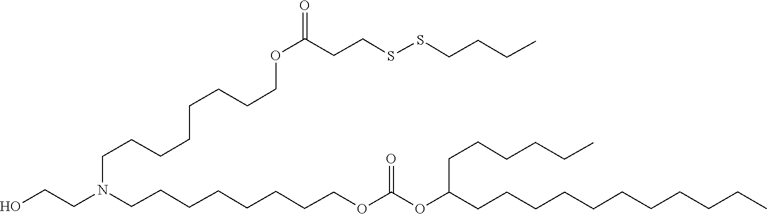 [SMILES: CCCCCCCCCCCC(CCCCCC)OC(=O)OCCCCCCCCN(CCO)CCCCCCCCOC(=O)CCSSCCCC]

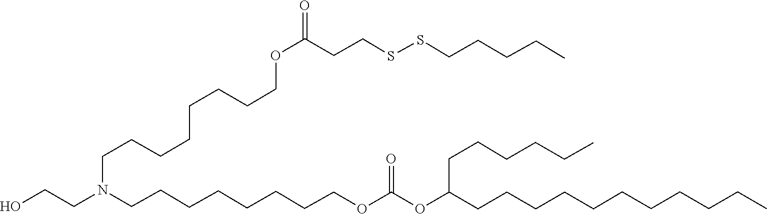 [SMILES: CCCCCCCCCCCC(CCCCCC)OC(=O)OCCCCCCCCN(CCO)CCCCCCCCOC(=O)CCSSCCCCC]

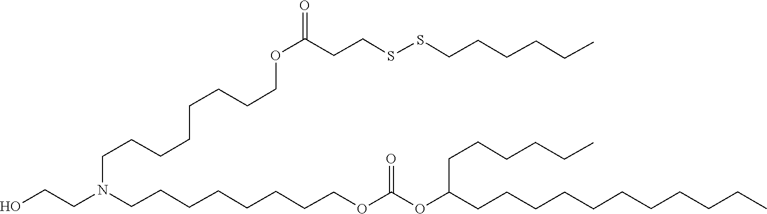 [SMILES: CCCCCCCCCCCC(CCCCCC)OC(=O)OCCCCCCCCN(CCO)CCCCCCCCOC(=O)CCSSCCCCCC]

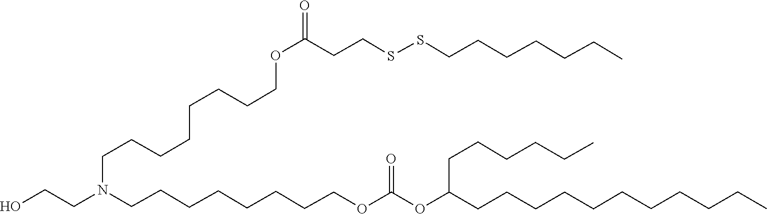 [SMILES: CCCCCCCCCCCC(CCCCCC)OC(=O)OCCCCCCCCN(CCO)CCCCCCCCOC(=O)CCSSCCCCCCC]

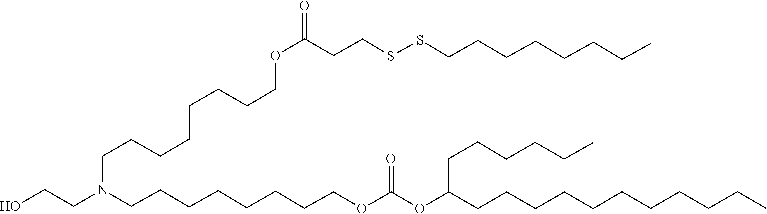 [SMILES: CCCCCCCCCCCC(CCCCCC)OC(=O)OCCCCCCCCN(CCO)CCCCCCCCOC(=O)CCSSCCCCCCCC]

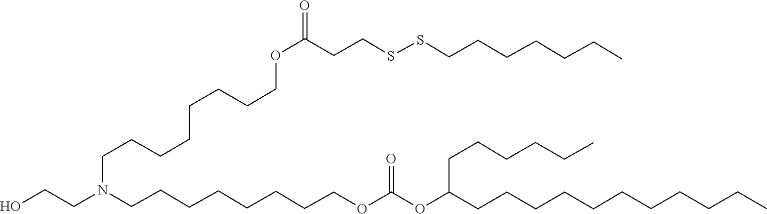 [SMILES: CCCCCCCCCCCC(CCCCCC)OC(=O)OCCCCCCCCN(CCO)CCCCCCCCOC(=O)CCSSCCCCCCC]

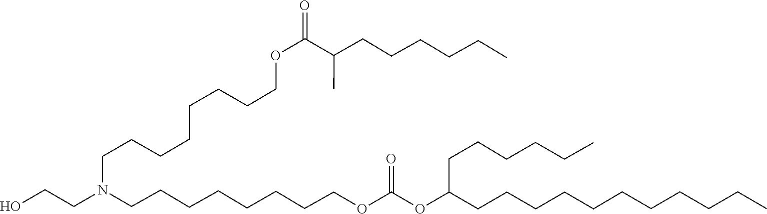 [SMILES: CCCCCCCCCCCC(CCCCCC)OC(=O)OCCCCCCCCN(CCO)CCCCCCCCOC(=O)C(C)CCCCCC]

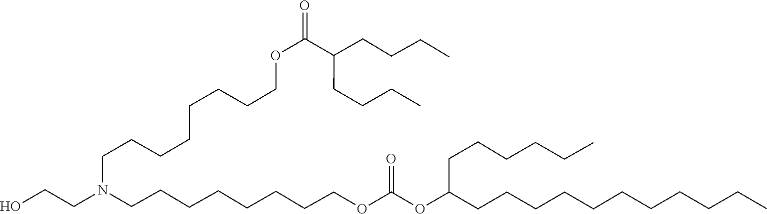 [SMILES: CCCCCCCCCCCC(CCCCCC)OC(=O)OCCCCCCCCN(CCO)CCCCCCCCOC(=O)C(CCCC)CCCC]

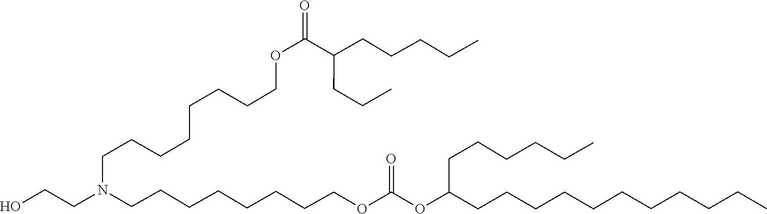 [SMILES: CCCCCCCCCCCC(CCCCCC)OC(=O)OCCCCCCCCN(CCO)CCCCCCCCOC(=O)C(CCC)CCCCC]

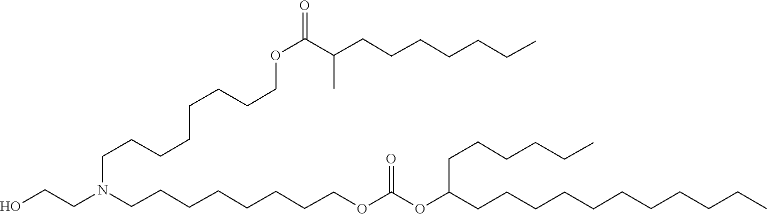 [SMILES: CCCCCCCCCCCC(CCCCCC)OC(=O)OCCCCCCCCN(CCO)CCCCCCCCOC(=O)C(C)CCCCCCC]

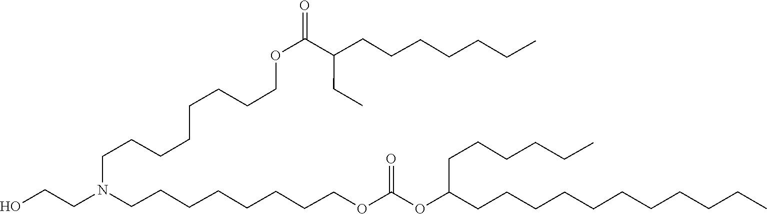 [SMILES: CCCCCCCCCCCC(CCCCCC)OC(=O)OCCCCCCCCN(CCO)CCCCCCCCOC(=O)C(CC)CCCCCCC]

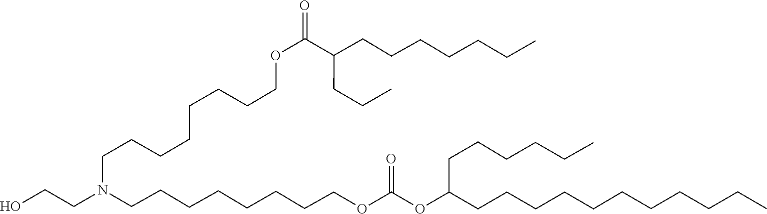 [SMILES: CCCCCCCCCCCC(CCCCCC)OC(=O)OCCCCCCCCN(CCO)CCCCCCCCOC(=O)C(CCC)CCCCCCC]

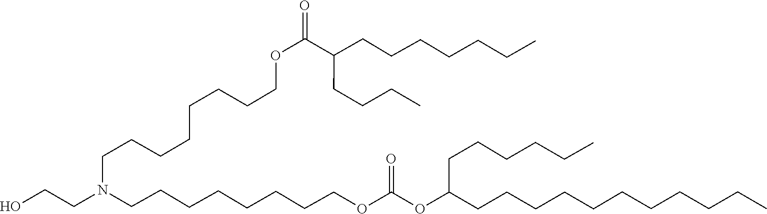 [SMILES: CCCCCCCCCCCC(CCCCCC)OC(=O)OCCCCCCCCN(CCO)CCCCCCCCOC(=O)C(CCCC)CCCCCCC]

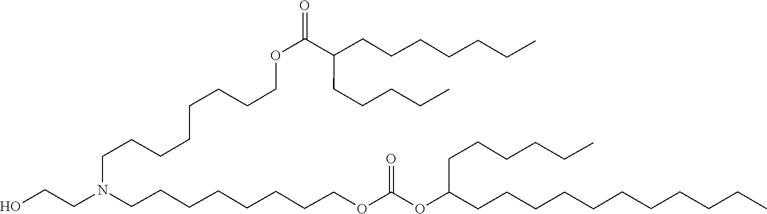 [SMILES: CCCCCCCCCCCC(CCCCCC)OC(=O)OCCCCCCCCN(CCO)CCCCCCCCOC(=O)C(CCCCC)CCCCCCC]

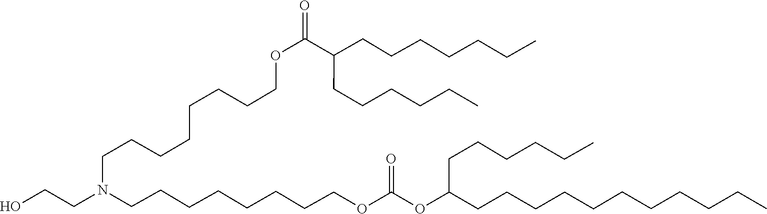 [SMILES: CCCCCCCCCCCC(CCCCCC)OC(=O)OCCCCCCCCN(CCO)CCCCCCCCOC(=O)C(CCCCCC)CCCCCCC]

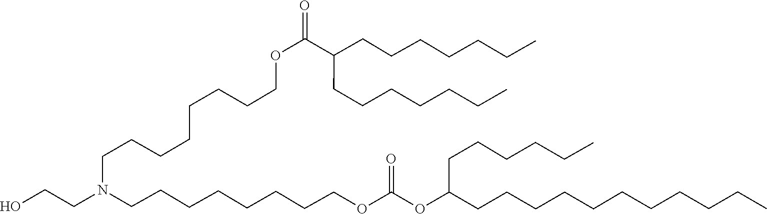 [SMILES: CCCCCCCCCCCC(CCCCCC)OC(=O)OCCCCCCCCN(CCO)CCCCCCCCOC(=O)C(CCCCCCC)CCCCCCC]

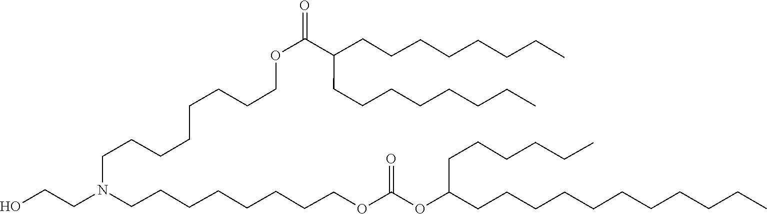 [SMILES: CCCCCCCCCCCC(CCCCCC)OC(=O)OCCCCCCCCN(CCO)CCCCCCCCOC(=O)C(CCCCCCCC)CCCCCCCC]

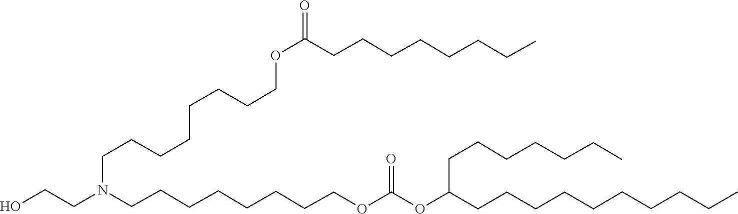 [SMILES: CCCCCCCCCCC(CCCCCCC)OC(=O)OCCCCCCCCN(CCO)CCCCCCCCOC(=O)CCCCCCCC]

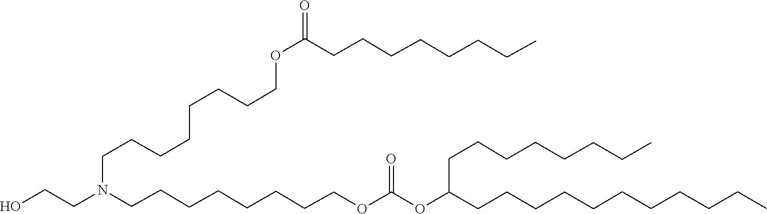 [SMILES: CCCCCCCCCCCC(CCCCCCCC)OC(=O)OCCCCCCCCN(CCO)CCCCCCCCOC(=O)CCCCCCCC]

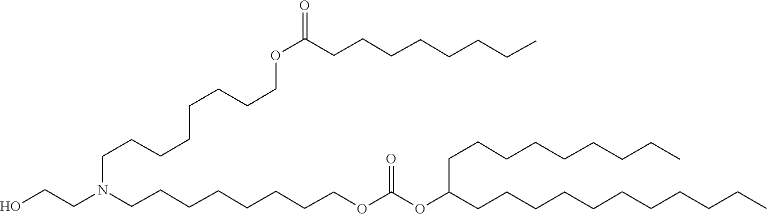 [SMILES: CCCCCCCCCCCC(CCCCCCCCC)OC(=O)OCCCCCCCCN(CCO)CCCCCCCCOC(=O)CCCCCCCC]

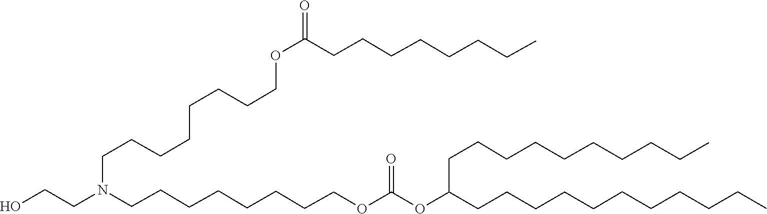 [SMILES: CCCCCCCCCCCC(CCCCCCCCCC)OC(=O)OCCCCCCCCN(CCO)CCCCCCCCOC(=O)CCCCCCCC]

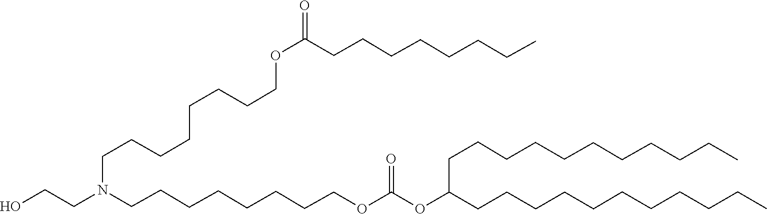 [SMILES: CCCCCCCCCCCC(CCCCCCCCCCC)OC(=O)OCCCCCCCCN(CCO)CCCCCCCCOC(=O)CCCCCCCC]

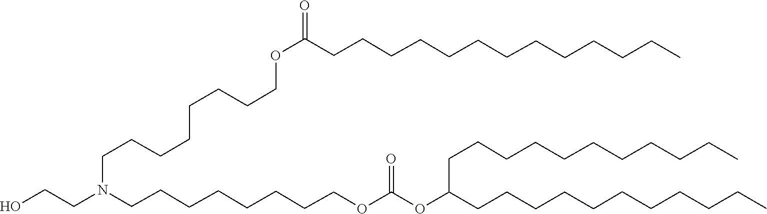 [SMILES: CCCCCCCCCCCCCC(=O)OCCCCCCCCN(CCO)CCCCCCCCOC(=O)OC(CCCCCCCCCCC)CCCCCCCCCCC]

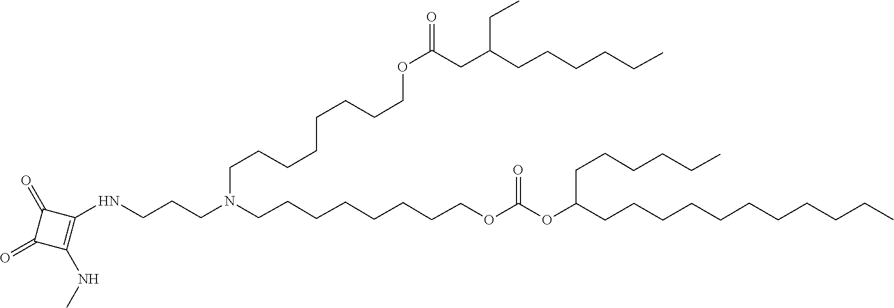 [SMILES: CCCCCCCCCCCC(CCCCCC)OC(=O)OCCCCCCCCN(CCCCCCCCOC(=O)CC(CC)CCCCCC)CCCNc1c(NC)c(=O)c1=O]

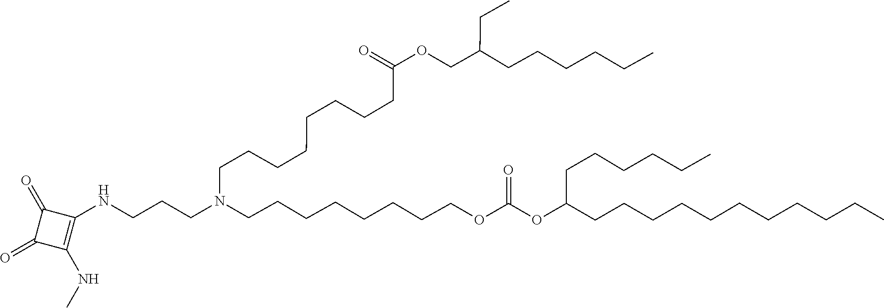 [SMILES: CCCCCCCCCCCC(CCCCCC)OC(=O)OCCCCCCCCN(CCCCCCCCC(=O)OCC(CC)CCCCCC)CCCNc1c(NC)c(=O)c1=O]

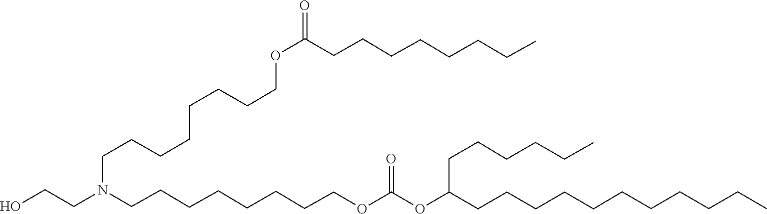 [SMILES: CCCCCCCCCCCC(CCCCCC)OC(=O)OCCCCCCCCN(CCO)CCCCCCCCOC(=O)CCCCCCCC]

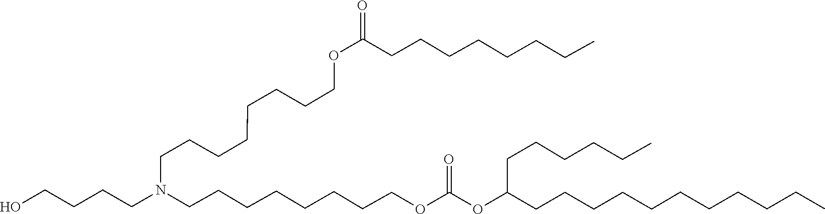 [SMILES: CCCCCCCCCCCC(CCCCCC)OC(=O)OCCCCCCCCN(CCCCO)CCCCCCCCOC(=O)CCCCCCCC]

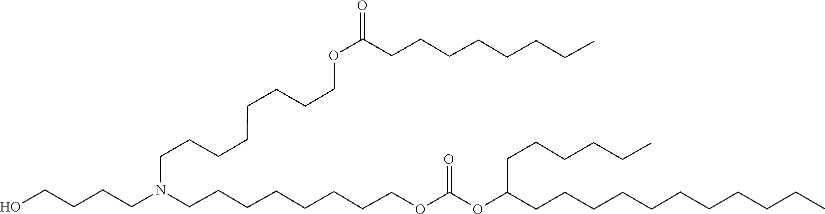 [SMILES: CCCCCCCCCCCC(CCCCCC)OC(=O)OCCCCCCCCN(CCCCO)CCCCCCCCOC(=O)CCCCCCCC]

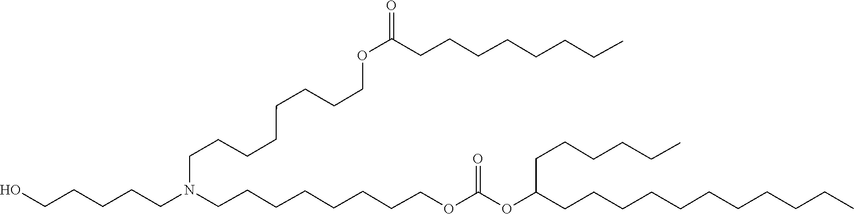 [SMILES: CCCCCCCCCCCC(CCCCCC)OC(=O)OCCCCCCCCN(CCCCCO)CCCCCCCCOC(=O)CCCCCCCC]

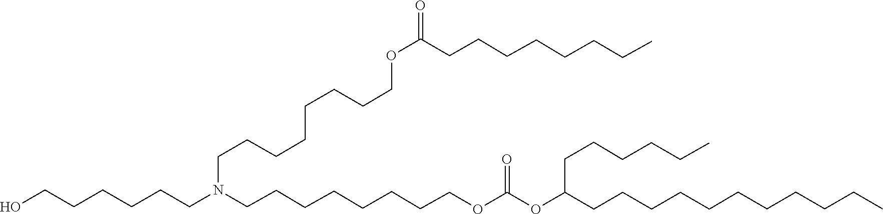 [SMILES: CCCCCCCCCCCC(CCCCCC)OC(=O)OCCCCCCCCN(CCCCCCO)CCCCCCCCOC(=O)CCCCCCCC]

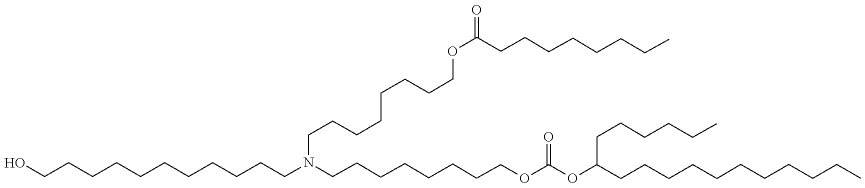 [SMILES: CCCCCCCCCCCC(CCCCCC)OC(=O)OCCCCCCCCN(CCCCCCCCCCCO)CCCCCCCCOC(=O)CCCCCCCC]

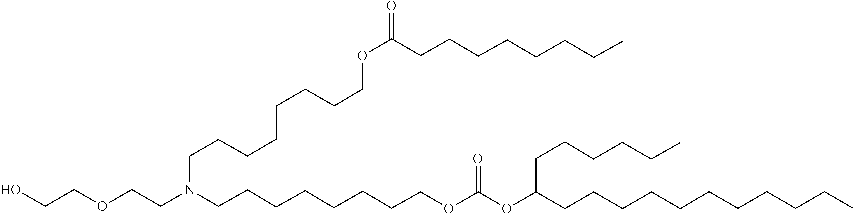 [SMILES: CCCCCCCCCCCC(CCCCCC)OC(=O)OCCCCCCCCN(CCCCCCCCOC(=O)CCCCCCCC)CCOCCO]

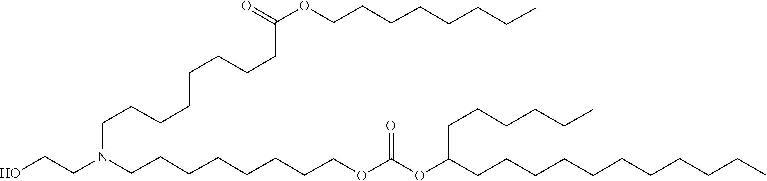 [SMILES: CCCCCCCCCCCC(CCCCCC)OC(=O)OCCCCCCCCN(CCO)CCCCCCCCC(=O)OCCCCCCCC]

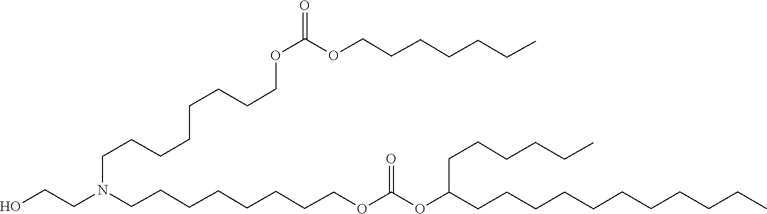 [SMILES: CCCCCCCCCCCC(CCCCCC)OC(=O)OCCCCCCCCN(CCO)CCCCCCCCOC(=O)OCCCCCCC]

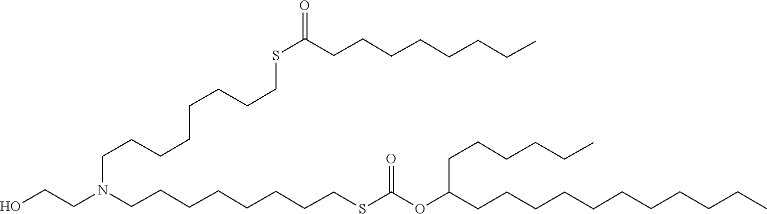 [SMILES: CCCCCCCCCCCC(CCCCCC)OC(=O)SCCCCCCCCN(CCO)CCCCCCCCSC(=O)CCCCCCCC]

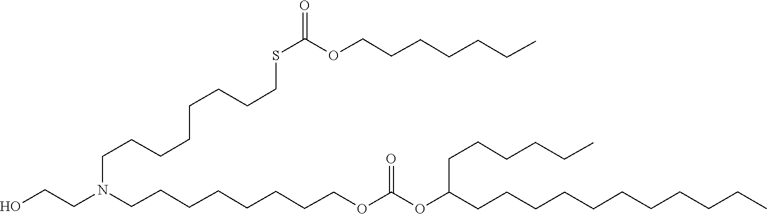 [SMILES: CCCCCCCCCCCC(CCCCCC)OC(=O)OCCCCCCCCN(CCO)CCCCCCCCSC(=O)OCCCCCCC]

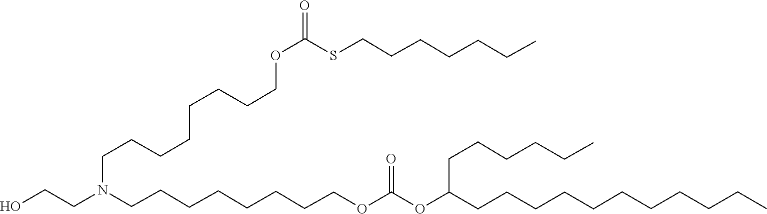 [SMILES: CCCCCCCCCCCC(CCCCCC)OC(=O)OCCCCCCCCN(CCO)CCCCCCCCOC(=O)SCCCCCCC]

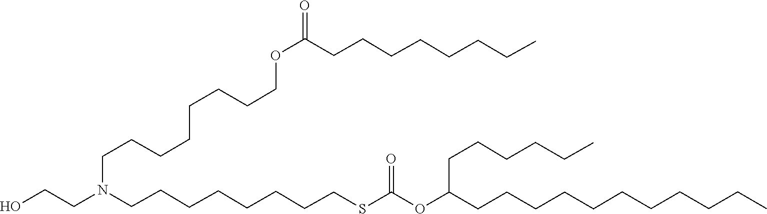 [SMILES: CCCCCCCCCCCC(CCCCCC)OC(=O)SCCCCCCCCN(CCO)CCCCCCCCOC(=O)CCCCCCCC]

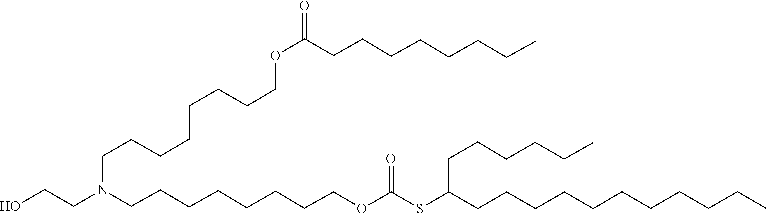 [SMILES: CCCCCCCCCCCC(CCCCCC)SC(=O)OCCCCCCCCN(CCO)CCCCCCCCOC(=O)CCCCCCCC]

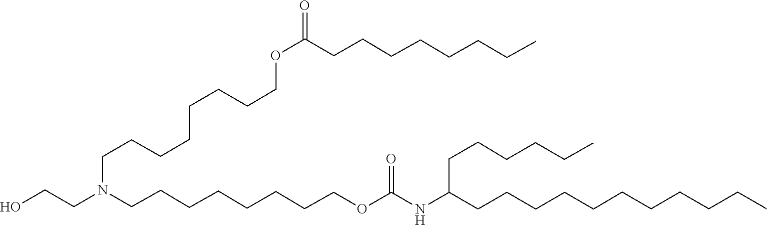 [SMILES: CCCCCCCCCCCC(CCCCCC)NC(=O)OCCCCCCCCN(CCO)CCCCCCCCOC(=O)CCCCCCCC]

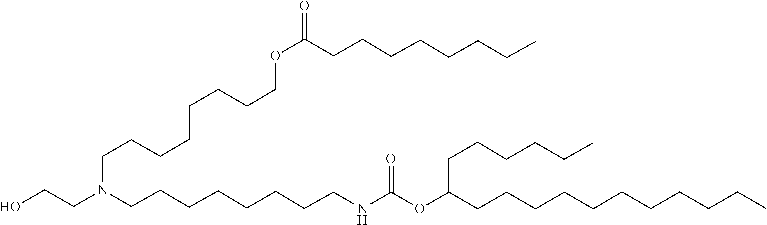 [SMILES: CCCCCCCCCCCC(CCCCCC)OC(=O)NCCCCCCCCN(CCO)CCCCCCCCOC(=O)CCCCCCCC]

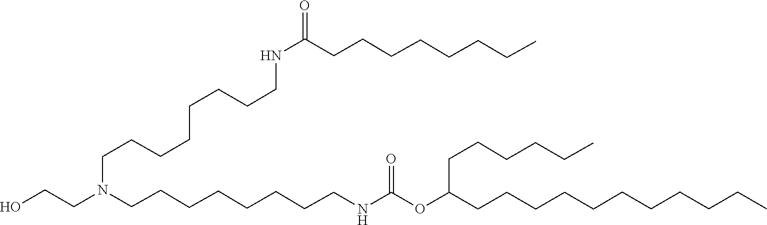 [SMILES: CCCCCCCCCCCC(CCCCCC)OC(=O)NCCCCCCCCN(CCO)CCCCCCCCNC(=O)CCCCCCCC]

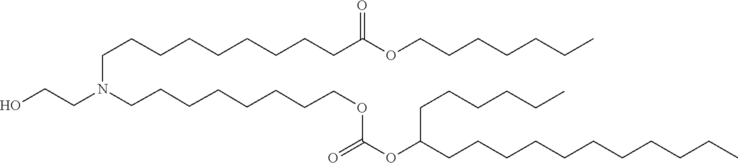 [SMILES: CCCCCCCCCCCC(CCCCCC)OC(=O)OCCCCCCCCN(CCO)CCCCCCCCCC(=O)OCCCCCCC]

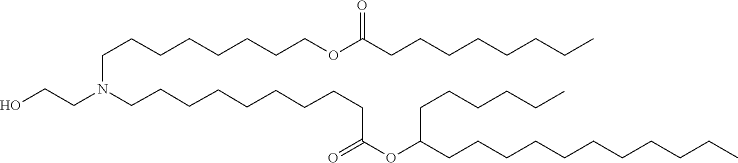 [SMILES: CCCCCCCCCCCC(CCCCCC)OC(=O)CCCCCCCCCN(CCO)CCCCCCCCOC(=O)CCCCCCCC]

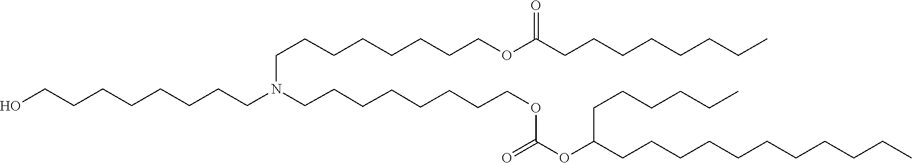 [SMILES: CCCCCCCCCCCC(CCCCCC)OC(=O)OCCCCCCCCN(CCCCCCCCO)CCCCCCCCOC(=O)CCCCCCCC]

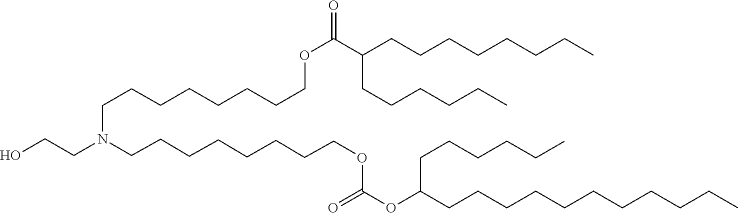 [SMILES: CCCCCCCCCCCC(CCCCCC)OC(=O)OCCCCCCCCN(CCO)CCCCCCCCOC(=O)C(CCCCCC)CCCCCCCC]

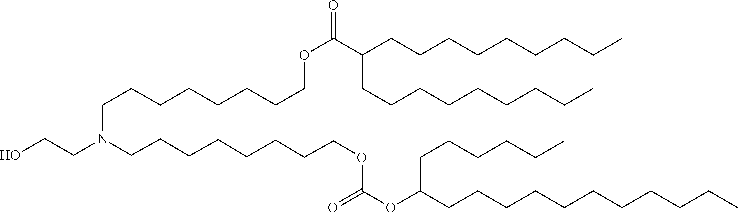 [SMILES: CCCCCCCCCCCC(CCCCCC)OC(=O)OCCCCCCCCN(CCO)CCCCCCCCOC(=O)C(CCCCCCCCC)CCCCCCCCC]

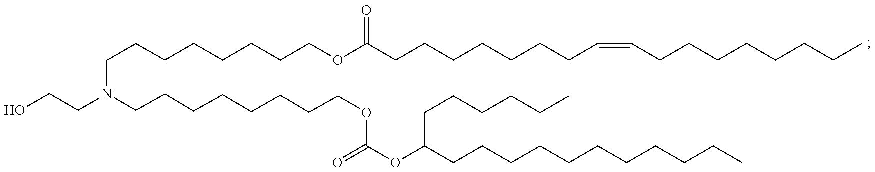 [SMILES: CCCCCCCC/C=C\CCCCCCCC(=O)OCCCCCCCCN(CCO)CCCCCCCCOC(=O)OC(CCCCCC)CCCCCCCCCCC]

or a salt thereof.

In any of the preceding embodiments, the salt of the lipidoid may be a pharmaceutically acceptable salt.

### Nanoparticle Compositions/Lipid Nanoformulations/Lipid-Based Carriers

In certain embodiments, provided herein is a nanoparticle composition comprising a plurality of a lipidoid of the disclosure, or a pharmaceutically acceptable salt thereof.

As used here, “nanoparticle composition” is used interchangeably with the terms “lipid-based carrier,” “lipid nanoformulation,” and “lipid nanoparticle.”

In certain embodiments, the nanoparticle composition further comprises a lipid. In certain such embodiments, the lipid is a cationic, anionic, ionizable, or zwitterionic lipid.

In some embodiments, compounds described herein are formulated into a lipid-based carrier (or lipid nanoformulation). In some embodiments, the lipid-based carrier (or lipid nanoformulation) is a liposome or a lipid nanoparticle (LNP). In one embodiment, the lipid-based carrier is an LNP.

In some embodiments, the lipid-based carrier (or lipid nanoformulation) comprises a cationic lipid (e.g., an ionizable lipid), a non-cationic lipid (e.g., phospholipid), a structural lipid (e.g., cholesterol), and a PEG-modified lipid. In some embodiments, the lipid-based carrier (or lipid nanoformulation) contains one or more compounds described herein, or a pharmaceutically acceptable salt thereof.

All above descriptions and all embodiments discussed in the above aspects relating to the aspects of the lipid compounds, including the compounds covered by formula (I), and the exemplary formulas for lipids having ionizable head groups are all applicable to these aspects of the invention relating to the lipid-based carriers (or a lipid nanoformulation).

As described herein, suitable compounds to be used in the lipid-based carrier (or lipid nanoformulation) include all the isomers and isotopes of the compounds described above, as well as all the pharmaceutically acceptable salts, solvates, or hydrates thereof, and all crystal forms, crystal form mixtures, and anhydrides or hydrates.

In addition to one or more compounds described herein, the lipid-based carrier (or lipid nanoformulation) may further include a second lipid. In some embodiments, the second lipid is a cationic lipid, a non-cationic (e.g., neutral, anionic, or zwitterionic) lipid, or an ionizable lipid.

One or more naturally occurring and/or synthetic lipid compounds may be used in the preparation of the lipid-based carrier (or lipid nanoformulation).

The lipid-based carrier (or lipid nanoformulation) may contain positively charged (cationic) lipids, neutral lipids, negatively charged (anionic) lipids, or a combination thereof.

In some embodiments, the lipid nanoparticle of the disclosure may be conjugated to a targeting moiety (e.g., an antibody or antigen-binding fragment thereof) through a linking group. Various linking groups known in the art may be used in the lipid nanoparticles of the disclosure, and can comprise one or more of optionally substituted alkylene, optionally substituted heteroalkylene, optionally substituted alkenylene, optionally substituted heteroalkenylene, optionally substituted alkynylene, optionally substituted heteroalkynylene, optionally substituted cycloalkylene, optionally substituted heterocycloalkylene, optionally substituted arylene, optionally substituted heteroarylene, a peptide moiety, a dipeptide moiety, —(C═O)—, a disulfide, a hydrazone, thioester, sulfone, sulfoxide, thiosulfinate, thiosulfonate, sulfate, sulfonate, sulfonylurea, ether, thioether, ester, amide, carbonate, carbamate, urea, sulfamide, succinimide, maleimide, phosphate, diphosphate, triazole, or a saccharide, or a combination thereof. Suitable linking groups are described, e.g., in WO 2024/015229, WO 2024/006272, and WO 2023/225359.

### Cationic Lipids (Positively Charged) and Ionizable Lipids

In some embodiments, the lipid-based carrier (or lipid nanoformulation) comprises one or more cationic lipids, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions.

Exemplary cationic lipids include one or more amine group(s) which bear the positive charge. Examples of positively charged (cationic) lipids include, but are not limited to, N,N′-dimethyl-N,N′-dioctacyl ammonium bromide (DDAB) and chloride DDAC), N-(1-(2,3-dioleyloxy)propyl)-N,N,N-trimethylammonium chloride (DOTMA), 3β-[N—(N′,N′-dimethylaminoethyl)carbamoyl) cholesterol (DC-chol), 1,2-dioleoyloxy-3-[trimethylammonio]-propane (DOTAP), 1,2-dioctadecyloxy-3-[trimethylammonio]-propane (DSTAP), and 1,2-dioleoyloxypropyl-3-dimethyl-hydroxy ethyl ammonium chloride (DORI), N,N-dioleyl-N,N-dimethylammonium chloride (DODAC), N,N-dimethyl-2,3-dioleyloxy)propylamine (DODMA), 1,2-Dioleoyl-3-Dimethylammonium-propane (DODAP), 1,2-Dioleoylcarbamyl-3-Dimethylammonium-propane (DOCDAP), 1,2-Dilineoyl-3-Dimethylammonium-propane (DLINDAP), 3-Dimethylamino-2-(Cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis, cis-9′,12′-octadecadienoxy)propane (CpLin DMA), N,N-Dimethyl-3,4-dioleyloxybenzylamine (DMOBA), and the cationic lipids described in e.g. Martin et al., Current Pharmaceutical Design, pages 1-394, which is herein incorporated by reference in its entirety. In some embodiments, the lipid-based carrier (or lipid nanoformulation) comprises more than one cationic lipid.

In some embodiments, the lipid-based carrier (or lipid nanoformulation) comprises a cationic lipid having an effective pKa over 6.0. In some embodiments, the lipid-based carrier (or lipid nanoformulation) further comprises a second cationic lipid having a different effective pKa (e.g., greater than the first effective pKa) than the first cationic lipid.

In some embodiments, cationic lipids that can be used in the lipid-based carrier (or lipid nanoformulation) include, for example those described in Table 4 of WO 2019/217941, which is incorporated by reference.

In some embodiments, the cationic lipid is an ionizable lipid (e.g., a lipid that is protonated at low pH, but that remains neutral at physiological pH). In some embodiments, the lipid-based carrier (or lipid nanoformulation) may comprise one or more additional ionizable lipids, different than the ionizable lipids described herein. Exemplary ionizable lipids include, but are not limited to,

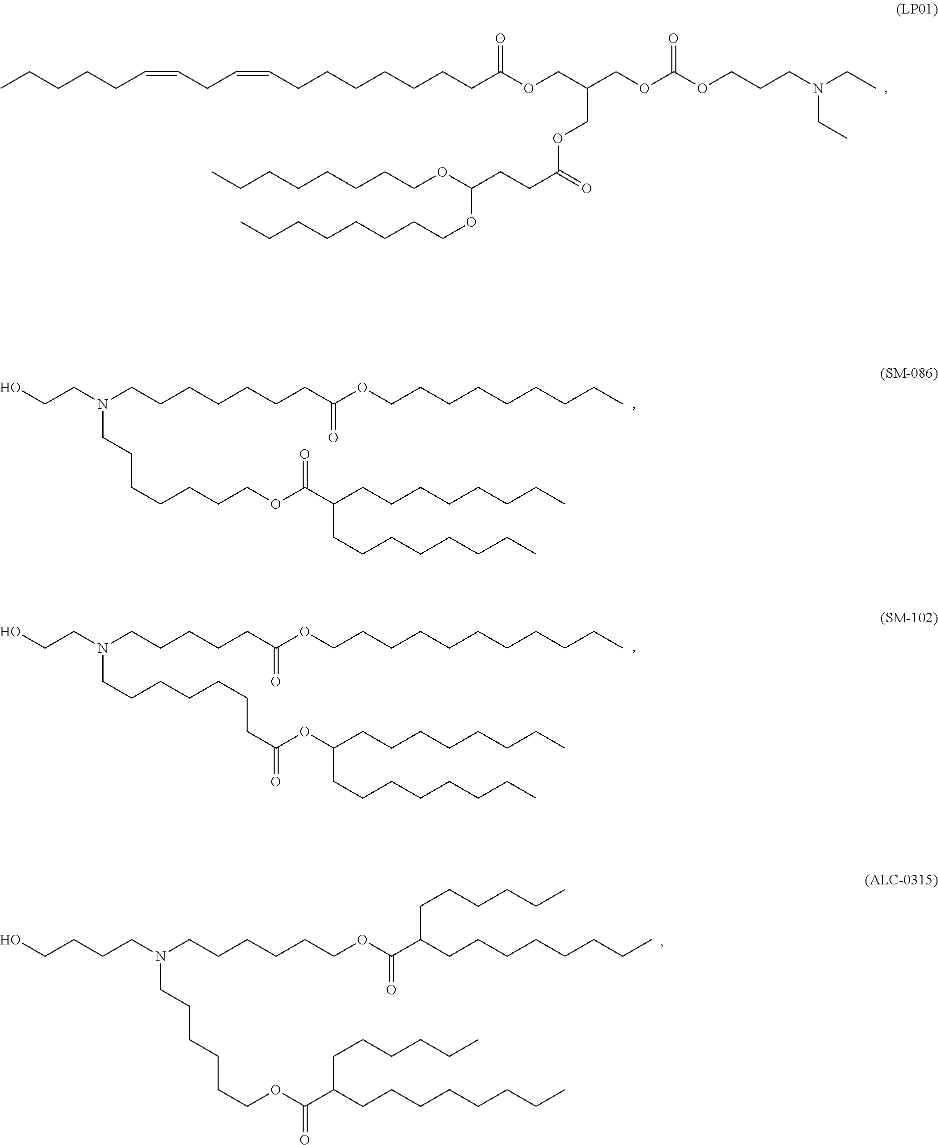 [SMILES: CCCCC/C=C\C/C=C\CCCCCCCC(=O)OCC(COC(=O)CCC(OCCCCCCCC)OCCCCCCCC)COC(=O)OCCCN(CC)CC ; CCCCCCCCCOC(=O)CCCCCCCN(CCO)CCCCCCCOC(=O)C(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CCCCCC)C(=O)OCCCCCCN(CCCCO)CCCCCCOC(=O)C(CCCCCC)CCCCCCCC]

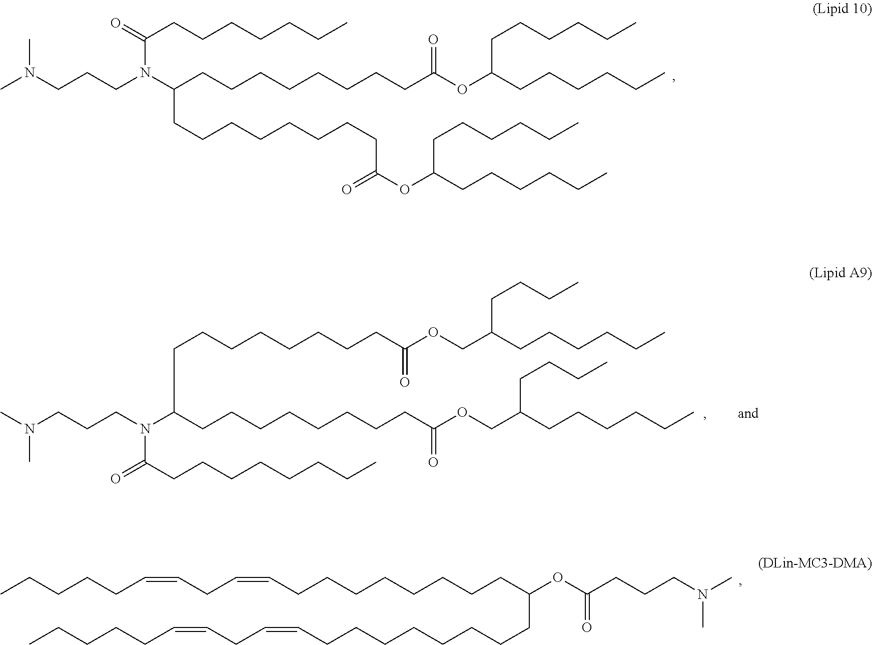

(see WO 2017/004143A1, which is incorporated herein by reference in its entirety).

In some embodiments, the lipid-based carrier (or lipid nanoformulation) further comprises one or more compounds described by WO 2021/113777 (e.g., a lipid of Formula (3) such as a lipid of Table 3 of WO 2021/113777), which is incorporated herein by reference in its entirety.

In one embodiment, the ionizable lipid is a lipid disclosed in Hou, X., et al. Nat Rev Mater 6, 1078-1094 (2021). https://doi.org/10.1038/s41578-021-00358-0 (e.g., L319, C12-200, and DLin-MC3-DMA), (which is incorporated by reference herein in its entirety).

Examples of other ionizable lipids that can be used in lipid-based carrier (or lipid nanoformulation) include, without limitation, one or more of the following formulas: X of US 2016/0311759; I of US 20150376115 or in US 2016/0376224; Compound 5 or Compound 6 in US 2016/0376224; I, IA, or II of U.S. Pat. No. 9,867,888; I, II or III of US 2016/0151284; I, IA, II, or IIA of US 2017/0210967; I-c of US 2015/0140070; A of US 2013/0178541; I of US 2013/0303587 or US 2013/0123338; I of US 2015/0141678; II, III, IV, or V of US 2015/0239926; I of US 2017/0119904; I or II of WO 2017/117528; A of US 2012/0149894; A of US 2015/0057373; A of WO 2013/116126; A of US 2013/0090372; A of US 2013/0274523; A of US 2013/0274504; A of US 2013/0053572; A of WO 2013/016058; A of WO 2012/162210; I of US 2008/042973; I, II, III, or IV of US 2012/01287670; I or II of US 2014/0200257; I, II, or III of US 2015/0203446; I or III of US 2015/0005363; I, IA, IB, IC, ID, II, IIA, IIB, IIC, IID, or III-XXIV of US 2014/0308304; of US 2013/0338210; I, II, III, or IV of WO 2009/132131; A of US 2012/01011478; I or XXXV of US 2012/0027796; XIV or XVII of US 2012/0058144; of US 2013/0323269; I of US 2011/0117125; I, II, or III of US 2011/0256175; I, II, III, IV, V, VI, VII, VIII, IX, X, XI, XII of US 2012/0202871; I, II, III, IV, V, VI, VII, VIII, X, XII, XIII, XIV, XV, or XVI of US 2011/0076335; I or II of US 2006/008378; I of WO2015/074085 (e.g., ATX-002); I of US 2013/0123338; I or X-A-Y—Z of US 2015/0064242; XVI, XVII, or XVIII of US 2013/0022649; I, II, or III of US 2013/0116307; I, II, or III of US 2013/0116307; I or II of US 2010/0062967; I-X of US 2013/0189351; I of US 2014/0039032; V of US 2018/0028664; I of US 2016/0317458; I of US 2013/0195920; 5, 6, or 10 of U.S. Pat. No. 10,221,127; III-3 of WO 2018/081480; I-5 or I-8 of WO 2020/081938; I of WO 2015/199952 (e.g., compound 6 or 22) and Table 1 therein; 18 or 25 of U.S. Pat. No. 9,867,888; A of US 2019/0136231; II of WO 2020/219876; 1 of US 2012/0027803; OF-02 of US 2019/0240349; 23 of U.S. Pat. No. 10,086,013; cKK-E12/A6 of Miao et al (2020); C12-200 of WO 2010/053572; 7C1 of Dahlman et al (2017); 304-013 or 503-013 of Whitehead et al; TS-P4C2 of U.S. Pat. No. 9,708,628; I of WO 2020/106946; I of WO 2020/106946; (1), (2), (3), or (4) of WO 2021/113777; and any one of Tables 1-16 of WO 2021/113777, all of which are incorporated herein by reference in their entirety.

In some embodiments, the lipid-based carrier (or lipid nanoformulation) further includes biodegradable ionizable lipids, for instance, (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). See, e.g., lipids of WO 2019/067992, WO 2017/173054, WO 2015/095340, and WO 2014/136086, which are incorporated herein by reference in their entirety.

In preferred embodiments, the cationic lipid is a lipidoid of any one of formulae (I), (II), (III), (IV), (V), (VI), (VII), or (VIII), disclosed herein, or an exemplary lipid of Table L, or a salt thereof.

Non-Cationic Lipids (e.g. Phospholipids)

In some embodiments, the lipid-based carrier (or lipid nanoformulation) further comprises one or more non-cationic lipids. In some embodiments, the non-cationic lipid is a phospholipid. In some embodiments, the non-cationic lipid is a phospholipid substitute or replacement. In some embodiments, the non-cationic lipid is a negatively charged (anionic) lipid.

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-phosphocholine (DLPC), Sodium 1,2-ditetradecanoyl-sn-glycero-3-phosphate (DMPA), phosphatidylcholine (lecithin), phosphatidylethanolamine, lysolecithin, lysophosphatidylethanolamine, phosphatidylserine, phosphatidylinositol, sphingomyelin, egg sphingomyelin (ESM), phosphatidylethanolamine (cephalin), cardiolipin, phosphatidic acid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, which is incorporated herein by reference. Such lipids include, in some embodiments, plant lipids found to improve liver transfection with mRNA (e.g., DGTS).

In some embodiments, the phospholipid is distearoylphosphatidylcholine (DSPC).

In some embodiments, the lipid-based carrier (or lipid nanoformulation) may comprise a combination of distearoylphosphatidylcholine/cholesterol, dipalmitoylphosphatidylcholine/cholesterol, dimyrystoylphosphatidylcholine/cholesterol, 1,2-Dioleoyl-sn-glycero-3-phosphocholine (DOPC)/cholesterol, or egg sphingomyelin/cholesterol.

Other examples of suitable non-cationic lipids include, without limitation, nonphosphorous lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO 2017/099823 or US 2018/0028664, which are incorporated herein by reference in their entirety.

In one embodiment, the lipid-based carrier (or lipid nanoformulation) further comprises one or more non-cationic lipid that is oleic acid or a compound of Formula I, II, or IV of US 2018/0028664, which is incorporated herein by reference in its entirety.

The non-cationic lipid content can be, for example, 0-30% (mol) of the total lipid components present. In some embodiments, the non-cationic lipid content is 5-20% (mol) or 10-15% (mol) of the total lipid components present.

In some embodiments, the lipid-based carrier (or lipid nanoformulation) further comprises a neutral lipid, and the molar ratio of an ionizable lipid to a neutral lipid ranges from about 2:1 to about 8:1 (e.g., about 2:1, 3:1, 4:1, 5:1, 6:1, 7:1, or 8:1).

In some embodiments, the lipid-based carrier (or lipid nanoformulation) does not include any phospholipids.

In some embodiments, the lipid-based carrier (or lipid nanoformulation) can further include one or more phospholipids, and optionally one or more additional molecules of similar molecular shape and dimensions having both a hydrophobic moiety and a hydrophilic moiety (e.g., cholesterol).

Exemplary anionic lipids include dimyrystoyl-, dipalmitoyl-, and distearoyl-phasphatidylglycerol; dimyrystoyl-, dipalmitoyl-, and dipalmitoyl-phosphatidic acid; dimyrystoyl-, dipalmitoyl-, and dipalmitoyl-phosphatidylethanolamine; and their unsaturated diacyl and mixed acyl chain counterparts as well as cardiolipin.

Exemplary neutral lipids include DLPC (1,2-dilauroyl-sn-glycero-3-phosphocholine), DMPC (1,2-dimyristoyl-sn-glycero-3-phosphocholine), DPPC (1,2-dipalmitoyl-sn-glycero-3-phosphocholine), DSPC (1,2-distearoyl-sn-glycero-3-phosphocholine), DOPC (1,2-dioleoyl-sn-glycero-3-phosphocholine), DMPA (Sodium 1,2-ditetradecanoyl-sn-glycero-3-phosphate), DPPE (1,2-Dipalmitoyl-sn-glycero-3-phosphoethanolamine), and DOPE (1,2-dioleoyl-sn-glycero-3-phosphoethanolamine).

Exemplary phospholipids include, but are not limited to, phosphatidylcholine (lecithin), lysolecithin, lysophosphatidylethanol-amine, phosphatidylserine, phosphatidylinositol, sphingomyelin, phosphatidylethanolamine (cephalin), cardiolipin, phosphatidic acid, cerebrosides, dicetylphosphate, phosphatidylcholine, and dipalmitoylphosphatidylglycerol.

### Structural Lipids

The lipid-based carrier (or lipid nanoformulation) described herein may further comprise one or more structural lipids. As used herein, the term “structural lipid” refers to sterols (e.g., cholesterol and derivatives thereof) and to lipids containing sterol moieties.

Incorporation of structural lipids in the lipid nanoparticle may help mitigate aggregation of other lipid in the particle. Structural lipids can be selected from the group including but not limited to, cholesterol or cholesterol derivative, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, structural lipids may be incorporated into the lipid-based carrier at molar ratios ranging from about 0.1 to 1.0 (cholesterol phospholipid).

In some embodiments, sterols, when present, can include one or more of cholesterol or cholesterol derivatives, such as those described in WO 2009/127060 or US 2010/0130588, which are incorporated herein by reference in their entirety. Additional exemplary sterols include phytosterols, including those described in Eygeris et al. (2020), Nano Lett. 2020; 20(6):4543-4549, incorporated herein by reference.

In some embodiments, the structural lipid is a cholesterol derivative. Non-limiting examples of cholesterol derivatives include polar analogues such as 5a-cholestanol, 53-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in WO 2009/127060 and US 2010/0130588, each of which is incorporated herein by reference in its entirety.

In some embodiments, the lipid-based carrier (or lipid nanoformulation) further comprises sterol in an amount of 0-50 mol % (e.g., 0-10 mol %, 10-20 mol %, 20-50 mol %, 20-30 mol %, 30-40 mol %, or 40-50 mol %) of the total lipid components.

### Polymers and Polyethylene Glycol (PEG) Lipids

In some embodiments, the lipid-based carrier (or lipid nanoformulation) may include one or more polymers or co-polymers, e.g., poly(lactic-co-glycolic acid) (PFAG) nanoparticles.

In some embodiments, the lipid-based carrier (or lipid nanoformulation) may include one or more polyethylene glycol (PEG) lipid (also referred to as a “PEGylated lipid”). Examples of useful PEG-lipids include, but are not limited to, 1,2-Diacyl-sn-Glycero-3-Phosphoethanolamine-N-[Methoxy(Polyethylene glycol)-350](mPEG 350 PE); 1,2-Diacyl-sn-Glycero-3-Phosphoethanolamine-N-[Methoxy(Polyethylene glycol)-550](mPEG 550 PE); 1,2-Diacyl-sn-Glycero-3-Phosphoethanolamine-N-[Methoxy(Polyethylene glycol)-750](mPEG 750 PE); 1,2-Diacyl-sn-Glycero-3-Phosphoethanolamine-N-[Methoxy(Polyethylene glycol)-1000](mPEG 1000 PE); 1,2-Diacyl-sn-Glycero-3-Phosphoethanolamine-N—[Methoxy(Polyethylene glycol)-2000](mPEG 2000 PE); 1,2-Diacyl-sn-Glycero-3-Phosphoethanolamine-N—[Methoxy(Polyethylene glycol)-3000](mPEG 3000 PE); 1,2-Diacyl-sn-Glycero-3-Phosphoethanolamine-N-[Methoxy(Polyethylene glycol)-5000](mPEG 5000 PE); N-Acyl-Sphingosine-1-[Succinyl(Methoxy Polyethylene Glycol) 750](mPEG 750 Ceramide); N-Acyl-Sphingosine-1-[Succinyl(Methoxy Polyethylene Glycol) 2000](mPEG 2000 Ceramide); and N-Acyl-Sphingosine-1-[Succinyl(Methoxy Polyethylene Glycol) 5000](mPEG 5000 Ceramide). In some embodiments, the PEG lipid is a polyethyleneglycol-diacylglycerol (i.e., polyethyleneglycol diacylglycerol (PEG-DAG), PEG-cholesterol, or PEG-DMB) conjugate. In some embodiments, the PEG lipid is DSPE-PEG2k-DBCO, a PEG lipid conjugate featuring a DSPE phospholipid and a DBCO group.

In some embodiments, the lipid-based carrier (or nanoformulation) includes one or more conjugated lipids (such as PEG-conjugated lipids or lipids conjugated to polymers described in Table 5 of WO 2019/217941, which is incorporated herein by reference in its entirety). In some embodiments, the one or more conjugated lipids is formulated with one or more ionic lipids (e.g., non-cationic lipid such as a neutral or anionic, or zwitterionic lipid); and one or more sterols (e.g., cholesterol).

The PEG conjugate can comprise a PEG-dilaurylglycerol (C12), a PEG-dimyristylglycerol (C14), a PEG-dipalmitoylglycerol (C16), a PEG-disterylglycerol (C18), PEG-dilaurylglycamide (C12), PEG-dimyristylglycamide (C14), PEG-dipalmitoylglycamide (C16), and PEG-disterylglycamide (C18).

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, and those described in Table 2 of WO 2019/051289 (which is herein incorporated by reference in its entirety), and combinations of the foregoing.

In some embodiments, the conjugated lipid is DMG-PEG2k.

In some embodiments, the conjugated lipid comprises GalNAC. In certain embodiments, the conjugated lipid comprising GalNAc is DSPE-PEG2k-GalNAc3.

Additional exemplary PEG-lipid conjugates are described, for example, in U.S. Pat. Nos. 5,885,613, 6,287,591, US 2003/0077829, US 2003/0077829, US 2005/0175682, US 2008/0020058, US 2011/0117125, US 2010/0130588, US 2016/0376224, US 2017/0119904, US 2018/0028664, and WO 2017/099823, all of which are incorporated herein by reference in their entirety.

In some embodiments, the PEG-lipid is a compound of Formula III, III-a-I, III-a-2, III-b-1, III-b-2, or V of US 2018/0028664, which is incorporated herein by reference in its entirety. In some embodiments, the PEG-lipid is of Formula II of US 2015/0376115 or US 2016/0376224, both of which are incorporated herein by reference in their entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. In some embodiments, the PEG-lipid includes one of the following:

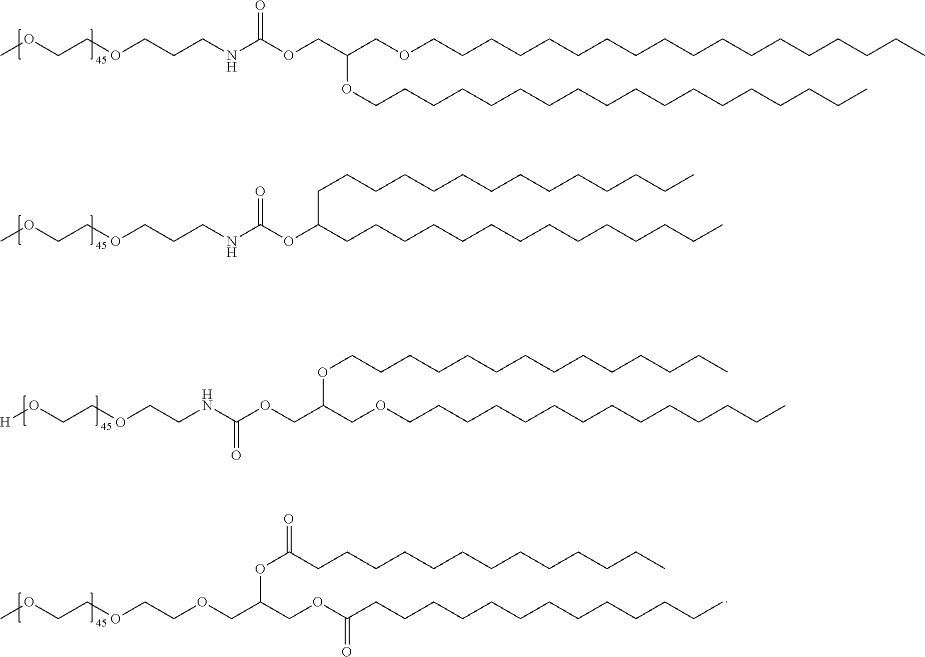

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, e.g., PEG-lipids, (POZ)-lipid conjugates, ATTA-lipid conjugates and cationic polymer-lipids, include those described in Table 2 of WO 2019/051289A9, which is incorporated herein by reference in its entirety.

In some embodiments, the conjugated lipid (e.g., the PEGylated lipid) can be present in an amount of 0-20 mol % of the total lipid components present in the lipid-based carrier (or lipid nanoformulation). In some embodiments, the conjugated lipid (e.g., the PEGylated lipid) content is 0.5-10 mol % or 2-5 mol % of the total lipid components.

When needed, the lipid-based carrier (or lipid nanoformulation) described herein may be coated with a polymer layer to enhance stability in vivo (e.g., sterically stabilized LNPs).

Examples of suitable polymers include, but are not limited to, poly(ethylene glycol), which may form a hydrophilic surface layer that improves the circulation half-life of liposomes and enhances the amount of lipid nanoformulations (e.g., liposomes or LNPs) that reach therapeutic targets. See, e.g., Working et al. J Pharmacol Exp Ther, 289: 1128-1133 (1999); Gabizon et al., J Controlled Release 53: 275-279 (1998); Adlakha Hutcheon et al., Nat Biotechnol 17: 775-779 (1999); and Koning et al., Biochim Biophys Acta 1420: 153-167 (1999), which are incorporated herein by reference in their entirety.

In certain embodiments, the nanoparticle composition further comprises a PEGylated lipid, a sterol, a phospholipid, and/or a neutral lipid.

### Percentages of Lipid Nanoformulation Components

In some embodiments, the lipid-based carrier (or lipid nanoformulation) comprises one or more of the compounds described herein, optionally a non-cationic lipid (e.g., a phospholipid), a sterol, a neutral lipid, and/or optionally conjugated lipid (e.g., a PEGylated lipid) that inhibits aggregation of particles. The relative amounts of these components can be varied independently and to achieve desired properties. For example, in some embodiments, the ionizable lipid including the lipid compounds described herein is present in an amount from about 20 mol % to about 100 mol % (e.g., 20-90 mol %, 20-80 mol %, 20-70 mol %, 25-100 mol %, 30-70 mol %, 30-60 mol %, 30-40 mol %, 40-50 mol %, or 50-90 mol %) of the total lipid and lipidoid components; a non-cationic lipid (e.g., phospholipid) is present in an amount from about 0 mol % to about 50 mol % (e.g., 0-40 mol %, 0-30 mol %, 5-50 mol %, 5-40 mol %, 5-30 mol %, or 5-10 mol %) of the total lipid and lipidoid components, a conjugated lipid (e.g., a PEGylated lipid) in an amount from about 0.5 mol % to about 20 mol % (e.g., 1-10 mol % or 5-10%) of the total lipid and lipidoid components, and a sterol in an amount from about 0 mol % to about 60 mol % (e.g., 0-50 mol %, 10-60 mol %, 10-50 mol %, 15-60 mol %, 15-50 mol %, 20-50 mol %, 20-40 mol %) of the total lipid and lipidoid components, provided that the total mol % of the lipid component does not exceed 100%.

As used herein, the term “total lipid component” of the lipid nanoparticle refers to the aggregated amounts of all lipid and lipidoid components present in the composition. For example, if a composition consisted of 25 mol % lipid A, 25 mol % lipid B, 25 mol % non-lipid C, and 25 mol % non-lipid D, then the total lipid component of the composition is 50 mol %, and each of lipid A and lipid B is 50 mol % of the total lipid component of the composition.

In some embodiments, the lipid-based carrier (or lipid nanoformulation) comprises about 25-100 mol % of the ionizable lipid including the lipid and lipidoid compounds described herein, about 0-50 mol % phospholipid, about 0-50 mol % sterol, and about 0-10 mol % PEGylated lipid. In some embodiments, the lipid-based carrier (or lipid nanoformulation) comprises about 32-39 mol % ionizable lipid including the lipid and lipidoid compounds described herein, about 25-30 mol % phospholipid, about 31-34 mol % sterol, and about 0.6-1.9% PEGylated lipid. In some embodiments, the lipid-based carrier (or lipid nanoformulation) comprises about 32-39 mol % ionizable lipid including the lipid and lipidoid compounds described herein, about 25-30 mol % DSPC, about 31-34 mol % cholesterol, and about 0.6-1.9% DMG-PEG2k.

In one embodiment, the lipid-based carrier (or lipid nanoformulation) comprises about 25-100 mol % of the ionizable lipid including the lipid and lipidoid compounds described herein; about 0-40 mol % phospholipid (e.g., DSPC), about 0-50 mol % sterol (e.g., cholesterol), and about 0-10 mol % PEGylated lipid.

In some embodiments, the lipid-based carrier (or lipid nanoformulation) comprises about 30-60 mol % (e.g., about 35-55 mol %, or about 40-50 mol %) of the ionizable lipid including the lipid and lipidoid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid.

In some embodiments, molar ratios of ionizable lipid/sterol/phospholipid (or another structural lipid)/PEG-lipid/additional components is varied in the following ranges: ionizable lipid (25-100%); phospholipid (DSPC) (0-40%); sterol (0-50%); and PEG lipid (0-5%).

In some embodiments, the lipid-based carrier (or lipid nanoformulation) comprises, by mol % or wt % of the total lipid and lipidoid components, 50-75% ionizable lipid (including the lipid and lipidoid compounds as described herein), 20-40% sterol (e.g., cholesterol or derivative), 0 to 10% non-cationic-lipid, and 1-10% conjugated lipid (e.g., the PEGylated lipid).

In some embodiments, the lipidoid compound described herein is a component of the lipid-based carrier (or lipid nanoformulation, or nanoparticle composition) and comprises from 10 mol % to 95 mol %, from 10 mol % to 90 mol %, from 10 mol % to 80 mol %, from 10 mol % to 70 mol %, from 10 mol % to 60 mol %, from 20 mol % to 55 mol %, from 20 mol % to 45 mol %, 20 mol % to 40 mol %, from 25 mol % to 50 mol %, from 25 mol % to 45 mol %, from 30 mol % to 50 mol %, from 30 mol % to 45 mol %, from 30 mol % to 40 mol %, from 32 mol % to 39 mol %, from 35 mol % to 45 mol %, or from 37 mol % to 42 mol % (or any fraction of these ranges) of the total lipid and lipidoid components.

In some embodiments, where the lipid-based carrier (or lipid nanoformulation) contains a mixture of phospholipid and sterol (e.g. cholesterol or derivative), the mixture may be present up to 40 mol %, 45 mol %, 50 mol %, 55 mol %, or 60 mol % of the total lipid and lipidoid components.

In some embodiments, the phospholipid component in the mixture may be present from 2 mol % to 20 mol %, from 2 mol % to 15 mol %, from 2 mol % to 12 mol %, from 4 mol % to 15 mol %, from 4 mol % to 10 mol %, from 5 mol % to 10 mol %, from 25 mol % to 30 mol %, (or any fraction of these ranges) of the total lipid and lipidoid components. In some embodiments, the lipid-based carrier (or lipid nanoformulation or nanoparticle composition) is substantially free of a phospholipid. In certain embodiments, the lipid-based carrier (or lipid nanoformulation or nanoparticle composition) is substantially free of distearolyphosphatidycholine (DSPC).

In some embodiments, the sterol component (e.g. cholesterol or derivative) in the mixture may comprise from 25 mol % to 45 mol %, from 25 mol % to 40 mol %, from 25 mol % to 35 mol %, from 25 mol % to 30 mol %, from 30 mol % to 45 mol %, from 30 mol % to 40 mol %, from 30 mol % to 35 mol %, from 31 mol % to 34 mol %, from 35 mol % to 40 mol %, from 27 mol % to 37 mol %, or from 27 mol % to 35 mol % (or any fraction of these ranges) of the total lipid and lipidoid components.

In some embodiments, the conjugated lipid component in the mixture may be present from 0.1 mol % to 10 mol %, from 0.1 mol % to 5 mol %, from 0.1 mol % to 2.5 mol %, from 0.1 mol % to 2.0 mol %, from 0.1 mol % to 1.5 mol %, from 0.2 mol % to 2.0 mol %, from 0.3 mol % to 1.5 mol %, from 0.4 mol % to 1.5 mol %, from 0.5 mol % to 1.5 mol %, from 0.5 mol % to 1.2 mol %, from 0.6 mol % to 1.2 mol %, or from 0.7 mol % to 1.1 mol % of the total lipid and lipidoid components.

In some embodiments, where the lipid-based carrier (or lipid nanoformulation) is phospholipid-free, the sterol component (e.g. cholesterol or derivative) may be present up to 25 mol %, 30 mol %, 35 mol %, 40 mol %, 45 mol %, 50 mol %, 55 mol %, or 60 mol % of the total lipid and lipidoid components. For instance, the sterol component (e.g. cholesterol or derivative) may be present from 25 mol % to 65 mol %, from 25 mol % to 60 mol %, from 25 mol % to 55 mol %, from 25 mol % to 50 mol %, from 25 mol % to 45 mol %, from 25 mol % to 40 mol %, from 30 mol % to 45 mol %, from 30 mol % to 40 mol %, from 35 mol % to 45 mol %, from 30 mol % to 35 mol %, or from 35 mol % to 40 mol % (or any fraction thereof or range therein) of the total lipid and lipidoid components.

In some embodiments, the non-ionizable lipid components in the lipid-based carrier (or lipid nanoformulation) may be present from 5 mol % to 90 mol %, from 10 mol % to 85 mol %, or from 20 mol % to 80 mol % (or any fraction of these ranges) of the total lipid and lipidoid components.

The ratio of total lipid components to the cargo (e.g., an encapsulated therapeutic agent such as a nucleic acid) can be varied as desired. For example, the total lipid components to the cargo (mass or weight) ratio can be from about 10:1 to about 30:1. In some embodiments, the total lipid and lipidoid components to the cargo ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of total lipid components and the cargo can be adjusted to provide a desired N/P ratio, for example, N/P ratio of 3, 4, 5, 6, 7, 8, 9, 10, 11, 12, 13, 14, 15, 16, 17, 18, 19, 20, 21, 22, 23, 24, 25, 26, 27, 28, 29, 30, or higher. Generally, the lipid-based carrier (or lipid nanoformulation)'s overall lipid content can range from about 5 mg/mL to about 30 mg/mL. Nitrogen:phosphate ratios (N:P ratio) is evaluated at values between 0.1 and 100.

In some embodiments, the lipid-based carrier (or lipid nanoformulation) includes the ionizable lipid compound as described herein, phospholipid, cholesterol, and a PEGylated lipid in a molar ratio of 50:10:38.5:1.5. In some embodiments, the lipid-based carrier (or lipid nanoformulation) includes the ionizable lipid compound as described herein, cholesterol and a PEGylated lipid in a molar ratio of 60:38.5:1.5.

In some embodiments of any of the aspects or embodiments herein, the lipid-based carrier (or lipid nanoformulation) further comprises a tissue targeting moiety. The tissue targeting moiety can be a peptide, oligosaccharide or the like, which can be used for the delivery of the lipid-based carrier (or lipid nanoformulation) to one or more specific tissues such as the liver. In some embodiments, the tissue targeting moiety is a ligand for liver specific receptors. In certain embodiments, the ligand of liver specific receptors used for liver targeting is an oligosaccharide such as N-Acetylgalactosamine (GalNAc) which is covalently attached to a component of a lipid-based carrier (or lipid nanoformulation), e.g., PEG-lipid conjugates or the like. In some embodiments, the GalNAc is covalently attached to, for example, PEG-lipid conjugate. In some embodiments, the GalNAc is conjugated to DSPE-PEG2000. In some embodiments, the GalNAc-PEG-lipid conjugate is present in the lipid-based carrier (or lipid nanoformulation) at a molar percentage of 1.5%, 1.4%, 1.3%, 1.2%, 1.1%, 1.0%, 0.9%, 0.8%, 0.7%, 0.6%, 0.5%, 0.4%, 0.3%, 0.2%, or 0.1% of the total lipid. In some embodiments, the GalNAc-PEG-lipid conjugate is present in the lipid-based carrier (or lipid nanoformulation) at a molar percentage of 0.2% of the total lipid. In some embodiments, the GalNAc-PEG-lipid conjugate is present in the lipid-based carrier (or lipid nanoformulation) at a molar percentage of 0.3% of the total lipid. In some embodiments, the GalNAc-PEG-lipid conjugate is present in the lipid-based carrier (or lipid nanoformulation) at a molar percentage of 0.4% of the total lipid. In some embodiments, the GalNAc-PEG-lipid conjugate is present in the lipid-based carrier (or lipid nanoformulation) at a molar percentage of 0.5% of the total lipid. In some embodiments, the GalNAc-PEG-lipid conjugate is present in the lipid-based carrier (or lipid nanoformulation) at a molar percentage of 0.6% of the total lipid. In some embodiments, the GalNAc-PEG-lipid conjugate is present in the lipid-based carrier (or lipid nanoformulation) at a molar percentage of 0.7% of the total lipid. In some embodiments, the GalNAc-PEG-lipid conjugate is present in the lipid-based carrier (or lipid nanoformulation) at a molar percentage of 0.8% of the total lipid. In some embodiments, the GalNAc-PEG-lipid conjugate is present in the lipid-based carrier (or lipid nanoformulation) at a molar percentage of 0.9% of the total lipid. In some embodiments, the GalNAc-PEG-lipid conjugate is present in the lipid-based carrier (or lipid nanoformulation) at a molar percentage of 1.0% of the total lipid. In some embodiments, the GalNAc-PEG-lipid conjugate is present in the lipid-based carrier (or lipid nanoformulation) at a molar percentage of about 1.5% of the total lipid. In some embodiments, the GalNAc-PEG-lipid conjugate is present in the lipid-based carrier (or lipid nanoformulation) at a molar percentage of 2.0% of the total lipid.

In certain embodiments, the disclosure provides a nanoparticle composition comprising:
i. a nucleic acid;
ii. a lipidoid of any one of formulae (I), (II), (III), (IV), (V), (VI), (VII), or (VIII), disclosed herein, or an exemplary lipid of Table L, or a salt thereof comprising from 32 mol % to 39 mol % of the total lipid component present in the nanoparticle;
iii. a phospholipid or derivative thereof comprising from 25 mol % to 30 mol % of the total lipid component present in the nanoparticle;
iv. a cholesterol or derivative thereof comprising from 31 mol % to 34 mol % of the total lipid component present in the nanoparticle; and
v. a conjugated lipid that inhibits aggregation of particles comprising from 0.7 mol % to 1.1 mol % of the total lipid component present in the nanoparticle.

In certain aspects, the nanoparticle composition further comprises GalNAc.

In certain aspects, the nanoparticle composition further comprises between 0.3 mol % to 0.7 mol % GalNAc.

In certain aspects, the nanoparticle composition further comprises 0.5 mol % GalNAc.

In some embodiments, the nanoparticle composition comprises:
i. a nucleic acid;
ii. a cationic lipid comprising 35.7 mol % of the total lipid component present in the nanoparticle;
iii. a phosopholipid comprising 29.9 mol % of the total lipid component present in the nanoparticle;
iv. a cholesterol comprising 33.1 mol % of the total lipid component present in the nanoparticle;
v. a conjugated lipid comprising 0.8 mol % of the total lipid component present in the nanoparticle; and
vi. a conjugated lipid comprising GalNAc that comprises 0.5 mol % of the total lipid component present in the nanoparticle.

In some embodiments, the nanoparticle composition comprises Lipid 1:

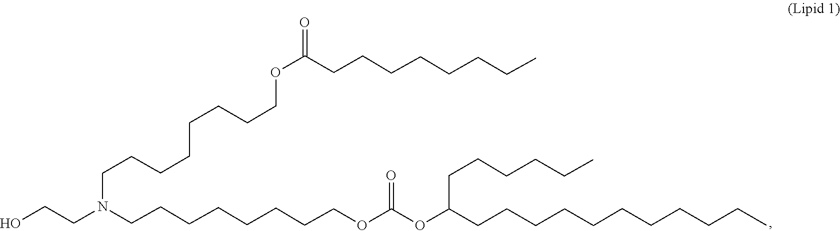 [SMILES: CCCCCCCCCCCC(CCCCCC)OC(=O)OCCCCCCCCN(CCO)CCCCCCCCOC(=O)CCCCCCCC]

or a pharmaceutically acceptable salt thereof.

In certain aspects, the phospholipid is distearoylphosphatidylcholine DSPC.

In certain aspects, the conjugated lipid is DMG-PEG2k.

In certain aspects, the conjugated lipid comprising GalNAc is DSPE-PEG2k-GalNAc3.

In some embodiments, the present disclosure provides a nanoparticle composition comprising:
i. a nucleic acid;
ii. 35.7 mol % of lipid 1 or a pharmaceutically acceptable salt thereof;
iii. 29.9 mol % of distearoylphosphatidylcholine (DSPC);
iv. 33.1 mol % of cholesterol;
v. 0.8 mol % of DMG-PEG2k; and
vi. 0.5 mol % of DSPE-PEG2k-GalNAc3.

In some embodiments, the compositions described herein are useful for delivering a payload to a liver cell. In some embodiments, the composition is selective for a liver cell. In some embodiments, the compositions described herein are useful for delivering a payload to a an organ. In some embodiments, the composition targets to the liver. In some embodiments, the composition targets the liver as compared to the spleen. In some embodiments, the composition increases expression of a payload in the liver as compared to the spleen. In some embodiments, the composition increases expression of a payload in the liver.

### Properties of Lipid Nanoformulations

In some embodiments, the average particle diameter of the lipid-based carrier (or lipid nanoformulation) may be between 10s of nm and 100s of nm, e.g., measured by dynamic light scattering (DLS). In some embodiments, the average particle diameter of the lipid-based carrier (or lipid nanoformulation) ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, from about 38 mm to about 42 mm, from about 40 nm to about 150 nm (such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm), from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm.

The lipid-based carrier or lipid nanoformulation (e.g., liposome or LNP) may, in some instances, be relatively homogenous. A polydispersity index may be used to indicate the homogeneity of a lipid nanoformulation (e.g., liposome or LNP), e.g., the particle size distribution of the liposome or LNP. A small (e.g., less than 0.3) polydispersity index generally indicates a narrow particle size distribution. A lipid-based carrier or lipid nanoformulation (e.g., liposome or LNP) may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of the lipid-based carrier or lipid nanoformulation (e.g., liposome or LNP) may be from about 0.10 to about 0.20.

The zeta potential of a lipid-based carrier or a lipid nanoformulation (e.g., liposome or LNP) may be used to indicate the electrokinetic potential of the composition. In some embodiments, the zeta potential may describe the surface charge of a liposome or LNP. Lipid nanoformulations (e.g., liposomes or LNP) with relatively low charges, positive or negative, are generally desirable, as more highly charged species may interact undesirably with cells, tissues, and other elements in the body. In some embodiments, the zeta potential of a liposome or LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a cargo such as a protein and/or nucleic acid, describes the amount of protein and/or nucleic acid that is encapsulated or otherwise associated with a lipid nanoformulation (e.g., liposome or LNP) after preparation, relative to the initial amount provided. The encapsulation efficiency is desirably high (e.g., at least 70%, 80%. 90%, 95%, close to 100%). The encapsulation efficiency may be measured, for example, by comparing the amount of protein or nucleic acid in a solution containing the liposome or LNP before and after breaking up the liposome or LNP with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the liposome or LNP described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

The lipid carrier or lipid nanoformulation may optionally include one or more coatings. In some embodiments, the lipid carrier or lipid nanoformulation (e.g., liposome or LNP) may be formulated in a capsule, film, or tablet having a coating. A capsule, film, or tablet including a composition described herein may have any useful size, tensile strength, hardness, or density.

Additional exemplary lipids, formulations, methods, and characterization of a lipid carrier or lipid nanoformulation (e.g., liposome or LNP) are taught by WO 2020/061457 and WO 2021/113777, which are incorporated herein by reference in their entirety. Further exemplary lipids, formulations, methods, and characterization of LNPs are taught by Hou et al. Lipid nanoparticles for mRNA delivery. Nat Rev Mater (2021). doi.org/10.1038/s41578-021-00358-0, which is incorporated herein by reference in its entirety (see, for example, exemplary lipids and lipid derivatives of FIG. 2 of Hou et al.).

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51(34):8529-8533 (2012), incorporated herein by reference in its entirety.

Lipid nanoformulations (e.g., liposome or LNP) optimized for the delivery of CRISPR-Cas systems, e.g., Cas9-gRNA RNP, gRNA, Cas9 mRNA, are described in WO 2019067992 and WO 2019067910, which are incorporated by reference in their entirety.

Additional specific lipid nanoformulations (e.g., liposome or LNP) useful for delivery of nucleic acid effector molecules are described in U.S. Pat. Nos. 8,158,601 and 8,168,775, which are incorporated by reference in their entirety.

A variety of methods can be used for preparing the lipid carrier or lipid nanoformulation (e.g., liposomes or LNPs) described herein. Such methods are known in the art or disclosed herein, for example, the methods described in Lichtenberg and Barenholz in Methods of Biochemical Analysis, 33:337-462 (1988), which is incorporated herein by reference in its entirety. See also Szoka et al., Ann. Rev. Biophys. Bioeng. 9:467 (1980); U.S. Pat. Nos. 4,235,871; 4,501,728; and 4,837,028; Liposomes, Marc J. Ostro, ed., Marcel Dekker, Inc., New York, 1983, Chapter 1; and Hope, et al., Chem. Phys. Lip. 40:89 (1986), which are incorporated herein by reference in their entirety. Small unilamellar vesicles (SUV, size <100 nm) can be prepared by a combination of standard methods of thin-film hydration and repeated extrusion.

Techniques for sizing the lipid carrier or lipid nanoformulations (e.g., liposomes or LNPs) to a desired size are well-known to one skilled in the art. See, e.g., U.S. Pat. No. 4,737,323, and Hope et al., Biochim. Biophys. Acta, 812: 55-65, which are incorporated by reference in their entirety. Sonicating a lipid nanoformulation (e.g., liposome or LNP) suspension either by bath or probe sonication produces a progressive size reduction down to small unilamellar vesicles less than about 50 nm in size. Homogenization or microfluidization are other methods which rely on shearing energy to fragment large lipid nanoformulations (e.g., liposomes or LNPs) into smaller ones. In a typical homogenization procedure, multilamellar vesicles are recirculated through a standard emulsion homogenizer until selected lipid nanoformulation (e.g., liposome or LNP) sizes, typically between about 100 and 500 nm, are observed. In both methods, the particle size distribution can be monitored by conventional laser-beam particle size discrimination.

Extrusion of lipid nanoformulations (e.g., liposomes or LNPs) through a small-pore polycarbonate membrane or an asymmetric ceramic membrane is a very effective method for reducing liposome or LNP sizes to a relatively well-defined size distribution. Typically, the suspension is cycled through the membrane one or more times until the desired liposome or LNP size distribution is achieved. The lipid-based carrier or lipid nanoformulations may be extruded through successively smaller-pore membranes, to achieve a gradual reduction in liposome or LNP size.

Any of the lipid-based carrier or lipid nanoformulations described herein can be analyzed by methods well-known to one skilled in the art to determine its physical and/or chemical features. For example, a phosphate assay can be used to determine the concentration of the lipid nanoformulations. One phosphate assay is based on the interaction between molybdate and malachite green dye. The main principle involves the reaction of inorganic phosphate with molybdate to form a colorless unreduced phosphomolybdate complex which is converted to a blue colored complex when reduced under acidic conditions. Phosphomolybdate gives 20 or 30 times more color when complexed with malachite green. The final product, reduced green soluble complex is measured by its absorbance at 620 nm and is a direct measure of inorganic phosphate in solution.

In some embodiments, the lipid-based carrier or lipid nanoformulations disclosed herein are tested for particle size, lipid concentration, and active agent encapsulation.

### Payloads and Therapeutic Agents

In certain embodiments, the lipid nanoparticle further comprises a payload. In some embodiments, the payload is a therapeutic agent. In certain embodiments, the term “effector” is used interchangeably with “payload.”

In certain embodiments, the present disclosure provides a method of delivering a therapeutic agent, comprising administering to a subject in need thereof an effective amount of the nanoparticle composition of comprising a therapeutic agent.

### Nucleic Acid Molecules

In some embodiments, the therapeutic agent is a nucleic acid molecule (i.e., a “therapeutic nucleic acid molecule”). The nucleic acid molecule may be any nucleic acid molecule that can function as a therapeutic or diagnostic agent. For instance, the nucleic acid molecule may be a DNA or RNA.

In some embodiments, the nucleic acid molecule is a nucleic acid selected from the group consisting of a plasmid, an immunostimulatory oligonucleotide, an antisense oligonucleotide, an antagomir, an aptamer, a deoxyribozyme (DNAzyme), and a ribozyme.

In certain embodiments, the therapeutic agent is a nucleic acid molecule, such as a plasmid, an immunostimulatory oligonucleotide, an antisense oligonucleotide, an antagomir, an aptamer, a deoxyribozyme (DNAzyme), or a ribozyme. In certain embodiments, the nucleic acid molecule is DNA or RNA. In certain embodiments, the nucleic acid molecule is DNA; and the DNA is a linear DNA, circular DNA, single stranded DNA, or double stranded DNA. In certain embodiments, the nucleic acid molecule is circular double stranded DNA. In alternative embodiments, the nucleic acid molecule is RNA. In certain embodiments, the RNA is messenger RNA (mRNA), miRNA, siRNA or siRNA precursor, RNA aptamer, linear RNA, circular RNA, single stranded RNA, double stranded RNA, tRNA, microRNA (miRNA) or miRNA precursor, Dicer substrate small interfering RNA (dsiRNA), Dicer substrate RNA (dsRNA), short hairpin RNA (shRNA), asymmetric interfering RNA (aiRNA), guide RNA (gRNA), lncRNA, ncRNA, sncRNA, rRNA, snRNA, piRNA, snoRNA, snRNA, scaRNA, exRNA, scaRNA, Y RNA, or hnRNA. In some embodiments, the RNA is a mRNA. In yet further embodiments, the nucleic acid molecule comprises a phosphoramide, a phosphorothioate, a phosphorodithioate, an O-methylphosphoroamidate, a morpholino, a locked nucleic acid (LNA), a glycerol nucleic acid (GNA), a threose nucleic acid (TNA), or a peptide nucleic acid (PNA).

In some embodiments, the therapeutic agent is DNA. The DNA may be selected by one skilled in the art. In some embodiments, the DNA is linear DNA, circular DNA, single stranded DNA, or double stranded DNA. In some embodiments, the therapeutic agent is linear DNA. In some embodiments, the therapeutic agent is circular DNA. In some embodiments, the therapeutic agent is single stranded DNA. In some embodiments, the therapeutic agent is double stranded DNA.

In some embodiments, the therapeutic agent is RNA. The RNA may be selected by one skilled in the art. In certain embodiments, the RNA is mRNA, miRNA, siRNA or siRNA precursor, RNA aptamer, linear RNA, circular RNA, single stranded RNA, double stranded RNA, tRNA, microRNA (miRNA) or miRNA precursor, a Dicer substrate small interfering RNA (dsiRNA), a short hairpin RNA (shRNA), an asymmetric interfering RNA (aiRNA), a guide RNA (gRNA), lncRNA, ncRNA, sncRNA, rRNA, snRNA, piRNA, snoRNA, snRNA, scaRNA, exRNA, Y RNA, or hnRNA.

In certain embodiments, the DNA or RNA encodes a polypeptide.

In some embodiments, the nucleic acid molecule comprises one or more nucleic acid analogs selected from the group consisting of a phosphoramide, a phosphorothioate, a phosphorodithioate, an O-methylphosphoroamidate, a morpholino, a locked nucleic acid (LNA), a glycerol nucleic acid (GNA), a threose nucleic acid (TNA), and a peptide nucleic acid (PNA).

In some embodiments, the therapeutic agent is an mRNA (messenger RNA). In some embodiments, the therapeutic agent is a miRNA (microRNA) or miRNA precursor. In some embodiments, the therapeutic agent is a siRNA (small interfering RNA) or siRNA precursor. In some embodiments, the therapeutic agent is a Dicer substrate small interfering RNA (dsiRNA). In some embodiments, the therapeutic agent is a short hairpin RNA (shRNA). In some embodiments, the therapeutic agent is an asymmetric interfering RNA (aiRNA). In some embodiments, the therapeutic agent is a guide RNA (gRNA). In some embodiments, the therapeutic agent is an RNA aptamer. In some embodiments, the therapeutic agent is a circular RNA, e.g., a circular RNA encoding a therapeutic polypeptide, or a non-coding circular RNA. In some embodiments, the therapeutic agent is a tRNA (transfer RNA). In some embodiments, the therapeutic agent is a rRNA (ribosomal RNA). In some embodiments, the therapeutic agent is a lncRNA (long non-coding RNA). In some embodiments, the therapeutic agent is a snRNA (small nuclear RNA). In some embodiments, the therapeutic agent is a ncRNA (non-coding RNA). In some embodiments, the therapeutic agent is a sncRNA (small noncoding RNA). In some embodiments, the therapeutic agent is a snoRNA (small nucleolar RNA). In some embodiments, the therapeutic agent is a piRNA (piwi-interacting RNA). In some embodiments, the therapeutic agent is a scaRNA (small cajal body-specific RNA). In some embodiments, the therapeutic agent is an exRNA (extracellular RNA). In some embodiments, the therapeutic agent is a Y RNA (small non-coding RNAs that are components of the Ro60 ribonucleoprotein particle). In some embodiments, the therapeutic agent is a hnRNA (heterogeneous nuclear RNA). In some embodiments, the therapeutic agent is a shRNA (small hairpin RNA).

In some embodiments, the therapeutic agent is an enzymatic nucleic acid molecule. The term “enzymatic nucleic acid molecule” refers to a nucleic acid molecule which has complementarity in a substrate binding region to a specified gene target, and also has an enzymatic activity which is active to specifically cleave target RNA. That is, the enzymatic nucleic acid molecule is able to intermolecularly cleave RNA and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% can also be useful in this invention (see for example Werner et al., Nucleic Acids Research 23:2092-2096 (1995); Hammann et al., Antisense and Nucleic Acid Drug Dev. 9:25-31 (1999), which are incorporated herein by reference in their entirety).

The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity.

In some embodiments, the therapeutic agent is an antisense nucleic acid. The term “antisense nucleic acid” refers to a non-enzymatic nucleic acid molecule that binds to target RNA by means of RNA-RNA or RNA-DNA or RNA-PNA (protein nucleic acid) interactions and alters the activity of the target RNA. Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both. In addition, antisense DNA can be used to target RNA by means of DNA-RNA interactions, thereby activating RNase H, which digests the target RNA in the duplex.

In some embodiments, the nucleic acid molecule may be a 2-5A antisense chimera. The term “2-5A antisense chimera” refers to an antisense oligonucleotide containing a 5′-phosphorylated 2′-5′-linked adenylate residue. These chimeras bind to target RNA in a sequence-specific manner and activate a cellular 2-5A-dependent ribonuclease which, in turn, cleaves the target RNA.

In some embodiments, the nucleic acid molecule may be a triplex forming oligonucleotide. The term “triplex forming oligonucleotide” refers to an oligonucleotide that can bind to a double-stranded DNA in a sequence-specific manner to form a triple-strand helix.

In some embodiments, the therapeutic nucleic acid molecule targets a host gene, e.g., the nucleic acid effector hybridizes to an endogenous gene.

In some embodiments, the nucleic acid molecule may be a decoy RNA. The term “decoy RNA” refers to a RNA molecule or aptamer that is designed to preferentially bind to a predetermined ligand. Such binding can result in the inhibition or activation of a target molecule.

In some embodiments, the nucleic acid molecule (e.g., RNA or DNA) encodes a therapeutic peptide or polypeptide. In some embodiments, the polypeptide is operably linked to a promoter for a DNA. In the case of a DNA, the nucleic acid comprises a promoter operably linked to the sequence encoding the therapeutic peptide or polypeptide. The therapeutic peptide or polypeptide may be, e.g., a transcription factor; a chromatin remodeling factor; an antigen; a hormone; an enzyme (such as a nuclease, e.g., an endonuclease, e.g., a nuclease element of a CRISPR system, e.g., a Cas9, dCas9, aCas9-nickase, CpfCas12a); a Crispr-linked enzyme, e.g., a base editor or prime editor; a mobile genetic element protein (e.g., a transposase, a retrotransposase, a recombinase, an integrase); a Gene Writer; a polymerase; a methylase; a demethylase; an acetylase; a deacetylase; a kinase; a phosphatase; a ligase; a deubiquitinase; an integrase; a recombinase; a topoisomerase; a gyrase; a helicase; a lysosomal acid hydrolase); an antibody; a receptor ligand; a receptor; a clotting factor; a membrane protein; a mitochondrial protein; a nuclear protein; an antibody or other protein scaffold binder such as a centyrin, darpin, or adnectin.

In some embodiments, the therapeutic agent is a vaccine. In some embodiments, the vaccine is a RNA vaccine, such as a RNA cancer vaccine or RNA vaccine for infectious disease (e.g., a virus, such as an influenza virus vaccine or a corona virus vaccine (e.g., COVID-19 vaccine).

In some embodiments, the nucleic acid molecule (e.g., a DNA or RNA) encodes (if DNA) or is (if RNA) a non-coding RNA, e.g., one or more of a siRNA, a miRNA, long non-coding RNA, a piRNA, a snoRNA, a scaRNA, a tRNA, a rRNA, a therapeutic RNA aptamer, and a snRNA.

In some embodiments, the nucleic acid molecule is an antisense RNA; a guide RNA; a nucleic acid that hybridizes to an exogenous nucleic acid such as a viral DNA or RNA, nucleic acid that hybridizes to an RNA; a nucleic acid that interferes with gene transcription; a nucleic acid that interferes with RNA translation; a nucleic acid that stabilizes RNA or destabilizes RNA such as through targeting for degradation; or a nucleic acid that modulates a DNA or RNA binding factor.

In some embodiments, the nucleic acid molecule targets a sense strand of a host gene. In some embodiments, the nucleic acid molecule targets an antisense strand of a host gene.

In some embodiments, the nucleic acid molecule is or encodes a guide RNA. Guide RNA sequences are generally designed to have a length of between 15-30 nucleotides (e.g., 17, 19, 20, 21, 24 nucleotides) and complementary to the targeted nucleic acid sequence. Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective in genome editing; see, for example, Hendel et al. (2015) Nature Biotechnol., 985-991. The gRNA may recognize specific DNA sequences (e.g., sequences adjacent to or within a promoter, enhancer, silencer, or repressor of a gene). In some embodiments, the gRNA is used as part of a CRISPR system for gene editing. For the purposes of gene editing, the ssDNA construct or sequence disclosed herein may be designed to include one or multiple sequences encoding guide RNA sequences corresponding to a desired target DNA sequence; see, for example, Cong et al. (2013) Science, 339:819-823; Ran et al. (2013) Nature Protocols, 8:2281-2308.

In some embodiments, the nucleic acid molecule can include a plurality of sequences. The plurality may be the same or different types. The plurality of sequences may be the same or different sequences of the same type.

All the nucleic acid molecules described herein can be chemically modified. The various modification strategy to the nucleic acid molecules are well known to one skilled in the art. In some embodiments, the nucleic acid molecule comprises one or more modifications selected from the group consisting of pseudouridine, 5-bromouracil, 5-methylcytosine, peptide nucleic acid, xeno nucleic acid, morpholinos, locked nucleic acids, glycol nucleic acids, threose nucleic acids, dideoxynucleotides, cordycepin, 7-deaza-GTP, florophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. In some embodiments, the antisense oligonucleotide may be a locked nucleic acid oligonucleotide (LNA). The term “locked nucleic acid (LNA)” refers to oligonucleotides that contain one or more nucleotide building blocks in which an extra methylene bridge fixes the ribose moiety either in the C3′-endo (beta-D-LNA) or C2′-endo (alpha-L-LNA) conformation (Grunweller A, Hartmann R K, BioDrugs, 21(4): 235-243 (2007)).

Additional examples of the nucleic acid molecules (including tumor suppressor genes, antisense oligonucleotides, siRNA, miRNA, or shRNA) may be found in U.S. Published Patent Application No. 2007/0065499 and U.S. Pat. No. 7,780,882, which are incorporated by reference herein in their entireties.

In some embodiments, the therapeutic agent can include a plurality of nucleic acid molecules, which may be the same or different types.

In some embodiments, the N:P ratio of the nucleic acid molecule-lipidoid composition ranges from 1:1 to 30:1, for instance from 3:1 to 20:1, from 3:1 to 15:1, from 3:1 to 10:1, or from 3:1 to 6:1. An N:P ratio refers to the molar ratio of the amines present in the plurality of lipidoids (e.g., the amines in the lipidoids) to the phosphates present in the nucleic acid molecule. It is a factor for efficient packaging and potency. In some embodiments, the N:P ratio of the nucleic acid molecule—lipidoid composition ranges from 3:1 to 15:1.

### Other Therapeutic Agents

In alternative embodiments, the therapeutic agent is a peptide or protein. In some embodiments, the therapeutic agent is a small molecule drug encapsulated in the lipidoid composition or on the surface of the lipidoid composition. The payload can contain two or more different therapeutic agents from the nucleic acid molecule, peptide or protein, and small molecule drug.

In some embodiments, the effector is a protein effector. In some embodiments, the protein effector may be any peptide or protein molecule that can function as a therapeutic or diagnostic agent. In some embodiments, the protein may be a peptide or polypeptide, e.g., a transcription factor; a chromatin remodeling factor; an antigen; a hormone; an enzyme (such as a nuclease, e.g., an endonuclease, e.g., a nuclease element of a CRISPR system, e.g., a Cas9, dCas9, aCas9-nickase, Cpf/Cas12a); a Crispr-linked enzyme, e.g., a base editor or prime editor; a mobile genetic element protein (e.g., a transposase, a retrotransposase, a recombinase, an integrase); a gene writer; a polymerase; a methylase; a demethylase; an acetylase; a deacetylase; a kinase; a phosphatase; a ligase; a deubiquitinase; an integrase; a recombinase; a topoisomerase; a gyrase; a helicase; a lysosomal acid hydrolase); an antibody; a receptor ligand; a receptor; a clotting factor; a membrane protein; a mitochondrial protein; a nuclear protein; an antibody or other protein scaffold binder such as a centyrin, darpin, or adnectin.

In some embodiments, the protein is a ribonucleoprotein (RNP) that forms a complex of ribonucleic acid and RNA-binding protein. In some embodiments, the protein is a recombinant cytokine.

In some embodiments, the nanoparticle composition can include a plurality of protein molecules, which may be the same or different types.

In some embodiments, the therapeutic agent is a small molecule drug, for instance, a small molecule drug approved for use in humans by an appropriate regulatory authority.

In some embodiments, the small molecule drug is an HDAC inhibitor, a kinase inhibitor, a cytotoxic molecule, a chromatin modulator, an RNAi modulator, transcription factor, an adjuvant, or a combination of two or more.

In some embodiments, the small molecule drug can be a small molecule that lacks cell permeability properties.

In some embodiments, the nanoparticle composition can include a plurality of small molecule drugs, which may be the same or different types.

In some embodiments, the therapeutic agent may be encapsulated in the LNP. For example, the therapeutic agent may be completely or partially located in the interior space of the LNPs, within the lipid layer/membrane, or associated with the exterior surface of the lipid layer/membrane. In some embodiments, incorporation of the therapeutic agent into the LNP protects the therapeutic agents from environments which may contain enzymes or chemicals or conditions that degrade the therapeutic agents and/or systems or receptors that cause the rapid excretion of the therapeutic agents. Moreover, incorporating the therapeutic agent into the LNP may promote uptake of the therapeutic agent, and hence, may enhance the therapeutic effect.

The ratio of total lipid and lipidoid components to the therapeutic agent can be varied as desired. For example, the total lipid and lipidoid components to the therapeutic agent (mass or weight) ratio can be from about 10:1 to about 30:1. In some embodiments, the total lipid and lipidoid components to the therapeutic agent ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of total lipid and lipidoid components and the therapeutic agent can be adjusted to provide a desired N:P ratio, for example, N:P ratio of 3, 4, 5, 6, 7, 8, 9, 10, 11, 12, 13, 14, 15, 16, 17, 18, 19, 20, 21, 22, 23, 24, 25, 26, 27, 28, 29, 30, or higher. Generally, the overall lipid and lipidoid content can range from about 5 mg/mL to about 30 mg/mL in the nanoparticle composition.

The nanoparticle composition may contain about 5 to about 95% by weight of the therapeutic agent. In some embodiments, the nanoparticle composition contains about 5%, about 10%, about 20, about 30, about 40, about 50, about 60, about 70, about 80, about 90, or about 95% by weight of the therapeutic agent. In some embodiments, the nanoparticle composition contains the therapeutic agent in an amount about 5-95%, about 5-90%, about 5-80%, about 5-70%, about 5-60%, about 5-50%, about 5-40%, about 5-30%, about 5-20%, about 5-10%, about 10-95%, about 10-90%, about 10-80%, about 10-70%, about 10-60%, about 10-50%, about 10-40%, about 10-30%, about 10-20%, about 20-95%, about 20-90%, about 20-80%, about 20-70%, about 20-60%, about 20-50%, about 20-40%, about 20-30%, about 30-95%, about 30-90%, about 30-80%, about 30-70%, about 30-60%, about 30-50%, about 30-40%, about 40-95%, about 40-90%, about 40-80%, about 40-70%, about 40-60%, about 40-50%, about 50-95%, about 50-90%, about 50-80%, about 50-70%, about 50-60%, about 60-95%, about 60-90%, about 60-80%, about 60-70%, about 70-95%, about 70-90%, about 70-80%, about 80-95%, about 80-90%, or about 90-95%, based on the weight of the nanoparticle composition.

In certain embodiments, the nanoparticle composition further comprises an antigen; wherein the antigen is a protein or a nucleic acid; the antigen is a protein; or the antigen is a nucleic acid.

In further embodiments, the nanoparticle composition further comprises an mRNA molecule comprising a nucleotide sequence that encodes an antigen.

The nanoparticle compositions described herein are useful for delivering a therapeutic agent. Accordingly, in certain embodiments, provided herein is a method of delivering a therapeutic agent, comprising administering to a subject in need thereof an effective amount of the nanoparticle composition of the disclosure that comprises a therapeutic agent.

The nanoparticle compositions described herein are useful for delivering an antigen. Accordingly, in certain embodiments, provided herein is a method of vaccination, comprising administering to a subject in need thereof an effective amount of a nanoparticle composition of the disclosure, wherein the nanoparticle composition comprises an antigen.

### Pharmaceutical Compositions

In certain aspects, the present disclosure provides a pharmaceutical composition comprising a nanoparticle composition of the disclosure, in combination with a pharmaceutically acceptable carrier.

As used throughout this section, a “lipidoid composition” can refer to a nanoparticle composition comprising a lipidoid compound, e.g., a lipidoid compound of the disclosure.

The compositions and methods of the present disclosure may be utilized to treat an individual in need thereof. The pharmaceutical composition described herein may comprise a therapeutic or prophylactic composition, or any combination thereof. In certain embodiments, the individual is a mammal such as a human, or a non-human mammal. When administered to an animal, such as a human, the nanoparticle composition or the lipidoid composition is preferably administered as a pharmaceutical composition comprising, for example, a lipidoid composition and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a lipidoid composition. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a lipidoid composition. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those lipidoid compositions, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The lipidoid composition may also be formulated for inhalation. In certain embodiments, a lipidoid composition may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the lipidoid composition which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active composition, such as a lipidoid (e.g., nanoparticle) composition as described herein, with the carrier and, optionally, one or more accessory ingredients. In general, the formulations are prepared by uniformly and intimately bringing into association a lipidoid (e.g., nanoparticle) composition as described herein with liquid carriers, or finely divided solid carriers, or both, and then, if necessary, shaping the product.

Formulations of the disclosure suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a lipidoid (e.g., nanoparticle) composition as described herein of the present disclosure as an active ingredient. Lipidoid compositions may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium lipidoid compositions; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered lipidoid composition moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in microencapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active lipidoid compositions, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active lipidoid composition may be mixed under sterile conditions with a pharmaceutically acceptable carrier, and with any preservatives, buffers, or propellants that may be required.

The ointments, pastes, creams and gels may contain, in addition to an active lipidoid composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active lipidoid composition, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a lipidoid composition to the body. Such dosage forms can be made by dissolving or dispersing the active lipidoid composition in the proper medium. Absorption enhancers can also be used to increase the flux of the lipidoid composition across the skin. The rate of such flux can be controlled by either providing a rate controlling membrane or dispersing the lipidoid composition in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active lipidoid compositions in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the lipidoid compositions in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this disclosure, active lipidoid compositions can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals.

A variety of biocompatible polymers (including hydrogels), including both biodegradable and non-degradable polymers, can be used to form an implant for the sustained release of a lipidoid composition at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular lipidoid composition or combination of lipidoid compositions employed, or the ester, salt or amide thereof, the route of administration, the time of administration, the rate of excretion of the particular lipidoid composition(s) being employed, the duration of the treatment, other drugs, lipidoid compositions and/or materials used in combination with the particular lipidoid composition(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or lipidoid composition at levels lower than that required in order to achieve the desired therapeutic effect and gradually increase the dosage until the desired effect is achieved. By “therapeutically effective amount” is meant the concentration of a lipidoid composition that is sufficient to elicit the desired therapeutic effect. It is generally understood that the effective amount of the lipidoid composition will vary according to the weight, sex, age, and medical history of the subject. Other factors which influence the effective amount may include, but are not limited to, the severity of the patient's condition, the disorder being treated, the stability of the lipidoid composition, and, if desired, another type of therapeutic agent being administered with the lipidoid composition of the disclosure.

A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active lipidoid composition used in the compositions and methods of the disclosure will be that amount of the lipidoid composition that is the lowest dose effective to produce a therapeutic effect. Such an effective dose will generally depend upon the factors described above.

If desired, the effective daily dose of the active lipidoid composition may be administered as one, two, three, four, five, six or more sub-doses administered separately at appropriate intervals throughout the day, optionally, in unit dosage forms. In certain embodiments of the present disclosure, the active lipidoid composition may be administered two or three times daily. In preferred embodiments, the active lipidoid composition will be administered once daily.

The patient receiving this treatment is any animal in need, including primates, in particular humans; and other mammals such as equines, cattle, swine, sheep, cats, and dogs; poultry; and pets in general.

In certain embodiments, lipidoid compositions of the disclosure may be used alone or conjointly administered with another type of therapeutic agent.

The present disclosure includes the use of pharmaceutically acceptable salts of lipidoids of the disclosure in the compositions and methods of the present disclosure. In certain embodiments, contemplated salts of the disclosure include, but are not limited to, alkyl, dialkyl, trialkyl or tetra-alkyl ammonium salts. In certain embodiments, contemplated salts of the disclosure include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the disclosure include, but are not limited to, Na, Ca, K, Mg, Zn or other metal salts. In certain embodiments, contemplated salts of the disclosure include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or using a device for such slow or extended release.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or a viral infection. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, heteroaryl, cycloalkyl, heterocycloalkyl, amino, alkylamino, dialkylamino, amido (—C(O)NH₂), carboxyl (—C(O)OH), cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted. A “linear” alkyl group refers to a straight-chain alkyl group without a branching point. A “branched” alkyl group refers to an alkyl group having at least one branch point. Examples of branched alkyl groups include, e.g., isopropyl, sec-butyl, and tert-butyl.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. Coalkyl indicates a hydrogen where the group is in a terminal position, a bond if internal. A C₁₋₆alkyl group, for example, contains from one to six carbon atoms in the chain.

The term “alkylene” refers to a divalent alkyl group. The term (hydroxyalkyl)-O-alkylene-, for example, refers to a divalent alkyl group (i.e., an alkylene), appended to the parent molecule at one of its open valencies, and attached to an oxygen atom at the other of its open valencies, which oxygen atom is substituted by a hydroxyalkyl group.

The term “alkenyl” as used herein means a straight or branched chain hydrocarbon radical containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl. The unsaturated bond(s) of the alkenyl group can be located anywhere in the moiety and can have either the (Z) or the (E) configuration about the double bond(s). A “linear” alkenyl group refers to a straight-chain alkenyl group without a branching point. A “branched” alkenyl group refers to an alkenyl group having at least one branch point.

The term “alkenylene” refers to a divalent alkenyl group.

The term “alkynyl” as used herein means a straight or branched chain hydrocarbon radical containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl. A “linear” alkynyl group refers to a straight-chain alkynyl group without a branching point. A “branched” alkynyl group refers to an alkynyl group having at least one branch point.

The term “alkynylene” refers to a divalent alkynyl group.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—, preferably alkylC(O)NH—.

The term “acyloxy” is art-recognized and refers to a group represented by the general formula hydrocarbylC(O)O—, preferably alkylC(O)O—.

The term “alkoxy” refers to an alkyl group appended to the parent molecular moiety through an oxygen atom. Representative alkoxy groups include methoxy, ethoxy, propoxy, tert-butoxy and the like.

The term “alkoxyalkyl” refers to an alkyl group substituted with an alkoxy group and may be represented by the general formula -alkyl-O-alkyl.

The term “alkylamino”, as used herein, refers to an amino group substituted with at least one alkyl group. A “dialkylamino” refers to an amino group substituted with two alkyl groups.

The term “alkylthio”, as used herein, refers to a thiol group substituted with an alkyl group and may be represented by the general formula alkylS-.

The term “amide”, as used herein, refers to a group

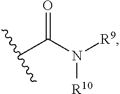

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure. In some embodiments, “amido” refers to the group —C(O)NH₂.

The terms “amine” and “amino” are art-recognized and refer to both unsubstituted and substituted amines and salts thereof, e.g., a moiety that can be represented by

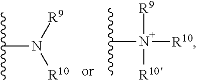

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure. In some embodiments, “amino” refers to the group —NH₂.

The term “aminoalkyl”, as used herein, refers to an alkyl group substituted with an amino group.

The term “arylalkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

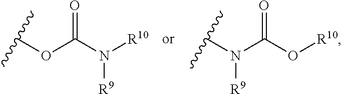

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy” or “carboxyl”, as used herein, refers to a group represented by the formula —CO₂H.

The term “cycloalkyl” includes substituted or unsubstituted non-aromatic single ring structures, preferably 4- to 8-membered rings, more preferably 4- to 6-membered rings. The term “cycloalkyl” also includes polycyclic ring systems having two or more cyclic rings in which two or more carbons are common to two adjoining rings wherein at least one of the rings is cycloalkyl and the substituent (e.g., R¹⁰⁰) is attached to the cycloalkyl ring, e.g., the other cyclic rings can be cycloalkyls, cycloalkenyls, cycloalkynyls, aryls, heteroaryls, and/or heterocyclyls. Cycloalkyl groups include, e.g., cyclopropyl, cyclobutyl, cyclopentyl, and cyclohexyl.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical.

Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “heteroaralkyl” and “heteroarylalkyl”, as used herein, refers to an alkyl group substituted with a heteroaryl group.

The term “heteroaryl” includes substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also includes polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocycloalkylalkyl” and “heterocyclylalkyl”, as used herein, refers to an alkyl group substituted with a heterocycle group.

The terms “heterocycloalkyl,” “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocycloalkyl,” “heterocyclyl”, “heterocycle”, and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocycloalkyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

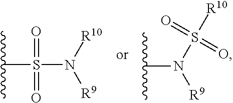

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹

wherein R⁹ represents a hydrocarbyl.

The term “thioether”, as used herein, is equivalent to an ether, wherein the oxygen is replaced with a sulfur.

The term “urea” is art-recognized and may be represented by the general formula

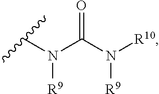

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds disclosed herein. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds disclosed herein. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

In certain embodiments, the lipidoid compositions (e.g., nanoparticles) useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Some of the lipidoid compositions (e.g., nanoparticles) may also comprise chemical compound which exist in tautomeric forms. Such forms, although not explicitly indicated in the formulae described herein, are intended to be included within the scope of the present disclosure.

The term “ionizable lipid” refers to a molecule having both an ionizable and lipophilic component. “Ionizable” means a group contained in the lipid (e.g., a head group) can be ionized, e.g., dissociated to produce one or more electrically charged species, under a given condition (e.g., pH). For instance, an ionizable lipid may carry a net positive charge at a selected pH, such as physiological pH (e.g., pH of about 7.0). In some embodiments, the hydrophilic component contains an ionizable amine. In some embodiments, the hydrophobic component contains one or more linear or branched lipids.

A “lipid nanoparticle” or “LNP” refers to a composition comprising a lipid or lipidoid (e.g., ionic (e.g., cationic or anionic), zwitterionic, or ionizable lipid) for encapsulation of a cargo. LNPs may also include neutral lipids such as phospholipid molecules belonging to the phosphatidylcholine (PC) class; sterols, such as cholesterol; and polyethylene glycol (PEG). LNPs may be taken up by cells via endocytosis and the ionizability of the lipids at low pH enables endosomal escape, which can allow release of cargo into the cytoplasm. LNPs are liposome-like structures. However, LNPs may not have a contiguous bilayer; some LNPs may have a single phospholipid outer layer encapsulating the interior assuming a micelle-like structure, which can have a non-aqueous core. Exemplary lipid nanoparticle composition are formulations of ionizable lipids, sterols (or hydrophobic molecules), structural lipids such as phospholipids, polyethyleneglycol (PEG) lipids, and potentially additional components (see Nature Nanotechnology 15:313-320 (2020), which is incorporated herein by reference in its entirety), or single molecules containing combinations of ionizable lipid, sterol, structural phospholipid, and shielding groups (see Nature Materials 20:701-710 (2021), which is incorporated herein by reference in its entirety). These components may be mixed with a therapeutic agent (e.g., nucleic acid molecules such as mRNA) to be formulated into LNP composition.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

“Nucleic acid” means an oligonucleotide or polynucleotide sequence. Non-limiting examples of oligonucleotide or polynucleotides are DNA, plasmid DNA, self-amplifying RNA, mRNA, siRNA and tRNA. The term also encompasses RNA/DNA hybrids.

Nucleotides are typically linked in a nucleic acid by phosphodiester bonds, although the term “nucleic acid” also encompasses nucleic acid analogs having other types of linkages or backbones (e.g., phosphoramide, phosphorothioate, phosphorodithioate, O-methylphosphoroamidate, morpholino, locked nucleic acid (LNA), glycerol nucleic acid (GNA), threose nucleic acid (TNA), and peptide nucleic acid (PNA) linkages or backbones, among others). The nucleic acids may be single-stranded, double-stranded, or contain portions of both single-stranded and double-stranded sequence. A nucleic acid can contain any combination of deoxyribonucleotides and ribonucleotides, as well as any combination of bases, including, for example, adenine, thymine, cytosine, guanine, uracil, and modified or non-canonical bases (including, e.g., hypoxanthine, xanthine, 7-methylguanine, 5,6-dihydrouracil, 5-methylcytosine, and 5 hydroxymethylcytosine).

As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-limiting group consisting of small interfering RNA (siRNA), asymmetrical interfering RNA (aiRNA), microRNA (miRNA), Dicer-substrate RNA (dsRNA), small hairpin RNA (shRNA), mRNA, and mixtures thereof.

In some embodiments, the therapeutic agent is an antisense nucleic acid. The term “antisense nucleic acid” refers to a non-enzymatic nucleic acid molecule that binds to target RNA by means of RNA-RNA or RNA-DNA or RNA-PNA (protein nucleic acid) interactions and alters the activity of the target RNA. Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both. In addition, antisense DNA can be used to target RNA by means of DNA-RNA interactions, thereby activating RNase H, which digests the target RNA in the duplex.

### Examples

The invention now being generally described, it will be more readily understood by reference to the following examples which are included merely for purposes of illustration of certain aspects and embodiments of the present invention, and they are not intended to limit the invention.

### Synthesis and Characterization of Lipid 1

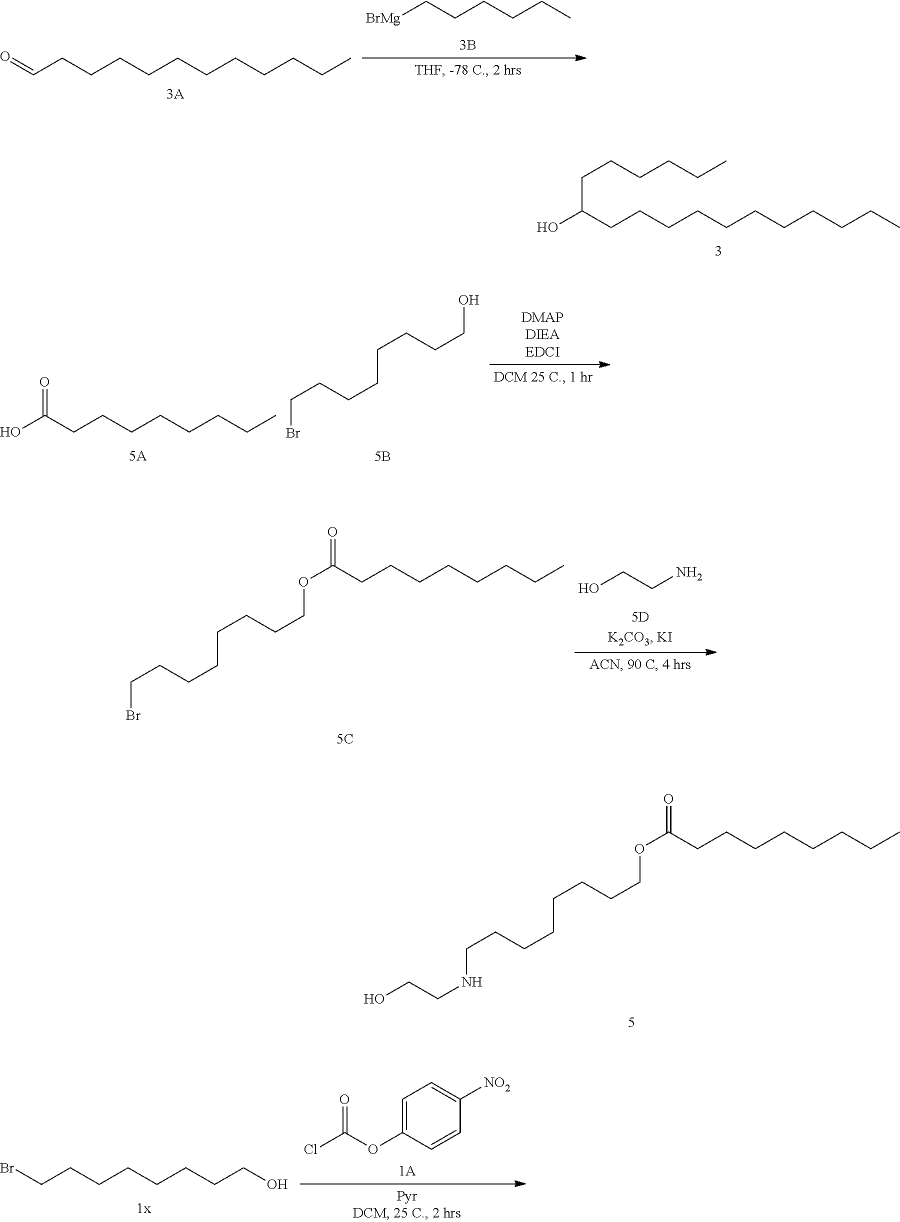

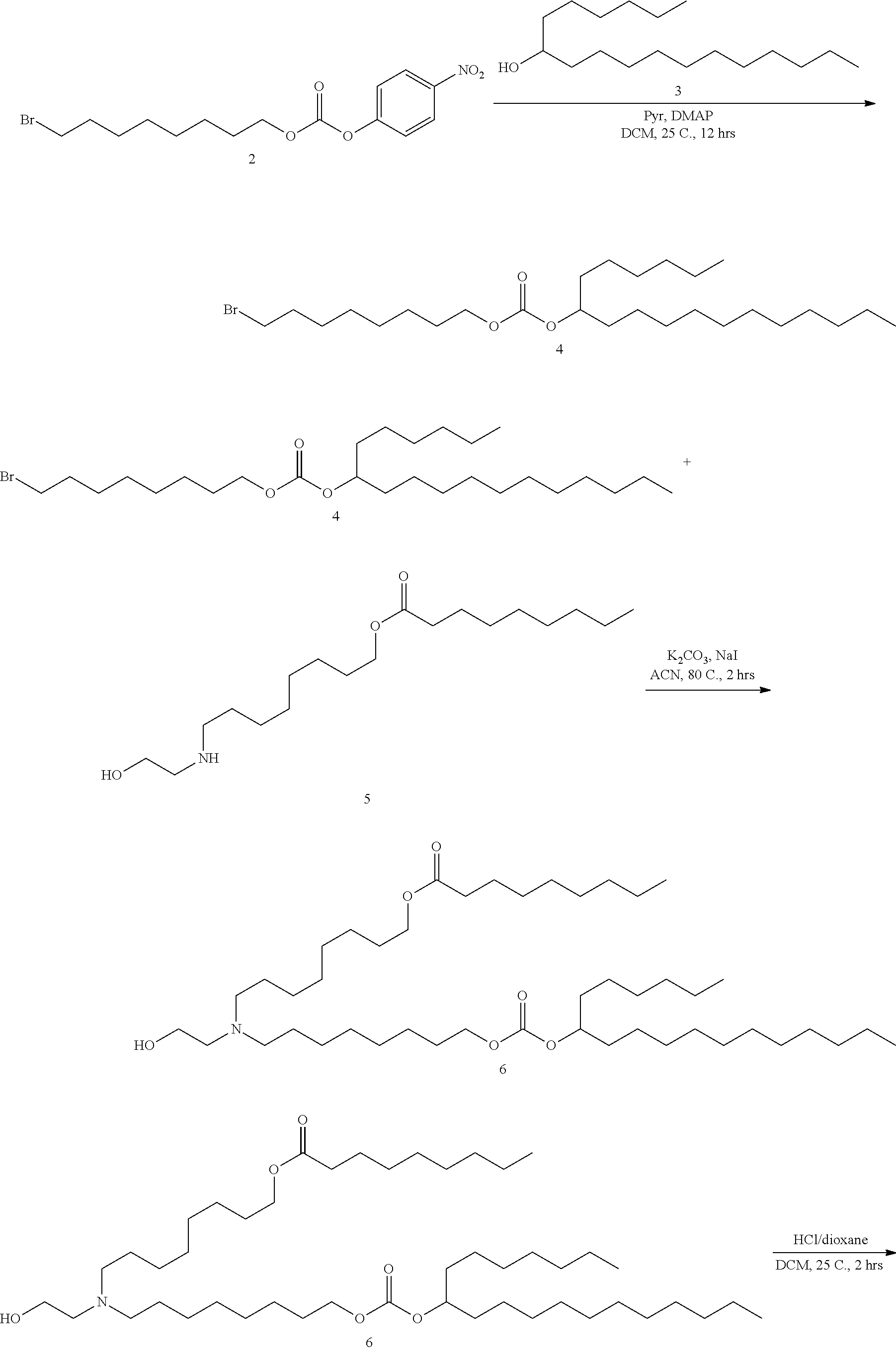

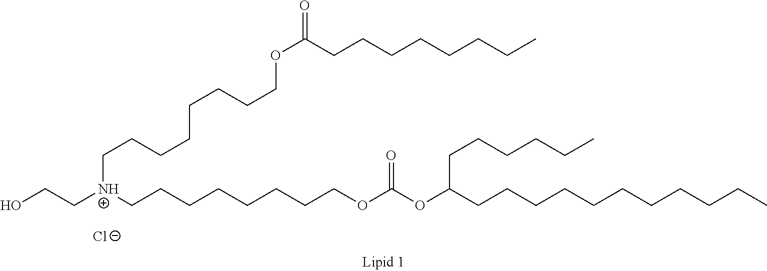 [SMILES: CCCCCCCCCCCC(CCCCCC)OC(=O)OCCCCCCCC[NH+](CCO)CCCCCCCCOC(=O)CCCCCCCC ; [Cl-]]

### General Procedure for Preparation of Compound 3:

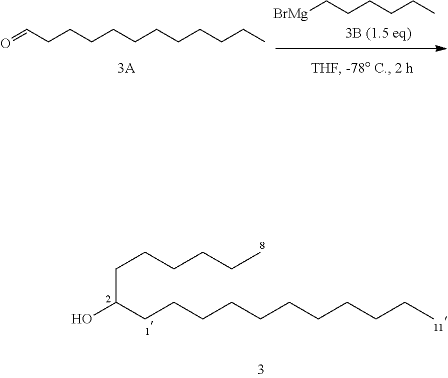

To a solution of Compound 3A (92 g, 499.14 mmol, 1 eq.) in THF (1000 mL) was added Compound 3B (94.52 g, 499.14 mmol, 97.45 mL, 1 eq.) at −78° C. The mixture was stirred at −78° C. for 2 hours. TLC (PE: EA=10:1, P1: Rf=0.4) indicated ˜30% of Compound 3A was remained, and one major new spot with larger polarity was detected. The reaction mixture was quenched by addition NH₄Cl (500 mL) at 0° C., and then diluted with H₂O (1000 mL) and extracted with ethyl acetate (1000 mL*3). The combined organic layers were washed with brine (500 mL*3), dried over Na₂SO₄, filtered and concentrated under reduced pressure to give a residue. The residue was purified by column chromatography (SiO₂, Petroleum ether: Ethyl acetate=100:1 to 30:1) to give Compound 3 (72 g, 266.18 mmol, 53.33% yield) as white solid.

¹H NMR (CHLOROFORM-d, 400 MHz) S=3.59 (br dd, 1H, J=4.4, 6.8 Hz), 1.2-1.5 (m, 30H), 0.8-1.0 (m, 6H) General procedure for preparation of compound 5C:

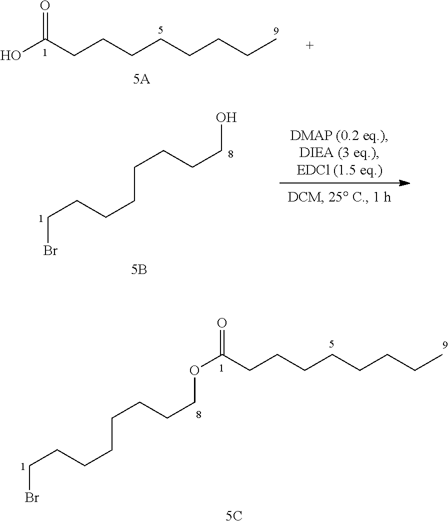 [SMILES: CCCCCCCCC(=O)O ; OCCCCCCCCBr ; CCCCCCCCC(=O)OCCCCCCCCBr]

A mixture of Compound 5A (35 g, 221.19 mmol, 1 eq.), Compound 5B (55.51 g, 265.42 mmol, 45.50 mL, 1.2 eq.), EDCI (63.60 g, 331.78 mmol, 1.5 eq.), DIEA (85.76 g, 663.56 mmol, 115.58 mL, 3 eq.) and DMAP (5.40 g, 44.24 mmol, 0.2 eq.) in DCM (350 mL) was degassed and purged with N₂ for 3 times, and then the mixture was stirred at 25° C. for 1 hour under N₂ atmosphere. TLC (Petroleum ether: Ethyl acetate=10:1, P1: R_{f}=0.66) indicated Compound 5A was consumed completely and one new spot formed. The reaction mixture was diluted with sat.NH₄Cl (400 mL) and extracted with DCM (400 mL*3). The combined organic layers were dried over Na₂SO₄ filtered and concentrated under reduced pressure to give a residue. The residue was purified by column chromatography (SiO₂, Petroleum ether: Ethyl acetate=100:0 to 80:1) to give Compound 5C (50 g, 136.64 mmol, 61.78% yield, 95.47% purity) as yellow oil.

LCMS [M+1]⁺=349.2

¹H NMR (400 MHz, CHLOROFORM-d) 8=4.05 (t, J=6.8 Hz, 2H), 3.40 (t, J=6.8 Hz, 2H), 2.28 (t, J=7.6 Hz, 2H), 1.90-1.71 (m, 2H), 1.61 (quin, J=6.8 Hz, 4H), 1.49-1.38 (m, 2H), 1.37-1.14 (m, 16H), 0.92-0.80 (m, 3H)

### General Procedure for Preparation of Compound 5:

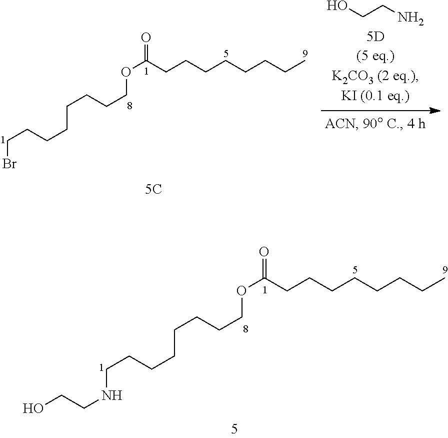

A mixture of Compound 5C (15 g, 42.94 mmol, 1 eq.), Compound 5D (13.11 g, 214.69 mmol, 12.96 mL, 5 eq.), KI (712.76 mg, 4.29 mmol, 0.1 eq.), K₂CO₃ (11.87 g, 85.87 mmol, 2 eq.) in ACN (150 mL) was degassed and purged with N₂ for 3 times, and then the mixture was stirred at 90° C. for 16 hours under N₂ atmosphere. TLC (DCM: ME=5:1, P1: R_{f}=0.44) indicated Compound 5C was consumed, and one major new spot with larger polarity was detected. The residue was diluted with ethyl acetate (100 mL) and extracted with H₂O (800 mL). The combined organic layers were dried over Na₂SO₄ filtered and concentrated under reduced pressure to give a residue. The residue was purified by column chromatography (SiO₂, Dichloromethane: Methanol=50:1 to 5:1) to give Compound 5 (6.3 g, 19.12 mmol, 44.53% yield, 100% purity) as white solid.

LCMS [M+1]⁺=330.3

¹H NMR (400 MHz, CHLOROFORM-d) S=4.06 (t, J=6.8 Hz, 2H), 3.71-3.61 (m, 2H), 2.89-2.74 (m, 2H), 2.64 (t, J=7.2 Hz, 2H), 2.38 (br s, 2H), 2.29 (t, J=7.6 Hz, 2H), 1.67-1.56 (m, 4H), 1.55-1.46 (m, 2H), 1.39-1.24 (m, 18H), 0.96-0.82 (m, 3H)

### General Procedure for Preparation of Compound 2:

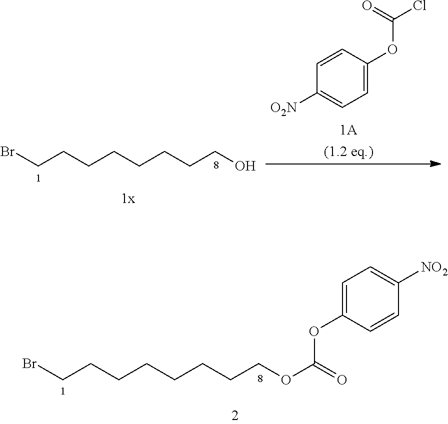 [SMILES: O=C(Cl)Oc1ccc([N+](=O)[O-])cc1 ; OCCCCCCCCBr ; O=C(OCCCCCCCCBr)Oc1ccc([N+](=O)[O-])cc1]

To a solution of Compound 1x (24 g, 114.76 mmol, 19.67 mL, 1 eq.) in DCM (300 mL) was added Py (13.62 g, 172.15 mmol, 13.89 mL, 1.5 eq.) and Compound 1A (27.76 g, 137.72 mmol, 1.2 eq.) in DCM (300 mL) was added dropwise at 0° C. The mixture was stirred at 25° C. for 2 hours. TLC (Petroleum ether: Ethyl acetate=5:1, P1: Rf=0.5) indicated Compound 1x was consumed completely and one new spot formed. The reaction mixture was diluted with H₂O (250 mL) and extracted with DCM (200 mL *3). The combined organic layers were dried over Na₂SO₄, filtered and concentrated under reduced pressure to give a residue. The residue was purified by column chromatography (SiO₂, Petroleum ether: Ethyl acetate=1:0 to 100:1) to give Compound 2 (32 g, 85.51 mmol, 74.51% yield was obtained as a colorless oil.

¹H NMR (CHLOROFORM-d, 400 MHz) S=8.2-8.3 (m, 2H), 7.3-7.4 (m, 2H), 4.2-4.3 (m, 2H), 3.4-3.5 (m, 2H), 1.8-1.9 (m, 2H), 1.7-1.8 (m, 2H), 1.3-1.5 (m, 8H) General procedure for preparation of compound 4:

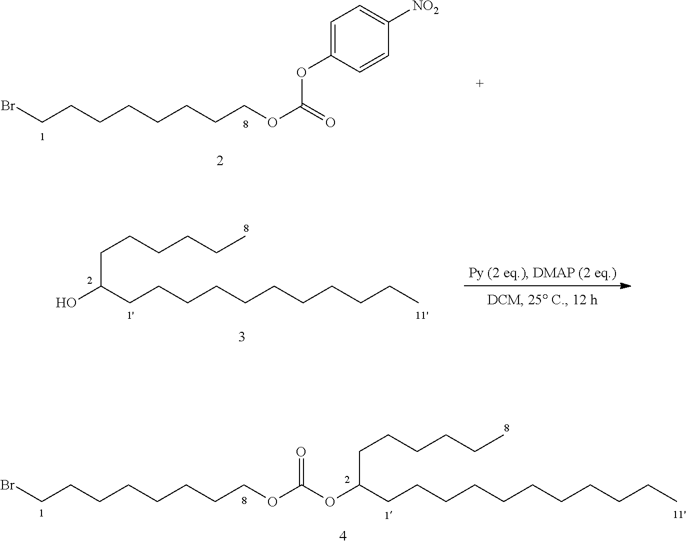 [SMILES: O=C(OCCCCCCCCBr)Oc1ccc([N+](=O)[O-])cc1 ; CCCCCCCCCCCC(O)CCCCCC ; CCCCCCCCCCCC(CCCCCC)OC(=O)OCCCCCCCCBr]

To a solution of Compound 2 (32 g, 85.51 mmol, 1 eq.) in DCM (300 mL) was added Py (13.53 g, 171.02 mmol, 13.80 mL, 2 eq.) and DMAP (20.89 g, 171.02 mmol, 2 eq.). Then Compound 3 (23.13 g, 85.51 mmol, 1 eq.) was added at 0° C. The mixture was stirred at 25° C. for 12 hours. TLC (Petroleum ether: Ethyl acetate=20:1, P1: Rf=0.5) indicated ˜30% of Compound 3 was remained, and one major new spot with lower polarity was detected.

The reaction was messy according to TLC. The reaction mixture was filtered and concentrated under reduced pressure to give a residue. The residue was purified by column chromatography (SiO₂, Petroleum ether: Ethyl acetate=0:1 to 160:1) to give Compound 4 (10.4 g, 20.57 mmol, 24.05% yield) was obtained as a white solid.

¹H NMR (CHLOROFORM-d, 400 MHz) S=4.6-4.7 (m, 1H), 4.12 (t, 2H, J=6.4 Hz), 3.40 (t, 2H, J=6.8 Hz), 1.8-1.9 (m, 2H), 1.5-1.7 (m, 6H), 1.2-1.5 (m, 34H), 0.88 (t, 6H, J=6.0 Hz) General procedure for preparation of compound 6:

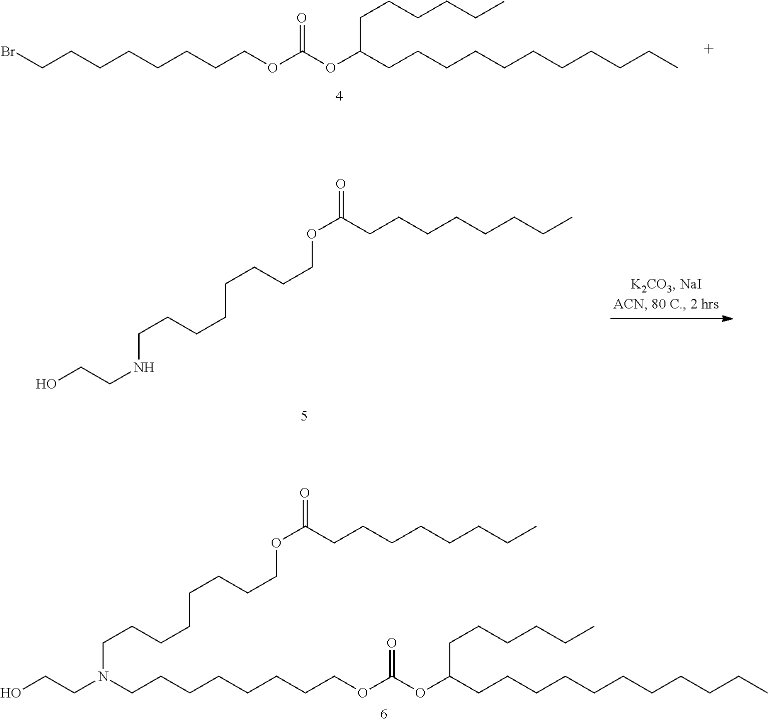 [SMILES: CCCCCCCCCCCC(CCCCCC)OC(=O)OCCCCCCCCBr ; CCCCCCCCC(=O)OCCCCCCCCNCCO ; CCCCCCCCCCCC(CCCCCC)OC(=O)OCCCCCCCCN(CCO)CCCCCCCCOC(=O)CCCCCCCC]

A mixture of Compound 5 (2.87 g, 8.71 mmol, 1 eq.), Compound 4 (4.40 g, 8.71 mmol, 1 eq.), K₂CO₃ (2.41 g, 17.42 mmol, 2 eq.), NaI (130.55 mg, 870.97 μmol, 0.1 eq.) in ACN (28 mL) was degassed and purged with N₂ for 3 times, and then the mixture was stirred at 80° C. for 16 hours under N₂ atmosphere. TLC (Dichloromethane: Methanol=8:1, R1: Rf=0.3, R2: Rf=0.9, P1: Rf=0.6) indicated Compound 5 was consumed completely and one new spot formed. The residue was diluted with EA (30 mL). The combined organic layers were washed with NH₄Cl (30 mL *3), dried over Na₂SO₄, filtered and concentrated under reduced pressure to give a residue. The residue was purified by column chromatography (SiO₂, Dichloromethane: Methanol=100:1 to 50:1) to give Compound 6 (5.9 g, 7.82 mmol, 29.94% yield, 100% purity) was obtained as a white solid.

LCMS [M+1]⁺=754.7

¹H NMR (400 MHz, CHLOROFORM-d) S=4.69 (br t, J=6.4 Hz, 1H), 4.27-4.01 (m, 4H), 3.91-3.61 (m, 2H), 3.20-2.40 (m, 6H), 2.30 (t, J=7.6 Hz, 2H), 1.70-1.52 (m, 18H), 1.39-1.16 (m, 52H), 0.89 (t, J=6.8 Hz, 9H)

### General Procedure for the Lipid 1 HCl-Salt:

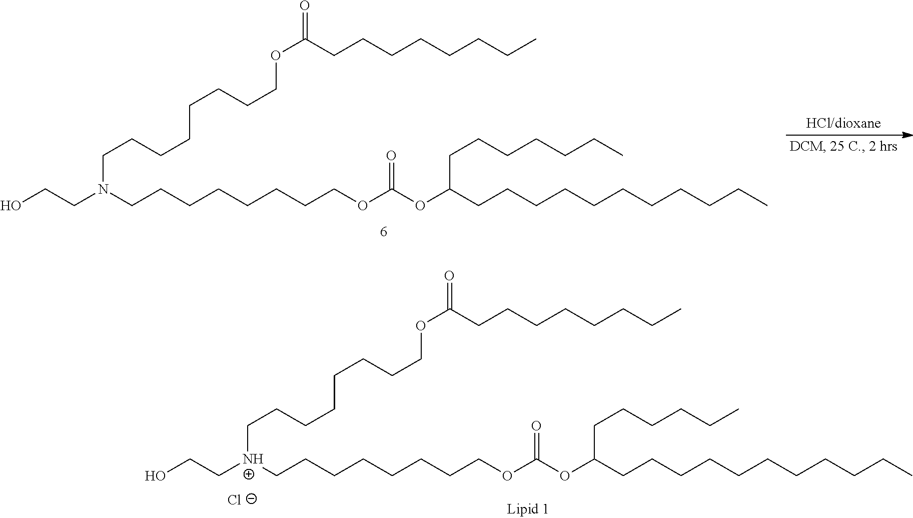 [SMILES: CCCCCCCCCCCC(CCCCCCC)OC(=O)OCCCCCCCCN(CCO)CCCCCCCCOC(=O)CCCCCCCC ; CCCCCCCCCCCC(CCCCCC)OC(=O)OCCCCCCCC[NH+](CCO)CCCCCCCCOC(=O)CCCCCCCC ; [Cl-]]

To a solution of Compound 6 (5.9 g, 7.82 mmol, 1 eq.) in DCM (59 mL) was added HCl/dioxane (0.5 M, 46.94 mL, 3 eq.). The mixture was stirred at 20° C. for 2 hours. The reaction mixture was concentrated under reduced pressure to give a residue. Then the residue was diluted with acetonitrile (5 mL) and H₂O (80 mL). The mixture was lyophilized to give Lipid 1 (5.01 g, 6.34 mmol, 81.00% yield, 100% purity, HCl) as yellow gum.

LCMS [M+1]⁺=754.7

¹H NMR (400 MHz, DMSO-d₆) δ=9.51 (br s, 1H), 5.30 (br s, 1H), 4.66-4.53 (m, 1H), 4.11-3.93 (m, 4H), 3.72 (br d, J=3.0 Hz, 2H), 3.19-2.99 (m, 6H), 2.26 (br t, J=7.2 Hz, 2H), 1.68-1.47 (m, 14H), 1.34-1.18 (m, 52H), 0.94-0.76 (m, 9H)

### General Procedure for the Synthesis of Compound 100:

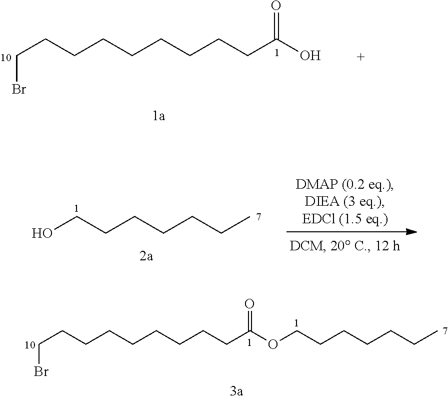

To a solution of Compound 1a (1 g, 3.98 mmol, 1 eq.) in DCM (10 mL) was added EDCI (1.14 g, 5.97 mmol, 1.5 eq.), DIEA (1.54 g, 11.94 mmol, 2.08 mL, 3 eq.), DMAP (97.28 mg, 796.31 μmol, 0.2 eq.) and Compound 2a (462.66 mg, 3.98 mmol, 562.84 μL, 1 eq.). The mixture was stirred at 20° C. for 12 hours. TLC (Petroleum ether: Ethyl acetate=3:1, R_{f}=0.51) indicated new spots were formed. The reaction mixture was concentrated under reduced pressure to give a residue. The residue was purified by column chromatography (SiO₂, Petroleum ether: Ethyl acetate=1:0 to 100:1) to give Compound 3a (510 mg, 1.46 mmol, 36.66% yield, 99.99% purity) as a colorless oil.

¹H NMR (400 MHz, CHLOROFORM-d) 8=4.07 (t, J=6.8 Hz, 2H), 3.41 (t, J=6.8 Hz, 2H), 2.30 (t, J=7.2 Hz, 2H), 1.86 (quin, J=7.2 Hz, 2H), 1.62 (br t, J=7.2 Hz, 4H), 1.45-1.23 (m, 18H), 0.95-0.81 (m, 3H).

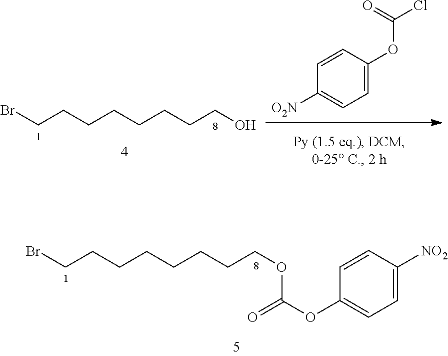 [SMILES: O=C(Cl)Oc1ccc([N+](=O)[O-])cc1 ; OCCCCCCCCBr ; O=C(OCCCCCCCCBr)Oc1ccc([N+](=O)[O-])cc1]

To a solution of Compound 4 (20 g, 95.64 mmol, 16.39 mL, 1 eq.) in DCM (200 mL) was added Pyridine (11.35 g, 143.46 mmol, 11.58 mL, 1.5 eq.). Then, 4-nitrophenyl carbonochloridate (23.13 g, 114.76 mmol, 1.2 eq.) in DCM (200 mL) was added dropwise at 0° C. The resulting mixture was stirred at 20° C. for 2 hours. TLC (Petroleum ether: Ethyl acetate=10:1, R_{f}=0.67) showed Compound 4 was consumed completely and new spots were formed. The reaction mixture was diluted with H₂O (100 mL) and extracted with DCM (50 mL×3). The combined organic layers were concentrated under reduced pressure to give a residue. The residue was purified by column chromatography (SiO₂, Petroleum ether: Ethyl acetate=20:1 to 10:1) to give Compound 5 (24.6 g, 64.73 mmol, 67.68% yield, 98.47% purity) as a white solid.

LCMS [M+1]⁺=396.1, 398.1

¹H NMR (CHLOROFORM-d, 400 MHz) δ=8.33-8.25 (m, 2H), 7.42-7.36 (m, 2H), 4.30 (t, J=6.8 Hz, 2H), 3.42 (t, J=6.8 Hz, 2H), 1.87 (quin, J=7.2 Hz, 2H), 1.81-1.73 (m, 2H), 1.51-1.35 (m, 8H).

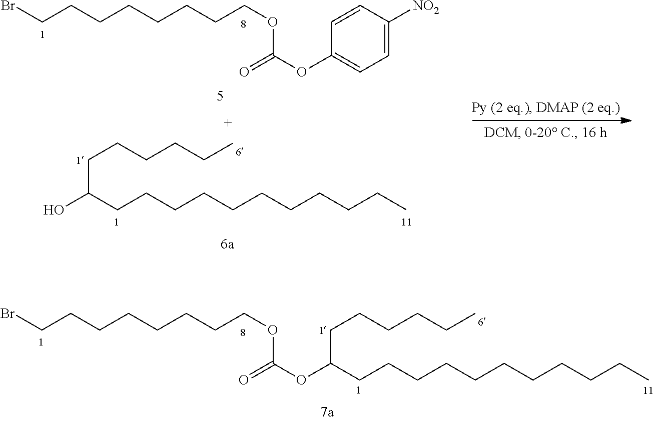 [SMILES: O=C(OCCCCCCCCBr)Oc1ccc([N+](=O)[O-])cc1 ; CCCCCCCCCCCC(O)CCCCCC ; CCCCCCCCCCCC(CCCCCC)OC(=O)OCCCCCCCCBr]

To a solution of Compound 5 (9.00 g, 24.05 mmol, 1.8 eq.) and Compound 6a (3.61 g, 13.36 mmol, 1 eq.) in DCM (50 mL) was added Pyridine (2.11 g, 26.72 mmol, 2.16 mL, 2 eq.) dropwise at 0° C. over 10 min. Then, DMAP (3.26 g, 26.72 mmol, 2 eq.) was added at 0° C. The resulting mixture was stirred at 20° C. for 16 hours. TLC (Petroleum ether: Ethyl acetate=10:1, R_{f}=0.53) indicated new spots were formed. The reaction mixture was concentrated under reduced pressure to give a residue. The residue was purified by column chromatography (SiO₂, Petroleum ether: Ethyl acetate=1:0 to 50:1) to give Compound 7a (1.38 g, 2.73 mmol, 20.40% yield, 99.84% purity) as a colorless oil.

¹H NMR (400 MHz, CHLOROFORM-d) 8=4.75-4.62 (m, 1H), 4.12 (t, J=6.8 Hz, 2H), 3.41 (t, J=6.8 Hz, 2H), 1.86 (quin, J=7.2 Hz, 2H), 1.73-1.62 (m, 2H), 1.62-1.56 (m, 2H), 1.56-1.49 (m, 2H), 1.48-1.19 (m, 34H), 0.94-0.83 (m, 6H)

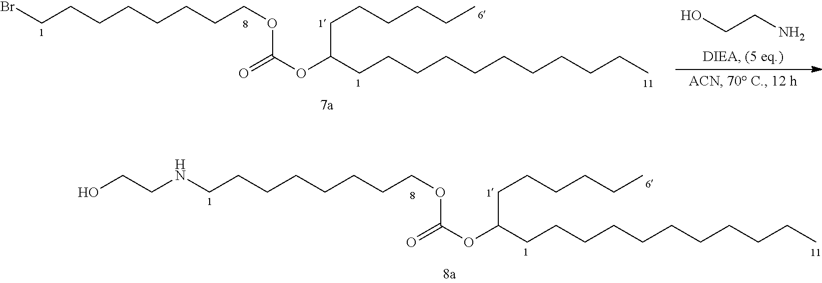

To a solution of Compound 7a (1.18 g, 2.33 mmol, 1 eq.) in acetonitrile (11 mL) was added DIEA (1.51 g, 11.67 mmol, 2.03 mL, 5 eq.) and 2-aminoethanol (712.78 mg, 11.67 mmol, 704.33 μL, 5 eq.). The mixture was stirred at 70° C. for 12 hours. TLC (Petroleum ether: Ethyl acetate=30:1 and DCM: MeOH=10:1, Rt=0.18) indicated Compound 7a was consumed completely and new spots formed. The reaction mixture was diluted with Ethyl acetate (40 mL) and washed with H₂O (20 mL×2), dried over Na₂SO₄, filtered and concentrated under reduced pressure to give a residue. The residue was purified by column chromatography (SiO₂, DCM: MeOH=100:1 to 5:1) to give Compound 8a (740 mg, 1.50 mmol, 64.15% yield, 98.28% purity) as a colorless oil.

LCMS [M+1]⁺=486.5

¹H NMR (400 MHz, CHLOROFORM-d) 8=4.74-4.62 (m, 1H), 4.12 (t, J=6.8 Hz, 2H), 3.72-3.64 (m, 2H), 2.87-2.77 (m, 2H), 2.65 (t, J=7.2 Hz, 2H), 1.72-1.63 (m, 2H), 1.62-1.48 (m, 6H), 1.40-1.23 (m, 34H), 0.94-0.80 (m, 6H).

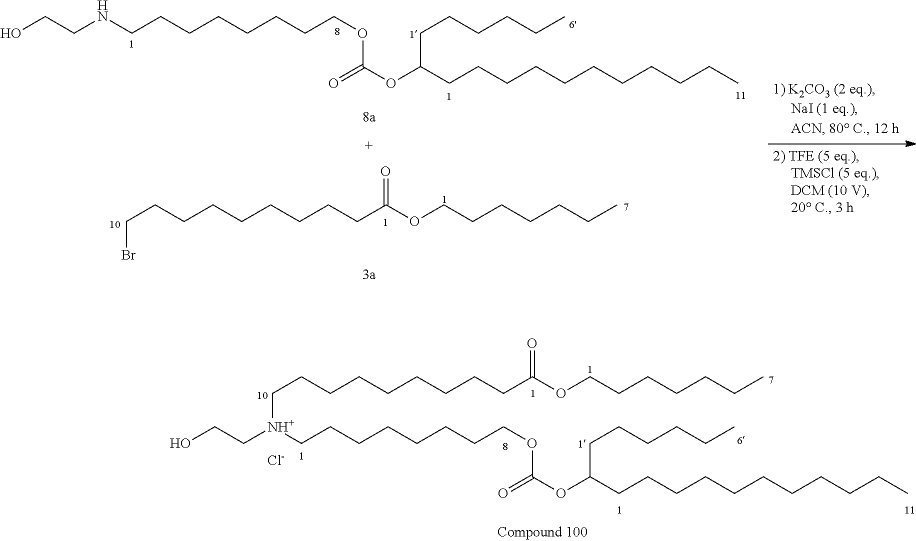 [SMILES: CCCCCCCCCCCC(CCCCCC)OC(=O)OCCCCCCCCNCCO ; CCCCCCCOC(=O)CCCCCCCCCBr ; CCCCCCCCCCCC(CCCCCC)OC(=O)OCCCCCCCC[NH+](CCO)CCCCCCCCCC(=O)OCCCCCCC ; [Cl-]]

A mixture of Compound 8a (500 mg, 1.03 mmol, 1 eq.), Compound 3a (359.57 mg, 1.03 mmol, 1 eq.), K₂CO₃ (284.50 mg, 2.06 mmol, 2 eq.) and NaI (154.28 mg, 1.03 mmol, 1 eq.) in acetonitrile (5 mL) was degassed and purged with N₂ 3 times, and the mixture was stirred at 80° C. for 16 hours under an N₂ atmosphere. TLC (DCM: MeOH=10:1, R_{f}=0.39) indicated that ˜16% of Compound 8a remained and new spots formed. The reaction mixture was diluted with H₂O (20 mL) and extracted with Ethyl acetate (20 mL×2), dried over Na₂SO₄, filtered and concentrated under reduced pressure to give a residue. The residue was purified by column chromatography (SiO₂, DCM: MeOH=80:1 to 20:1) to give crude product. The crude product (500 mg) was re-purified by column chromatography (SiO₂, DCM: MeOH=80:1 to 20:1) to give Compound 100 (140 mg, 181.93 μmol, 17.68% yield, 98.01% purity) as a light yellow oil.

LCMS [M+1]⁺=754.7

¹H NMR (400 MHz, CHLOROFORM-d) 8=4.76-4.62 (m, 1H), 4.18-4.00 (m, 4H), 3.74-3.44 (m, 2H), 2.75-2.37 (m, 5H), 2.30 (t, J=7.6 Hz, 2H), 1.73-1.59 (m, 8H), 1.53-1.41 (m, 4H), 1.28 (br d, J=12.4 Hz, 52H), 0.96-0.81 (m, 9H).

A solution of TMSCl (100.83 mg, 928.12 μmol, 117.80 μL, 5 eq.) in TFE (92.85 mg, 928.12 μmol, 66.75 μL, 5 eq.) was stirred at 20° C. for 1 hour. Then Compound 100 (140 mg, 185.62 μmol, 1 eq.) in DCM (1.4 mL) was added dropwise at 20° C. The resulting mixture was stirred at 20° C. for 2 hours. The mixture was concentrated to give Compound 100 (100.37 mg, 125.48 μmol, 67.60% yield, 98.85% purity, 1.0 HCl) as a light yellow oil.

LCMS [M+1]⁺=754.7

¹H NMR (400 MHz, CHLOROFORM-d) 8=11.61 (s, 1H), 5.03-4.86 (m, 1H), 4.76-4.63 (m, 1H), 4.19-4.04 (m, 4H), 4.04-3.96 (m, 2H), 3.25-2.96 (m, 6H), 2.30 (t, J=7.6 Hz, 2H), 1.97-1.75 (m, 4H), 1.73-1.58 (m, 8H), 1.51 (br s, 2H), 1.44-1.20 (m, 52H), 0.96-0.79 (m, 9H).

### General Procedure for the Synthesis of Compound 101:

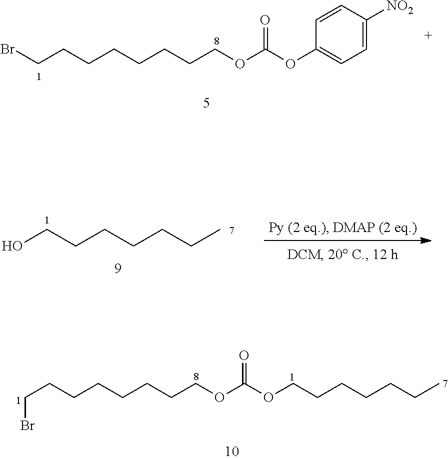

To a solution of Compound 5 (2 g, 5.34 mmol, 1 eq.) in DCM (20 mL) was added pyridine (845.47 mg, 10.69 mmol, 862.73 μL, 2 eq.), DMAP (1.31 g, 10.69 mmol, 2 eq.). Then, Compound 9 (621.02 mg, 5.34 mmol, 755.50 μL, 1 eq.) was added dropwise at 0° C. The resulting mixture was stirred at 20° C. for 12 hours. TLC (Petroleum ether: Ethyl acetate=10:1, R_{f}=0.73) showed Compound 5 was consumed completely, and new spots were formed. The reaction mixture was filtered and concentrated under reduced pressure to give a residue. The residue was purified by column chromatography (SiO₂, Petroleum ether: Ethyl acetate=1:0 to 100:1) to give Compound 10 (340 mg, 967.78 μmol, 18.11% yield) as a colorless oil.

LCMS [M+1]⁺=351.2, 353.2

¹H NMR (400 MHz, CHLOROFORM-d) 8=4.13 (t, J=6.8 Hz, 4H), 3.41 (t, J=6.8 Hz, 2H), 1.86 (quin, J=7.2 Hz, 2H), 1.71-1.63 (m, 4H), 1.47-1.28 (m, 16H), 0.92-0.85 (m, 3H)

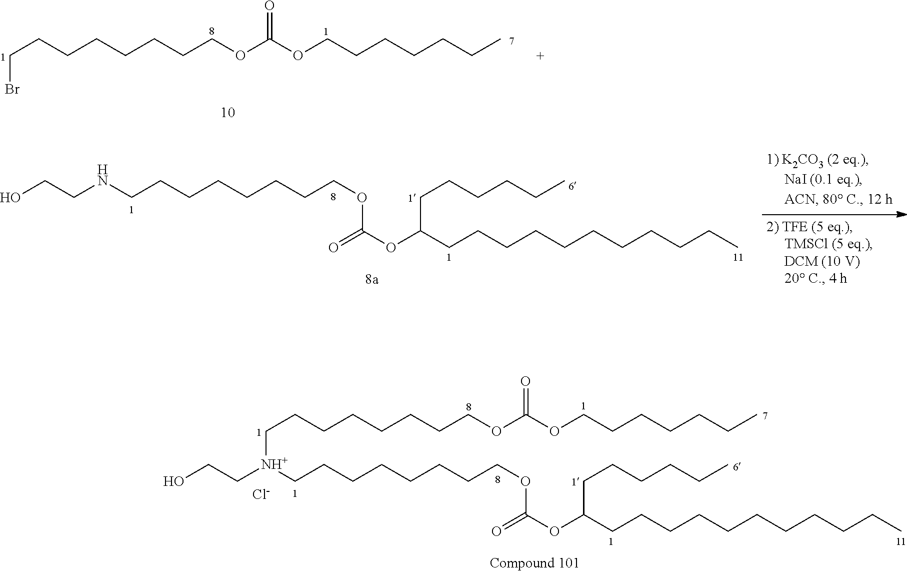 [SMILES: CCCCCCCOC(=O)OCCCCCCCCBr ; CCCCCCCCCCCC(CCCCCC)OC(=O)OCCCCCCCCNCCO ; CCCCCCCCCCCC(CCCCCC)OC(=O)OCCCCCCCC[NH+](CCO)CCCCCCCCOC(=O)OCCCCCCC ; [Cl-]]

A mixture of Compound 10 (260 mg, 740.07 μmol, 1 eq.), Compound 8a (359.51 mg, 740.07 μmol, 1 eq.), K₂CO₃ (204.56 mg, 1.48 mmol, 2 eq.), and NaI (110.93 mg, 740.07 μmol, 1 eq.) in acetonitrile (4 mL) was degassed and purged with N₂ 3 times. Then, the mixture was stirred at 80° C. for 6 hours under an N₂ atmosphere. TLC (Petroleum ether: Ethyl acetate=10:1, R_{f}=0.24) showed Compound 8a was consumed completely and new spots were detected. The reaction mixture was diluted with H₂O (10 mL) and extracted with DCM (10 mL×3). The combined organic layers were concentrated under reduced pressure to give a residue. The residue was purified by column chromatography (SiO₂, Dichloromethane: Methanol=1:0 to 100:1) to give Compound 101 (550 mg, 720.13 μmol, 97.31% yield, 99.01% purity) as a colorless oil.

LCMS [M+1]⁺=756.7

¹H NMR (400 MHz, CHLOROFORM-d) 8=4.75-4.63 (m, 1H), 4.17-4.09 (m, 6H), 3.59 (br s, 2H), 2.72-2.44 (m, 6H), 1.67 (quin, J=7.2 Hz, 6H), 1.56 (br d, J=6.8 Hz, 4H), 1.49 (br d, J=5.6 Hz, 4H), 1.39-1.25 (m, 50H), 0.95-0.85 (m, 9H).

A mixture of TFE (198.44 mg, 1.98 mmol, 142.66 μL, 5 eq.) in TMSCl (215.51 mg, 1.98 mmol, 251.76 μL, 5 eq.) was stirred at 20° C. for 1 hour under an N₂ atmosphere. Then, Compound 101 (300 mg, 396.73 μmol, 1 eq.) in DCM (3 mL) was added, and the mixture was stirred at 20° C. for 3 hours under an N₂ atmosphere. The reaction mixture was concentrated under reduced pressure to give Compound 101 (310 mg, 391.96 μmol, 98.80% yield, 99.30% purity, 0.8 HCl) as a light yellow oil.

LCMS [M+1]⁺=756.7

¹H NMR (400 MHz, DMSO-d₆) δ=11.43 (br d, J=4.4 Hz, 1H), 4.93 (br t, J=7.2 Hz, 1H), 4.69 (quin, J=6.4 Hz, 1H), 4.16-4.08 (m, 6H), 4.00 (br s, 2H), 3.16 (br s, 2H), 3.06 (br dd, J=5.6, 10.4 Hz, 4H), 1.93-1.78 (m, 4H), 1.71-1.63 (m, 6H), 1.61 (br s, 2H), 1.56 (br s, 2H), 1.39-1.25 (m, 50H), 0.89 (t, J=6.4 Hz, 9H).

### General Procedure for the Synthesis of Compound 102:

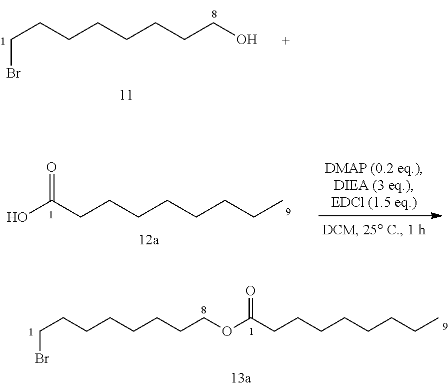 [SMILES: OCCCCCCCCBr ; CCCCCCCCC(=O)O ; CCCCCCCCC(=O)OCCCCCCCCBr]

To a solution of Compound 12a (10 g, 63.20 mmol, 1 eq.) in DCM (100 mL) was added DIEA (24.50 g, 189.59 mmol, 33.02 mL, 3 eq.), EDCI (18.17 g, 94.79 mmol, 1.5 eq.), DMAP (1.54 g, 12.64 mmol, 0.2 eq.) and Compound 11 (13.22 g, 63.20 mmol, 10.83 mL, 1 eq.). The mixture was stirred at 20° C. for 2 hours. LCMS showed Compound 12a was consumed completely and one main peak with the desired MS was detected. The reaction mixture was concentrated under reduced pressure to give a residue. The residue was diluted with brine (80 mL) and extracted with ethyl acetate (25 mL×4). The combined organic layers were dried over Na₂SO₄, filtered and concentrated under reduced pressure to give a residue. The residue was purified by column chromatography (SiO₂, petroleum ether: ethyl acetate=100:0 to 97:3) to give Compound 13a (10.86 g, 31.09 mmol, 49.19% yield, 100% purity) as a colorless oil.

LCMS [M+1]⁺=349.2, 351.2

¹H NMR (400 MHz, CHLOROFORM-d) S=4.06 (t, J=6.8 Hz, 2H), 3.42 (t, J=6.8 Hz, 2H), 2.30 (t, J=7.6 Hz, 2H), 1.86 (quin, J=7.2 Hz, 2H), 1.62 (quin, J=6.8 Hz, 4H), 1.44 (quin, J=7.2 Hz, 2H), 1.39-1.23 (m, 16H), 0.96-0.81 (m, 3H)

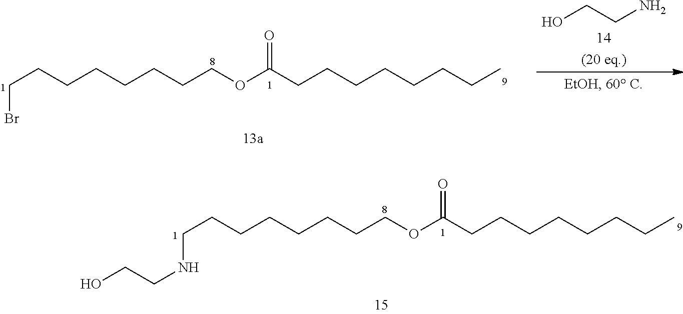

To a solution of Compound 13a (2 g, 5.72 mmol, 1 eq.) in EtOH (10 mL) was added Compound 14 (6.99 g, 114.50 mmol, 6.91 mL, 20 eq.). The mixture was stirred at 60° C. for 12 hours. LCMS showed Compound 13a was consumed completely and one main peak with desired MS was detected. The reaction mixture was concentrated under reduced pressure to give a residue. The residue was diluted with H₂O (30 mL) and extracted with ethyl acetate (10 mL×3). The combined organic layers were dried over Na₂SO₄, filtered and concentrated under reduced pressure to give a residue. The residue was purified by column chromatography (SiO₂, DCM: MeOH=100:0 to 93:7) to give Compound 15 (469 mg, 1.24 mmol, 21.63% yield, 87% purity) as a light yellow solid.

LCMS [M+1]⁺=330.3

¹H NMR (400 MHz, CHLOROFORM-d) S=4.06 (t, J=6.8 Hz, 2H), 3.78-3.55 (m, 2H), 2.86-2.76 (m, 2H), 2.63 (t, J=7.2 Hz, 2H), 2.29 (t, J=7.6 Hz, 2H), 1.70-1.57 (m, 4H), 1.56-1.44 (m, 2H), 1.41-1.21 (m, 18H), 0.88 (br t, J=6.8 Hz, 3H).

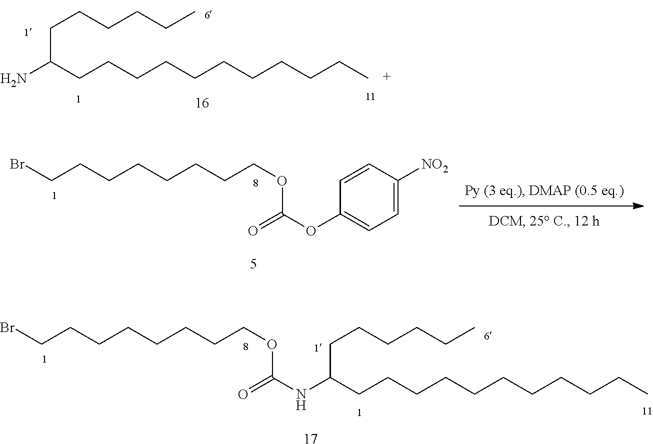

To a solution of Compound 16 (0.5 g, 1.86 mmol, 1 eq.) in DCM (5 mL) was added Compound 5 (694.28 mg, 1.86 mmol, 1 eq.), DMAP (113.32 mg, 927.62 μmol, 0.5 eq.) and pyridine (440.25 mg, 5.57 mmol, 449.23 μL, 3 eq.). The mixture was stirred at 30° C. for 16 hours. TLC (petroleum ether: ethyl acetate=3:1, R_{f}=0.8) indicated that Compound 16 remained and many new spots formed. The residue was diluted with H₂O (30 mL) and extracted with DCM (25 mL×2). The combined organic layers were dried over Na₂SO₄, filtered and concentrated under reduced pressure to give a residue. The residue was purified by column chromatography (SiO₂, petroleum ether:ethyl acetate=100:0 to 98:2) to give Compound 17 (530 mg, 901.14 μmol, 48.57% yield, 85.8% purity) as a light yellow solid.

LCMS [M+1]⁺=504.4, 506.4

¹H NMR (400 MHz, CHLOROFORM-d) S=4.34 (br d, J=9.2 Hz, 1H), 4.04 (br t, J=6.4 Hz, 2H), 3.57 (br dd, J=6.8, 17.6 Hz, 1H), 3.41 (t, J=6.8 Hz, 2H), 1.86 (td, J=7.2, 14.4 Hz, 2H), 1.60 (br s, 2H), 1.54-1.42 (m, 4H), 1.40-1.14 (m, 34H), 1.01-0.79 (m, 6H).

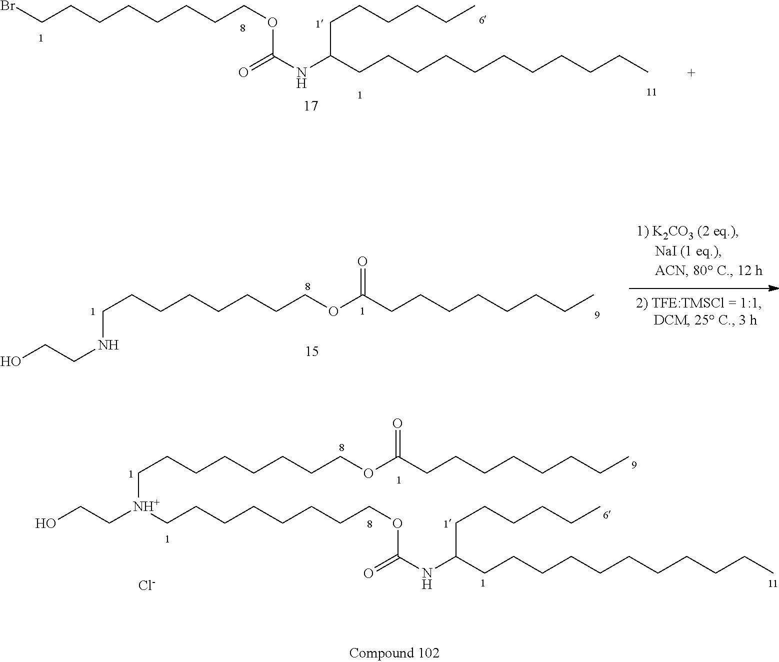 [SMILES: CCCCCCCCCCCC(CCCCCC)NC(=O)OCCCCCCCCBr ; CCCCCCCCC(=O)OCCCCCCCCNCCO ; CCCCCCCCCCCC(CCCCCC)NC(=O)OCCCCCCCC[NH+](CCO)CCCCCCCCOC(=O)CCCCCCCC ; [Cl-]]

To a solution of Compound 17 (420 mg, 832.30 μmol, 1 eq.) in ACN (5 mL) was added Compound 15 (274.26 mg, 832.30 μmol, 1 eq.), K₂CO₃ (230.06 mg, 1.66 mmol, 2 eq.) and NaI (124.76 mg, 832.30 μmol, 1 eq.). The mixture was stirred at 80° C. for 12 hours. TLC (dichloromethane: methanol=10:1, R_{f}=0.4) indicated that Compound 17 remained and many new spots formed. The reaction mixture was diluted with H₂O (30 mL) and extracted with DCM (25 mL×2). The combined organic layers were dried over Na₂SO₄, filtered and concentrated under reduced pressure to give a residue. The residue was purified by column chromatography (SiO₂, dichloromethane: methanol=100:0 to 95:5) to give Compound 102 (500 mg, 655.84 μmol, 78.80% yield, 98.8% purity) as a light yellow oil.

LCMS [M+1]⁺=753.7

¹H NMR (400 MHz, CHLOROFORM-d) S=4.41 (br d, J=9.2 Hz, 1H), 4.10-3.98 (m, 4H), 3.61 (br t, J=5.2 Hz, 3H), 2.68 (br t, J=4.8 Hz, 2H), 2.55 (br t, J=7.6 Hz, 4H), 2.29 (t, J=7.6 Hz, 2H), 1.62-1.57 (m, 4H), 1.55-1.44 (m, 6H), 1.39-1.20 (m, 56H), 0.88 (br t, J=6.8 Hz, 9H).

A solution of TFE (199.22 mg, 1.99 mmol, 143.22 μL, 5 eq.) in TMSCl (216.35 mg, 1.99 mmol, 252.75 μL, 5 eq.) was stirred at 20° C. for 1 hour. Then, Compound 102 (300 mg, 398.29 μmol, 1 eq.) in DCM (3 mL) was added. The mixture was stirred at 20° C. for 3 hours.

The reaction mixture was concentrated under reduced pressure to give Compound 102 (300 mg, 381.66 μmol, 95.83% yield, 0.9 HCl) as a light yellow oil.

LCMS [M+1]⁺=753.7

¹H NMR (400 MHz, CHLOROFORM-d) S=11.63-11.18 (m, 1H), 4.94 (br t, J=6.4 Hz, 1H), 4.39 (br d, J=9.2 Hz, 1H), 4.14-3.90 (m, 6H), 3.65-3.42 (m, 1H), 3.26-2.99 (m, 6H), 2.30 (t, J=7.6 Hz, 2H), 1.86 (br d, J=5.6 Hz, 4H), 1.68-1.61 (m, 4H), 1.45 (br s, 2H), 1.43-1.09 (m, 56H), 0.89 (t, J=6.8 Hz, 9H).

### General Procedure for the Synthesis of Compound 103:

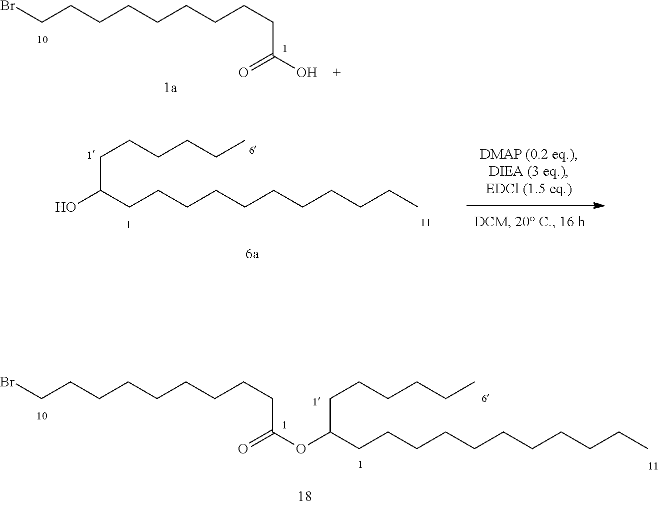 [SMILES: O=C(O)CCCCCCCCCBr ; CCCCCCCCCCCC(O)CCCCCC ; CCCCCCCCCCCC(CCCCCC)OC(=O)CCCCCCCCCBr]

To a solution of Compound 1a (1 g, 3.98 mmol, 1 eq.) in DCM (10 mL) was added DMAP (97.28 mg, 796.31 μmol, 0.2 eq.), EDCI (1.14 g, 5.97 mmol, 1.5 eq.) and DIEA (1.54 g, 11.94 mmol, 2.08 mL, 3 eq.) at 20° C. Then, Compound 6a (1.08 g, 3.98 mmol, 1 eq.) was added. The mixture was stirred at 20° C. for 16 hours. LCMS showed Compound 1a was consumed completely, and one main peak with the desired MS was detected. The reaction mixture was concentrated under reduced pressure to give a residue. The residue was purified by column chromatography (SiO₂, petroleum ether: ethyl acetate=80:1 to 20:1) to give Compound 18 (950 mg, 1.89 mmol, 86.24% purity) as a yellow oil.

LCMS [M/2+1]⁺=251.1, 253.1

¹H NMR (400 MHz, CHLOROFORM-d) S=4.87 (quin, J=6.4 Hz, 1H), 3.41 (t, J=6.8 Hz, 2H), 2.29 (t, J=7.6 Hz, 2H), 1.86 (quin, J=7.2 Hz, 2H), 1.68-1.59 (m, 2H), 1.51 (br d, J=5.6 Hz, 4H), 1.46-1.38 (m, 2H), 1.38-1.17 (m, 33H), 0.94-0.85 (m, 6H).

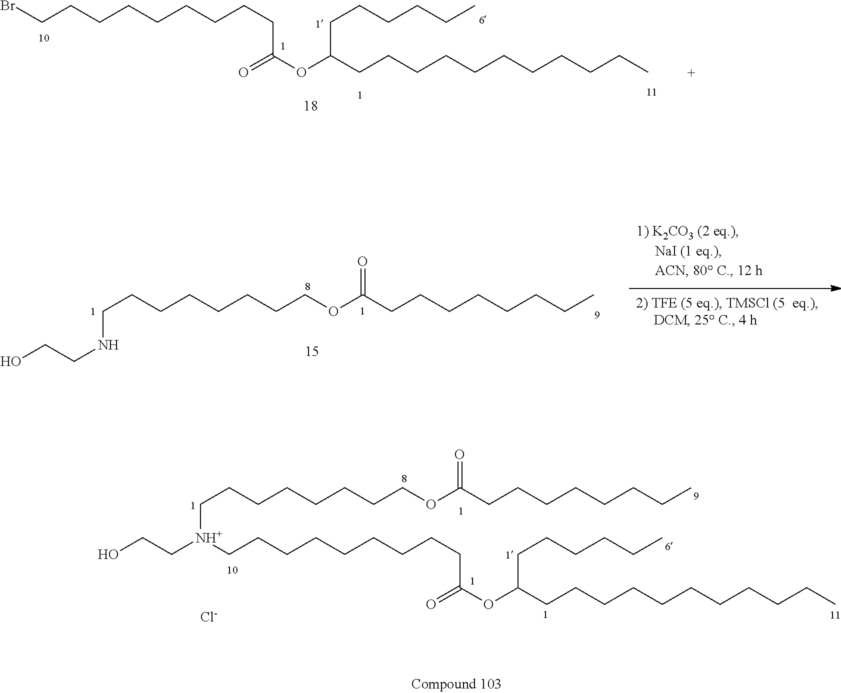 [SMILES: CCCCCCCCCCCC(CCCCCC)OC(=O)CCCCCCCCCBr ; CCCCCCCCC(=O)OCCCCCCCCNCCO ; CCCCCCCCCCCC(CCCCCC)OC(=O)CCCCCCCCC[NH+](CCO)CCCCCCCCOC(=O)CCCCCCCC ; [Cl-]]

To a solution of Compound 18 (500 mg, 992.78 μmol, 1 eq.) in ACN (5 mL) was added NaI (148.81 mg, 992.78 μmol, 1 eq.), K2CO₃ (274.41 mg, 1.99 mmol, 2 eq.) and Compound 15 (327.14 mg, 992.78 μmol, 1 eq.). The mixture was stirred at 80° C. for 12 hours. LCMS showed Compound 18 was consumed completely and one main peak with the desired MS was detected. The reaction mixture was partitioned between H₂O (10 mL) and Ethyl acetate (10 mL). The organic phase was separated, dried over Na₂SO₄, filtered and concentrated under reduced pressure to give a residue. The residue was purified by prep-HPLC (column: X-Select CSH Phenyl-Hexyl 100×305u; mobile phase: [H₂O (0.04% HCl)-ACN: THF=1:1]; gradient: 40%-90% B over 14.0 min) to give Compound 103 (300 mg, 395.78 μmol, 39.87% yield, 99.24% purity) as a colorless oil.

LCMS [M+1]⁺=752.7

¹H NMR (400 MHz, CHLOROFORM-d) S=4.87 (quin, J=6.4 Hz, 1H), 4.06 (t, J=6.8 Hz, 2H), 3.54 (br s, 2H), 2.59 (br s, 2H), 2.46 (br s, 4H), 2.35-2.22 (m, 4H), 1.64-1.57 (m, 6H), 1.51 (br d, J=5.6 Hz, 4H), 1.45 (br s, 4H), 1.37-1.23 (m, 54H), 0.89 (t, J=6.4 Hz, 9H).

A mixture of TFE (199.48 mg, 1.99 mmol, 143.41 μL, 5 eq.) and TMSCl (216.64 mg, 1.99 mmol, 253.08 μL, 5 eq.) was degassed and purged with N₂ 3 times, and the mixture was stirred at 20° C. for 1 hour under N₂ atmosphere. Then, Compound 103 (300 mg, 398.81 μmol, 1 eq.) in DCM (3 mL) was added at 20° C. The mixture was stirred at 20° C. for 3 hours.

The reaction mixture was concentrated under reduced pressure to give Compound 103 (300 mg, 378.28 μmol, 99.45% purity, 0.9 HCl) as a white solid.

LCMS [M+1]⁺=752.7

¹H NMR (400 MHz, CHLOROFORM-d) S=11.40 (br s, 1H), 4.86 (quin, J=6.4 Hz, 1H), 4.14-4.03 (m, 2H), 4.00 (br d, J=4.0 Hz, 2H), 3.25-3.14 (m, 2H), 3.12-2.97 (m, 4H), 2.35-2.22 (m, 4H), 1.92-1.78 (m, 4H), 1.62 (br t, J=6.8 Hz, 6H), 1.51 (br d, J=5.6 Hz, 4H), 1.44-1.11 (m, 54H), 0.88 (t, J=6.8 Hz, 9H).

### General Procedure for the Synthesis of Compound 104:

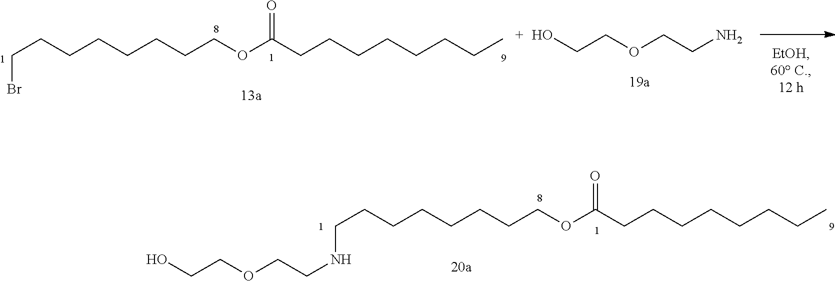

To a solution of Compound 13a (2 g, 5.72 mmol, 1 eq.) in EtOH (10 mL) was added Compound 19a (6.02 g, 57.25 mmol, 5.74 mL, 10 eq.) at 20° C. The mixture was stirred at 60° C. for 12 hours. LCMS showed Compound 13a was consumed completely and one main peak with the desired MS was detected. The reaction mixture was concentrated under reduced pressure to give a residue. The residue was diluted with H₂O (30 mL) and extracted with ethyl acetate (10 mL×3). The combined organic layers were dried over Na₂SO₄, filtered and concentrated under reduced pressure to give a residue. The residue was purified by column chromatography (SiO₂, DCM: MeOH=100:0 to 95:5) to give Compound 20a (1.06 g, 2.66 mmol, 46.49% yield, 93.8% purity) as a colorless oil.

LCMS [M+1]⁺=374.4

¹H NMR (400 MHz, CHLOROFORM-d) S=4.06 (t, J=6.8 Hz, 2H), 3.78-3.69 (m, 2H), 3.68-3.56 (m, 4H), 2.88-2.77 (m, 2H), 2.68-2.58 (m, 2H), 2.49-2.33 (m, 2H), 2.29 (t, J=7.6 Hz, 2H), 1.67-1.57 (m, 4H), 1.55-1.45 (m, 2H), 1.39-1.22 (m, 18H), 0.94-0.81 (m, 3H).

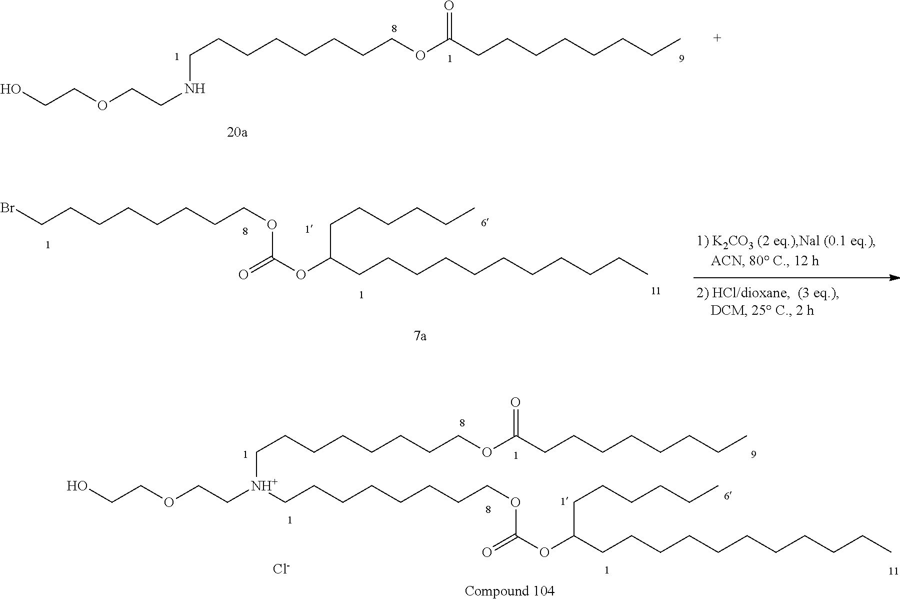 [SMILES: CCCCCCCCC(=O)OCCCCCCCCNCCOCCO ; CCCCCCCCCCCC(CCCCCC)OC(=O)OCCCCCCCCBr ; CCCCCCCCCCCC(CCCCCC)OC(=O)OCCCCCCCC[NH+](CCCCCCCCOC(=O)CCCCCCCC)CCOCCO ; [Cl-]]

To a solution of Compound 20a (600 mg, 1.61 mmol, 1 eq.) in acetonitrile (6 mL) was added K₂CO₃ (443.95 mg, 3.21 mmol, 2 eq.), NaI (240.75 mg, 1.61 mmol, 1 eq.), and Compound 7a (812.08 mg, 1.61 mmol, 1 eq.). The mixture was stirred at 80° C. for 12 hours.

LCMS showed Compound 7a was consumed completely and one main peak with desired MS was detected. The mixture was filtered, and the filtrate was concentrated to give a residue. The residue was purified by column chromatography (SiO₂, Petroleum ether: Ethyl acetate=10:1 to 0:1) to give Compound 104 (307 mg, 382.28 μmol, 23.80% yield, 99.40% purity) as a light yellow oil.

LCMS [M+1]⁺=798.7

¹H NMR (400 MHz, CHLOROFORM-d) S=4.74-4.63 (m, 1H), 4.18-3.99 (m, 4H), 3.71 (br s, 4H), 3.65-3.60 (m, 2H), 2.97-2.40 (m, 6H), 2.30 (t, J=7.6 Hz, 2H), 1.77-1.56 (m, 14H), 1.39-1.22 (m, 52H), 0.89 (t, J=6.4 Hz, 9H).

A solution of TMSCl (208.91 mg, 1.92 mmol, 244.05 μL, 5 eq.) in TFE (192.37 mg, 1.92 mmol, 138.29 μL, 5 eq.) was stirred at 20° C. for 1 hour. Then, Compound 104 (307 mg, 384.58 μmol, 1 eq.) in DCM (3 mL) was added to the mixture at 20° C. The resulting mixture was stirred at 20° C. for 3 hours. The reaction mixture was concentrated to give Compound 104 (317 mg, 380.17 μmol, 98.85% yield, 99.67% purity, 0.9 HCl) as a light yellow gum.

LCMS [M+1]⁺=798.7

¹H NMR (400 MHz, CHLOROFORM-d) S=11.79-11.57 (m, 1H), 4.74-4.63 (m, 1H), 4.15-4.02 (m, 4H), 3.93 (br s, 2H), 3.83-3.74 (m, 2H), 3.68-3.60 (m, 2H), 3.26-3.00 (m, 6H), 2.30 (t, J=7.6 Hz, 2H), 1.73-1.50 (m, 12H), 1.42-1.22 (m, 54H), 0.89 (t, J=6.8 Hz, 9H).

### General Procedure for the Synthesis of Compound 105:

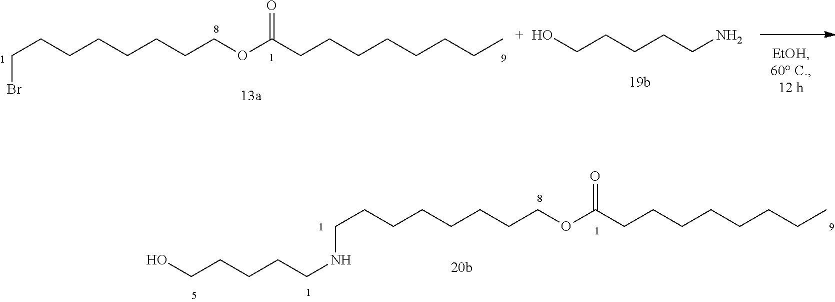

To a solution of Compound 13a (2 g, 5.72 mmol, 1 eq.) in EtOH (10 mL) was added Compound 19b (2.95 g, 28.62 mmol, 5 eq.). The mixture was stirred at 60° C. for 16 hours.

LCMS showed that the desired MS was detected. The reaction mixture was concentrated under reduced pressure to give a residue. The residue was purified by column chromatography (SiO₂, DCM: MeOH=100:1 to 93:7) to give Compound 20b (1.21 g, 3.08 mmol, 53.87% yield, 94.71% purity) as a light yellow oil.

LCMS [M+1]⁺=372.4

¹H NMR (400 MHz, CHLOROFORM-d) S=4.06 (t, J=6.8 Hz, 2H), 3.66 (t, J=6.4 Hz, 2H), 2.76-2.57 (m, 4H), 2.30 (t, J=7.6 Hz, 2H), 1.66-1.40 (m, 14H), 1.35-1.26 (m, 17H), 0.95-0.83 (m, 3H)

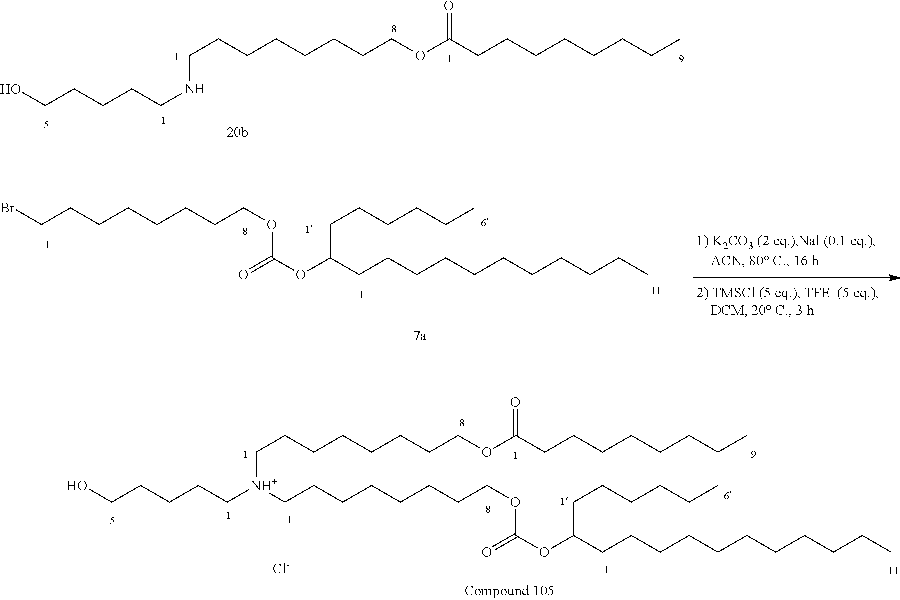 [SMILES: CCCCCCCCC(=O)OCCCCCCCCNCCCCCO ; CCCCCCCCCCCC(CCCCCC)OC(=O)OCCCCCCCCBr ; CCCCCCCCCCCC(CCCCCC)OC(=O)OCCCCCCCC[NH+](CCCCCO)CCCCCCCCOC(=O)CCCCCCCC ; [Cl-]]

To a solution of Compound 20b (600 mg, 1.61 mmol, 1 eq.) and Compound 7a (816.39 mg, 1.61 mmol, 1 eq.) in acetonitrile (6 mL) was added K₂CO₃ (446.32 mg, 3.23 mmol, 2 eq.) and NaI (242.02 mg, 1.61 mmol, 1 eq.). The mixture was stirred at 80° C. for 16 hours.

TLC (DCM: MeOH=10:1, R_{f}=0.54) indicated one new spot was formed. The reaction mixture was concentrated under reduced pressure to give a residue. The residue was purified by column chromatography (SiO₂, DCM: MeOH=100:1 to 97:3) to give Compound 105 (788 mg, 987.90 μmol, 61.18% yield, 99.83% purity) as a colorless oil.

LCMS [M+1]⁺=796.8

¹H NMR (400 MHz, CHLOROFORM-d) S=4.69 (br t, J=6.4 Hz, 1H), 4.21-4.02 (m, 4H), 3.66 (q, J=6.4 Hz, 2H), 2.56-2.32 (m, 6H), 2.30 (t, J=7.6 Hz, 2H), 1.78 (t, J=5.6 Hz, 1H), 1.71-1.58 (m, 12H), 1.51-1.21 (m, 60H), 0.89 (t, J=6.8 Hz, 9H).

A solution of TFE (188.45 mg, 1.88 mmol, 135.48 μL, 5 eq.) in TMSCl (204.65 mg, 1.88 mmol, 239.07 μL, 5 eq.) was stirred at 20° C. for 1 hour, and then Compound 105 (0.3 g, 376.75 μmol, 1 eq.) in DCM (3 mL) was added. The resulting mixture was stirred at 20° C. for 2 hours. The reaction mixture was concentrated under reduced pressure to give Compound 105 (311 mg, 371.91 μmol, 98.72% yield, 99.15% purity, 0.9 HCl) as a colorless oil.

LCMS [M+1]⁺=796.8

¹H NMR (400 MHz, CHLOROFORM-d) S=12.13-11.87 (m, 1H), 4.69 (t, J=6.0 Hz, 1H), 4.19-3.96 (m, 4H), 3.69 (t, J=6.0 Hz, 2H), 2.98 (br d, J=2.0 Hz, 6H), 2.30 (t, J=7.6 Hz, 2H), 1.96-1.73 (m, 6H), 1.70-1.61 (m, 8H), 1.58-1.44 (m, 6H), 1.41-1.21 (m, 52H), 0.89 (t, J=6.8 Hz, 9H).

### General Procedure for the Synthesis of Compound 106:

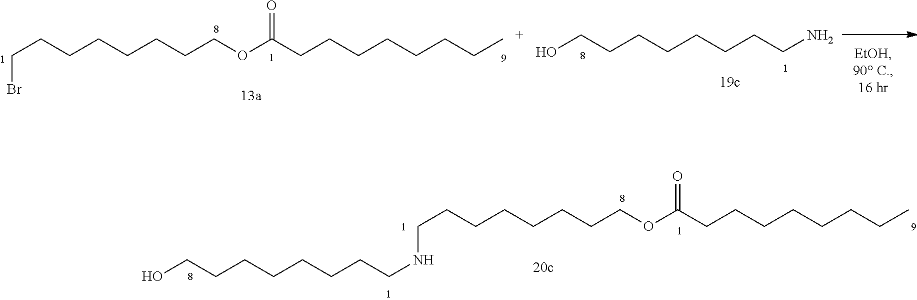 [SMILES: CCCCCCCCC(=O)OCCCCCCCCBr ; NCCCCCCCCO ; CCCCCCCCC(=O)OCCCCCCCCNCCCCCCCCO]

To a solution of Compound 13a (2 g, 5.72 mmol, 1 eq.) EtOH (10 mL) was added Compound 19c (4.16 g, 28.62 mmol, 5 eq.). The mixture was stirred at 90° C. for 16 hours.

LCMS showed that the desired MS was detected. The reaction mixture was concentrated under reduced pressure to give a residue. The residue was purified by column chromatography (SiO₂, DCM: MeOH=100:1 to 95:5) to give Compound 20c (901 mg, 2.12 mmol, 36.99% yield, 97.23% purity) as a light yellow oil.

LCMS [M+1]⁺=414.4

¹H NMR (400 MHz, CHLOROFORM-d) S=4.05 (t, J=6.8 Hz, 2H), 3.64 (t, J=6.8 Hz, 2H), 2.59 (t, J=7.2 Hz, 4H), 2.29 (t, J=7.6 Hz, 2H), 1.64-1.44 (m, 12H), 1.39-1.25 (m, 25H), 1.01-0.79 (m, 3H).

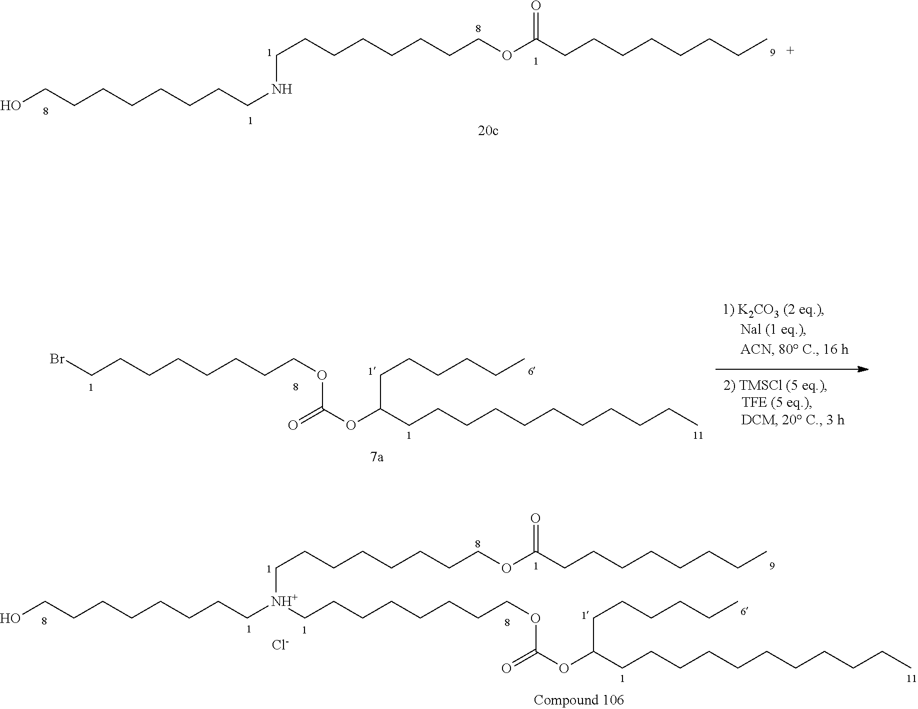 [SMILES: CCCCCCCCC(=O)OCCCCCCCCNCCCCCCCCO ; CCCCCCCCCCCC(CCCCCC)OC(=O)OCCCCCCCCBr ; CCCCCCCCCCCC(CCCCCC)OC(=O)OCCCCCCCC[NH+](CCCCCCCCO)CCCCCCCCOC(=O)CCCCCCCC ; [Cl-]]

To a solution of Compound 20c (600 mg, 1.45 mmol, 1 eq.) and Compound 7a (733.34 mg, 1.45 mmol, 1 eq.) in acetonitrile (6 mL) was added K₂CO₃ (400.92 mg, 2.90 mmol, 2 eq.) and NaI (217.40 mg, 1.45 mmol, 1 eq.). The mixture was stirred at 80° C. for 16 hours. TLC (DCM: MeOH=10:1, R_{f}=0.54) indicated one new spot was formed. The reaction mixture was concentrated under reduced pressure to give a residue. The residue was purified by column chromatography (SiO₂, DCM: MeOH=100:1 to 98:2) to give Compound 106 (517 mg, 611.37 μmol, 42.15% yield, 99.14% purity) as a colorless oil.

LCMS [M+1]⁺=838.8

¹H NMR (400 MHz, CHLOROFORM-d) S=4.84-4.59 (m, 1H), 4.21-3.98 (m, 4H), 3.76-3.55 (m, 2H), 2.50-2.32 (m, 6H), 2.30 (t, J=7.6 Hz, 2H), 1.72-1.60 (m, 6H), 1.56-1.49 (m, 4H), 1.46-1.16 (m, 68H), 0.89 (t, J=6.8 Hz, 9H).

A solution of TFE (178.99 mg, 1.79 mmol, 128.68 μL, 5 eq.) in TMSCl (194.38 mg, 1.79 mmol, 227.08 μL, 5 eq.) was stirred at 20° C. for 1 hour, and then Compound 106 (300 mg, 357.84 μmol, 1 eq.) in DCM (3 mL) was added. The resulting mixture was stirred at 20° C. for 2 hours. The reaction mixture was concentrated under reduced pressure to give Compound 106 (311 mg, 371.91 μmol, 98.72% yield, 99.15% purity, 0.9 HCl) as a colorless oil.

LCMS [M+1]⁺=838.8

¹H NMR (400 MHz, CHLOROFORM-d) S=12.15-11.97 (m, 1H), 4.74-4.63 (m, 1H), 4.20-3.99 (m, 4H), 3.65 (t, J=6.4 Hz, 2H), 3.03-2.88 (m, 6H), 2.35-2.24 (m, 2H), 1.86-1.73 (m, 6H), 1.71-1.56 (m, 12H), 1.39-1.22 (m, 60H), 0.89 (t, J=6.8 Hz, 9H).

### General Procedure for the Synthesis of Compound 107:

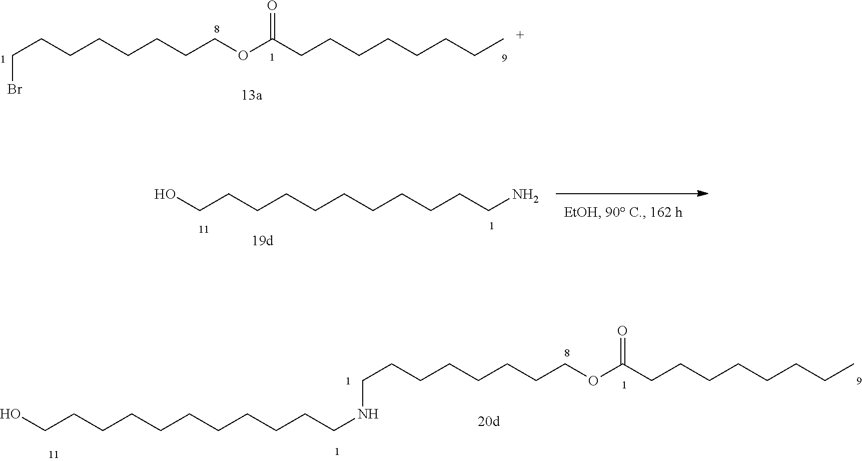 [SMILES: CCCCCCCCC(=O)OCCCCCCCCBr ; NCCCCCCCCCCCO ; CCCCCCCCC(=O)OCCCCCCCCNCCCCCCCCCCCO]

To a solution of Compound 13a (700.00 mg, 2.00 mmol, 1 eq.) in EtOH (7 mL) was added Compound 19d (1.5 g, 8.01 mmol, 4 eq.). The mixture was stirred at 90° C. for 16 hours.

LCMS showed that the desired MS was detected. The reaction mixture was concentrated under reduced pressure to give a residue. The residue was diluted with H₂O (20 mL) and extracted with ethyl acetate (20 mL×3). The combined organic layers were washed with brine (30 mL), dried over Na₂SO₄, filtered and concentrated under reduced pressure to give a residue. The residue was purified by column chromatography (SiO₂, dichloromethane: methanol=150:1 to 10:1) to give Compound 20d (330 mg, 664.34 μmol, 33.19% yield, 91.75% purity) as a colorless oil.

LCMS [M+1]⁺=456.5

¹H NMR (400 MHz, CHLOROFORM-d) S=4.06 (t, J=6.8 Hz, 2H), 3.64 (t, J=6.4 Hz, 2H), 2.59 (t, J=7.2 Hz, 4H), 2.29 (t, J=7.6 Hz, 2H), 1.65-1.45 (m, 12H), 1.30 (br d, J=12.8 Hz, 32H), 0.96-0.82 (m, 3H).

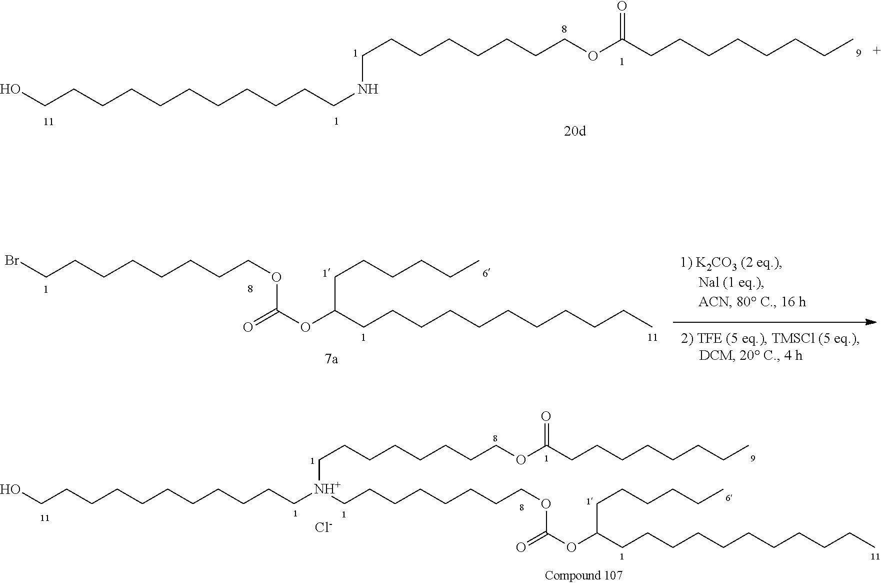 [SMILES: CCCCCCCCC(=O)OCCCCCCCCNCCCCCCCCCCCO ; CCCCCCCCCCCC(CCCCCC)OC(=O)OCCCCCCCCBr ; CCCCCCCCCCCC(CCCCCC)OC(=O)OCCCCCCCC[NH+](CCCCCCCCCCCO)CCCCCCCCOC(=O)CCCCCCCC ; [Cl-]]

To a solution of Compound 20d (330 mg, 724.07 μmol, 1 eq.) in acetonitrile (3.5 mL) was added Compound 7a (366.10 mg, 724.07 μmol, 1 eq.), NaI (108.53 mg, 724.07 μmol, 1 eq.) and K₂CO₃ (200.15 mg, 1.45 mmol, 2 eq.). The mixture was stirred at 80° C. for 16 hours. LCMS showed that the desired MS was detected. The reaction mixture was diluted with ethyl acetate (10 mL) and filtered. The filtrate was concentrated under reduced pressure to give a residue (700 mg). The residue was purified by flash silica gel chromatography (ISCO®; 20 g SepaFlash® Silica Flash Column, eluent of 1-4% Methanol: Dichloromethane gradient @80 mL/min) to give crude Compound 107. The crude Compound 107 (140 mg, purity: 91.05%) was purified by prep-HPLC (column: X-Select CSH Phenyl-Hexyl 100×30 5u; mobile phase: [H₂O (0.04% HCl)-ACN: THF=1:1]; gradient: 50%-90% B over 14.0 min) and neutralized with saturated NaHCO₃ (˜15 drops). The mixture was concentrated to remove acetonitrile, diluted with H₂O (10 mL) and extracted with DCM (20 mL×2). The combined organic layers were dried over Na₂SO₄, filtered and concentrated under reduced pressure to give Compound 107 (90 mg, 100.68 μmol, 13.90% yield, 98.49% purity) as a colorless oil.

LCMS [M+1]⁺=880.9

¹H NMR (400 MHz, CHLOROFORM-d) S=4.69 (br t, J=6.0 Hz, 1H), 4.21-3.98 (m, 4H), 3.64 (t, J=6.8 Hz, 2H), 2.38 (br s, 5H), 2.29 (t, J=7.6 Hz, 2H), 1.75-1.51 (m, 14H), 1.44-1.22 (m, 72H), 0.88 (br t, J=6.4 Hz, 9H).

A mixture of TFE (119.30 mg, 1.19 mmol, 85.77 μL, 5 eq.) and TMSCl (129.56 mg, 1.19 mmol, 151.36 μL, 5 eq.) was stirred at 25° C. for 1 hour. Then the solution of Compound 107 (210 mg, 238.51 μmol, 1 eq.) in DCM (2.5 mL) was added to the mixture. The resulting mixture was stirred at 25° C. for 3 hours under N₂ atmosphere. The reaction mixture was concentrated under reduced pressure to give Compound 107 (200 mg, 218.99 μmol, 91.82% yield, 0.9 HCl) as a light-yellow oil.

LCMS [M+1]⁺=880.9

¹H NMR (400 MHz, CHLOROFORM-d) S=12.06 (br s, 1H), 4.77-4.60 (m, 1H), 4.20-3.95 (m, 4H), 3.65 (t, J=6.4 Hz, 2H), 2.95 (br s, 6H), 2.29 (t, J=7.6 Hz, 2H), 1.80 (br s, 6H), 1.69-1.50 (m, 14H), 1.38-1.23 (m, 64H), 0.88 (t, J=6.8 Hz, 9H).

### General Procedure for the Synthesis of Compound 108:

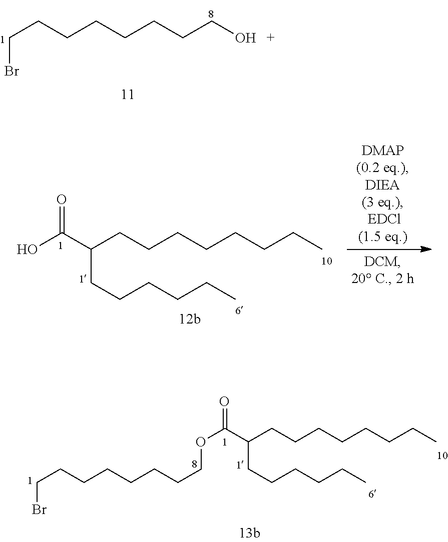 [SMILES: OCCCCCCCCBr ; CCCCCCCCC(CCCCCC)C(=O)O ; CCCCCCCCC(CCCCCC)C(=O)OCCCCCCCCBr]

To a solution of Compound 12b (1.23 g, 4.78 mmol, 1 eq.) in DCM (12 mL) was added DMAP (116.84 mg, 956.37 μmol, 0.2 eq.), EDCI (1.38 g, 7.17 mmol, 1.5 eq.), DIEA (1.85 g, 14.35 mmol, 2.50 mL, 3 eq.) and Compound 11 (1 g, 4.78 mmol, 819.67 μL, 1 eq.). The mixture was stirred at 20° C. for 2 hours. TLC (Petroleum ether: Ethyl acetate=10:1, R_{f}=0.68) showed Compound 12b was consumed completely and new spots were detected. The reaction mixture was concentrated under reduced pressure to give a residue. The residue was purified by column chromatography (SiO₂, Petroleum ether: Ethyl acetate=1:0 to 100:1) to give Compound 13b (730 mg, 1.48 mmol, 31.02% yield, 90.94% purity) as colorless oil.

LCMS [M+1]⁺=447.3, 449.3

¹H NMR (CHLOROFORM-d, 400 MHz) δ=4.07 (t, J=6.4 Hz, 2H), 3.41 (t, J=6.8 Hz, 2H), 2.36-2.27 (m, 1H), 1.86 (quin, J=7.2 Hz, 2H), 1.68-1.57 (m, 4H), 1.49-1.39 (m, 4H), 1.38-1.23 (m, 26H), 0.88 (t, J=6.4 Hz, 6H).

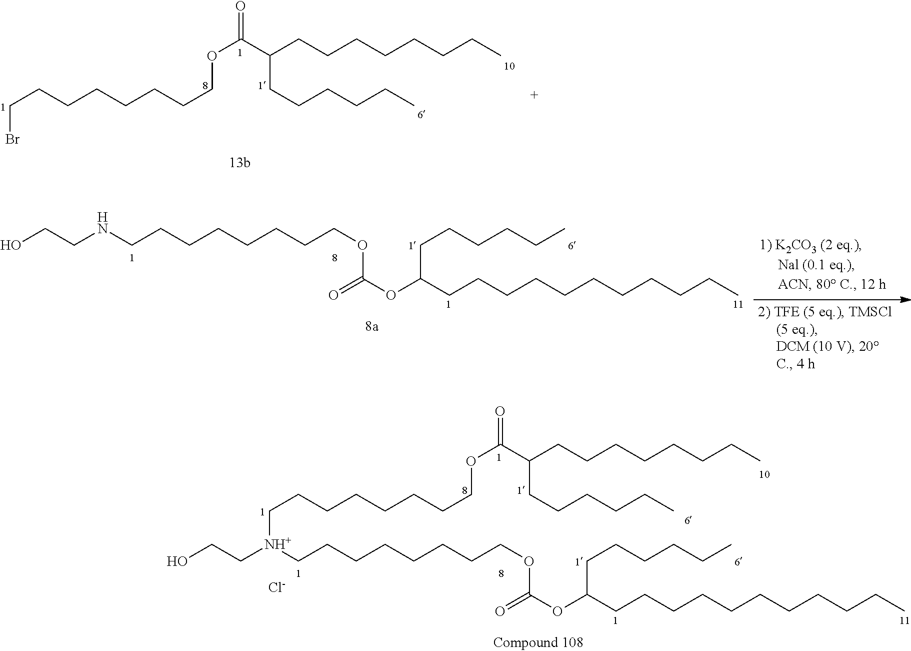 [SMILES: CCCCCCCCC(CCCCCC)C(=O)OCCCCCCCCBr ; CCCCCCCCCCCC(CCCCCC)OC(=O)OCCCCCCCCNCCO ; CCCCCCCCCCCC(CCCCCC)OC(=O)OCCCCCCCC[NH+](CCO)CCCCCCCCOC(=O)C(CCCCCC)CCCCCCCC ; [Cl-]]

A mixture of Compound 13b (500 mg, 1.12 mmol, 1 eq.), Compound 8a (542.73 mg, 1.12 mmol, 1 eq.), K₂CO₃ (308.82 mg, 2.23 mmol, 2 eq.) and NaI (167.47 mg, 1.12 mmol, 1 eq.) in ACN (5 mL) was degassed and purged with N₂ for 3 times, and then the mixture was stirred at 80° C. for 12 hours under N₂ atmosphere. TLC (Petroleum ether: Ethyl acetate=10:1, R_{f}=0.16) showed Compound 8a was consumed completely and new spots were detected. The combined reaction mixture were diluted with H₂O (10 mL) and extracted with DCM (10 mL×3). The combined organic layers were concentrated under reduced pressure to give a residue. The residue was purified by column chromatography (SiO₂, Dichloromethane: Methanol=1:0 to 100:1) to give Compound 108 (420 mg, 492.73 μmol, 44.10% yield, 100% purity) as colorless oil.

LCMS [M+1]⁺=852.8

¹H NMR (400 MHz, CHLOROFORM-d) 8=4.69 (quin, J=6.4 Hz, 1H), 4.09 (td, J=6.8, 19.4 Hz, 4H), 3.56 (br d, J=1.2 Hz, 2H), 2.76-2.38 (m, 6H), 2.37-2.26 (m, 1H), 1.73-1.58 (m, 8H), 1.48-1.41 (m, 4H), 1.39-1.18 (m, 66H), 0.93-0.83 (m, 12H).

A mixture of TFE (176.04 mg, 1.76 mmol, 126.56 μL, 5 eq.) and TMSCl (191.18 mg, 1.76 mmol, 223.34 μL, 5 eq.) was stirred at 20° C. for 1 hour under N₂ atmosphere. Then the solution of Compound 108 (300 mg, 351.95 μmol, 1 eq.) in DCM (3 mL) was added to the mixture. The resulting mixture was stirred at 20° C. for 3 hours under N₂ atmosphere. The reaction mixture was concentrated under reduced pressure to give Compound 108 (300 mg, 338.57 μmol, 96.20% yield, 99.49% purity, 0.8 HCl) as a light-yellow oil.

LCMS [M+1]⁺=852.8

¹H NMR (400 MHz, CHLOROFORM-d) S=11.47 (br s, 1H), 5.02-4.85 (m, 1H), 4.75-4.63 (m, 1H), 4.09 (td, J=6.8, 19.6 Hz, 4H), 4.00 (br d, J=2.4 Hz, 2H), 3.16 (br s, 2H), 3.11-2.98 (m, 4H), 2.37-2.25 (m, 1H), 1.85 (br d, J=3.6 Hz, 4H), 1.70-1.62 (m, 4H), 1.57-1.50 (m, 4H), 1.47-1.23 (m, 66H), 0.92-0.85 (m, 12H).

### General Procedure for the Synthesis of Compound 109:

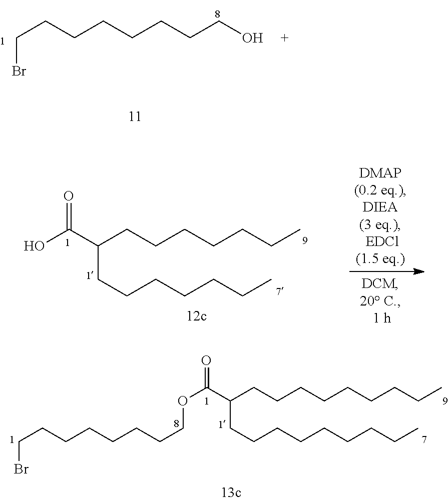 [SMILES: OCCCCCCCCBr ; CCCCCCCC(CCCCCCC)C(=O)O ; CCCCCCCCCC(CCCCCCCCC)C(=O)OCCCCCCCCBr]

To a solution of Compound 12c (1.23 g, 4.78 mmol, 1 eq.) in DCM (10 mL) was added DMAP (116.84 mg, 956.37 μmol, 0.2 eq.), EDCI (1.38 g, 7.17 mmol, 1.5 eq.), DIEA (1.85 g, 14.35 mmol, 2.50 mL, 3 eq.) and Compound 11 (1 g, 4.78 mmol, 819.67 μL, 1 eq.). The mixture was stirred at 20° C. for 1 hour. TLC (Petroleum ether: Ethyl acetate=10:1, R_{f}=0.8) indicated Compound 11 remained and many new spots formed. The reaction mixture was diluted with H₂O (30 mL) and extracted with DCM (25 mL×2). The combined organic layers were dried over Na₂SO₄, filtered and concentrated under reduced pressure to give a residue. The residue was purified by column chromatography (SiO₂, Petroleum ether: Ethyl acetate=100:0 to 98:2) to give Compound 13c (1.3 g, 2.53 mmol, 52.85% yield, 87% purity) as light-yellow oil.

LCMS [M+1]⁺=447.3, 449.3

¹H NMR (400 MHz, CHLOROFORM-d) S=4.07 (t, J=6.4 Hz, 2H), 3.41 (t, J=6.8 Hz, 2H), 2.37-2.27 (m, 1H), 1.86 (quin, J=7.2 Hz, 2H), 1.67-1.57 (m, 4H), 1.48-1.39 (m, 4H), 1.40-1.22 (m, 26H), 0.88 (t, J=6.8 Hz, 6H)

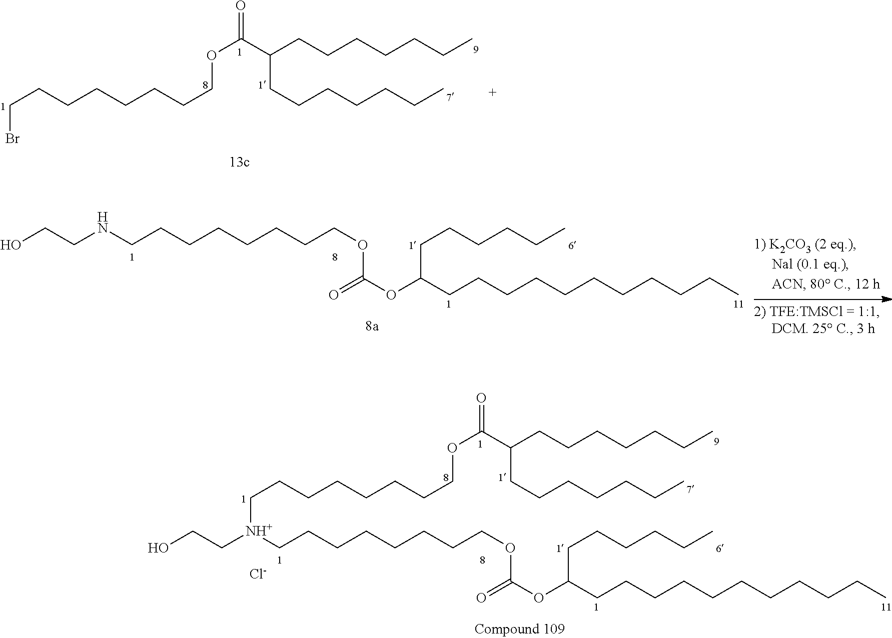 [SMILES: CCCCCCCC(CCCCCCC)C(=O)OCCCCCCCCBr ; CCCCCCCCCCCC(CCCCCC)OC(=O)OCCCCCCCCNCCO ; CCCCCCCCCCCC(CCCCCC)OC(=O)OCCCCCCCC[NH+](CCO)CCCCCCCCOC(=O)C(CCCCCCC)CCCCCCC ; [Cl-]]

To a solution of Compound 13c (500 mg, 1.12 mmol, 1 eq.) in ACN (5 mL) was added Compound 8a (542.73 mg, 1.12 mmol, 1 eq.), K₂CO₃ (617.64 mg, 4.47 mmol, 4 eq.) and NaI (167.47 mg, 1.12 mmol, 1 eq.). The mixture was stirred at 90° C. for 3 hours. TLC (Dichloromethane: Methanol=10:1, R_{f}=0.4) indicated Compound 13c remained and many new spots formed. The reaction mixture was diluted with H₂O (30 mL) and extracted with DCM (25 mL×2). The combined organic layers were dried over Na₂SO₄, filtered and concentrated under reduced pressure to give a residue. The residue was purified by column chromatography (SiO₂, Dichloromethane: Methanol=100:0 to 96:4) to give Compound 109 (820 mg, 961.99 μmol, 86.10% yield, Free) as a light-yellow oil.

LCMS [M+1]⁺=852.8

¹H NMR (400 MHz, CHLOROFORM-d) S=4.74-4.63 (m, 1H), 4.09 (td, J=6.8, 19.2 Hz, 4H), 3.57 (br s, 2H), 2.69-2.58 (m, 2H), 2.50 (br s, 4H), 2.36-2.27 (m, 1H), 1.71-1.55 (m, 10H), 1.50-1.41 (m, 6H), 1.37-1.17 (m, 62H), 0.96-0.81 (m, 12H).

A mixture of TMSCl (254.91 mg, 2.35 mmol, 297.79 μL, 5 eq.) and TFE (234.72 mg, 2.35 mmol, 168.75 μL, 5 eq.) was stirred at 20° C. for 1 hour. The solution of Compound 109 (400 mg, 469.26 μmol, 1 eq.) in DCM (4 mL) was added to the mixture. The resulting mixture was stirred at 20° C. for 3 hours. The reaction mixture was concentrated under reduced pressure to give Compound 109 (400 mg, 451.87 μmol, 96.29% yield, 0.9 HCl) as a light-yellow oil.

LCMS [M+1]⁺=852.8

¹H NMR (400 MHz, CHLOROFORM-d) S=11.72-11.14 (m, 1H), 4.93 (br s, 1H), 4.76-4.55 (m, 1H), 4.18-3.92 (m, 6H), 3.27-2.93 (m, 6H), 2.38-2.21 (m, 1H), 1.96-1.74 (m, 4H), 1.64 (br dd, J=6.8, 18.0 Hz, 4H), 1.57-1.51 (m, 4H), 1.49-1.18 (m, 66H), 0.88 (t, J=6.8 Hz, 12H).

### General Procedure for the Synthesis of Compound 110:

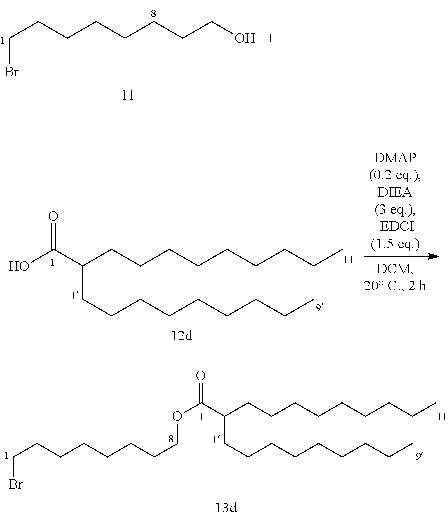 [SMILES: OCCCCCCCCBr ; CCCCCCCCCC(CCCCCCCCC)C(=O)O ; CCCCCCCCCC(CCCCCCCCC)C(=O)OCCCCCCCCBr]

To a solution of Compound 12d (1.49 g, 4.78 mmol, 1 eq.) in DCM (15 mL) was added DMAP (116.84 mg, 956.37 μmol, 0.2 eq.), EDCI (1.38 g, 7.17 mmol, 1.5 eq.), DIEA (1.85 g, 14.35 mmol, 2.50 mL, 3 eq.) and Compound 11 (1 g, 4.78 mmol, 819.67 μL, 1 eq.). The mixture was stirred at 20° C. for 2 hours. TLC (Petroleum ether: Ethyl acetate=10:1, R_{f}=0.68) indicated Compound 12d was consumed completely and new spots were detected. The reaction mixture was diluted with Sat·NH₄Cl (20 mL) and extracted with DCM (20 mL*2).

The combined organic layers were concentrated under reduced pressure to give a residue.

The residue was purified by column chromatography (SiO₂, Petroleum ether: Ethyl acetate=1:0 to 100:1) to give Compound 13d (1 g, 1.79 mmol, 37.45% yield, 90.18% purity) as colorless oil.

LCMS [M+1]⁺=503.4, 505.4

¹H NMR (400 MHz, CHLOROFORM-d) 8=4.07 (t, J=6.8 Hz, 2H), 3.41 (t, J=6.8 Hz, 2H), 2.37-2.26 (m, 1H), 1.86 (quin, J=7.2 Hz, 2H), 1.67-1.57 (m, 4H), 1.49-1.40 (m, 4H), 1.36-1.23 (m, 34H), 0.89 (t, J=6.8 Hz, 6H).

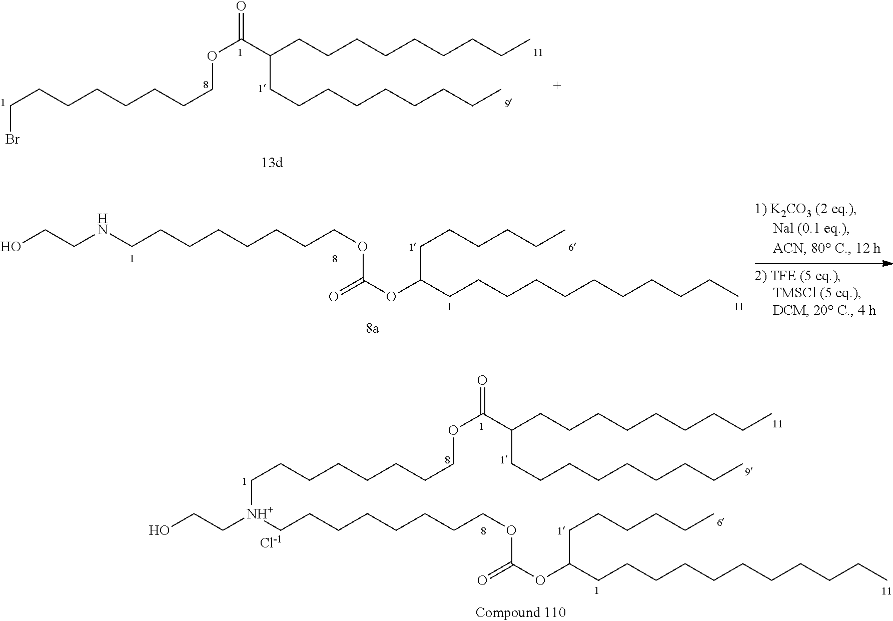 [SMILES: CCCCCCCCCC(CCCCCCCCC)C(=O)OCCCCCCCCBr ; CCCCCCCCCCCC(CCCCCC)OC(=O)OCCCCCCCCNCCO ; CCCCCCCCCCCC(CCCCCC)OC(=O)OCCCCCCCC[NH+](CCO)CCCCCCCCOC(=O)C(CCCCCCCCC)CCCCCCCCC ; [Cl-]]

To a solution of Compound 13d (500 mg, 992.78 μmol, 1 eq.) in ACN (6 mL) was added K₂CO₃ (274.41 mg, 1.99 mmol, 2 eq.), NaI (148.81 mg, 992.78 μmol, 1 eq.) and Compound 8a (482.27 mg, 992.78 μmol, 1 eq.). The mixture was stirred at 80° C. for 16 hours. LCMS showed Compound 13d was consumed completely and one main peak with desired MS was detected. The reaction mixture was partitioned between H₂O (10 mL) and Ethyl acetate (10 mL). The organic phase was separated, dried over Na₂SO₄, filtered and concentrated under reduced pressure to give a residue. The residue was purified by column chromatography (SiO₂, Petroleum ether: Ethyl acetate=30:1 to Dichloromethane: Methanol=20:1) to give Compound 110 (400 mg, 433.06 μmol, 43.62% yield, 98.36% purity) as yellow oil.

LCMS [M+1]⁺=908.9

¹H NMR (400 MHz, CHLOROFORM-d) S=4.76-4.63 (m, 1H), 4.09 (td, J=6.8, 20.0 Hz, 4H), 3.54 (br s, 2H), 2.72-2.54 (m, 2H), 2.52-2.39 (m, 4H), 2.37-2.25 (m, 1H), 1.73-1.60 (m, 6H), 1.51-1.06 (m, 80H), 0.89 (t, J=6.8 Hz, 12H).

A mixture of TFE (220.23 mg, 2.20 mmol, 158.32 μL, 5 eq.) and TMSCl (239.17 mg, 2.20 mmol, 279.40 μL, 5 eq.) was stirred at 20° C. for 1 hour under N₂ atmosphere. Then the solution of Compound 110 (400 mg, 440.28 μmol, 1 eq.) in DCM (4 mL) was added to the mixture at 20° C. The resulting mixture was stirred at 20° C. for 3 hours. The reaction mixture was concentrated under reduced pressure to give Compound 110 (360 mg, 375.14 μmol, 98.47% purity, 0.9 HCl) as a light-yellow gum.

LCMS [M+1]⁺=908.9

¹H NMR (400 MHz, CHLOROFORM-d) S=11.49 (br dd, J=2.0, 3.2 Hz, 1H), 5.21-4.78 (m, 1H), 4.69 (quin, J=6.4 Hz, 1H), 4.17-4.04 (m, 4H), 4.03-3.93 (m, 2H), 3.25-3.12 (m, 2H), 3.12-2.94 (m, 4H), 2.40-2.26 (m, 1H), 2.01-1.77 (m, 4H), 1.70-1.62 (m, 4H), 1.56 (br s, 4H), 1.46-1.19 (m, 74H), 0.88 (t, J=6.8 Hz, 12H).

### General Procedure for the Synthesis of Compound 111:

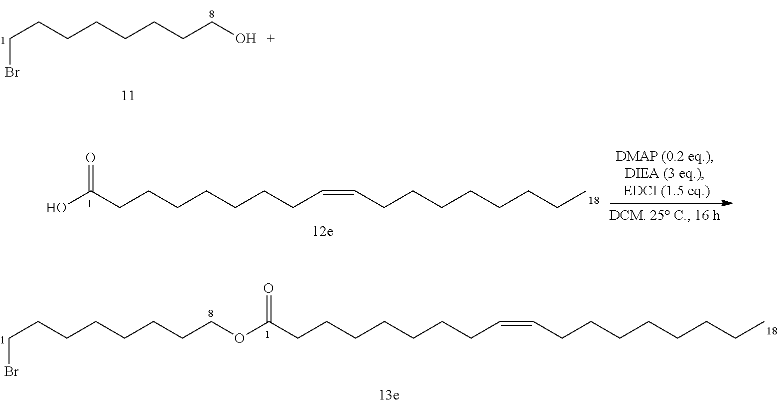 [SMILES: OCCCCCCCCBr ; CCCCCCCC/C=C\CCCCCCCC(=O)O ; CCCCCCCC/C=C\CCCCCCCC(=O)OCCCCCCCCBr]

To a solution of Compound 12e (1.35 g, 4.78 mmol, 1.35 mL, 1 eq.) in DCM (10 mL) was added DMAP (116.84 mg, 956.37 μmol, 0.2 eq.), EDCI (1.38 g, 7.17 mmol, 1.5 eq.), DIEA (1.85 g, 14.35 mmol, 2.50 mL, 3 eq.) and Compound 11 (1 g, 4.78 mmol, 819.67 μL, 1 eq.) at 25° C. The mixture was stirred at 25° C. for 16 hours. TLC (Petroleum ether: Ethyl acetate=5:1, R_{f}=0.65) indicated Compound 11 was consumed completely, and one major new spot was detected. The reaction mixture was diluted with Sat.NH₄Cl (200 mL) and extracted with DCM (80 mL*3). The combined organic layers were dried over Na₂SO₄, filtered and concentrated under reduced pressure to give a residue. The residue was purified by flash silica gel chromatography (ISCO®; 80 g SepaFlash® Silica Flash Column, Eluent of 0-50% Ethyl acetate/Petroleum ether gradient @100 mL/min) to give Compound 13e (746 mg, 1.58 mmol, 32.94% yield, 100% purity) as a colorless oil.

LCMS [M+1]⁺=473.4, 475.3

¹H NMR (400 MHz, CHLOROFORM-d) S=5.43-5.28 (m, 2H), 4.06 (t, J=6.8 Hz, 2H), 3.42 (t, J=6.8 Hz, 2H), 2.30 (t, J=7.6 Hz, 2H), 2.11-1.92 (m, 4H), 1.86 (quin, J=7.2 Hz, 2H), 1.70-1.58 (m, 4H), 1.49-1.41 (m, 2H), 1.40-1.20 (m, 26H), 0.96-0.84 (m, 3H).

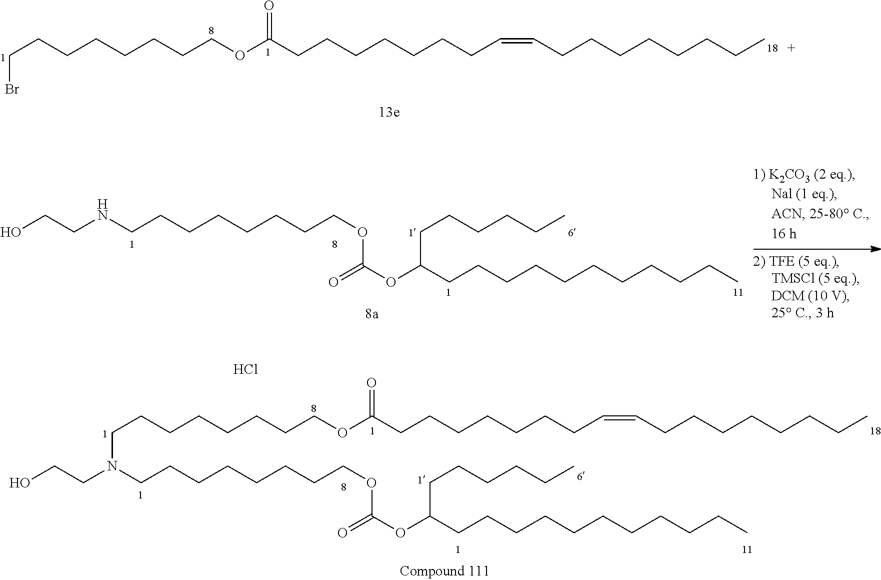 [SMILES: CCCCCCCC/C=C\CCCCCCCC(=O)OCCCCCCCCBr ; CCCCCCCCCCCC(CCCCCC)OC(=O)OCCCCCCCCNCCO ; Cl ; CCCCCCCC/C=C\CCCCCCCC(=O)OCCCCCCCCN(CCO)CCCCCCCCOC(=O)OC(CCCCCC)CCCCCCCCCCC]

To a solution of Compound 13e (500 mg, 1.06 mmol, 1 eq.) in ACN (5 mL) was added K₂CO₃ (291.84 mg, 2.11 mmol, 2 eq.), NaI (158.26 mg, 1.06 mmol, 1 eq.) and Compound 8a (512.89 mg, 1.06 mmol, 1 eq.) at 25° C. The mixture was stirred at 80° C. for 16 hours. TLC (Dichloromethane: Methanol=10:1, R_{f}=0.58) indicated Compound 8a remained and one major new spot was detected. The mixture was filtered and the filtrate was concentrated to give a residue. The residue was purified by column chromatography (SiO₂, Dichloromethane: Methanol=100:1 to 10:1) to give Compound 111 (718 mg, 807.55 μmol, 76.49% yield, 98.8% purity) as a colorless oil.

LCMS [M+1]⁺=878.8

¹H NMR (400 MHz, CHLOROFORM-d) S=5.35 (td, J=2.8, 6.0 Hz, 2H), 4.78-4.61 (m, 1H), 4.16-3.99 (m, 4H), 3.89-3.37 (m, 2H), 2.90-2.34 (m, 6H), 2.30 (t, J=7.6 Hz, 2H), 2.09-1.95 (m, 4H), 1.72-1.55 (m, 14H), 1.39-1.20 (m, 62H), 0.96-0.81 (m, 9H).

A mixture of TMSCl (185.51 mg, 1.71 mmol, 216.72 μL, 5 eq.) and TFE (170.83 mg, 1.71 mmol, 122.81 μL, 5 eq.) stirred at 20° C. for 1 hour under N₂ atmosphere. Then Compound 111 (300 mg, 341.52 μmol, 1 eq.) in DCM (3 mL) was added to the mixture at 20° C. The resulting mixture was stirred at 20° C. for 2 hours under N₂ atmosphere. The mixture was concentrated under reduced pressure to give Compound 111 (300 mg, 326.97 μmol, 95.74% yield, 98.92% purity, 0.8 HCl) as a light-yellow gum.

LCMS [M+1]⁺=878.8

¹H NMR (400 MHz, CHLOROFORM-d) S=11.57-11.36 (m, 1H), 5.35 (td, J=3.2, 5.6 Hz, 2H), 4.94 (s, 11H), 4.69 (br t, J=6.4 Hz, 1H), 4.18-4.03 (m, 4H), 4.00 (br s, 2H), 3.24-2.99 (m, 6H), 2.30 (t, J=7.6 Hz, 2H), 2.08-1.93 (m, 4H), 1.93-1.76 (m, 4H), 1.70-1.58 (m, 8H), 1.56 (br s, 2H), 1.41-1.24 (m, 62H), 0.97-0.78 (m, 9H)

General procedure for the synthesis of compounds Compound 108 to Compound 130:

| Compound | |
|---|---|
| Number | Structure |
| Compound 112 | 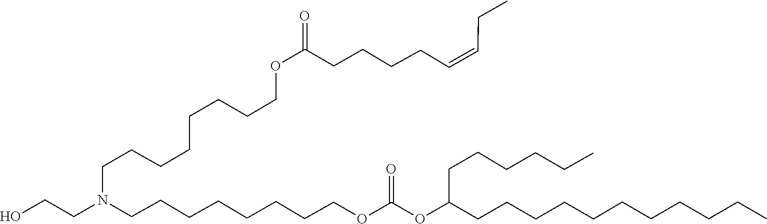 [SMILES: CC/C=C\CCCCC(=O)OCCCCCCCCN(CCO)CCCCCCCCOC(=O)OC(CCCCCC)CCCCCCCCCCC] |
| Compound 113 | 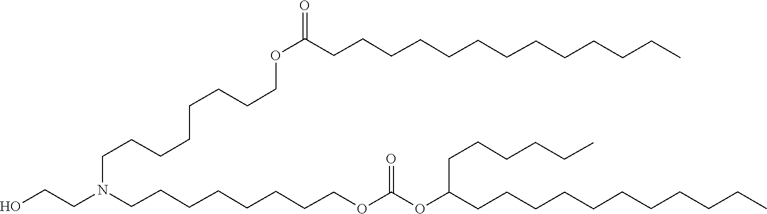 [SMILES: CCCCCCCCCCCCCC(=O)OCCCCCCCCN(CCO)CCCCCCCCOC(=O)OC(CCCCCC)CCCCCCCCCCC] |
| Compound 114 | 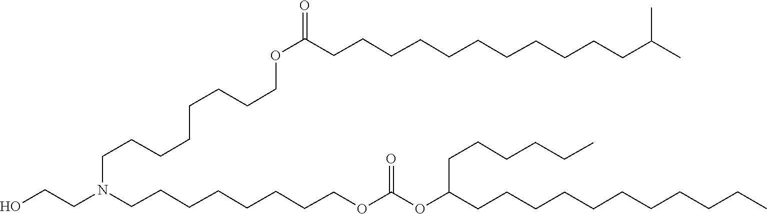 [SMILES: CCCCCCCCCCCC(CCCCCC)OC(=O)OCCCCCCCCN(CCO)CCCCCCCCOC(=O)CCCCCCCCCCCC(C)C] |
| Compound 115 | 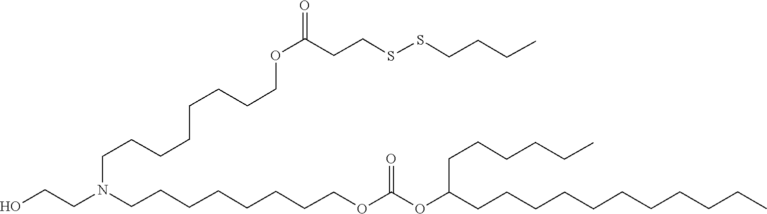 [SMILES: CCCCCCCCCCCC(CCCCCC)OC(=O)OCCCCCCCCN(CCO)CCCCCCCCOC(=O)CCSSCCCC] |
| Compound 116 | 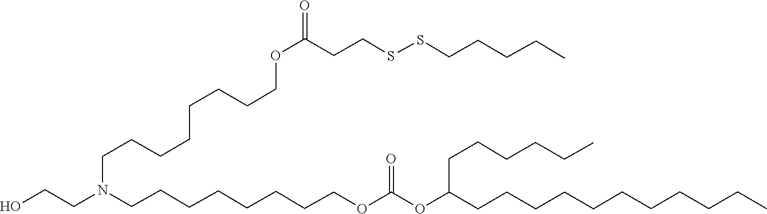 [SMILES: CCCCCCCCCCCC(CCCCCC)OC(=O)OCCCCCCCCN(CCO)CCCCCCCCOC(=O)CCSSCCCCC] |
| Compound 117 | 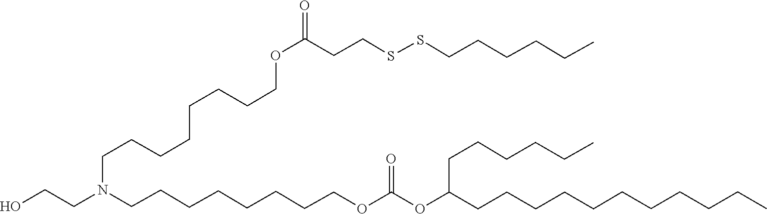 [SMILES: CCCCCCCCCCCC(CCCCCC)OC(=O)OCCCCCCCCN(CCO)CCCCCCCCOC(=O)CCSSCCCCCC] |
| Compound 118 | 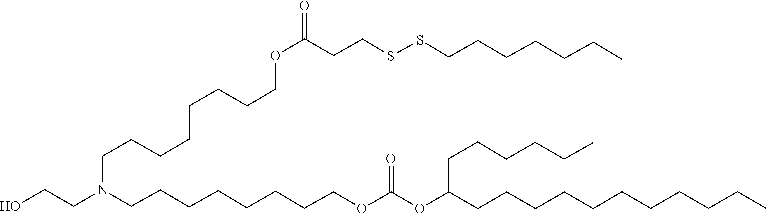 [SMILES: CCCCCCCCCCCC(CCCCCC)OC(=O)OCCCCCCCCN(CCO)CCCCCCCCOC(=O)CCSSCCCCCCC] |
| Compound 119 | 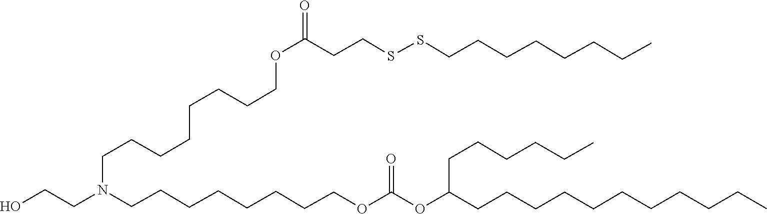 [SMILES: CCCCCCCCCCCC(CCCCCC)OC(=O)OCCCCCCCCN(CCO)CCCCCCCCOC(=O)CCSSCCCCCCCC] |
| Compound 120 | 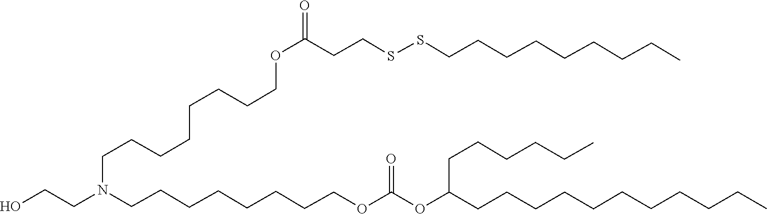 [SMILES: CCCCCCCCCCCC(CCCCCC)OC(=O)OCCCCCCCCN(CCO)CCCCCCCCOC(=O)CCSSCCCCCCCCC] |
| Compound 121 | 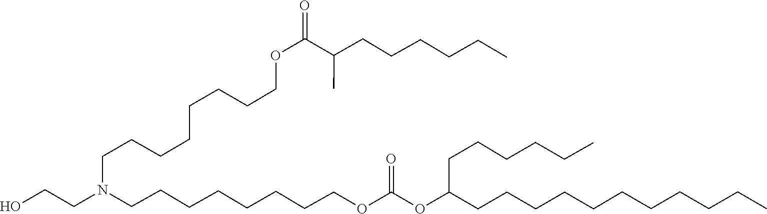 [SMILES: CCCCCCCCCCCC(CCCCCC)OC(=O)OCCCCCCCCN(CCO)CCCCCCCCOC(=O)C(C)CCCCCC] |
| Compound 122 | 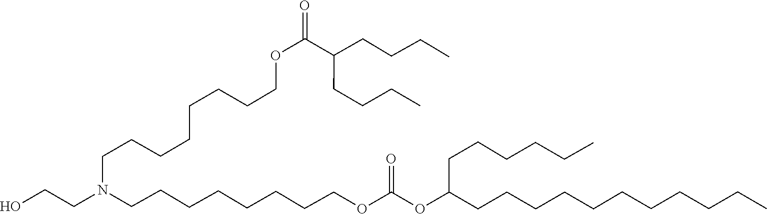 [SMILES: CCCCCCCCCCCC(CCCCCC)OC(=O)OCCCCCCCCN(CCO)CCCCCCCCOC(=O)C(CCCC)CCCC] |
| Compound 123 | 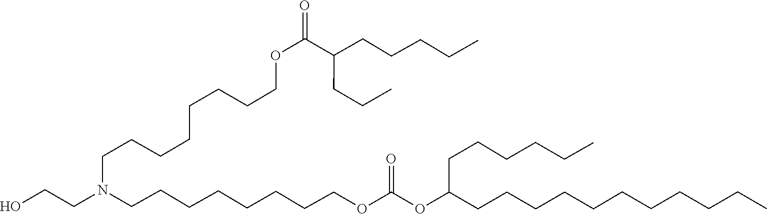 [SMILES: CCCCCCCCCCCC(CCCCCC)OC(=O)OCCCCCCCCN(CCO)CCCCCCCCOC(=O)C(CCC)CCCCC] |
| Compound 124 | 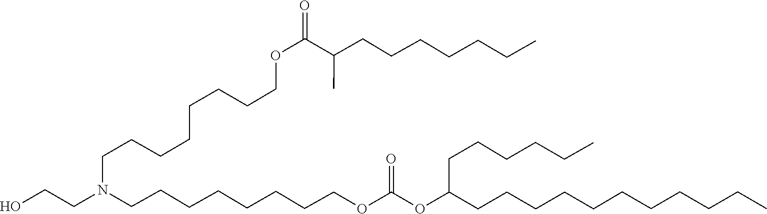 [SMILES: CCCCCCCCCCCC(CCCCCC)OC(=O)OCCCCCCCCN(CCO)CCCCCCCCOC(=O)C(C)CCCCCCC] |
| Compound 125 | 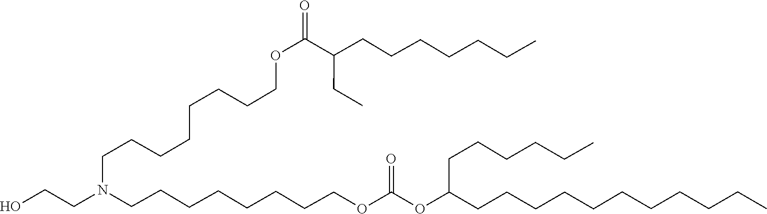 [SMILES: CCCCCCCCCCCC(CCCCCC)OC(=O)OCCCCCCCCN(CCO)CCCCCCCCOC(=O)C(CC)CCCCCCC] |
| Compound 126 | 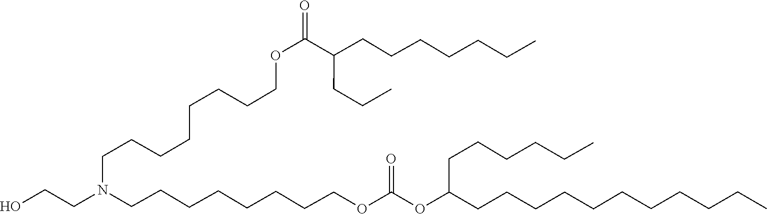 [SMILES: CCCCCCCCCCCC(CCCCCC)OC(=O)OCCCCCCCCN(CCO)CCCCCCCCOC(=O)C(CCC)CCCCCCC] |
| Compound 127 | 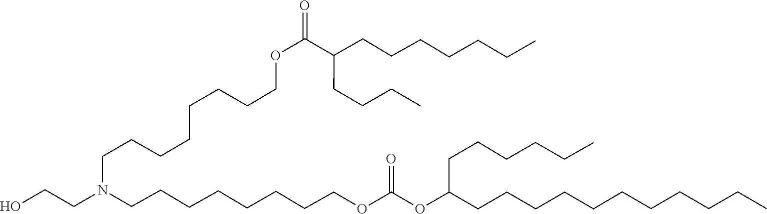 [SMILES: CCCCCCCCCCCC(CCCCCC)OC(=O)OCCCCCCCCN(CCO)CCCCCCCCOC(=O)C(CCCC)CCCCCCC] |
| Compound 128 | 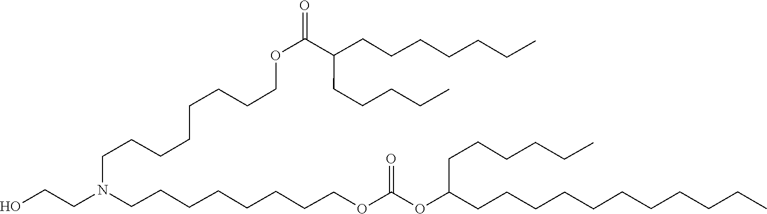 [SMILES: CCCCCCCCCCCC(CCCCCC)OC(=O)OCCCCCCCCN(CCO)CCCCCCCCOC(=O)C(CCCCC)CCCCCCC] |
| Compound 129 | 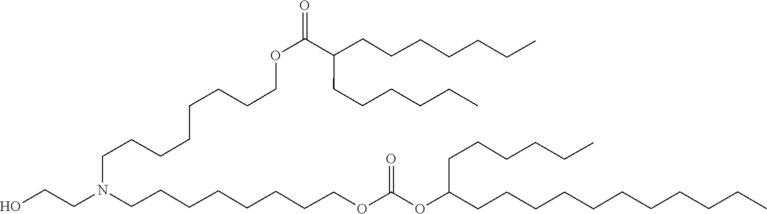 [SMILES: CCCCCCCCCCCC(CCCCCC)OC(=O)OCCCCCCCCN(CCO)CCCCCCCCOC(=O)C(CCCCCC)CCCCCCC] |
| Compound 130 | 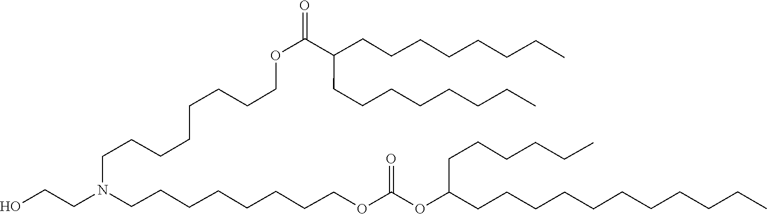 [SMILES: CCCCCCCCCCCC(CCCCCC)OC(=O)OCCCCCCCCN(CCO)CCCCCCCCOC(=O)C(CCCCCCCC)CCCCCCCC] |

The compounds, Compound 108 to Compound 123 and Compound 125 to Compound 130 can be synthesized by following synthetic schemes. The key intermediate 8a that will be used for the synthesis of Compound 108 to Compound 123 and Compound 125 to Compound 130, can be synthesized according to the following synthetic scheme.

### Synthesis of Compound 8a (Common Intermediate)

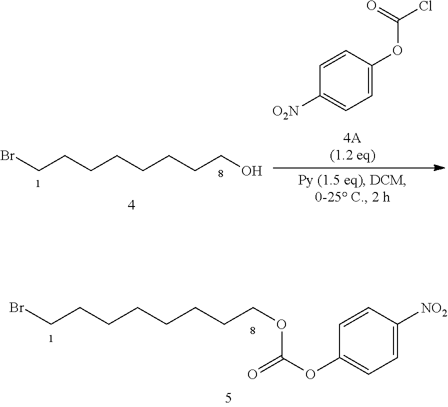 [SMILES: O=C(Cl)Oc1ccc([N+](=O)[O-])cc1 ; OCCCCCCCCBr ; O=C(OCCCCCCCCBr)Oc1ccc([N+](=O)[O-])cc1]

Pyridine is added to a solution of Compound 4 in DCM is added. Then, Compound 4A in DCM is added dropwise at 0° C. The resulting mixture is stirred at 20° C. for 2 hours. TLC assesses the extent of the reaction. The reaction mixture is diluted with H₂O and extracted with DCM. The combined organic layers are concentrated under reduced pressure to give a residue. The residue is purified by column chromatography to give Compound 5 as a white solid.

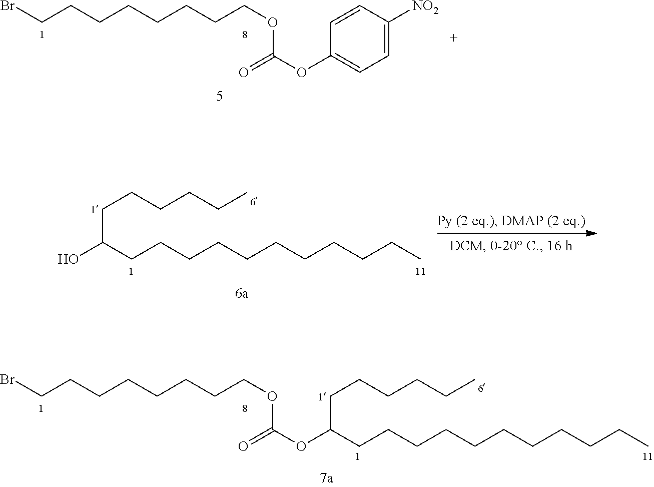 [SMILES: O=C(OCCCCCCCCBr)Oc1ccc([N+](=O)[O-])cc1 ; CCCCCCCCCCCC(O)CCCCCC ; CCCCCCCCCCCC(CCCCCC)OC(=O)OCCCCCCCCBr]

To a solution of Compound 5 and Compound 6a in DCM is added Pyridine dropwise at 0° C. Then, DMAP is added at 0° C. The resulting mixture is stirred at 20° C. TLC assesses the extent of the reaction. The reaction mixture is concentrated under reduced pressure to give a residue. The residue is purified by column chromatography to give Compound 7a as a colorless oil.

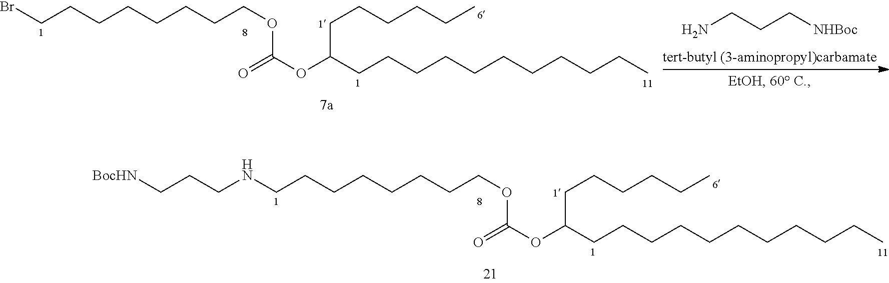 [SMILES: CCCCCCCCCCCC(CCCCCC)OC(=O)OCCCCCCCCBr ; CC(C)(C)OC(=O)NCCCN ; CCCCCCCCCCCC(CCCCCC)OC(=O)OCCCCCCCCNCCCNC(=O)OC(C)(C)C]

To a solution of Compound 7a in acetonitrile is added DIEA and 2-aminoethanol. The mixture is stirred at 70° C. TLC assesses the extent of the reaction. The reaction mixture is diluted with Ethyl acetate, washed with H₂O dried over Na₂SO₄, filtered and concentrated under reduced pressure to give a residue. The residue is purified by column chromatography to give Compound 8a as a colorless oil.

### General Synthesis of Compound 108 to Compound 123; Compound 125 to Compound 130

For representative synthesis with characterization, please see Synthesis of Compound 108 vide supra

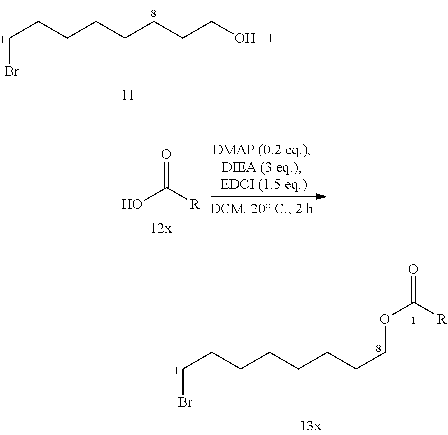

To a solution of Compound 12x (see table below for structure) in DCM is added DMAP, EDCI, DIEA and Compound 11. TLC assesses the extent of the reaction. The reaction mixture is concentrated under reduced pressure to give a residue. The residue is purified by column chromatography to give Compound 13x.

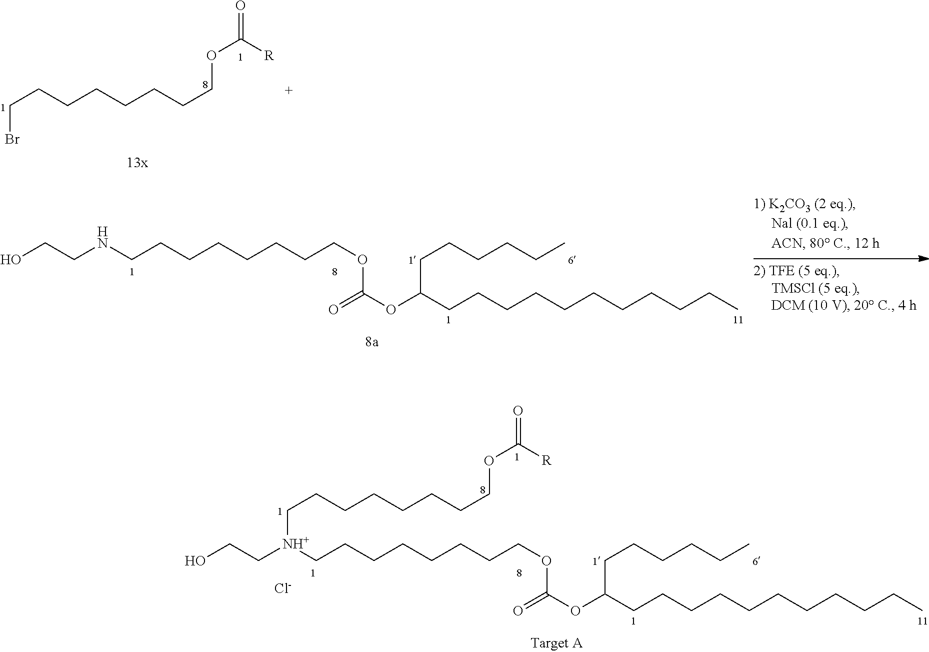

A mixture of Compound 13x, Compound 8a, K2CO₃ and NaI in acetonitrile is degassed and purged with N₂ 3 times, and the mixture is stirred at 80° C. under N₂ atmosphere. TLC assesses the extent of the reaction. The combined reaction mixture is diluted with H₂O and extracted with DCM. The combined organic layers are concentrated under reduced pressure to give a residue. The residue is purified by column chromatography to give Target (see table below) as a colorless oil.

For general synthesis above, the following table indicates the intermediate carboxylic acid 12x that will give “Target” Compound 108 through Compound 123; Compound 124 to Compound 129

| | Intermediate | |
|---|---|---|
| Target A | 12x = | Structure |
| Compound 108 | 12b | 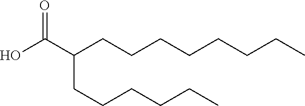 [SMILES: CCCCCCCCC(CCCCCC)C(=O)O] |
| Compound 109 | 12c | 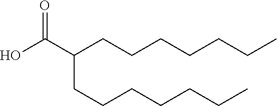 [SMILES: CCCCCCCC(CCCCCCC)C(=O)O] |
| Compound 110 | 12d | 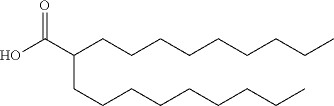 [SMILES: CCCCCCCCCC(CCCCCCCCC)C(=O)O] |
| Compound 111 | 12e | 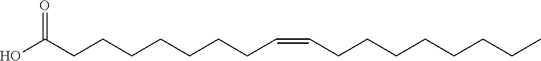 [SMILES: CCCCCCCC/C=C\CCCCCCCC(=O)O] |
| Compound 112 | 12f | 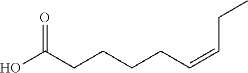 |
| Compound 113 | 12g | 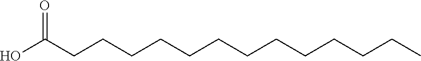 [SMILES: CCCCCCCCCCCCCC(=O)O] |
| Compound 114 | 12h | 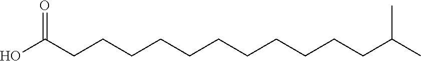 [SMILES: CC(C)CCCCCCCCCCCC(=O)O] |
| Compound 115 | 12i | 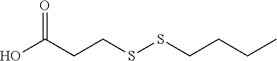 |
| Compound 116 | 12j | 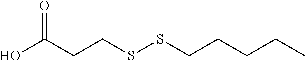 |
| Compound 117 | 12k | 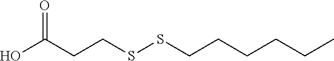 |
| Compound 118 | 12l | 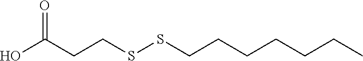 |
| Compound 119 | 12m | 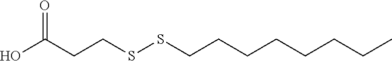 |
| Compound 120 | 12n | 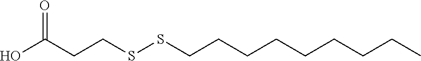 [SMILES: CCCCCCCCCSSCCC(=O)O] |
| Compound 121 | 12o | 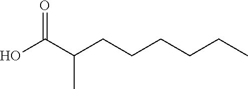 |
| Compound 122 | 12p | 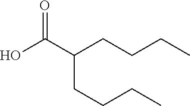 |
| Compound 123 | 12q | 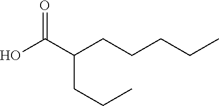 |
| Compound 124 | 12r | 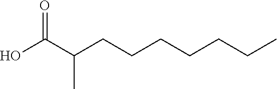 |
| Compound 125 | 12s | 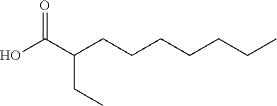 |
| Compound 126 | 12t | 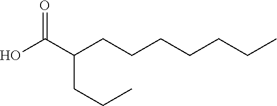 |
| Compound 127 | 12u | 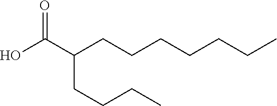 |
| Compound 128 | 12v | 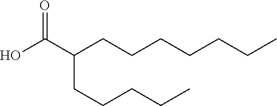 [SMILES: CCCCCCCC(CCCCC)C(=O)O] |
| Compound 129 | 12w | 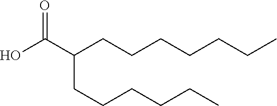 [SMILES: CCCCCCCC(CCCCCC)C(=O)O] |
| Compound 130 | 12x | 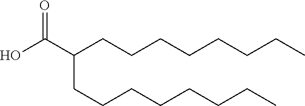 [SMILES: CCCCCCCCC(CCCCCCCC)C(=O)O] |

### General Procedure for the Synthesis of Compound 131 to Compound 136

| Compound | |
|---|---|
| Number | Structure |
| Compound 131 | 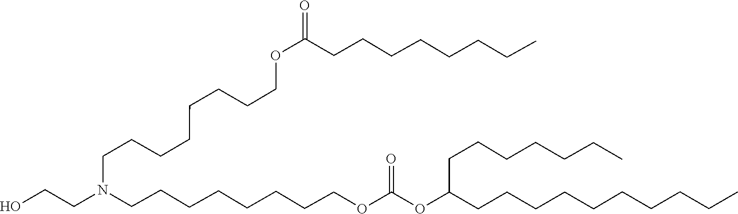 [SMILES: CCCCCCCCCCC(CCCCCCC)OC(=O)OCCCCCCCCN(CCO)CCCCCCCCOC(=O)CCCCCCCC] |
| Compound 132 | 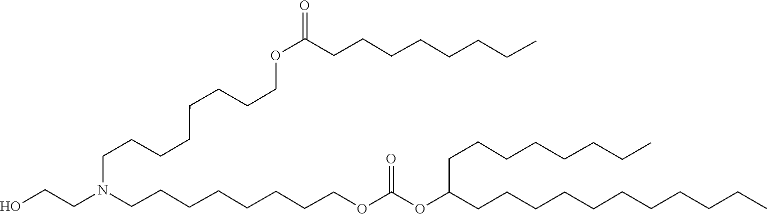 [SMILES: CCCCCCCCCCCC(CCCCCCCC)OC(=O)OCCCCCCCCN(CCO)CCCCCCCCOC(=O)CCCCCCCC] |
| Compound 133 | 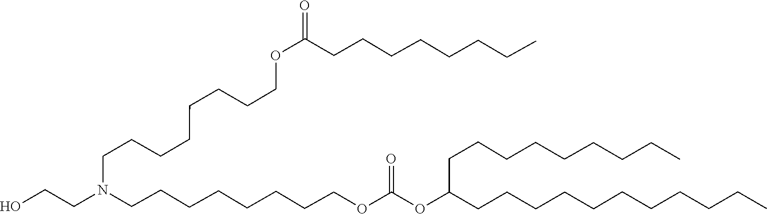 [SMILES: CCCCCCCCCCCC(CCCCCCCCC)OC(=O)OCCCCCCCCN(CCO)CCCCCCCCOC(=O)CCCCCCCC] |
| Compound 134 | 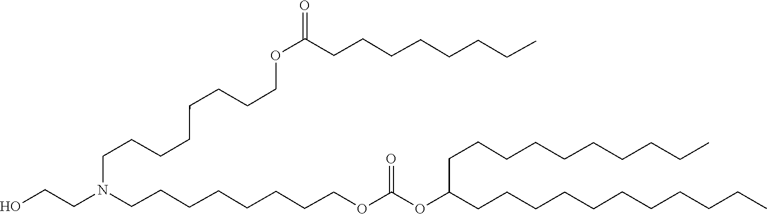 [SMILES: CCCCCCCCCCCC(CCCCCCCCCC)OC(=O)OCCCCCCCCN(CCO)CCCCCCCCOC(=O)CCCCCCCC] |
| Compound 135 | 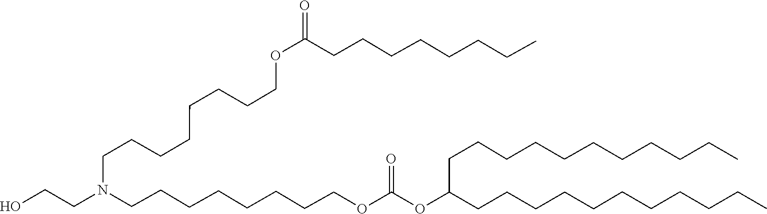 [SMILES: CCCCCCCCCCCC(CCCCCCCCCCC)OC(=O)OCCCCCCCCN(CCO)CCCCCCCCOC(=O)CCCCCCCC] |
| Compound 136 | 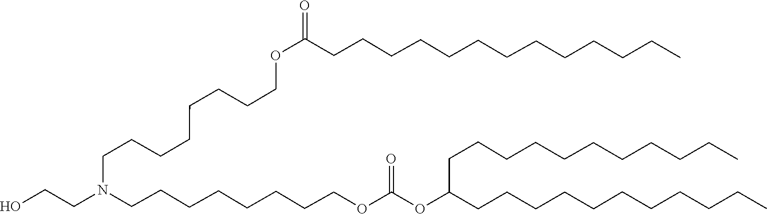 [SMILES: CCCCCCCCCCCCCC(=O)OCCCCCCCCN(CCO)CCCCCCCCOC(=O)OC(CCCCCCCCCCC)CCCCCCCCCCC] |

The alcohols 6x in the table below are used for the synthesis of Compounds 8x, which be synthesized according to the following synthetic scheme. Then, Compounds 8c can be used to synthesize Compound 131 to Compound 136

| Target B | 6x = | Structure |
|---|---|---|
| Compound 131 | 6a | 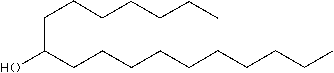 [SMILES: CCCCCCCCCCC(O)CCCCCCC] |
| Compound 132 | 6b | 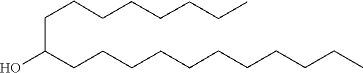 [SMILES: CCCCCCCCCCCC(O)CCCCCCCC] |
| Compound 133 | 6c | 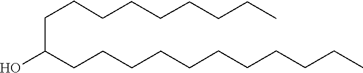 [SMILES: CCCCCCCCCCCC(O)CCCCCCCCC] |
| Compound 134 | 6d | 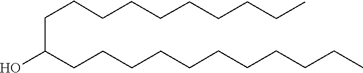 [SMILES: CCCCCCCCCCCC(O)CCCCCCCCCC] |
| Compound 135 | 6e | 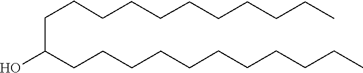 [SMILES: CCCCCCCCCCCC(O)CCCCCCCCCCC] |
| Compound 136 | 6f | 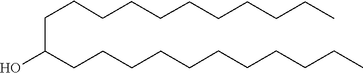 [SMILES: CCCCCCCCCCCC(O)CCCCCCCCCCC] |

For representative synthesis with characterization, please see Synthesis of Compound 100 (for Synthesis of Compound 8a) and Synthesis of Compound 102 (for Synthesis of Compound 13a) vide supra.

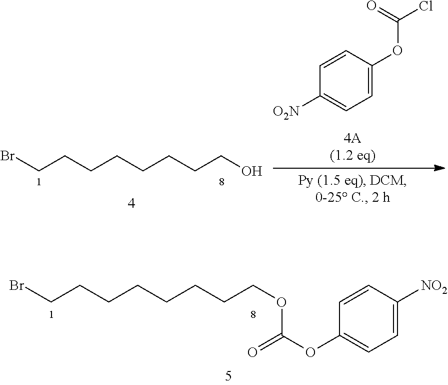 [SMILES: O=C(Cl)Oc1ccc([N+](=O)[O-])cc1 ; OCCCCCCCCBr ; O=C(OCCCCCCCCBr)Oc1ccc([N+](=O)[O-])cc1]

To a solution of Compound 4 in DCM is added Pyridine. Then, Compound 4A in DCM is added dropwise at 0° C. The resulting mixture is stirred at 20° C. TLC assesses the extent of the reaction. The reaction mixture is diluted with H₂O and extracted with DCM (50 mL×3).

The combined organic layers are concentrated under reduced pressure to give a residue. The residue is purified by column chromatography to give Compound 5 as a white solid.

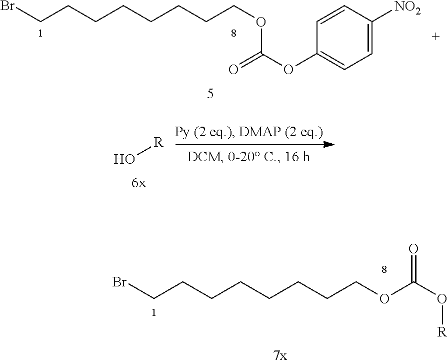

To a solution of Compound 5 and Compound 6a in DCM is added Pyridine dropwise at 0° C. Then, DMAP is added at 0° C. The resulting mixture is stirred at 20° C. TLC assesses the extent of the reaction. The reaction mixture is concentrated under reduced pressure to give a residue. The residue is purified by column chromatography to give Compound 7x.

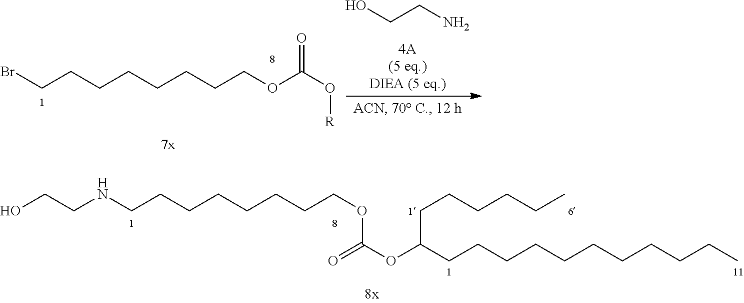

To a solution of Compound 7x in acetonitrile is added DIEA and 2-aminoethanol. The mixture is stirred at 70° C. for 12 hours. TLC assesses the extent of the reaction. The reaction mixture is diluted with Ethyl acetate, washed with H₂O, dried over Na₂SO₄, filtered and concentrated under reduced pressure to give a residue. The residue is purified by column chromatography to give Compound 8a.

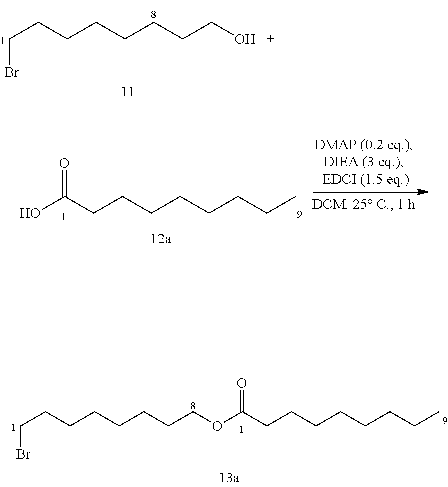 [SMILES: OCCCCCCCCBr ; CCCCCCCCC(=O)O ; CCCCCCCCC(=O)OCCCCCCCCBr]

To a solution of Compound 12a in DCM is added DIEA, EDCI, DMAP and Compound 11b. The mixture is stirred at 20° C. LCMS assesses the extent of the reaction. The reaction mixture is concentrated under reduced pressure to give a residue. The residue is diluted with brine (80 mL) and extracted with ethyl acetate (25 mL×4). The combined organic layers are dried over Na₂SO₄, filtered and concentrated under reduced pressure to give a residue. The residue is purified by column chromatography to give Compound 13a as a colorless oil.

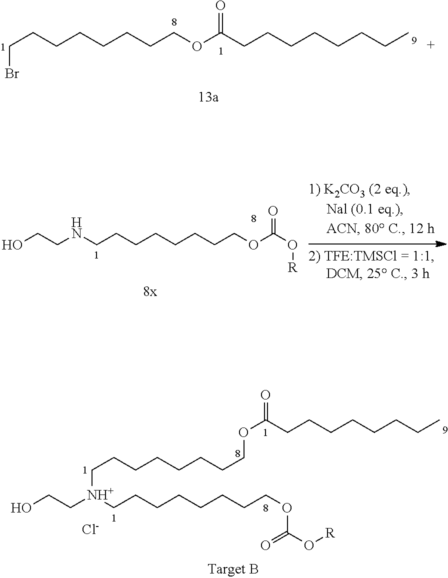

A mixture of Compound 13a, Compound 4, K2CO₃, and NaI in acetonitrile is degassed and purged with N₂ 3 times. The mixture is stirred at 80° C. under an N₂ atmosphere. TLC assesses the extent of the reaction. The residue is diluted with ethyl acetate. The combined organic layers are washed with NH₄Cl (30 mL×3), dried over Na₂SO₄, filtered and concentrated under reduced pressure to give a residue. The residue is purified by column chromatography to give Target B.

To a solution of Target B in DCM is added HCl/dioxane. The mixture is stirred at 20° C. for 2 hours. The reaction mixture is concentrated under reduced pressure to give a residue. The residue is diluted with acetonitrile and H₂O. The mixture is lyophilized to give Target B.

### General Procedure for the Synthesis of Compounds 137 and 138

### General Procedure for the Synthesis of Compound 21

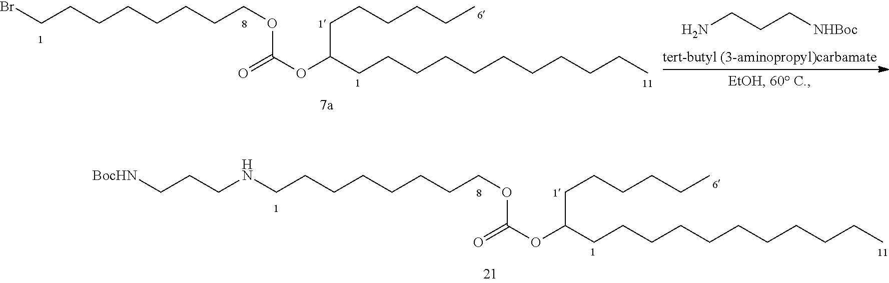 [SMILES: CCCCCCCCCCCC(CCCCCC)OC(=O)OCCCCCCCCBr ; CC(C)(C)OC(=O)NCCCN ; CCCCCCCCCCCC(CCCCCC)OC(=O)OCCCCCCCCNCCCNC(=O)OC(C)(C)C]

To a solution of Compound 7a in acetonitrile (11 mL) is added tert-butyl (3-aminopropyl)carbamate in ethanol. The mixture is stirred at 60° C. TLC assesses the extent of the reaction. The reaction mixture is diluted with ethyl acetate, washed with H₂O, dried over Na₂SO₄, filtered and concentrated under reduced pressure to give a residue. The residue is purified by column chromatography to give Compound 21.

### General Procedure for the Synthesis of Compound 137

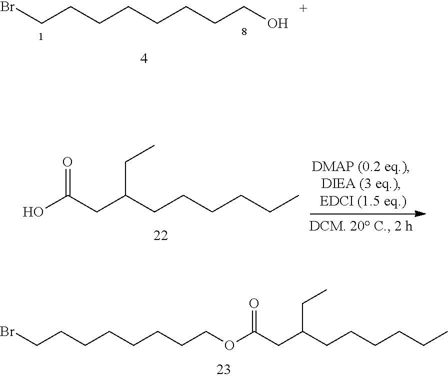 [SMILES: OCCCCCCCCBr ; CCCCCCC(CC)CC(=O)O ; CCCCCCC(CC)CC(=O)OCCCCCCCCBr]

To a solution of Compound 4 in DCM is added DMAP, EDCI, DIEA and Compound 22 and the reaction is stirred at 20° C. TLC assesses the extent of the reaction. The reaction mixture is concentrated under reduced pressure to give a residue. The residue is purified by column chromatography to give Compound 23.

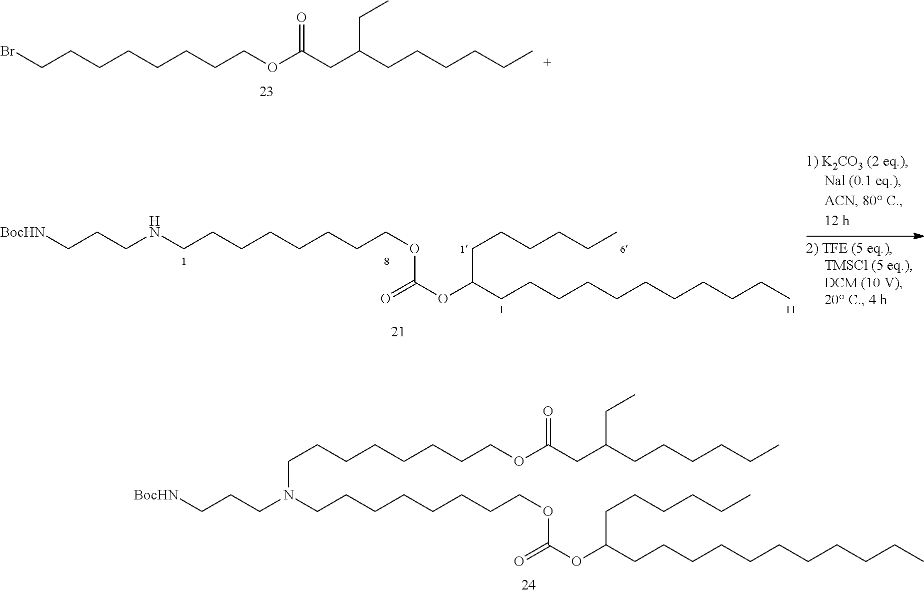 [SMILES: CCCCCCC(CC)CC(=O)OCCCCCCCCBr ; CCCCCCCCCCCC(CCCCCC)OC(=O)OCCCCCCCCNCCCNC(=O)OC(C)(C)C ; CCCCCCCCCCCC(CCCCCC)OC(=O)OCCCCCCCCN(CCCCCCCCOC(=O)CC(CC)CCCCCC)CCCNC(=O)OC(C)(C)C]

A mixture of Compound 23, Compound 21, K2CO₃ and NaI in acetonitrile is degassed and purged with N₂ 3 times, and the mixture is stirred at 80° C. under N₂ atmosphere. TLC assesses the extent of the reaction. The combined reaction mixture is diluted with H₂O and extracted with DCM. The combined organic layers are concentrated under reduced pressure to give a residue. The residue is purified by column chromatography to give Compound 24 as a colorless oil.

A solution of TMSCl in TFE is stirred at 20° C. Then Compound 24 is added dropwise at 20° C. The resulting mixture is stirred at 20° C. The mixture is concentrated to give Compound 24 as a light yellow oil.

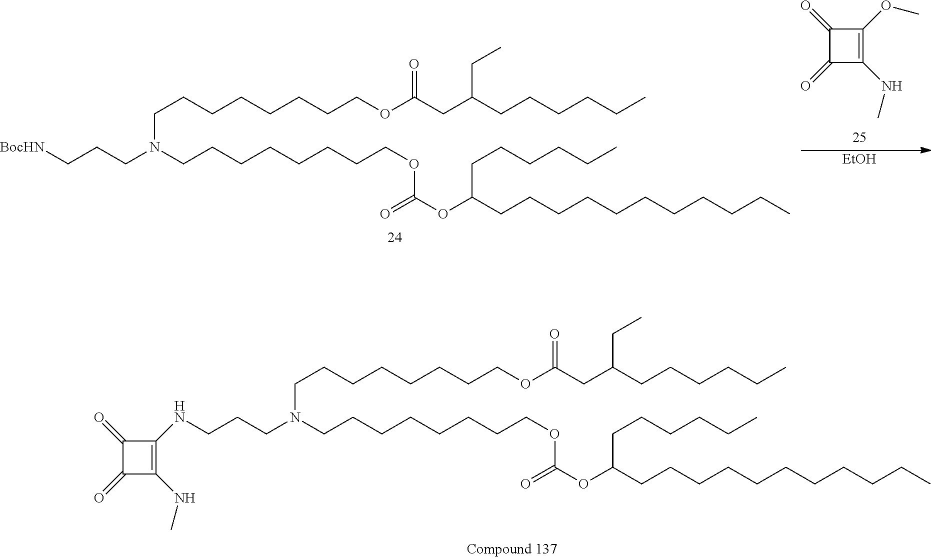

Compound 24 is dissolved in ethanol, and Compound 25 is added with stirring at room temperature. TLC assesses the extent of the reaction. The combined reaction mixture is diluted with H₂O and extracted with DCM. The combined organic layers are concentrated under reduced pressure to give a residue. The residue is purified by column chromatography to give Compound 137 as a colorless oil.

### General Procedure for the Synthesis of Compound 138

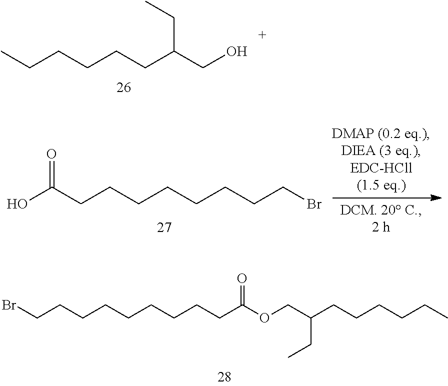 [SMILES: CCCCCCC(CC)CO ; O=C(O)CCCCCCCCBr ; CCCCCCC(CC)COC(=O)CCCCCCCCCBr]

To a solution of Compound 26 in DCM is added DMAP, EDCI, DIEA and Compound 27 and the reaction is stirred at 20° C. TLC assesses the extent of the reaction. The reaction mixture is concentrated under reduced pressure to give a residue. The residue is purified by column chromatography to give Compound 28.

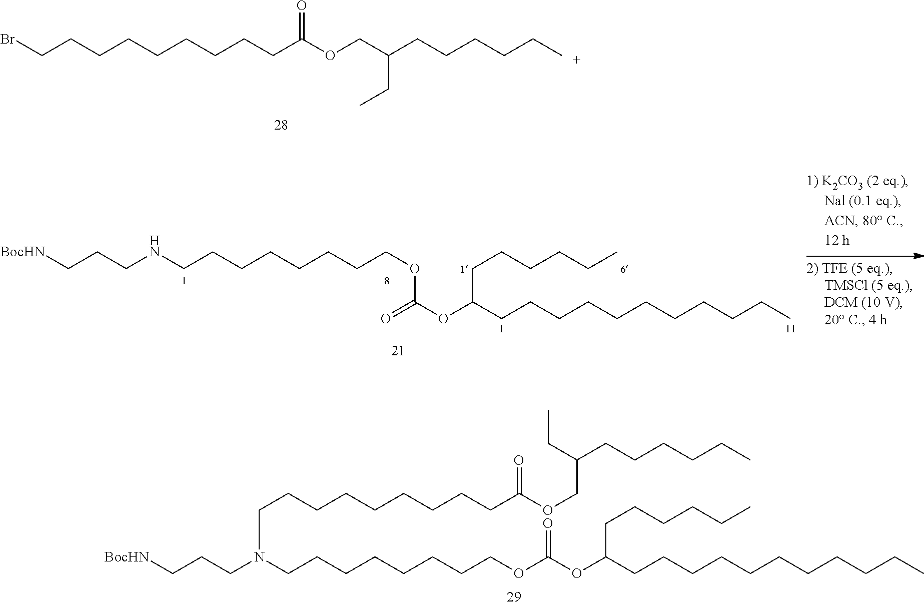

A mixture of Compound 28, Compound 21, K2CO₃ and NaI in acetonitrile is degassed and purged with N₂ 3 times, and the mixture is stirred at 80° C. under N₂ atmosphere. TLC assesses the extent of the reaction. The combined reaction mixture is diluted with H₂O and extracted with DCM. The combined organic layers are concentrated under reduced pressure to give a residue. The residue is purified by column chromatography to give Compound 29 as a colorless oil.

A solution of TMSCl in TFE is stirred at 20° C. Then Compound 29 is added dropwise at 20° C. The resulting mixture is stirred at 20° C. The mixture is concentrated to give Compound 29 as a light yellow oil.

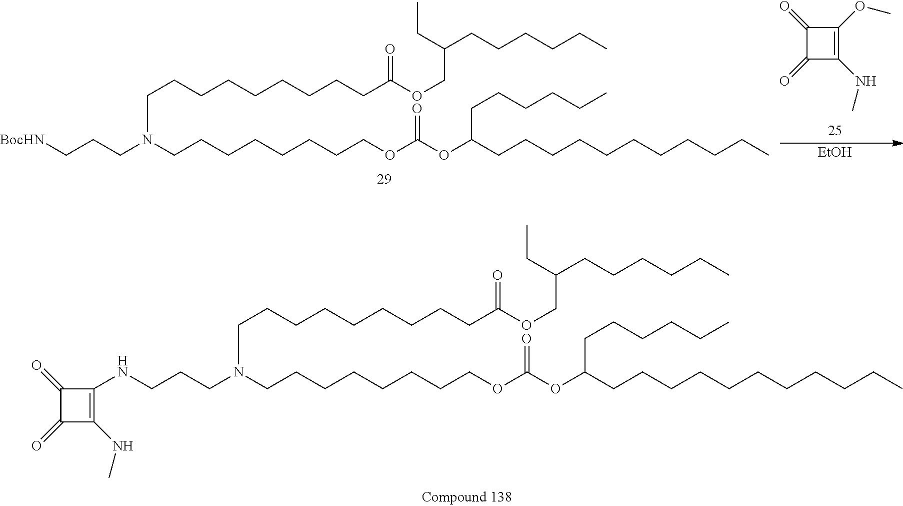

Compound 29 is dissolved in ethanol, and Compound 25 is added with stirring at room temperature. TLC assesses the extent of the reaction. The combined reaction mixture is diluted with H₂O and extracted with DCM. The combined organic layers are concentrated under reduced pressure to give a residue. The residue is purified by column chromatography to give Compound 138 as a colorless oil.

### General Procedure for the Synthesis of Compound 104 to Compound 107 and Compound 140 to 142

| Com- | |
|---|---|
| pound | |
| Num- | |
| ber | Structure |
| Com- pound 140 | 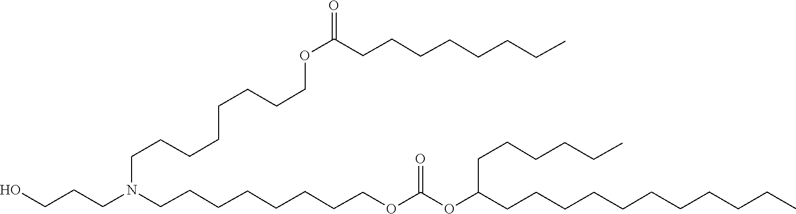 [SMILES: CCCCCCCCCCCC(CCCCCC)OC(=O)OCCCCCCCCN(CCCO)CCCCCCCCOC(=O)CCCCCCCC] |
| Com- pound 141 | 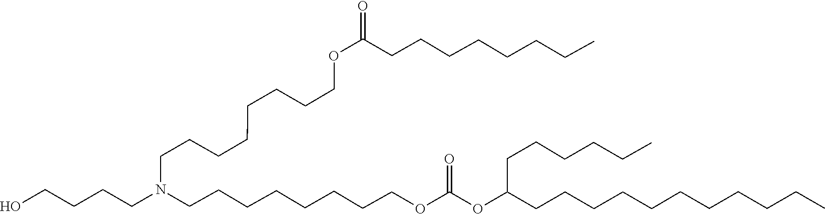 [SMILES: CCCCCCCCCCCC(CCCCCC)OC(=O)OCCCCCCCCN(CCCCO)CCCCCCCCOC(=O)CCCCCCCC] |
| Com- pound 142 | 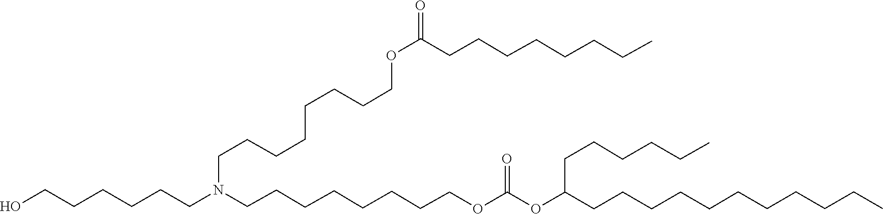 [SMILES: CCCCCCCCCCCC(CCCCCC)OC(=O)OCCCCCCCCN(CCCCCCO)CCCCCCCCOC(=O)CCCCCCCC] |

The amino alcohols 19x in the table below are used for the synthesis of Compound 104 to Compound 107 and Compound 140 to 142 For a representative synthesis, please see the synthesis of Compound 104, 105 and 107, vide supra.

| Target C | Intermediate 19x = | Structure |
|---|---|---|
| Compound 104 | 19a | 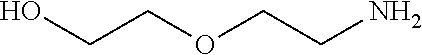 |
| Compound 105 | 19b | 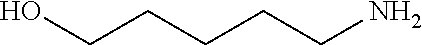 |
| Compound 106 | 19c | 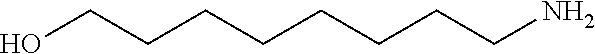 |
| Compound 107 | 19d | 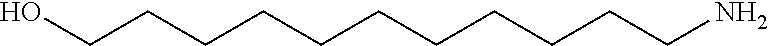 [SMILES: NCCCCCCCCCCCO] |

Compound 139 is identical to Compound 1

| | | |
|---|---|---|
| Compound 140 | 19e | 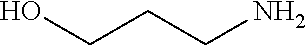 |
| Compound 141 | 19f | 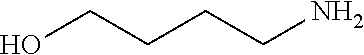 |
| Compound 142 | 19g | 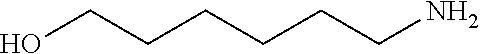 |

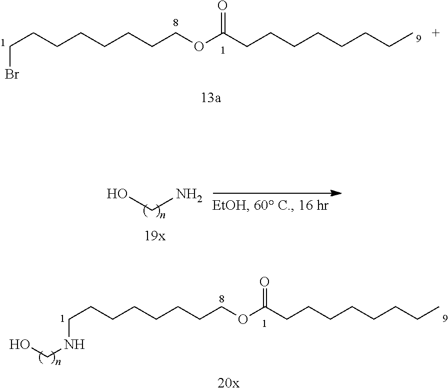

To a solution of Compound 13a in EtOH is added Compound 19x (see table above). The mixture is stirred at 90° C. The reaction mixture is concentrated under reduced pressure to give a residue. The residue is diluted with H₂O and extracted with ethyl acetate. The combined organic layers are washed with brine, dried over Na₂SO₄, filtered and concentrated under reduced pressure to give a residue. The residue is purified by column chromatography to give Compound 20x.

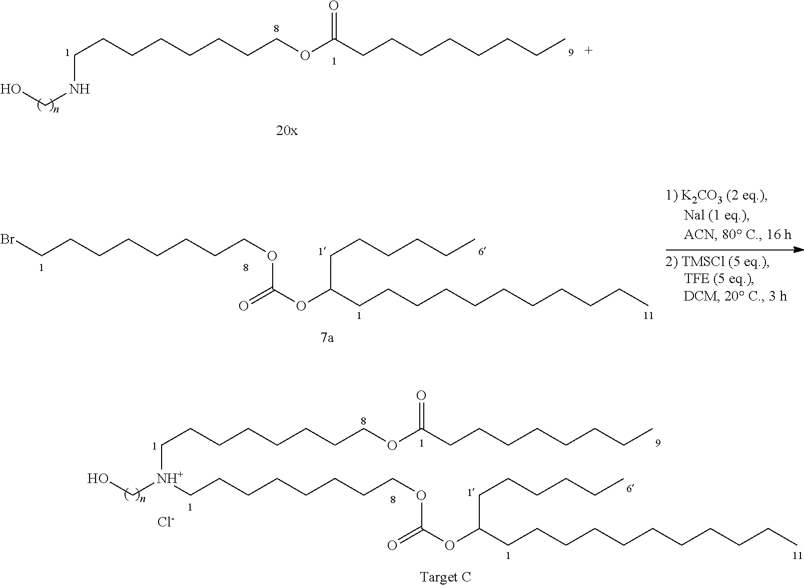

To a solution of Compound 20x in acetonitrile is added Compound 7a, NaI, and K2CO₃. The mixture is stirred at 80° C. The reaction mixture is diluted with ethyl acetate and filtered. The filtrate was concentrated under reduced pressure to give a residue. The residue is purified by flash silica gel chromatography to give crude Target C, which is purified by prep-HPLC and neutralized with saturated NaHCO₃. The mixture is concentrated to remove acetonitrile, diluted with H₂O and extracted with DCM. The combined organic layers are dried over Na₂SO₄, filtered and concentrated under reduced pressure to give Target C.

### General Procedure for the Synthesis of Compound 143 to Compound 144

| Com- | |
|---|---|
| pound | |
| Number | Structure |
| Com- pound 143 | 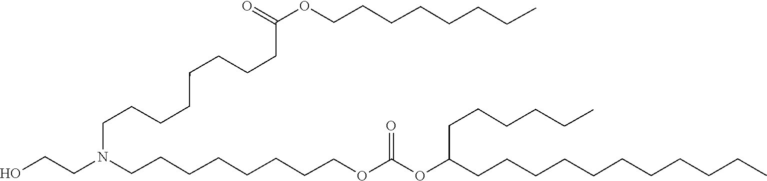 [SMILES: CCCCCCCCCCCC(CCCCCC)OC(=O)OCCCCCCCCN(CCO)CCCCCCCCC(=O)OCCCCCCCC] |
| Com- pound 144 | 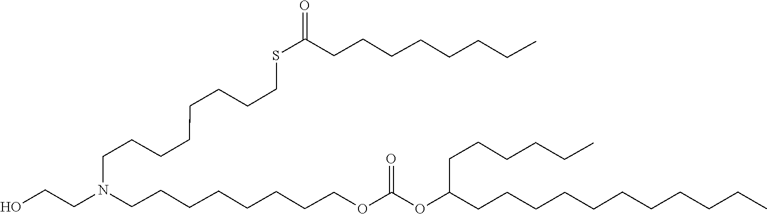 [SMILES: CCCCCCCCCCCC(CCCCCC)OC(=O)OCCCCCCCCN(CCO)CCCCCCCCSC(=O)CCCCCCCC] |

For a representative synthesis, please see the synthesis of Compound 100, vide supra.

| | Cmpd | | Cmpd | | Intermediate |
|---|---|---|---|---|---|
| Target D | 30x = | Structure | 31x = | Structure | Cmpd 32x = |
| Cmpd 143 | 30a | 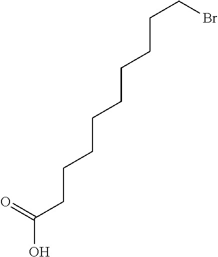 [SMILES: O=C(O)CCCCCCCCCBr] | 31a | 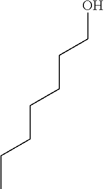 | 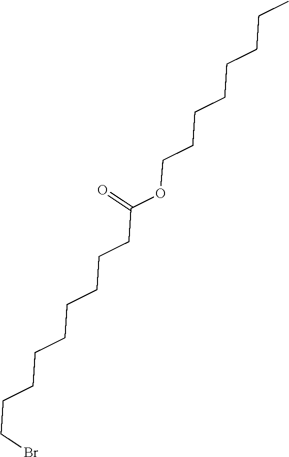 [SMILES: CCCCCCCCOC(=O)CCCCCCCCCBr] |
| Cmpd 144 | 30b | 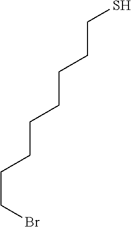 | 31b | 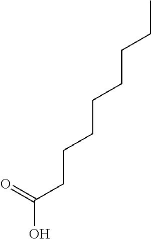 | 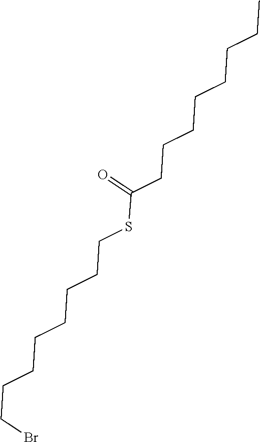 [SMILES: CCCCCCCCC(=O)SCCCCCCCCBr] |

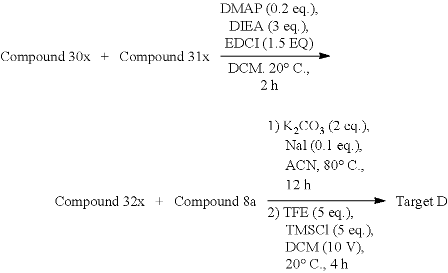

To a solution of Compound 30x in DCM is added DMAP, EDCI, DIEA and Compound 31x and the reaction is stirred at 20° C. TLC assesses the extent of the reaction. The reaction mixture is concentrated under reduced pressure to give a residue. The residue is purified by column chromatography to give Compound 32x.

To a solution of Compound 32x in acetonitrile is added Compound 8a, NaI, and K₂CO₃. The mixture is stirred at 80° C. The reaction mixture is diluted with ethyl acetate and filtered. The filtrate was concentrated under reduced pressure to give a residue. The residue is purified by flash silica gel chromatography to give crude Target D, which is purified by prep-HPLC and neutralized with saturated NaHCO₃. The mixture is concentrated to remove acetonitrile, diluted with H₂O and extracted with DCM. The combined organic layers are dried over Na₂SO₄, filtered and concentrated under reduced pressure to give Target D.

A solution of TMSCl in TFE is stirred at 20° C. Then Target D is added dropwise at 20° C. The resulting mixture is stirred at 20° C. The mixture is concentrated to give Target D as a light yellow oil.

General procedure for the synthesis of Compound 145 to Compound 146

| Compound | | | | | |
|---|---|---|---|---|---|
| Number | Structure | | | | |
| Compound 145 | 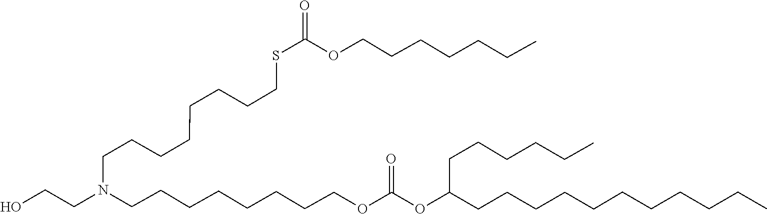 [SMILES: CCCCCCCCCCCC(CCCCCC)OC(=O)OCCCCCCCCN(CCO)CCCCCCCCSC(=O)OCCCCCCC] | | | | |
| Compound 146 | 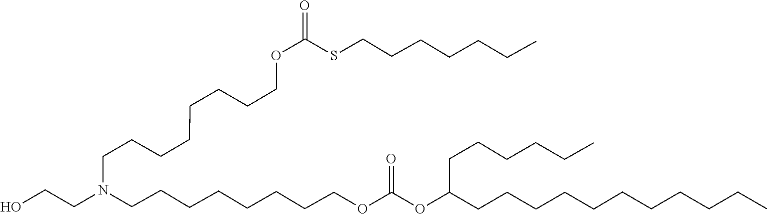 [SMILES: CCCCCCCCCCCC(CCCCCC)OC(=O)OCCCCCCCCN(CCO)CCCCCCCCOC(=O)SCCCCCCC] | | | | |
| | Cmpd | | Cmpd | | Intermediate |
| Target E | 33x = | Structure | 34x = | Structure | Cmpd 36x = |
| Cmpd 145 | 33a | 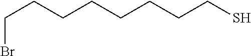 | 34a | 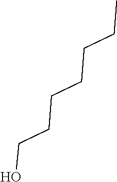 | 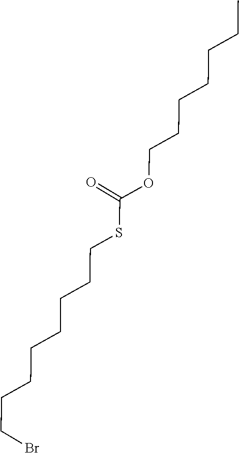 [SMILES: CCCCCCCOC(=O)SCCCCCCCCBr] |
| Cmpd 146 | 33b | 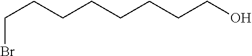 | 34b | 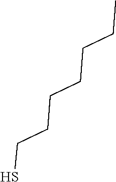 | 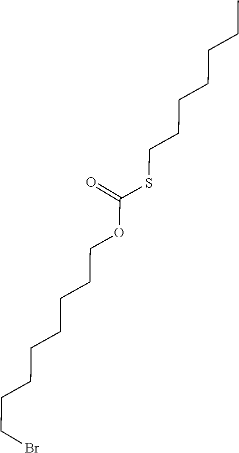 [SMILES: CCCCCCCSC(=O)OCCCCCCCCBr] |

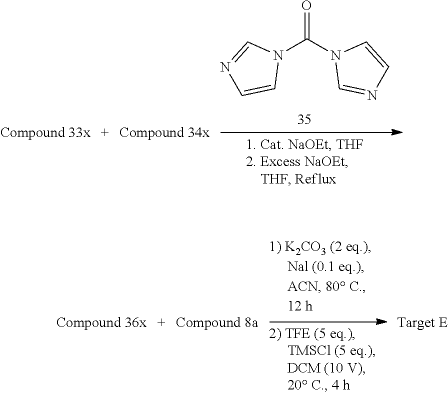 [SMILES: O=C(n1ccnc1)n1ccnc1]

To a solution of Compound 33x in THF is added Compound 34x and Compound 35. Catalytic sodium ethoxide is added with stirring at room temperature. Then, excess sodium ethoxide is added and the mixture is brought to reflux. TLC assesses the extent of the reaction. The reaction mixture is concentrated under vacuum, diluted with H₂O and extracted with DCM. The residue is purified by flash silica gel chromatography to give crude Compound 36x. The mixture is concentrated to remove acetonitrile, diluted with H₂O and extracted with DCM. The combined organic layers are dried over Na₂SO₄, filtered and concentrated under reduced pressure to give Compound 36x.

To a solution of Compound 36x in acetonitrile is added Compound 8a, NaI, and K2CO₃. The mixture is stirred at 80° C. The reaction mixture is diluted with ethyl acetate and filtered. The filtrate was concentrated under reduced pressure to give a residue. The residue is purified by flash silica gel chromatography to give crude Target E, which is purified by prep-HPLC and neutralized with saturated NaHCO₃. The mixture is concentrated to remove acetonitrile, diluted with H₂O and extracted with DCM. The combined organic layers are dried over Na₂SO₄, filtered and concentrated under reduced pressure to give Target E.

### General Procedure for the Synthesis of Compound 147 to Compound 148

| Compound | |
|---|---|
| Number | Structure |
| Compound 147 | 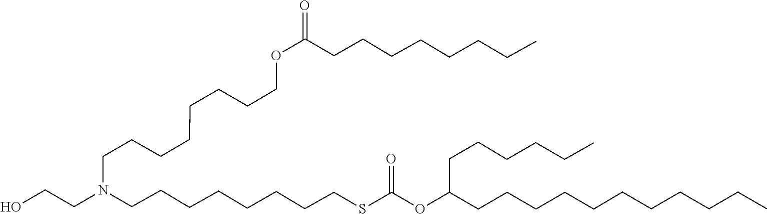 [SMILES: CCCCCCCCCCCC(CCCCCC)OC(=O)SCCCCCCCCN(CCO)CCCCCCCCOC(=O)CCCCCCCC] |
| Compound 148 | 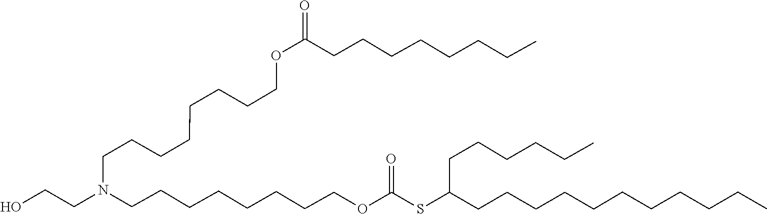 [SMILES: CCCCCCCCCCCC(CCCCCC)SC(=O)OCCCCCCCCN(CCO)CCCCCCCCOC(=O)CCCCCCCC] |

For a representative synthesis, please see the synthesis of Compound 100, vide-supra

| | Cmpd | | Cmpd | | Cmpd | |
|---|---|---|---|---|---|---|
| Target F | 37x = | Structure | 38x = | Structure | 39x = | Structure |
| Cmpd 147 | 37a | 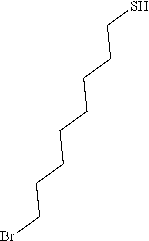 | 38a | 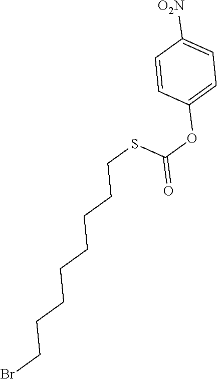 [SMILES: O=C(Oc1ccc([N+](=O)[O-])cc1)SCCCCCCCCBr] | 39a | 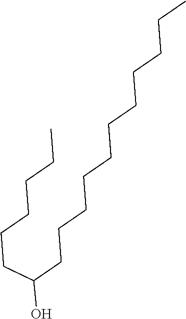 [SMILES: CCCCCCCCCCCC(O)CCCCCC] |
| Cmpd 148 | 37b | 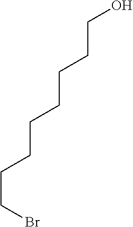 | 38b | 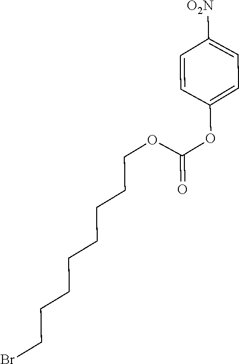 [SMILES: O=C(OCCCCCCCCBr)Oc1ccc([N+](=O)[O-])cc1] | 39b | 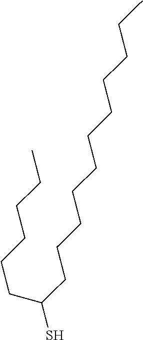 [SMILES: CCCCCCCCCCCC(S)CCCCCC] |

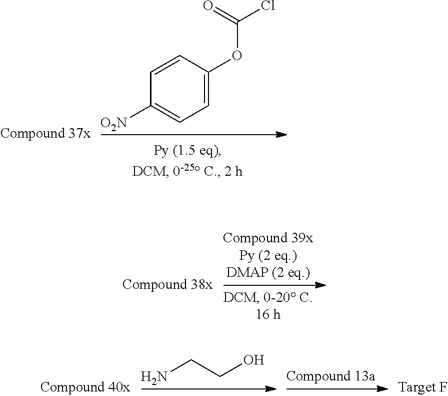

To a solution of Compound 37x in DCM is added Pyridine. Then, 4-nitrophenyl carbonochloridate in DCM is added dropwise at 0° C. The resulting mixture is stirred at 20° C. TLC assesses the extent of the reaction. The reaction mixture is diluted with H₂O and extracted with DCM. The combined organic layers are concentrated under reduced pressure to give Compound 38x.

To a solution of Compound 38x and Compound 39x in DCM was added Pyridine dropwise at 0° C. Then, DMAP was added at 0° C. The resulting mixture was stirred at 20° C. TLC assesses the extent of the reaction. The reaction mixture is concentrated under reduced pressure to give a residue that is purified by column chromatography to give Compound 40x as a colorless oil.

To a solution of Compound 40x in acetonitrile was added DIEA and 2-aminoethanol. The mixture was stirred at 70° C. TLC assesses the extent of the reaction. The reaction mixture is diluted with Ethyl acetate and washed with H₂O, dried over Na₂SO₄, filtered and concentrated under reduced pressure to give a residue. The residue is combined with Compound 13a in ethanol and stirred at 60° C. The reaction mixture was concentrated under reduced pressure to give a residue. The residue was diluted with H₂O and extracted with ethyl acetate. The combined organic layers were dried over Na₂SO₄, filtered and concentrated under reduced pressure to give a residue. The residue was purified by column chromatography to give Target F.

### General Procedure for the Synthesis of Compound 149

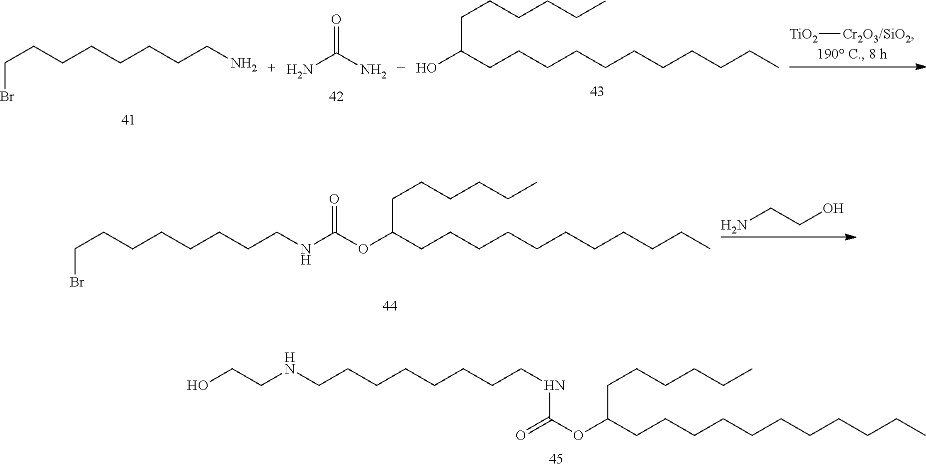

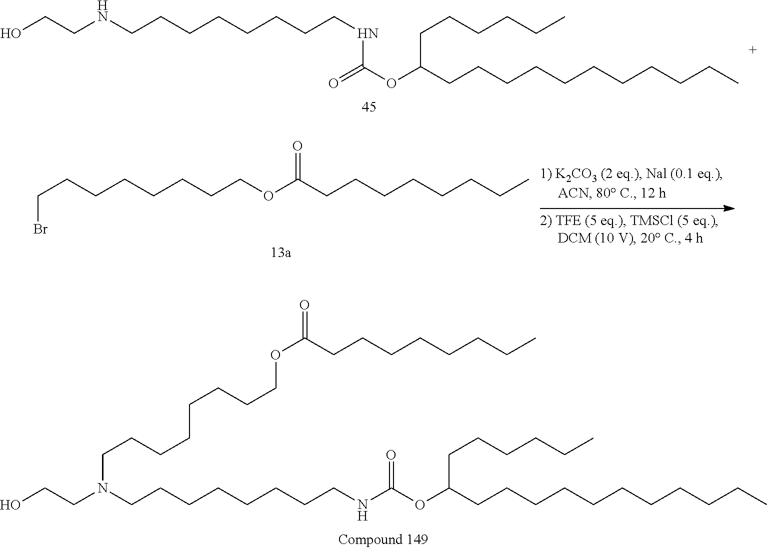 [SMILES: CCCCCCCCCCCC(CCCCCC)OC(=O)NCCCCCCCCNCCO ; CCCCCCCCC(=O)OCCCCCCCCBr ; CCCCCCCCCCCC(CCCCCC)OC(=O)NCCCCCCCCN(CCO)CCCCCCCCOC(=O)CCCCCCCC]

Compound 44 can be obtained by a coupling reaction of Compound 41, Compound 42, and Compound 43 with a catalytic amount of TiO₂—Cr₂O₃. Compound 45 can be obtained by stirring Compound 43 with DIEA and 2-aminoethanol at room temperature.

To a solution of Compound 45 in acetonitrile is added Compound 13a, NaI, and K2CO₃. The mixture is stirred at 80° C. The reaction mixture is diluted with ethyl acetate and filtered. The filtrate was concentrated under reduced pressure to give a residue. The residue is purified by flash silica gel chromatography to give crude Compound 149, which is purified by prep-HPLC and neutralized with saturated NaHCO₃. The mixture is concentrated to remove acetonitrile, diluted with H₂O and extracted with DCM. The combined organic layers are dried over Na₂SO₄, filtered and concentrated under reduced pressure to give Compound 149.

A solution of TMSCl in TFE is stirred at 20° C. Then Compound 149 is added dropwise at 20° C. The resulting mixture is stirred at 20° C. The mixture is concentrated to give Compound 149 as a light yellow oil.

### General Procedure for the Synthesis of Compound 150

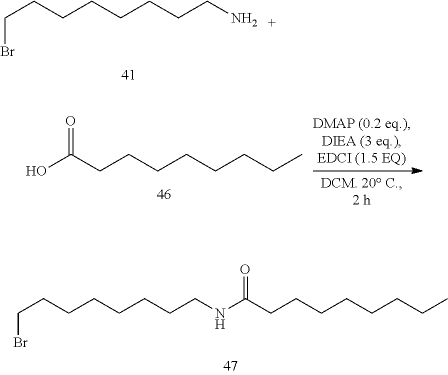 [SMILES: NCCCCCCCCBr ; CCCCCCCCC(=O)O ; CCCCCCCCC(=O)NCCCCCCCCBr]

To a solution of Compound 41 and Compound 46 in DCM was added Pyridine dropwise at 0° C. Then, DMAP was added at 0° C. The resulting mixture was stirred at 20° C. TLC assesses the extent of the reaction. The reaction mixture is concentrated under reduced pressure to give a residue that is purified by column chromatography to give Compound 47 as a colorless oil.

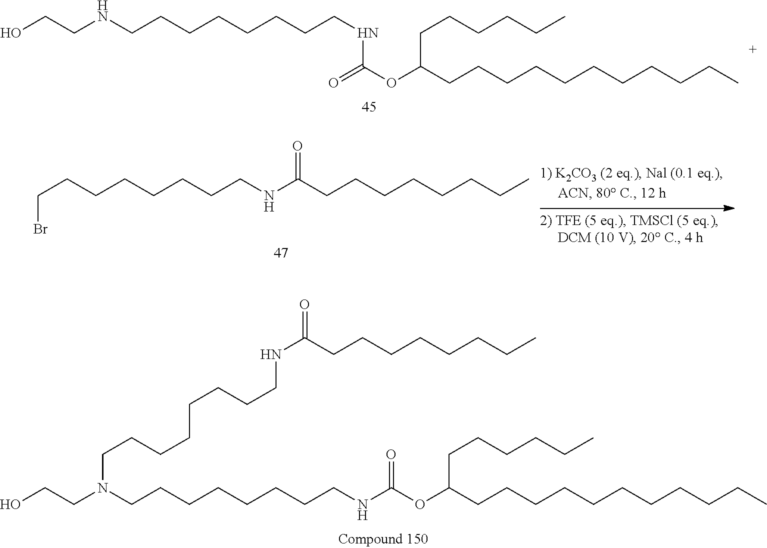 [SMILES: CCCCCCCCCCCC(CCCCCC)OC(=O)NCCCCCCCCNCCO ; CCCCCCCCC(=O)NCCCCCCCCBr ; CCCCCCCCCCCC(CCCCCC)OC(=O)NCCCCCCCCN(CCO)CCCCCCCCNC(=O)CCCCCCCC]

To a solution of Compound 45 in acetonitrile is added Compound 47, NaI, and K2CO₃. The mixture is stirred at 80° C. The reaction mixture is diluted with ethyl acetate and filtered. The filtrate was concentrated under reduced pressure to give a residue. The residue is purified by flash silica gel chromatography to give crude Compound 150, which is purified by prep-HPLC and neutralized with saturated NaHCO₃. The mixture is concentrated to remove acetonitrile, diluted with H₂O and extracted with DCM. The combined organic layers are dried over Na₂SO₄, filtered and concentrated under reduced pressure to give Compound 150.

A solution of TMSCl in TFE is stirred at 20° C. Then Compound 150 is added dropwise at 20° C. The resulting mixture is stirred at 20° C. The mixture is concentrated to give Compound 150 as a light yellow oil.

### Example 2. Lipid Screening by In Vivo Expression Evaluation in Lipid Nanoparticle Formulations

In this example, messenger RNA molecules encoding hEPO proteins were formulated in lipid nanoparticles for delivery in vivo. The lipid nanoparticle formulations comprised of a lipid composition of ionizable lipid:helper lipid:cholesterol:DMG-PEG2k at 50:10:38.5:1.5 mol %. The lipid mixture in ethanol was mixed with hEPO mRNA in RNA acidifying buffer (10 mM citrate, pH 4) at an ionizable-lipid-nitrogen-to-RNA-phosphate ratio (N:P) of 6 using a microfluidic device (Precision NanoSystems, Inc.) at a combined flow rate of 10 mL/min (7.5 mL/min for aqueous buffer, RNA and 2.5 mL/min for ethanol, lipid mix). The resulting particles were neutralized by buffer exchange into Dulbecco's phosphate buffer solution via PD-10 desalting column. The neutralized particles were concentrated using 100 kDa AMICON® Ultra centrifugal filters and sterile filtered using 0.2 um syringe filters. Samples were then characterized and diluted as needed (3 replicates).

LNPs were characterized by dynamic light scattering (DLS) measurement for measuring its hydrodynamic radius and polydispersity index (PDI). Encapsulation efficiency (EE %) and total RNA concentration were quantitated using a fluorescence based Ribogreen assay. The measurements for each of the LNPs are shown in Table 1 below.

TABLE 1
LNP Formulations for in vivo studies

| | lipid:DSPC: | | | | | | |
|---|---|---|---|---|---|---|---|
| | Cholesterol: | | | | | Zeta | |
| Ionizable | DMG-PEG2k | | N:P | Size | | potential | Encapsulation |
| Lipid | (mol %) | mRNA | ratio | (nm) | PDI | (mV) | Efficiency % |
| SM-102 | 50:10:38.5:1.5 | hEPO mRNA | 6 | 81.8 | 0.074 | −3.729 | 94.9 |
| Lipid 1(1) | 50:10:38.5:1.5 | hEPO mRNA | 6 | 65.2 | 0.04 | −10.3 | 97.6 |
| Lipid 1(2) | 50:10:38.5:1.5 | hEPO mRNA | 6 | 69.6 | 0.101 | −8.3 | 95.9 |
| Lipid 1(3) | 50:10:38.5:1.5 | hEPO mRNA | 6 | 68.9 | 0.108 | −7.1 | 98.6 |

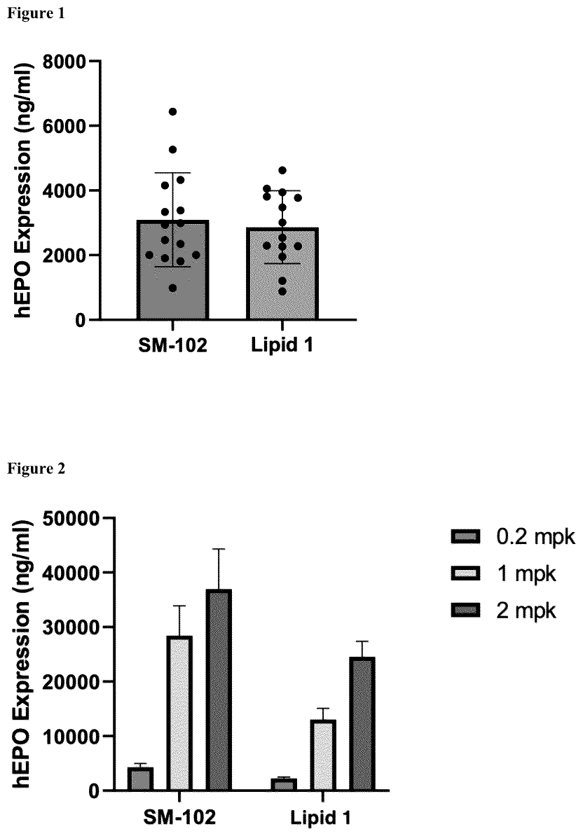

The in vivo studies were performed in C57BL/6 female mice at 6 to 8 weeks weighing in at approximately 20 g. The LNPs formulated with different ionizable lipids at 0.2 mg/kg of hEPO mRNA were administered by tail vein injection and animals were euthanized at 6 h post-administration for blood serum sample collection. The hEPO levels from the samples were analyzed and cross-compared by enzyme-linked immunoassay (ELISA) according to manufacturer's protocol. The hEPO expression levels for each of the LNPs was compared to a control LNP comprising SM102 (DC Chemicals Cat. No. DC52025) (FIG. 1)

### Example 3: In Vivo Blood Cytokine, AST and ALT Levels Evaluation of LNPs to Assess Tolerability in Mice

In this example, messenger RNA molecules encoding hEPO proteins were formulated in LNPs for delivery in vivo. The lipid nanoparticle formulations comprise a lipid composition of lipid 1:helper lipid:cholesterol:DMG-PEG2k at 50:10:38.5:1.5 mol %.

The lipid mixture in ethanol was mixed with hEPO mRNA in RNA acidifying buffer (10 mM citrate, pH 4) at a N-to-P ratio of 6 using a microfluidic device (Precision NanoSystems, Inc.) at a combined flow rate of 10 mL/min (7.5 mL/min for aqueous buffer, RNA and 2.5 mL/min for ethanol, lipid mix). The resulting particles were neutralized by buffer exchange into Dulbecco's phosphate buffer solution via PD-10 desalting column. The neutralized particles were concentrated using 100 kDa AMICON® Ultra centrifugal filters and sterile filtered using 0.2 um syringe filters.

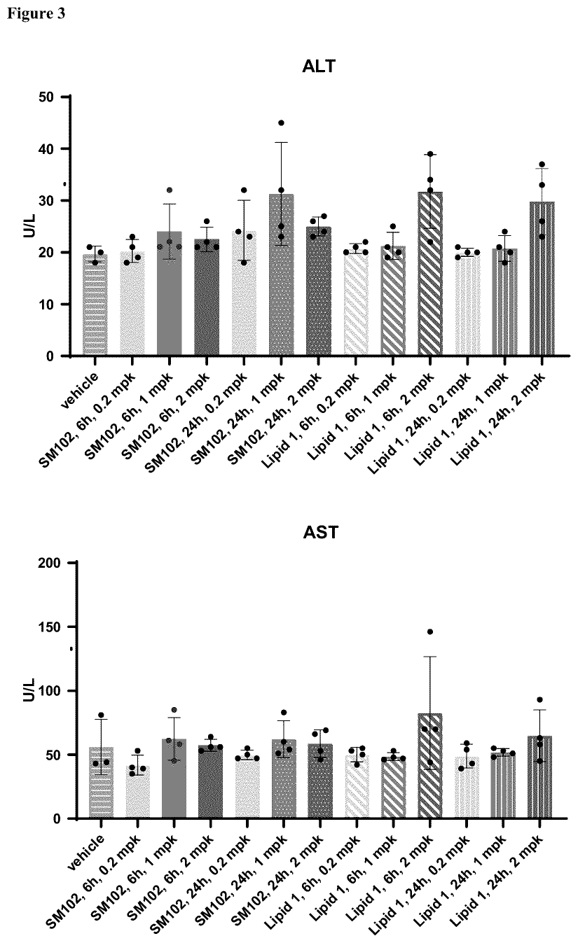

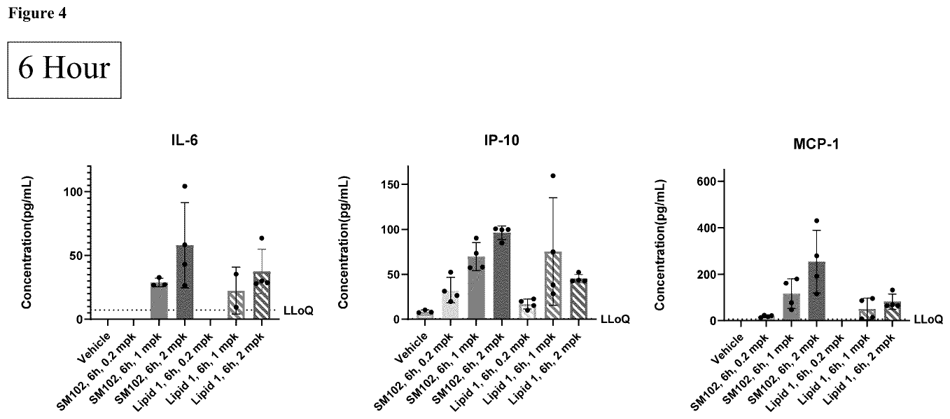

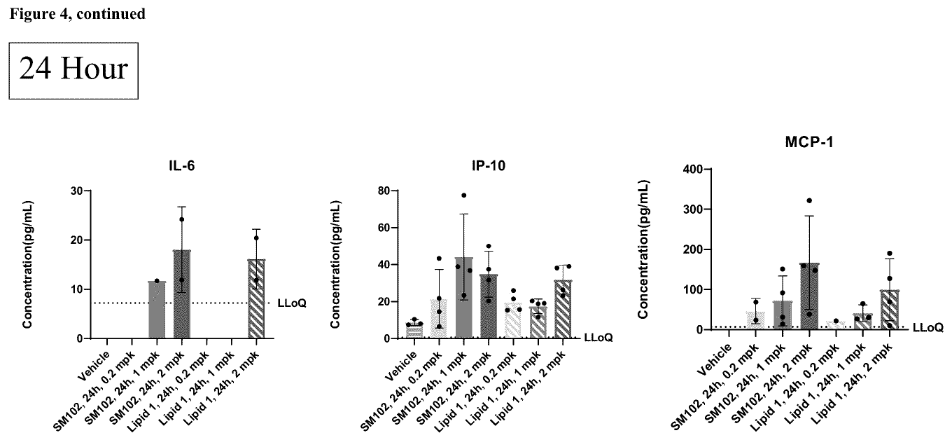

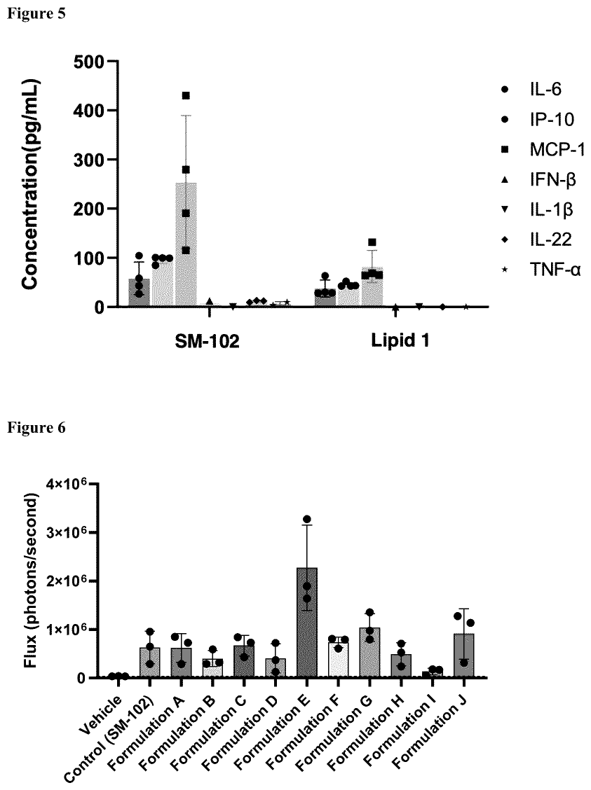

The in vivo studies were performed in C57BL/6 female mice at 6 to 8 weeks weighing in at approximately 20 g. The LNPs were formulated with different ionizable lipids at 0.2, 1 and 2 mg/kg of hEPO mRNA and were administered by tail vein injection. A subset of animals were euthanized at 6 h and another subset at 24 h post-administration for blood serum sample collection. The hEPO levels (FIG. 2) from the 6 hr samples were analyzed and cross-compared by enzyme-linked immunoassay (ELISA) according to manufacturer's protocol. ALT, AST and Cytokine analyses were conducted for 6 and 24 hr timepoints (FIGS. 3, 4, 5). The ALT, AST, and Cytokine levels for each of the LNPs were compared to a control LNP comprising SM102 (DC Chemicals Cat. No. DC52025).

For cytokine profiling of the tissue sample, the blood samples were processed and analyzed using a custom ProcartaPlex kit in a Luminex 200. The kit is customized to detect and quantitate cytokines such as IL-6, IP-10 (CXCL10), MCP-1 (CCL2), IFN-beta, IL-1 beta, IL-22, and TNF alpha in mouse serum. Serum levels of alanine aminotransferase (ALT) activity and aspartate aminotransferase (AST) activity are determined enzymatically using reagents designed for the AU680 Chemistry System (Beckman Coulter) and following the manufacturer's instructions.

### Example 4. Composition Screen by In Vivo Expression Evaluation in Lipid Nanoparticle Formulations

In this example, circular double stranded DNA (cdsDNA) molecules encoding fLuc and GFP proteins were formulated in lipid nanoparticles for delivery in vivo. The lipid nanoparticle formulations comprised of a lipid composition of ionizable lipid:helper lipid:cholesterol:DMG-PEG2k:DSPE-PEG2k-Tri-GalNAc:DiR at varying molar ratios shown in Table 2 below. The lipid mixture in ethanol was mixed with fLuc/GFP DNA in nucleic acid acidifying buffer (10 mM citrate, pH 4) at an ionizable-lipid-nitrogen-to-cdsDNA-phosphate ratio (N:P) of 6 using a microfluidic device (Precision NanoSystems, Inc.) at a combined flow rate of 10 mL/min (7.5 mL/min for aqueous buffer, DNA and 2.5 mL/min for ethanol, lipid mix). The resulting particles were neutralized by buffer exchange into Dulbecco's phosphate buffer solution via PD-10 desalting column. The neutralized particles were concentrated using 100 kDa AMICON® Ultra centrifugal filters and sterile filtered using 0.2 μm syringe filters. Samples were then characterized and diluted as needed.

LNPs were characterized by dynamic light scattering (DLS) measurement for measuring its hydrodynamic radius and polydispersity index (PDI). Encapsulation efficiency (EE %) and total RNA concentration were quantitated using a fluorescence based Ribogreen assay. The measurements for each of the LNPs are shown in Table 2 below. LNP zeta potential was measured via electrophoretic light scattering.

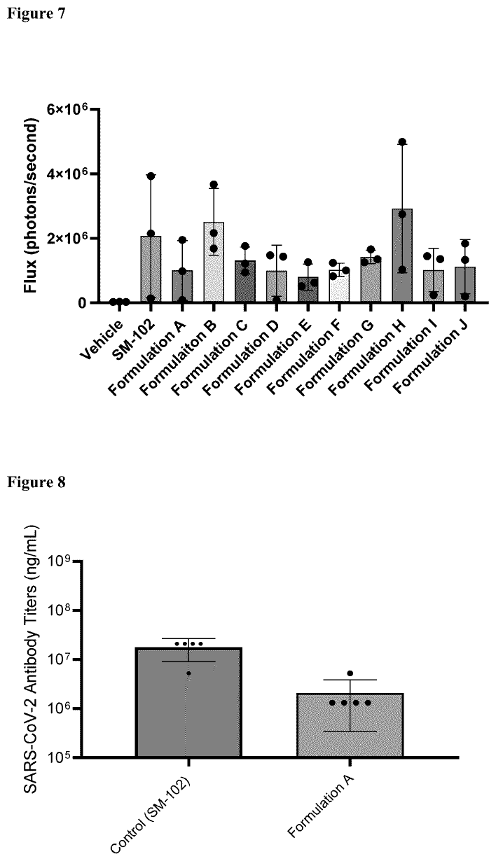

The in vivo studies were performed in C57BL/6 female mice at 6 to 8 weeks weighing in at approximately 20 g. The LNPs formulated with different ionizable lipids at 0.5 mg/kg of fLuc/GFP cdsDNA were administered by tail vein injection, and at 7 days mice were euthanized for ex vivo luminescence readout. The total flux of the liver and spleen of each mouse were measured via In Vivo Imaging System (IVIS), and luminescence of each formulation was compared to a control formulation comprising SM102 (DC Chemicals Cat. No. DC52025) (FIGS. 6 and 7).

TABLE 2
LNP Formulations for in vivo studies

| | | lipid:DSPC:Cholesterol: | | | | | Zeta | |
|---|---|---|---|---|---|---|---|---|
| | Ionizable | DMG-PEG2k:DSPE-PEG2k- | | N:P | Size | | potential | Encapsulation |
| Formulation | Lipid | GalNAc:DiR (mol %) | cdsDNA | ratio | (nm) | PDI | (mV) | Efficiency % |
| Control | SM-102 | 49.98:10:38.48:1.40:0.1:0.05 | fLuc/GFP cdsDNA | 6 | 75.0 | 0.066 | −1.6 | 98.2 |
| A | Lipid 1 | 51.22:2.69:44.26:1.29:0.5:0.05 | fLuc/GFP cdsDNA | 6 | 72.9 | 0.062 | −3.8 | 97.8 |
| B | Lipid 1 | 26.46:3.99:67.2:1.8:0.5:0.05 | fLuc/GFP cdsDNA | 6 | 92.9 | 0.225 | −3.5 | 97.2 |
| C | Lipid 1 | 39.54:0.9:58.41:0.6:0.5:0.05 | fLuc/GFP cdsDNA | 6 | 95.0 | 0.141 | −6.8 | 96.7 |
| D | Lipid 1 | 45.88:20.89:30.18:2.5:0.5:0.05 | fLuc/GFP cdsDNA | 6 | 63.5 | 0.085 | −10.2 | 95.9 |
| E | Lipid 1 | 35.75:29.86:33.05:0.8:0.5:0.05 | fLuc/GFP cdsDNA | 6 | 72.0 | 0.045 | −7.1 | 97.3 |
| F | Lipid 1 | 25.06:24.06:49.13:1.2:0.5:0.05 | fLuc/GFP cdsDNA | 6 | 68.2 | 0.043 | −5.4 | 97.3 |
| G | Lipid 1 | 49.93:9.99:38.04:1.5:0.5:0.05 | fLuc/GFP cdsDNA | 6 | 82.0 | 0.063 | −7.1 | 97.6 |
| H | Lipid 1 | 63.01:0.1:34.25:2.1:0.5:0.05 | fLuc/GFP cdsDNA | 6 | 105.5 | 0.038 | −12.5 | 90.9 |
| I | Lipid 1 | 31.65:10.28:55.22:2.3:0.5:0.05 | fLuc/GFP cdsDNA | 6 | 60.0 | 0.060 | −2.2 | 97.6 |
| J | Lipid 1 | 38.54:17.27:41.74:1.9:0.5:0.05 | fLuc/GFP cdsDNA | 6 | 65.5 | 0.093 | −2.5 | 97.7 |

### Example 5. Vaccine

In this example, messenger RNA molecules encoding SARS-CoV-2 spike protein were formulated in lipid nanoparticles for delivery in vivo and antibody titers were assayed at 28 days after administration. The lipid nanoparticle formulations comprised a lipid composition of ionizable lipid:helper lipid:cholesterol:DMG-PEG2k at 50:10:38.5:1.5 mol %. The lipid mixture in ethanol was mixed with SARS-CoV-2 mRNA in nucleic acid acidifying buffer (10 mM citrate, pH 4) at an ionizable-lipid-nitrogen-to-mRNA-phosphate ratio (N:P) of 6 using a microfluidic device (Precision NanoSystems, Inc.) at a combined flow rate of 10 mL/min (7.5 mL/min for aqueous buffer, RNA and 2.5 mL/min for ethanol, lipid mix). The resulting particles were neutralized by buffer exchange into 8% sucrose 20 mM Tris pH 7.5 solution via PD-10 desalting column. The neutralized particles were concentrated using 100 kDa AMICON® Ultra centrifugal filters and sterile filtered using 0.2 μm syringe filters. Samples were then characterized and diluted as needed.

Resulting LNPs were characterized by dynamic light scattering (DLS) measurement for measuring its hydrodynamic radius and polydispersity index (PDI). Encapsulation efficiency (EE %) and total RNA concentration were quantitated using a fluorescence based Ribogreen assay. The measurements for each of the LNPs are shown in Table 3 below. LNP zeta potential was measured via electrophoretic light scattering.

The in vivo studies were performed in BALB/c female mice at 6 to 8 weeks weighing in at approximately 20 g. The LNPs formulated with different ionizable lipids at 0.25 mg/kg of SARS-CoV-2 mRNA were administered intramuscularly on the right side of the mouse's quadricep, and blood samples were collected on day 14. On day 15, LNPs formulated with different ionizable lipids at 0.25 mg/kg of SARS-CoV-2 mRNA were administered intramuscularly on the right side of the mouse's quadricep. Blood was drawn on day 29 and tested for an antibody titer assay using an ELISA. Each experimental group was compared with SM102 (DC Chemicals Cat. No. DC52025) (FIG. 8).

TABLE 3
LNP Formulations for in vivo SARS-CoV-2 LNP

| | | | | | Zeta | |
|---|---|---|---|---|---|---|
| | Ionizable | | Size | | potential | Encapsulation |
| Formulation | Lipid | mRNA | (nm) | PDI | (mV) | Efficiency % |
| Control | SM-102 | SARS-CoV-2 | 88.4 | 0.127 | −4.1 | 97.8 |
| | | Spike mRNA | | | | |
| A | Lipid 1 | SARS-CoV-2 | 87 | 0.045 | −5.7 | 98.6 |
| | | Spike mRNA | | | | |

### Example 6. In Vivo Expression Evaluation of Head Group Modification on Lipids in Lipid Nanoparticle Formulations

In this example, messenger RNA molecules encoding hEPO proteins were formulated in LNPs for delivery in vivo. The lipid nanoparticle formulations comprised of a lipid composition of a lipid:helper lipid:cholesterol:DMG-PEG2k at 50:10:38.5:1.5 mol %. The lipid mixture in ethanol was mixed with hEPO mRNA in RNA acidifying buffer (10 mM citrate, pH 4) at a N-to-P ratio of 6 using a microfluidic device (Precision NanoSystems, Inc.) at a combined flow rate of 10 mL/min (7.5 mL/min for aqueous buffer, RNA and 2.5 mL/min for ethanol, lipid mix). The resulting particles were neutralized by buffer exchange into 20 mM HEPES (pH 7.8), 25 mM sodium chloride, 7.5% (w/v) sucrose solution via PD-10 desalting column. The neutralized particles were concentrated using 100 kDa AMICON® Ultra centrifugal filters and sterile filtered using 0.2 um syringe filters. LNPs were then stored at −80C until use. Prior to in vivo dosing, LNPs were thawed at room temperature and diluted 2-fold with 20 mM HEPES (pH 7.8), resulting in a final dosing buffer of 20 mM HEPES (pH 7.8), 25 mM sodium chloride, 7.5% (w/v) sucrose. Resulting LNPs were characterized by dynamic light scattering (DLS) measurement for measuring its hydrodynamic diameter and polydispersity index (PDI) and the Zeta potential was measured using a Zetasizer (Malvern). Encapsulation efficiency (EE %) and total RNA concentration were quantitated using a fluorescence based Ribogreen assay. The formulation and measurements for each of the LNPs are shown in Table 4 below.

TABLE 4
LNP formulations for in vivo studies

| | Lipid:DSPC: | | | | | Zeta | |
|---|---|---|---|---|---|---|---|
| Ionizable | Cholesterol:DMG- | | N:P | Size | | potential | Encapsulation |
| Lipid | PEG2k (mol %) | mRNA | ratio | (nm) | PDI | (mV) | Efficiency % |
| SM-102 | 50:10:38.5:1.5 | hEPO | 6 | 73.4 | 0.060 | −3.959 | 96.3 |
| Lipid 1 | 50:10:38.5:1.5 | hEPO | 6 | 84.1 | 0.103 | −2.525 | 94.6 |
| Compound 100 | 50:10:38.5:1.5 | hEPO | 6 | 88.2 | 0.085 | −4.814 | 93.0 |
| Compound 101 | 50:10:38.5:1.5 | hEPO | 6 | 96.4 | 0.079 | −5.203 | 91.7 |
| Compound 102 | 50:10:38.5:1.5 | hEPO | 6 | 128.9 | 0.178 | −6.371 | 76.4 |
| Compound 103 | 50:10:38.5:1.5 | hEPO | 6 | 74.6 | 0.038 | −4.452 | 95.9 |
| Compound 104 | 50:10:38.5:1.5 | hEPO | 6 | 91.3 | 0.020 | −7.369 | 95.2 |
| Compound 105 | 50:10:38.5:1.5 | hEPO | 6 | 105.3 | 0.075 | −2.246 | 84.7 |
| Compound 106 | 50:10:38.5:1.5 | hEPO | 6 | 71.4 | 0.263 | −11.09 | 93.2 |
| Compound 107 | 50:10:38.5:1.5 | hEPO | 6 | 68.9 | 0.073 | −9.455 | 85.1 |
| Compound 108 | 50:10:38.5:1.5 | hEPO | 6 | 71.4 | 0.215 | −6.128 | 96.0 |
| Compound 109 | 50:10:38.5:1.5 | hEPO | 6 | 64.1 | 0.088 | −9.403 | 95.7 |
| Compound 110 | 50:10:38.5:1.5 | hEPO | 6 | 58.2 | 0.071 | −9.102 | 96.7 |
| Compound 111 | 50:10:38.5:1.5 | hEPO | 6 | 77.8 | 0.227 | −4.14 | 97.5 |

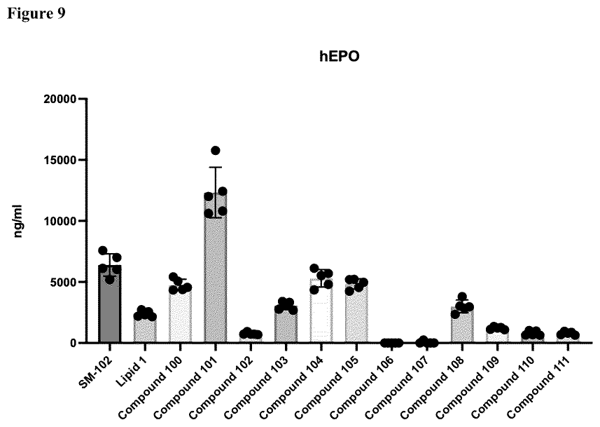

The in vivo studies were performed in C57BL/6 female mice at 6 to 8 weeks weighing in at approximately 20 g. The LNPs formulated with different ionizable lipids at 0.3 mg/kg of hEPO mRNA were administered by tail vein injection and animals were euthanized at 6 h post-administration for blood serum sample collection. The hEPO levels from the samples were analyzed and cross-compared by enzyme-linked immunoassay (ELISA) according to manufacturer's protocol. The hEPO expression levels for each of the LNPs shown in Table 4 were compared to a control LNP comprising SM-102 (DC Chemicals Cat. No. DC52025) (FIG. 9).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A lipidoid of formula (VIII), or a pharmaceutically acceptable salt thereof; 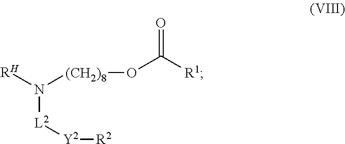
wherein:
R^{H} is hydroxyalkyl or (hydroxyalkyl)-O-alkylene-;
L² represents —(CH₂)ₘ;
m is an integer selected from 8-10;
Y² is *—O(C═O)O—, *—O(C═O)NF—, or *—(C═O)O—;
wherein the asterisk (*) indicates the point of attachment to L²;
R¹ is linear (C₆-C₁₀)alkyl, branched (C₁₀-C₂₀)alkyl, or linear (C₁₀-C₂₀)alkenyl; and
R² is branched (C₁₀-C₂₀)alkyl; wherein:
a branched (C₁₀-C₂₀)alkyl may comprise more than one branch point; and
a linear (C₁₀-C₂₀)alkenyl may comprise more than one carbon-carbon double bond.

2. The lipidoid of claim 1, wherein R^{H} is hydroxyalkyl.

3. The lipidoid of claim 1, wherein Y² is *—O(C═O)O—.

4. The lipidoid of claim 1, wherein L² is —(CH₂)₈—.

5. The lipidoid of claim 1, wherein R¹ is linear (C₆-C₁₀)alkyl.

6. A lipidoid of formula (I), or a pharmaceutically acceptable salt thereof; 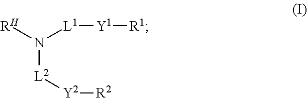
wherein:
R^{H} is hydroxyalkyl, (hydroxyalkyl)-O-alkylene-, or alkyl substituted by 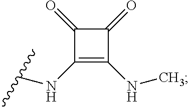 [SMILES: *Nc1c(NC)c(=O)c1=O]
L¹ and L² each represent —(CH₂)ₘ—;
m is independently for each occurrence an integer selected from 1-20;
Y¹ and Y² are each independently *—O(C═O)—, *—S(C═O)—, *—NH(C═O)—, *—NH(C═S)—, *—O(C═O)O—, *—O(C═S)O—, *—S(C═O)O—, *—O(C═O)S—, *—S(C═S)O—, *—NH(C═O)O—, *—O(C═O)NH—, *—(C═O)O—, *—(C═S)O—, *—O(P(O)OR²O)O—, 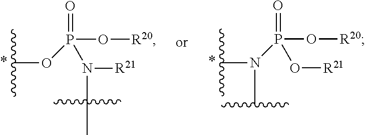
wherein the asterisk (*) indicates the point of attachment to L¹ or L²;
R²⁰ is independently for each occurrence an optionally substituted linear or branched (C₁-C₁₅)alkyl;
R²¹ is independently for each occurrence H, optionally substituted (C₁-C₅)alkyl, or optionally substituted (C₃-C₈)cycloalkyl; and
R¹ and R² are each independently optionally substituted linear (C₄-C₃₀)alkyl, branched (C₄-C₃₀)alkyl, linear (C₄-C₃₀)alkenyl, branched (C₄-C₃₀)alkenyl, linear (C₄-C₃₀)alkynyl, or branched (C₄-C₃₀)alkynyl; wherein:
a branched (C₄-C₃₀)alkyl, a branched (C₄-C₃₀)alkenyl, or a branched (C₄-C₃₀)alkynyl may comprise more than one branch point;
a linear or branched (C₄-C₃₀)alkenyl may comprise more than one carbon-carbon double bond;
a linear or branched (C₄-C₃₀)alkynyl may comprise more than one carbon-carbon triple bond; and
in any linear (C₄-C₃₀)alkyl, branched (C₄-C₃₀)alkyl, linear (C₄-C₃₀)alkenyl, branched (C₄-C₃₀)alkenyl, linear (C₄-C₃₀)alkynyl, or branched (C₄-C₃₀)alkynyl group, a —CH₂CH₂— moiety may be replaced by a —S—S-moiety.

7. The lipidoid of claim 6, wherein R^{H} is hydroxyalkyl.

8. The lipidoid of claim 7, wherein R^{H} is —CH₂CH₂OH.

9. The lipidoid of claim 6, wherein m is independently for each occurrence an integer selected from 4-20, preferably 8-10.

10. The lipidoid of claim 6, wherein L¹ and L² each represent —(CH₂)₈—.

11. The lipidoid of claim 6, wherein Y¹ and Y² are each independently *—O(C═O)—, *—S(C═O)—, *—NH(C═O)—, *—O(C═O)O—, *—S(C═O)O—, *—O(C═O)S—, *—NH(C═O)O—, *—O(C═O)NIH—, or *—(C═O)O—.

12. The lipidoid of claim 6, wherein Y¹ is *—O(C═O)—, *—S(C═O)—, *—NH(C═O)—, or *—(C═O)O—.

13. The lipidoid of claim 12, wherein Y¹ is *—O(C═O)—.

14. The lipidoid of claim 6, wherein Y² is *—O(C═O)O—, *—S(C═O)O—, *—O(C═O)S—, *—NH(C═O)O—, or *—O(C═O)NH—.

15. The lipidoid of claim 14, wherein Y² is *—O(C═O)O—.

16. The lipidoid of claim 6, wherein Y¹ and Y² are different.

17. The lipidoid of claim 6, wherein Y¹ and Y² are the same.

18. The lipidoid of claim 6, wherein R¹ is linear (C₄-C₃₀)alkyl or branched (C₄-C₃₀)alkyl.

19. The lipidoid of claim 18, wherein R¹ is linear (C₆-C₂₀)alkyl or branched (C₆-C₂₀)alkyl.

20. The lipidoid of claim 19, wherein R¹ is linear (C₆-C₁₄)alkyl or branched (C₆-C₁₄)alkyl.

21. The lipidoid of claim 20, wherein R¹ is linear (C₈)alkyl.

22. The lipidoid of claim 6, wherein R¹ is linear (C₈-C₂₀)alkenyl.

23. The lipidoid of claim 6, wherein R² is linear (C₄-C₃₀)alkyl or branched (C₄-C₃₀)alkyl.

24. The lipidoid of claim 23, wherein R² is branched (C₆-C₃₀)alkyl.

25. The lipidoid of claim 24, wherein R² is branched (C₁₆-C₂₆)alkyl.

26. The lipidoid of claim 25, wherein R² is branched (C₁₈-C₂₄)alkyl.

27. The lipidoid of claim 26, wherein R² is branched (C₁₈)alkyl.

28. The lipidoid of claim 6, wherein R¹ and R² are different.

29. The lipidoid of claim 6, selected from the following table:

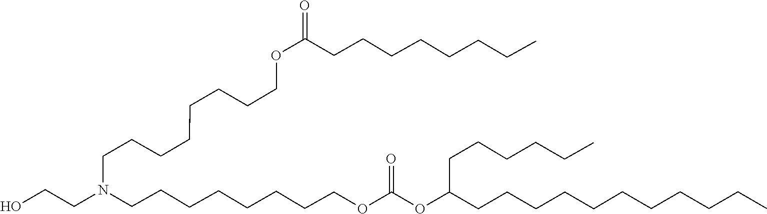 [SMILES: CCCCCCCCCCCC(CCCCCC)OC(=O)OCCCCCCCCN(CCO)CCCCCCCCOC(=O)CCCCCCCC]

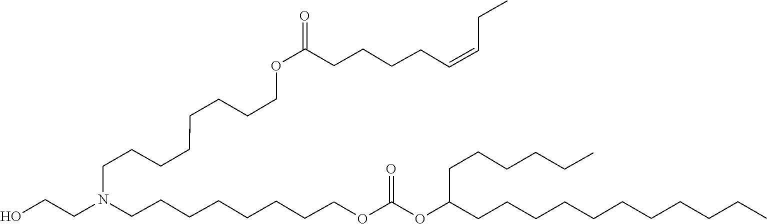 [SMILES: CC/C=C\CCCCC(=O)OCCCCCCCCN(CCO)CCCCCCCCOC(=O)OC(CCCCCC)CCCCCCCCCCC]

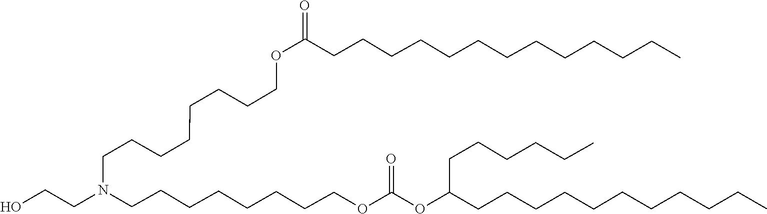 [SMILES: CCCCCCCCCCCCCC(=O)OCCCCCCCCN(CCO)CCCCCCCCOC(=O)OC(CCCCCC)CCCCCCCCCCC]

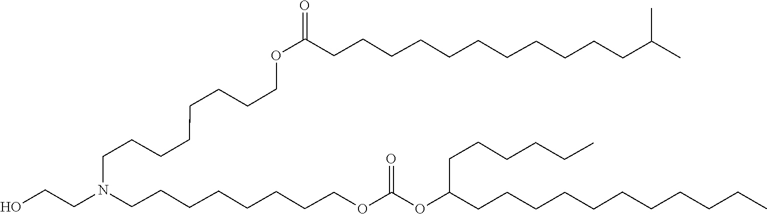 [SMILES: CCCCCCCCCCCC(CCCCCC)OC(=O)OCCCCCCCCN(CCO)CCCCCCCCOC(=O)CCCCCCCCCCCC(C)C]

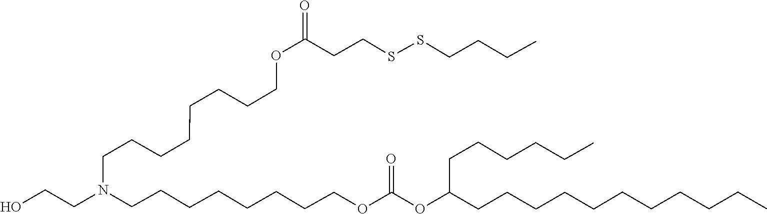 [SMILES: CCCCCCCCCCCC(CCCCCC)OC(=O)OCCCCCCCCN(CCO)CCCCCCCCOC(=O)CCSSCCCC]

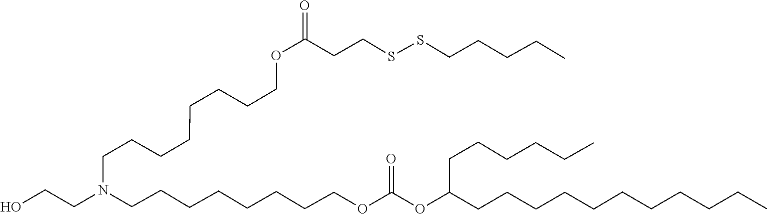 [SMILES: CCCCCCCCCCCC(CCCCCC)OC(=O)OCCCCCCCCN(CCO)CCCCCCCCOC(=O)CCSSCCCCC]

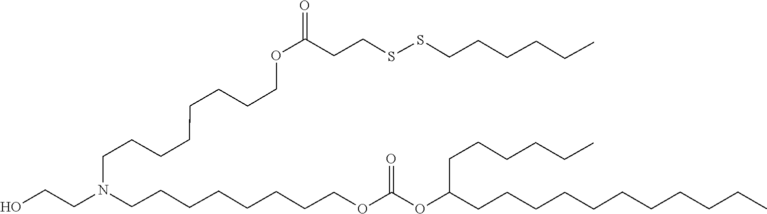 [SMILES: CCCCCCCCCCCC(CCCCCC)OC(=O)OCCCCCCCCN(CCO)CCCCCCCCOC(=O)CCSSCCCCCC]

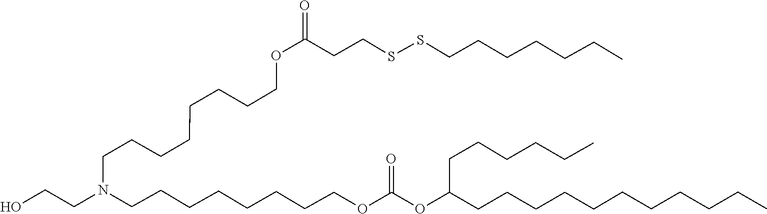 [SMILES: CCCCCCCCCCCC(CCCCCC)OC(=O)OCCCCCCCCN(CCO)CCCCCCCCOC(=O)CCSSCCCCCCC]

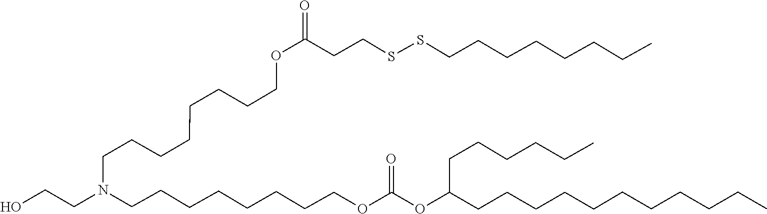 [SMILES: CCCCCCCCCCCC(CCCCCC)OC(=O)OCCCCCCCCN(CCO)CCCCCCCCOC(=O)CCSSCCCCCCCC]

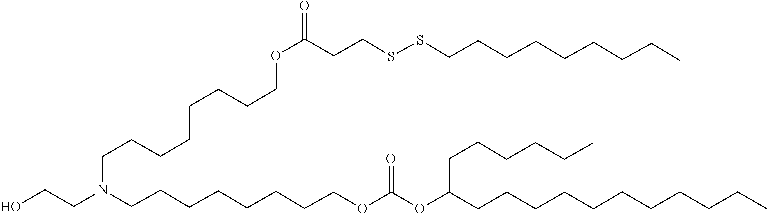 [SMILES: CCCCCCCCCCCC(CCCCCC)OC(=O)OCCCCCCCCN(CCO)CCCCCCCCOC(=O)CCSSCCCCCCCCC]

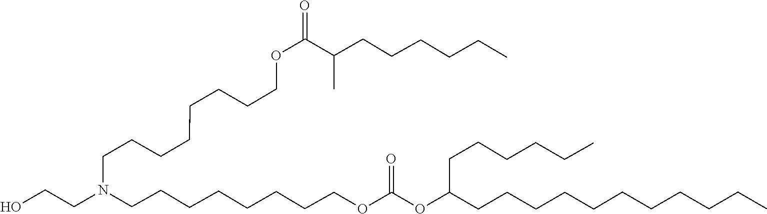 [SMILES: CCCCCCCCCCCC(CCCCCC)OC(=O)OCCCCCCCCN(CCO)CCCCCCCCOC(=O)C(C)CCCCCC]

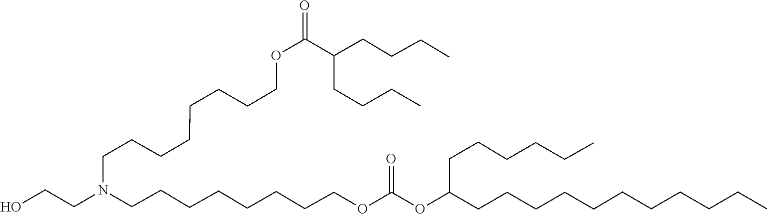 [SMILES: CCCCCCCCCCCC(CCCCCC)OC(=O)OCCCCCCCCN(CCO)CCCCCCCCOC(=O)C(CCCC)CCCC]

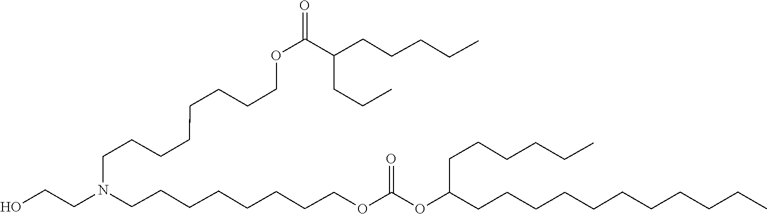 [SMILES: CCCCCCCCCCCC(CCCCCC)OC(=O)OCCCCCCCCN(CCO)CCCCCCCCOC(=O)C(CCC)CCCCC]

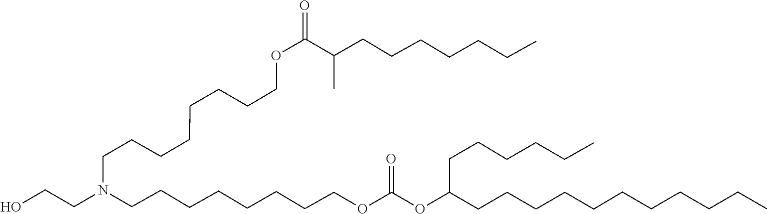 [SMILES: CCCCCCCCCCCC(CCCCCC)OC(=O)OCCCCCCCCN(CCO)CCCCCCCCOC(=O)C(C)CCCCCCC]

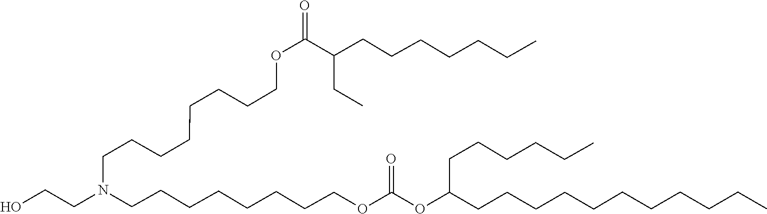 [SMILES: CCCCCCCCCCCC(CCCCCC)OC(=O)OCCCCCCCCN(CCO)CCCCCCCCOC(=O)C(CC)CCCCCCC]

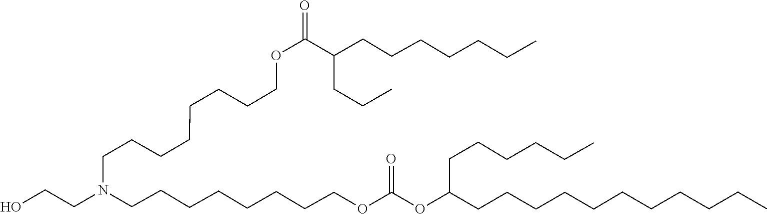 [SMILES: CCCCCCCCCCCC(CCCCCC)OC(=O)OCCCCCCCCN(CCO)CCCCCCCCOC(=O)C(CCC)CCCCCCC]

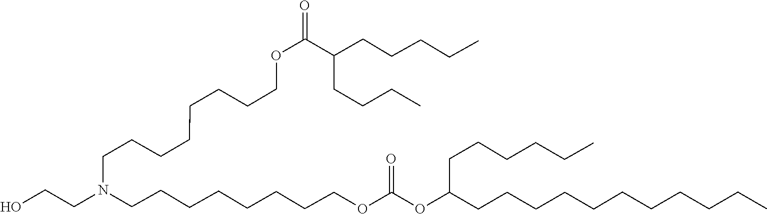 [SMILES: CCCCCCCCCCCC(CCCCCC)OC(=O)OCCCCCCCCN(CCO)CCCCCCCCOC(=O)C(CCCC)CCCCC]

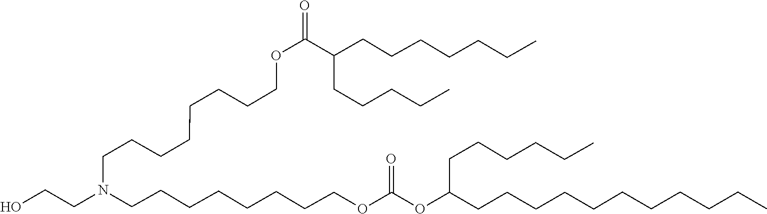 [SMILES: CCCCCCCCCCCC(CCCCCC)OC(=O)OCCCCCCCCN(CCO)CCCCCCCCOC(=O)C(CCCCC)CCCCCCC]

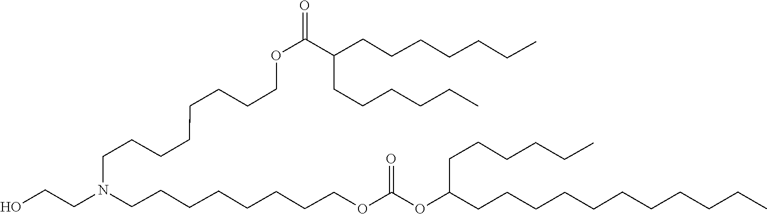 [SMILES: CCCCCCCCCCCC(CCCCCC)OC(=O)OCCCCCCCCN(CCO)CCCCCCCCOC(=O)C(CCCCCC)CCCCCCC]

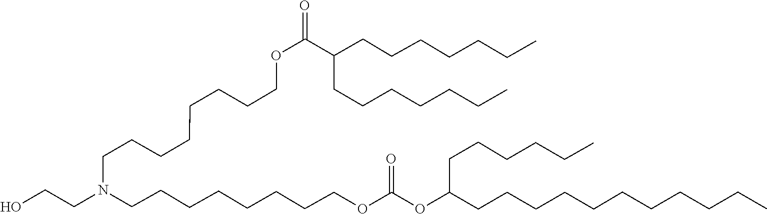 [SMILES: CCCCCCCCCCCC(CCCCCC)OC(=O)OCCCCCCCCN(CCO)CCCCCCCCOC(=O)C(CCCCCCC)CCCCCCC]

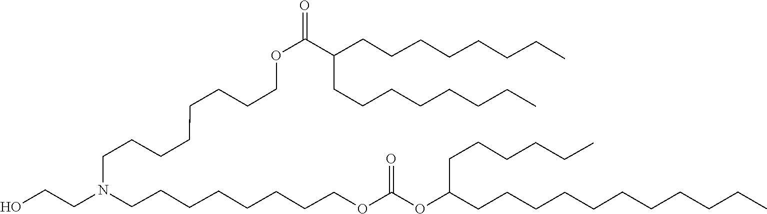 [SMILES: CCCCCCCCCCCC(CCCCCC)OC(=O)OCCCCCCCCN(CCO)CCCCCCCCOC(=O)C(CCCCCCCC)CCCCCCCC]

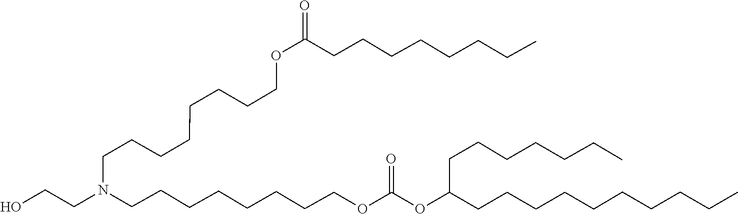 [SMILES: CCCCCCCCCCC(CCCCCCC)OC(=O)OCCCCCCCCN(CCO)CCCCCCCCOC(=O)CCCCCCCC]

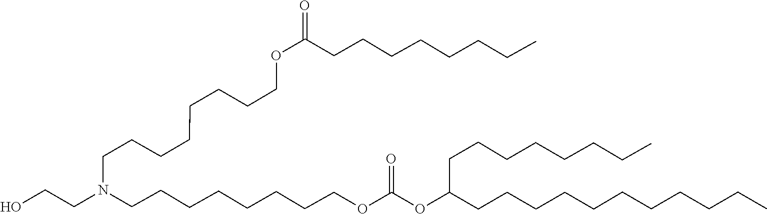 [SMILES: CCCCCCCCCCCC(CCCCCCCC)OC(=O)OCCCCCCCCN(CCO)CCCCCCCCOC(=O)CCCCCCCC]

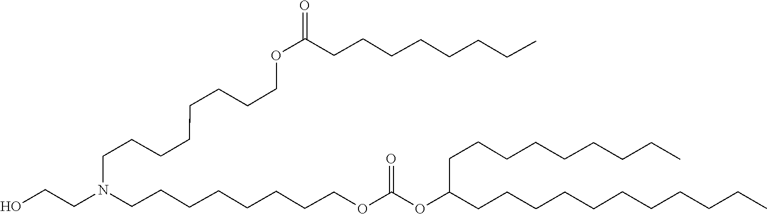 [SMILES: CCCCCCCCCCCC(CCCCCCCCC)OC(=O)OCCCCCCCCN(CCO)CCCCCCCCOC(=O)CCCCCCCC]

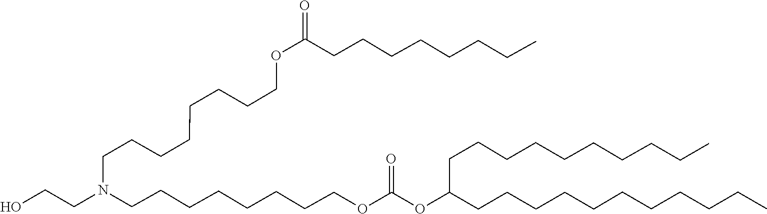 [SMILES: CCCCCCCCCCCC(CCCCCCCCCC)OC(=O)OCCCCCCCCN(CCO)CCCCCCCCOC(=O)CCCCCCCC]

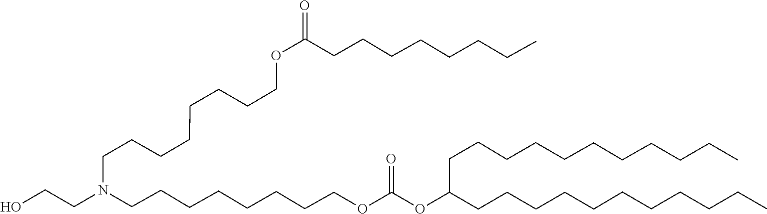 [SMILES: CCCCCCCCCCCC(CCCCCCCCCCC)OC(=O)OCCCCCCCCN(CCO)CCCCCCCCOC(=O)CCCCCCCC]

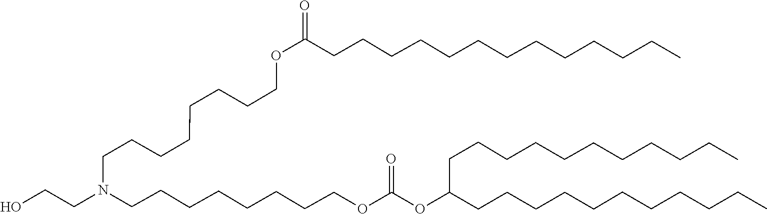 [SMILES: CCCCCCCCCCCCCC(=O)OCCCCCCCCN(CCO)CCCCCCCCOC(=O)OC(CCCCCCCCCCC)CCCCCCCCCCC]

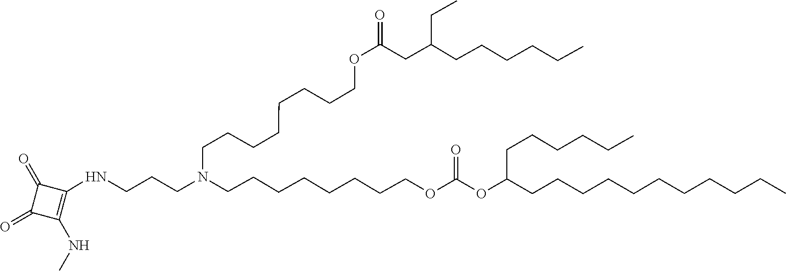 [SMILES: CCCCCCCCCCCC(CCCCCC)OC(=O)OCCCCCCCCN(CCCCCCCCOC(=O)CC(CC)CCCCCC)CCCNc1c(NC)c(=O)c1=O]

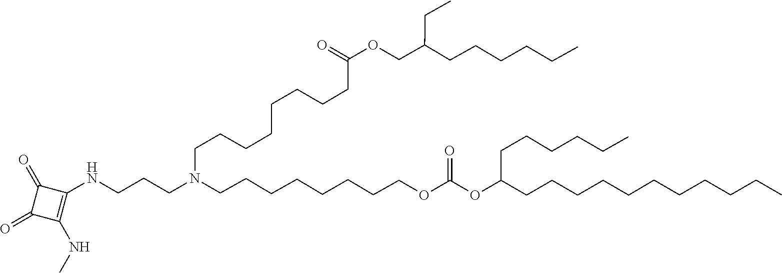 [SMILES: CCCCCCCCCCCC(CCCCCC)OC(=O)OCCCCCCCCN(CCCCCCCCC(=O)OCC(CC)CCCCCC)CCCNc1c(NC)c(=O)c1=O]

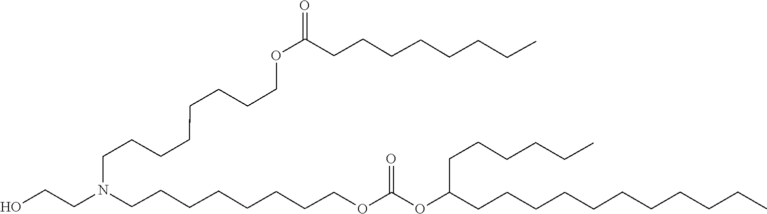 [SMILES: CCCCCCCCCCCC(CCCCCC)OC(=O)OCCCCCCCCN(CCO)CCCCCCCCOC(=O)CCCCCCCC]

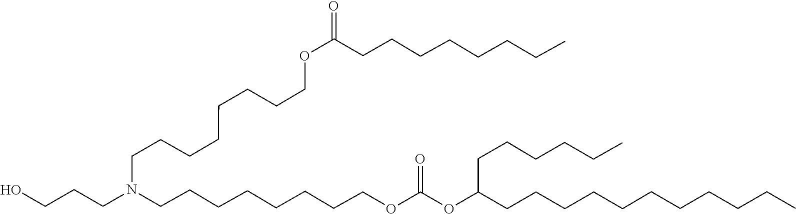 [SMILES: CCCCCCCCCCCC(CCCCCC)OC(=O)OCCCCCCCCN(CCCO)CCCCCCCCOC(=O)CCCCCCCC]

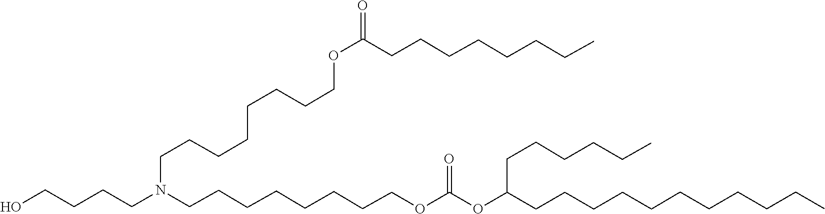 [SMILES: CCCCCCCCCCCC(CCCCCC)OC(=O)OCCCCCCCCN(CCCCO)CCCCCCCCOC(=O)CCCCCCCC]

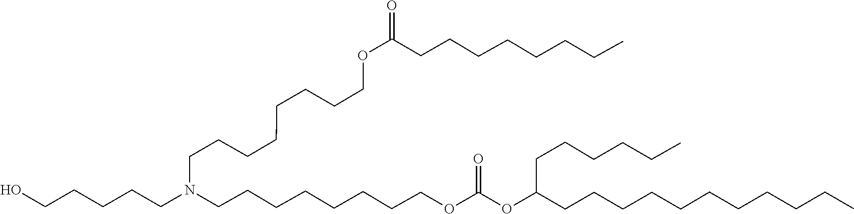 [SMILES: CCCCCCCCCCCC(CCCCCC)OC(=O)OCCCCCCCCN(CCCCCO)CCCCCCCCOC(=O)CCCCCCCC]

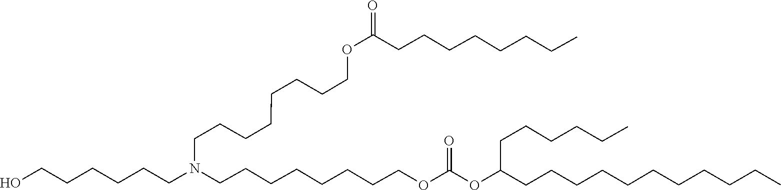 [SMILES: CCCCCCCCCCCC(CCCCCC)OC(=O)OCCCCCCCCN(CCCCCCO)CCCCCCCCOC(=O)CCCCCCCC]

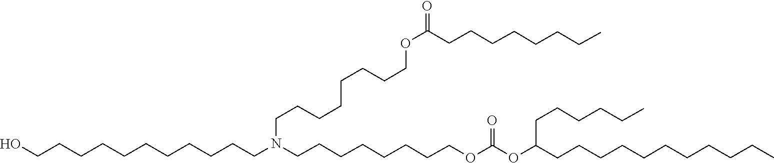 [SMILES: CCCCCCCCCCCC(CCCCCC)OC(=O)OCCCCCCCCN(CCCCCCCCCCCO)CCCCCCCCOC(=O)CCCCCCCC]

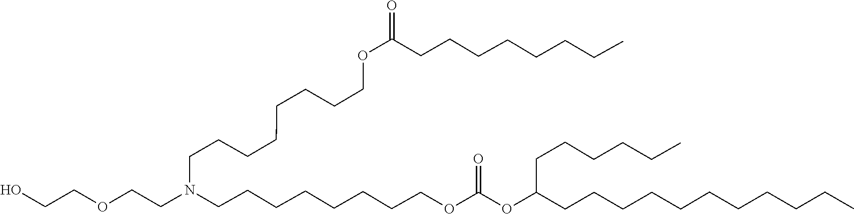 [SMILES: CCCCCCCCCCCC(CCCCCC)OC(=O)OCCCCCCCCN(CCCCCCCCOC(=O)CCCCCCCC)CCOCCO]

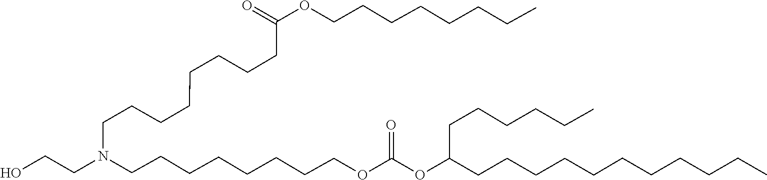 [SMILES: CCCCCCCCCCCC(CCCCCC)OC(=O)OCCCCCCCCN(CCO)CCCCCCCCC(=O)OCCCCCCCC]

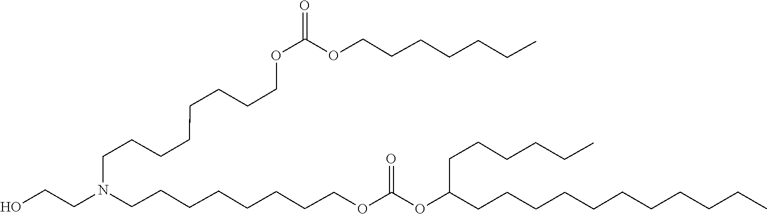 [SMILES: CCCCCCCCCCCC(CCCCCC)OC(=O)OCCCCCCCCN(CCO)CCCCCCCCOC(=O)OCCCCCCC]

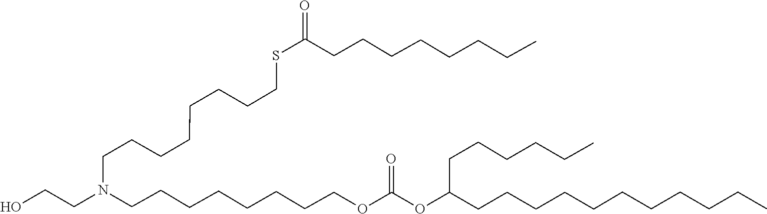 [SMILES: CCCCCCCCCCCC(CCCCCC)OC(=O)OCCCCCCCCN(CCO)CCCCCCCCSC(=O)CCCCCCCC]

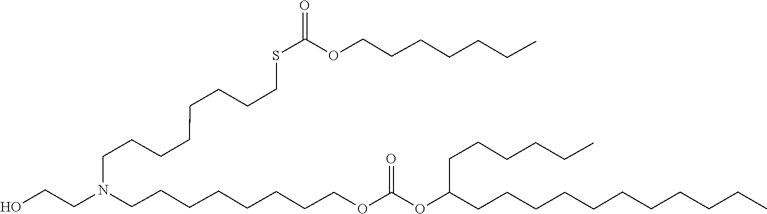 [SMILES: CCCCCCCCCCCC(CCCCCC)OC(=O)OCCCCCCCCN(CCO)CCCCCCCCSC(=O)OCCCCCCC]

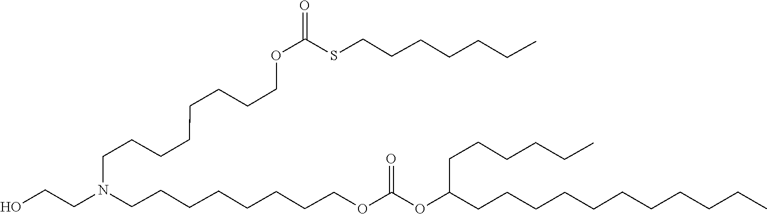 [SMILES: CCCCCCCCCCCC(CCCCCC)OC(=O)OCCCCCCCCN(CCO)CCCCCCCCOC(=O)SCCCCCCC]

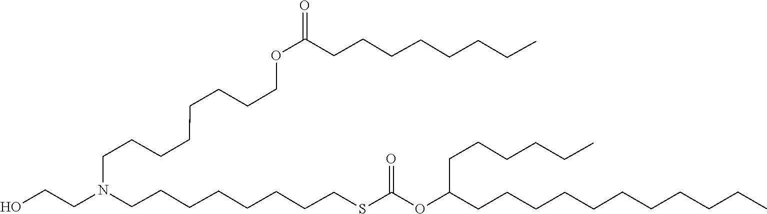 [SMILES: CCCCCCCCCCCC(CCCCCC)OC(=O)SCCCCCCCCN(CCO)CCCCCCCCOC(=O)CCCCCCCC]

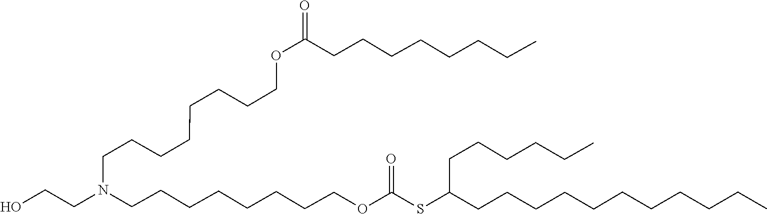 [SMILES: CCCCCCCCCCCC(CCCCCC)SC(=O)OCCCCCCCCN(CCO)CCCCCCCCOC(=O)CCCCCCCC]

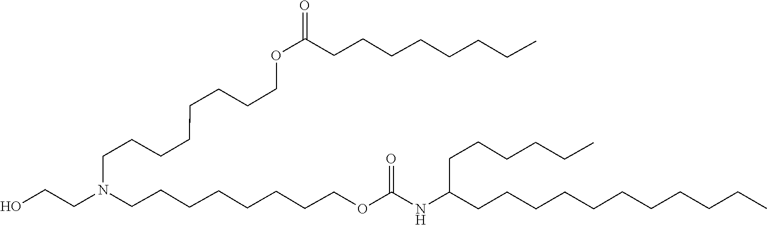 [SMILES: CCCCCCCCCCCC(CCCCCC)NC(=O)OCCCCCCCCN(CCO)CCCCCCCCOC(=O)CCCCCCCC]

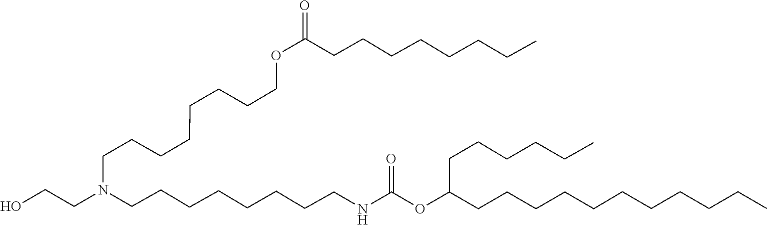 [SMILES: CCCCCCCCCCCC(CCCCCC)OC(=O)NCCCCCCCCN(CCO)CCCCCCCCOC(=O)CCCCCCCC]

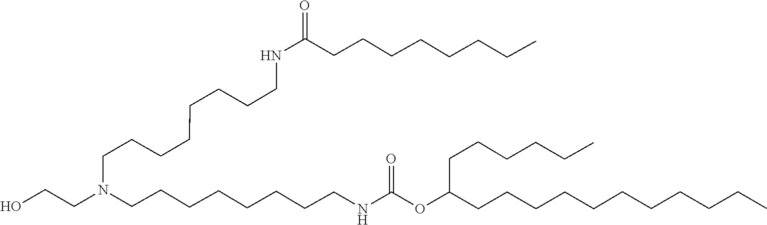 [SMILES: CCCCCCCCCCCC(CCCCCC)OC(=O)NCCCCCCCCN(CCO)CCCCCCCCNC(=O)CCCCCCCC]

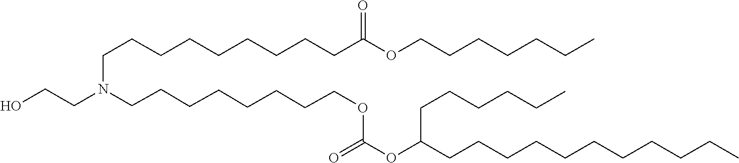 [SMILES: CCCCCCCCCCCC(CCCCCC)OC(=O)OCCCCCCCCN(CCO)CCCCCCCCCC(=O)OCCCCCCC]

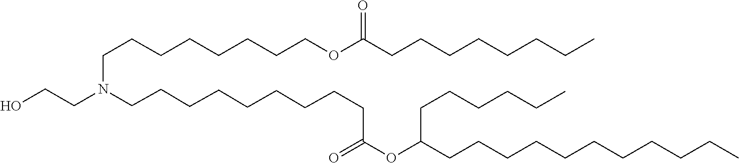 [SMILES: CCCCCCCCCCCC(CCCCCC)OC(=O)CCCCCCCCCN(CCO)CCCCCCCCOC(=O)CCCCCCCC]

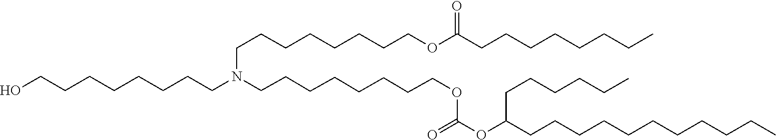 [SMILES: CCCCCCCCCCCC(CCCCCC)OC(=O)OCCCCCCCCN(CCCCCCCCO)CCCCCCCCOC(=O)CCCCCCCC]

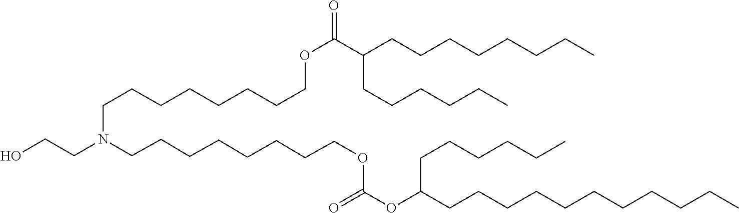 [SMILES: CCCCCCCCCCCC(CCCCCC)OC(=O)OCCCCCCCCN(CCO)CCCCCCCCOC(=O)C(CCCCCC)CCCCCCCC]

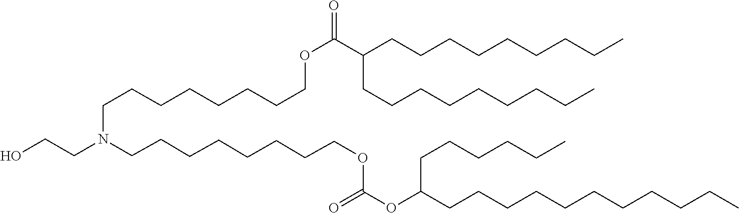 [SMILES: CCCCCCCCCCCC(CCCCCC)OC(=O)OCCCCCCCCN(CCO)CCCCCCCCOC(=O)C(CCCCCCCCC)CCCCCCCCC]

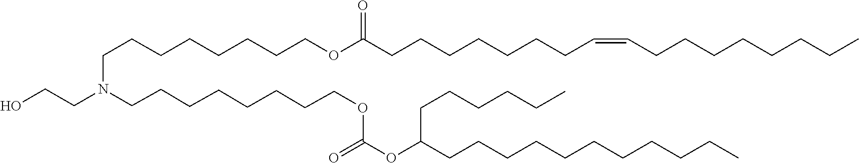 [SMILES: CCCCCCCC/C=C\CCCCCCCC(=O)OCCCCCCCCN(CCO)CCCCCCCCOC(=O)OC(CCCCCC)CCCCCCCCCCC]

30. A nanoparticle composition, comprising a plurality of a lipidoid of claim 1, or a pharmaceutically acceptable salt thereof.

31. The nanoparticle composition of claim 30, further comprising a PEGylated lipid, a sterol, a phospholipid, and/or a neutral lipid.

32. The nanoparticle composition of claim 30, further comprising a therapeutic agent.

33. The nanoparticle composition of claim 30, further comprising an antigen; wherein the antigen is a protein or a nucleic acid; the antigen is a protein; or the antigen is a nucleic acid.

34. The nanoparticle composition of claim 30, further comprising an mRNA molecule that encodes an antigen.

35. A nanoparticle composition comprising:
i. a nucleic acid;
ii. a lipidoid of claim 1, comprising from 32 mol % to 39 mol % of the total lipid component present in the nanoparticle;
iii. a phospholipid or derivative thereof comprising from 25 mol % to 30 mol % of the total lipid component present in the nanoparticle;
iv. a cholesterol or derivative thereof comprising from 31 mol % to 34 mol % of the total lipid component present in the nanoparticle; and
v. a conjugated lipid that inhibits aggregation of particles comprising from 0.7 mol % to 1.1 mol % of the total lipid component present in the nanoparticle.

36. A nanoparticle composition comprising:
i. a nucleic acid;
ii. a cationic lipid comprising 35.7 mol % of the total lipid component present in the nanoparticle;
iii. a phospholipid comprising 29.9 mol % of the total lipid component present in the nanoparticle;
iv. a cholesterol comprising 33.1 mol % of the total lipid component present in the nanoparticle;
v. a conjugated lipid comprising 0.8 mol % of the total lipid component present in the nanoparticle; and
vi. a conjugated lipid comprising GalNAc that comprises 0.5 mol % of the total lipid component present in the nanoparticle.

37. A pharmaceutical composition, comprising a nanoparticle composition of claim 30, and a pharmaceutically acceptable carrier.

38. A method of delivering a therapeutic agent, comprising administering to a subject in need thereof an effective amount of the nanoparticle composition of claim 32.

39. A method of vaccination, comprising administering to a subject in need thereof an effective amount of the nanoparticle composition of claim 34.
